(19) United States
(12) Patent Application Publication
(10) Pub. No.: US 2025/0250585 A1
(43) Pub. Date: Aug. 7, 2025

(54) CAPSIDS FOR PLAKOPHILLIN-2 GENE THERAPY

(71) Applicant: Tenaya Therapeutics, Inc., South San Francisco, CA (US)

(72) Inventors: Zhihong Jane YANG, Menlo Park, CA (US); Ze Cheng, South San Francisco, CA (US)

(21) Appl. No.: 18/855,606

(22) PCT Filed: Apr. 10, 2023
(86) PCT No.: PCT/US2023/018092

§ 371 Date: Oct. 9, 2024

Related U.S. Application Data
(60) Provisional application No. 63/329,787, filed on Apr. 11, 2022.

(51) Int. Cl. C12N 15/86; A61K 38/17; A61K 48/00; A61P 9/00; C07K 14/47

(52) U.S. Cl. CPC C12N 15/86; A61K 38/1709; A61K 48/0058; A61P 9/00; C07K 14/47; C12N 2750/14122; C12N 2750/14143; C12N 2830/008; C12N 2830/48; C12N 2830/50

(57) ABSTRACT

Provided herein are capsids for plakophilin-2 (PKP2) gene therapy, including recombinant adeno-associated virus (rAAV) virions with an engineered capsid protein for treating heart diseases such as arrhythmogenic right ventricular cardiomyopathy (ARVC) or arrhythmogenic cardiomyopathy (ACM). In particular, the disclosure provides AAV9 virions encoding PKP2 with engineered AAV9 capsid, AAV5/9 chimeric capsid, or combinatory capsid that achieves increased transduction efficiency in heart, increased heart-to-liver ratio, and/or other desirable properties.

### CROSS-REFERENCE

This application claims the benefit of U.S. Provisional Application No. 63/329,787, filed Apr. 11, 2023, which is incorporated herein by reference in its entirety.

### BACKGROUND

Arrhythmogenic right ventricular cardiomyopathy (ARVC) or arrhythmogenic cardiomyopathy (ACM) is an inherited cardiac disease found in 1/2000 to 1=5000 people. ARVC is characterized by fibrofatty tissue replacement in the myocardium, myocardial atrophy, predominant right ventricular dilation, ventricular arrhythmias, and sudden cardiac death (Wang et al., 2018). The disease is difficult to diagnose by conventional imaging and ECG particularly at its early stage due to its subclinical presentations. At the late stage, the disease progresses to more overt manifestations such as ventricular arrhythmias and morphological abnormalities in the ventricle. Sudden cardiac arrest in the young and athletes is found to be associated with ARVC and exercise-related cardiac wall stress. So far, there is no effective treatment of ARVC (Wang et al., 2018).

Adeno-associated virus (AAV) holds promise for gene therapy and other biomedical applications. In particular. AAV can be used to deliver gene products to various tissues and cells, both in vitro and in vivo. The capsid proteins of AAV largely determine the immunogenicity and tropism of AAV vectors.

For cardiac tissues, AAV subtype 9 (AAV9) is a preferred AAV vector due to its ability to transduce the heart following systemic delivery. While AAV9 can achieve moderate transduction of the heart, the majority of vector traffics to the liver. Moreover, in order to achieve therapeutic levels of transduction in the heart, relatively high systemic doses are required, potentially leading to systemic inflammation and in turn, toxicity.

There is a need for developing an Adeno-associated virus with engineered capsid protein that achieves improved cardiac tropism, and optionally improved selectivity of cardiac tissues over liver. The present disclosure provides variants of the AAV9 capsid and/or chimeric AAV5/AAV9 capsid that form rAAV virions capable of transducing cardiac tissues and/or cell types for more efficiently and/or with more selectivity than rAAV virions comprising wild-type AAV9 capsid proteins, which can be used for safe and efficacious cardiac gene therapy.

### SUMMARY

Provided herein are recombinant adeno-associated viruses (rAAV) comprising any plakophilin-2 (PKP2) expression cassette provided herein and encoding any rAAV capsid protein provided herein.

Also provided herein are method of treating a heart disease or disorder in an individual in need thereof comprising administering the rAAV of the disclosure to the individual.

Further provided herein are rAAV of the disclosure for use in treating a heart disease or disorder in an individual.

Additionally provided herein are compositions comprising the rAAV of the disclosure and a pharmaceutically acceptable buffer.

In one aspect, provided herein are recombinant adeno-associated virus (rAAV) virions, comprising a capsid protein and a plakophilin-2 (PKP2) expression cassette, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1: an amino acid insertion at position 584, or between positions 583 and 584, comprising one or more of an asparagine (N), a threonine (T), a tyrosine (Y), phenylalanine (F), and an alanine (A); an amino acid insertion at position 585, or between positions 584 and 585, comprising one or more of a histidine (H) and a methionine (M); an amino acid insertion at position 586, or between positions 585 and 586, comprising one or more of a histidine (H), a tyrosine (Y), a valine (V), a threonine (T), an alanine (A), an isoleucine (I), a tryptophan (W), a methionine (M), and a leucine (L); an amino acid insertion at position 587, or between positions 586 and 587, comprising one or more of an isoleucine (1) and a proline (P); an amino acid insertion at position 588, or between positions 587 and 588, comprising one or more of an isoleucine (I), a threonine (T), and a proline (P); an amino acid insertion at position 589, or between positions 588 and 589, comprising one or more of a glycine (G) and a glutamine (Q); one or more amino acid substitutions selected from the group consisting of N452K, N452A, N452V, N452I, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y. H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D. In some embodiments, the capsid protein comprises one, two, three, four or more substitutions or insertions in the VR-VIII site. In some embodiments, the capsid protein comprises, relative to reference SEQ ID NO:1, one, two, three, four or more substitutions or insertions at positions from 584 to 590 in the VR-VIII site, or one, two, three, four or more substitutions or insertions at positions from 585 to 590 in the VR-VIII site. In some embodiments, the capsid protein comprises, relative to reference sequence SEQ ID NO: 1: (i) one or more amino acid substitutions selected from the group consisting of T582D, T582E, N583V, H584Q, S586K, A587P, A587S, Q588G, Q588M, A589S, A591I, GS94Q, and G594D; (ii) one or more amino acid substitutions selected from the group consisting of T582L, T582A, T582F, T582R. T582P, H584R, H584K, H584V, H584Y, H584M, H584Q, H584W, H584E, H584D, Q585T, Q585N, Q585M, Q585E, Q585V, Q585H, S586T, S586O. S586Q, S586L S586L, S586F, S586D, S586R, S586M, A587F, A587I, A587H, A587M, A587N, A587W, Q588Y, Q588S, Q588T, and Q588R; (iii) one or more amino acid substitutions selected from the group consisting of Q585C, Q585S, S586I, A587V and A587O; or (iv) one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589L, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L. In some embodiments, the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence. In some embodiments, the capsid protein comprises, relative to reference sequence SEQ ID NO: 1, one or more amino acid substitutions selected from the group consisting of N452K, N452A, N452V, N452I, G453A, G453N, S454T, S454D, C455N, Q456L, Q456K, N457L, N457V, Q458I, and Q458I. In some embodiments, the capsid protein comprises, relative to reference sequence SEQ ID NO: 1, at position 452 an amino acid selected from the group consisting of: K and N. In some embodiments, the capsid protein comprises, relative to reference sequence SEQ ID NO: 1, an amino acid substitution N452K. In some embodiments, the capsid protein comprises, relative to reference sequence SEQ ID NO: 1: at position 584 an amino acid selected from the group consisting of: R and H; at position 585 an amino acid selected from the group consisting of: N, M, C, E, G, S, V, A, T, H, L and Q; at position 586 an amino acid selected from the group consisting of: M, D, N, G, A, T, R, I and S; at position 587 an amino acid selected from the group consisting of: T, N, V, L, I, S, R, P and A; at position 588 an amino acid selected from the group consisting of: Y, T, S, I, V, F, L, R, N, D, G and Q; at position 589 an amino acid selected from the group consisting of: L, I, R, S, G, N, T, V, Q, F, E, Y and A; and/or at position 590 an amino acid selected from the group consisting of: G, R, S, I, H, N, Y, L, M and Q. In some embodiments, the capsid protein comprises, relative to reference sequence SEQ ID NO: 1: at position 452 an amino acid selected from the group consisting of: K and N; at position 584 an amino acid selected from the group consisting of: R and R; at position 585 an amino acid selected from the group consisting of: N, M, C, E, G, S, V, A, T, H, L and Q; at position 586 an amino acid selected from the group consisting of: M, D, N, G, A, T, R, I and S; at position 587 an amino acid selected from the group consisting of: T, N, V, L, I, S, R, P and A; at position 588 an amino acid selected from the group consisting of: Y, T, S, I, V, F, L, R, N, D, G and Q; at position 589 an amino acid selected from the group consisting of: L, I, R, S, G, N, T, V, Q, F, E, Y and A; and at position 590 an amino acid selected from the group consisting of: G, R, S, I, H, N, Y, L, M and Q. In some embodiments, the capsid protein comprises, relative to reference sequence SEQ ID NO: 1: at position 584 amino acid R; at position 585 an amino acid selected from the group consisting of: N, M, C, E, G, S, V, A, T, H and, L; at position 586 an amino acid selected from the group consisting of: M, D, N, G, A, T, R, and I; at position 587 an amino acid selected from the group consisting of: T, N, V, L, I, S, R, and P: at position 588 an amino acid selected from the group consisting of: Y, T, S, I, V, F, L, R, N, D, and G; at position 589 an amino acid selected from the group consisting of: L, I, R, S, G, N, T, V, Q, F, E, and Y; and/or at position 590 an amino acid selected from the group consisting of: G, R, S, I, H, N, Y, L, and M. In some embodiments, the capsid protein comprises, relative to reference sequence SEQ ID NO: 1, at least two, three, four, five, six, seven or all eight of any of the following: (i) at position 452 amino acid K; (ii) at position 584 amino acid R; (iii) at position 585 an amino acid selected from the group consisting of: N, M, C, E, G, S, V, A, T, H, and L; (iv) at position 586 an amino acid selected from the group consisting of: M, D, N, G, A, T, R, and I; (v) at position 587 an amino acid selected from the group consisting of: T, N, V, L, I, S, R, and P; (vi) at position 588 an amino acid selected from the group consisting of: Y, T, S, I, V, F, L, R, N, D, and G; (vii) at position 589 an amino acid selected from the group consisting of: L, I, R, S, G, N, T, V, Q, F, E, and Y; and (viii) at position 590 an amino acid selected from the group consisting of: G, R, S, I, H, N, Y, L, and M. In some embodiments, the capsid protein comprises, relative to reference sequence SEQ ID NO: 1: at position 585 an amino acid selected from the group consisting of: E, N, G, M, C, V, T and Q; at position 586 an amino acid selected from the group consisting of: N, T, M, G, D, and S; at position 587 an amino acid selected from the group consisting of: T, L, I, K, S, N, V and A; at position 588 an amino acid selected from the group consisting of: V, F, Y, L, T, S, I, R and Q; at position 589 an amino acid selected from the group consisting of: S, N, L, T, I, R and A; and/or at position 590 an amino acid selected from the group consisting of: I, S, G, H, R and Q. In some embodiments, the capsid protein comprises, relative to reference sequence SEQ ID NO: 1: at position 452 an amino acid selected from the group consisting of: K and N; at position 585 an amino acid selected from the group consisting of: E, N, G, M, C, V, T and Q; at position 586 an amino acid selected from the group consisting of: N, T, M, G, D, and S; at position 587 an amino acid selected from the group consisting of: T, L, I, K, S, N, V and A; at position 588 an amino acid selected from the group consisting of: V, F, Y, L, T, S, I, R and Q; at position 589 an amino acid selected from the group consisting of: S, N, L, T, I, R and A; and at position 590 an amino acid selected from the group consisting of: I, S, G, H, R and Q. In some embodiments, the capsid protein comprises, relative to reference sequence SEQ ID NO: 1: at position $85 an amino acid selected from the group consisting of: E, N, G, M, C, V and T; at position 586 an amino acid selected from the group consisting of: N, T, M, G, and D; at position 587 an amino acid selected from the group consisting of: T, L, I, K, S, N and V; at position 588 an amino acid selected from the group consisting of: V, F, Y, L, T, S, I and R; at position 589 an amino acid selected from the group consisting of: S, N, L, T, I and R; and/or at position 590 an amino acid selected from the group consisting of: I, S, G, H and R. In some embodiments, the capsid protein comprises, relative to reference sequence SEQ ID NO: 1, at least two, three, four, five, six or all seven of any of the following: (i) at position 452 amino acid K; (ii) at position 585 an amino acid selected from the group consisting of: E, N, G, M, C, V and T; (iii) at position 586 an amino acid selected from the group consisting of: N, T, M, G, and D; (iv) at position 587 an amino acid selected from the group consisting of: T, L, I, K, S, N and V; (v) at position 588 an amino acid selected from the group consisting of: V, F, Y, L, T, S, I and R; (vi) at position 589 an amino acid selected from the group consisting of: S, N, L, T, I and R; and (vii) at position 590 an amino acid selected from the group consisting of: I, S, G, H and R. In some embodiments, the capsid protein comprises, relative to reference sequence SEQ ID NO: 1: at position 585 an amino acid selected from the group consisting of: E, N, M, C, and Q; at position 586 an amino acid selected from the group consisting of: A, M, G, D, N and S; at position 587 an amino acid selected from the group consisting of: T, N, V and A; at position 588 an amino acid selected from the group consisting of: V, Y, T, S, I and Q; at position 589 an amino acid selected from the group consisting of: S, G, L, I, R and A; and/or at position 590 an amino acid selected from the group consisting of: I, S, G, R and Q. In some embodiments, the capsid protein comprises, relative to reference sequence SEQ ID NO: 1: at position 452 an amino acid selected from the group consisting of: K and N; at position 585 an amino acid selected from the group consisting of: E, N, M, C, and Q; at position 586 an amino acid selected from the group consisting of: A, M, G, D, N and S; at position 587 an amino acid selected from the group consisting of: T, N, V and A; at position 588 an amino acid selected from the group consisting of: V, Y, T, S, I and Q; at position 589 an amino acid selected from the group consisting of: S, G, L, I, R and A; and at position 590 an amino acid selected from the group consisting of: I, S, G, R and Q. In some embodiments, the capsid protein comprises, relative to reference sequence SEQ ID NO: 1: at position 585 an amino acid selected from the group consisting of: E, N, M, and C; at position 586 an amino acid selected from the group consisting of: A, M, G, D, and N; at position 587 an amino acid selected from the group consisting of: T, N, and V; at position 588 an amino acid selected from the group consisting of: V, Y, T, S, and I; at position 589 an amino acid selected from the group consisting of: S, G, L, I and R; and/or at position 590 an amino acid selected from the group consisting of: I, S, G, and R. In some embodiments, the capsid protein comprises, relative to reference sequence SEQ ID NO: 1, at least two, three, four, five, six or all seven of any of the following: (i) at position 452 amino acid K; (ii) at position 585 an amino acid selected from the group consisting of: E, N, M, and C; (iii) at position 586 an amino acid selected from the group consisting of: A, M, G, D, and N; (iv) at position 587 an amino acid selected from the group consisting of: T, N, and V; (v) at position 588 an amino acid selected from the group consisting of: V, Y, T, S, and I; (vi) at position 589 an amino acid selected from the group consisting of: S, G, L, I and R; and (vii) at position 590 an amino acid selected from the group consisting of: I, S, G, and R. In some embodiments, the capsid protein comprises, relative to reference sequence SEQ ID NO: 1: at position 452 an amino acid selected from the group consisting of: K and N; and at position 587 amino acid substitution A587T; and optionally comprises amino acid N or R at one, two or more positions selected from the group consisting of: 584, 585, 586, 588, 589, and 590. In some embodiments, the capsid protein comprises, relative to reference sequence SEQ ID NO: 1: at position 452 an amino acid selected from the group consisting of: K and N; and amino acid N or R at one, two or more positions selected from the group consisting of: 584, 585, 586, 588, 589, and 590. In some embodiments, the capsid protein comprises, relative to reference sequence SEQ ID NO: 1: at position 452 an amino acid selected from the group consisting of: K and N; and amino acid S at two or more positions selected from the group consisting of: 585, 586, 587, 588, 589 and 590. In some embodiments, the capsid protein comprises, relative to reference sequence SEQ ID NO: 1: at position 452 an amino acid selected from the group consisting of: K and N; and at three, four, five or six positions in the region 585-590 of the VR-VIII site, amino acids selected from the group consisting of: N, S, T, R, and I. In some embodiments, the capsid protein comprises, relative to reference sequence SEQ ID NO: 1: at three, four, five or six positions in the region 585-590 of the VR-VIII site, amino acids selected from the group consisting of: N, S, T, and R. In some embodiments, the capsid protein comprises, relative to reference sequence SEQ ID NO: 1, amino acid substitutions Q585E, S586N, A587T, Q588V, A589S, Q590I, and N452K. In some embodiments, the capsid protein comprises, relative to reference sequence SEQ ID NO: 1, amino acid substitutions S586T, A587L, Q588F, A589N, Q590S, and N452K. In some embodiments, the capsid protein comprises, relative to reference sequence SEQ ID NO: 1, amino acid substitutions Q585N, A587T, Q588Y, A589L, Q590G, and N452K. In some embodiments, the capsid protein comprises, relative to reference sequence SEQ ID NO: 1, amino acid substitutions Q585G, A587I, Q588L, A589T, Q590H, and N452K. In some embodiments, the capsid protein comprises, relative to reference sequence SEQ ID NO: 1, amino acid substitutions Q585M, S586M, A587T, Q588T, and Q590R; and amino acid N at position 452. In some embodiments, the capsid protein comprises, relative to reference sequence SEQ ID NO: 1, amino acid substitutions Q585N, A587T, Q588Y, A589L, and Q590G; and amino acid N at position 452. In some embodiments, the capsid protein comprises, relative to reference sequence SEQ ID NO: 1, amino acid substitutions Q585C, A587T, Q588S, A589I, and Q590R; and amino acid N at position 452. In some embodiments, the capsid protein comprises, relative to reference sequence SEQ ID NO: 1, amino acid substitutions Q585E, S586D, A587N, Q588I, A589R, and Q590S; and amino acid N at position 452. In some embodiments, the capsid protein comprises, relative to reference sequence SEQ ID NO: 1, amino acid substitutions Q585E, S586D, A587N, Q588I, A589R, Q590S, and N452K. In some embodiments, the capsid protein comprises, relative to reference sequence SEQ ID NO: 1, amino acid substitutions Q585N, S586N, A587V, Q588I, A589S, Q590G, and N452K. In some embodiments, the capsid protein comprises, relative to reference sequence SEQ ID NO: 1, amino acid substitutions S586G and Q588Y; and amino acid N at position 452. In some embodiments, the capsid protein comprises, relative to reference sequence SEQ ID NO: 1, amino acid substitutions S586A, A587N, Q588Y, A589G, and N452K. In some embodiments, the capsid protein comprises, relative to reference sequence SEQ ID NO:1, amino acids ATN at positions 581-583, and amino acids AQTG at positions 591-594. In some embodiments, the capsid protein comprises, relative to reference sequence SEQ ID NO:1, amino acids ATNH at positions 581-584, and amino acids AQTG at positions 591-594. In some embodiments, the capsid protein comprises, relative to reference sequence SEQ TID NO:1: (i) amino acid sequence ATNHENTVSIAQTG at the VR-VIII positions 581-594, and amino acid K at the VR-IV position 452; (ii) amino acid sequence ATNHQTLFNSAQTG at the VR-VIII positions 581-594, and amino acid K at the VR-IV position 452; (iii) amino acid sequence ATNHNSTYLGAQTG at the VR-VIII positions 581-594, and amino acid K at the VR-TV position 452; (iv) amino acid sequence ATNHGSILTHAQTG at the VR-VIII positions 581-594, and amino acid K at the VR-IV position 452; (v) amino acid sequence ATNHMMTTARAQTG at the VR-VIII positions 581-594, and amino acid N at the VR-IV position 452; (vi) amino acid sequence ATNHNSTYLGAQTG at the VR-VIII positions 581-594, and amino acid N at the VR-IV position 452; (vii) amino acid sequence ATNHCSTSIRAQTG at the VR-VIII positions 581-594, and amino acid N at the VR-IV position 452; (viii) amino acid sequence ATNHEDNIRSAQTG at the VR-VIII positions 581-594, and amino acid N at the VR-IV position 452; (ix) amino acid sequence ATNHEDNIRSAQTG at the VR-VIII positions 581-594, and amino acid K at the VR-IV position 452; (x) amino acid sequence ATNHNNVISGAQTG at the VR-VIII positions 581-594, and amino acid K at the VR-IV position 452; (xi) amino acid sequence ATNHQGAYAQAQTG at the VR-VIII positions 581-594, and amino acid N at the VR-IV position 452; (xii) amino acid sequence ATNHQANYGQAQTG at the VR-VIII positions 581-594, and amino acid K at the VR-IV position 452; (xiii) amino acid sequence ATNHNMNRVNAQTG at the VR-VIII positions 581-594, and amino acid N at the VR-IV position 452; (xiv) amino acid sequence ATNHNNVISGAQTG at the VR-VIII positions 581-594, and amino acid N at the VR-IV position 452; (xv) amino acid sequence ATNHSNSVQSAQTG at the VR-VIII positions 581-594, and amino acid N at the VR-IV position 452; (xvi) amino acid sequence ATNHSSTFQGAQTG at the VR-VIII positions 581-594, and amino acid N at the VR-IV position 452; (xvii) amino acid sequence ATNHVSSFTSAQTG at the VR-VIII positions 581-594, and amino acid N at the VR-IV position 452; (xviii) amino acid sequence ATNHSTTNFRAQTG at the VR-VIII positions 581-594, and amino acid N at the VR-IV position 452; (xix) amino acid sequence ATNHSSIFNSAQTG at the VR-VIII positions 581-594, and amino acid N at the VR-IV position 452; (xx) amino acid sequence ATNHAGNYNNAQTG at the VR-VIII positions 581-594, and amino acid N at the VR-IV position 452; (xxi) amino acid sequence ATNHTSVISIAQTG at the VR-VIII positions 581-594, and amino acid N at the VR-IV position 452; (xxii) amino acid sequence ATNHHSRVEIAQTG at the VR-VIII positions 581-594, and amino acid N at the VR-IV position 452; (xxiii) amino acid sequence ATNHSSIIYSAQTG at the VR-VIII positions 581-594, and amino acid N at the VR-IV position 452; (xxiv) amino acid sequence ATNHSGRDSYAQTG at the VR-VIII positions 581-594, and amino acid N at the VR-IV position 452; (xxv) amino acid sequence ATNHSSSYNNAQTG at the VR-VIII positions 581-594, and amino acid N at the VR-IV position 452; (xxvi) amino acid sequence ATNHHNPSINAQTG at the VR-VIII positions 581-594, and amino acid N at the VR-IV position 452; (xxvii) amino acid sequence ATNHNRNGLLAQTG at the VR-VIII positions 581-594, and amino acid N at the VR-IV position 452; (xxviii) amino acid sequence ATNHESTSVRAQTG at the VR-VIII positions 581-594, and amino acid N at the VR-IV position 452; (xxix) amino acid sequence ATNHNIRTEMAQTG at the VR-VIII positions 581-594, and amino acid N at the VR-IV position 452; (xxx) amino acid sequence ATNHQTLFNSAQTG at the VR-VIII positions 581-594, and amino acid N at the VR-IV position 452; (xxxi) amino acid sequence ATNHLSVSSiAQTG at the VR-VIII positions 581-594, and amino acid N at the VR-IV position 452; (xxxii) amino acid sequence ATNHEDIIRSAQTG at the VR-VIII positions 581-594, and amino acid N at the VR-IV position 452; (xxxiii) amino acid sequence ATNRQTAQAQAQTG at the VR-VIII positions 581-594, and amino acid N at the VR-IV position 452; or (xxxiv) amino acid sequence ATNRQIAQAQAQTG at the VR-VIII positions 581-594, and amino acid N at the VR-TV position 452. In some embodiments, the capsid protein comprises, relative to reference sequence SEQ ID NO: 1: (i) an amino acid insertion at position 584 comprising one or more of an asparagine (N), a threonine (T), a tyrosine (Y), phenylalanine (F), and an alanine (A); (ii) an amino acid insertion at position 585 comprising one or more of a histidine (H) and a methionine (M); (iii) an amino acid insertion at position 586 comprising one or more of a histidine (H), a tyrosine (Y), a valine (V), a threonine (T), an alanine (A), an isoleucine (I), a tryptophan (W), a methionine (M), and a leucine; (iv) an amino acid insertion at position 587 comprising one or more of an isoleucine (I) and a proline (P): (v) an amino acid insertion at position 588 comprising one or more of an isoleucine (I), a threonine (T), and a proline (P); and/or (vi) an amino acid insertion at position 589 comprising one or more of a glycine (G) and a glutamine (Q). In some embodiments, the capsid protein comprises, relative to reference sequence SEQ ID NO: 1: (i) an amino acid insertion at position 584 consisting of a TY, FN, or AT; (ii) an amino acid insertion at position 585 consisting of MH; (iii) an amino acid insertion at position 586 consisting of HY, VT, AI, WM, or ML; (iv) an amino acid insertion at position 587 consisting of PI; and/or (v) an amino acid insertion at position 588 consisting of IT or PT. In some embodiments, the capsid protein shares, or comprises a sequence sharing, at least 90%, at least 95%, at least 96%, at least 97%, at least 990%, or 100% amino acid sequence identity to an AAV9 VP3 sequence according to SEQ ID NO: 487, except for the specified modifications. In some embodiments, the capsid protein shares, or comprises a sequence sharing, at least 90%, at least 95%, at least 96%, at least 97%, at least 99%, or 100% amino acid sequence identity to an AAV9 VP2 sequence according to SEQ ID NO: 486, except for the specified modifications. In some embodiments, the capsid protein shares, or comprises a sequence sharing, at least 90%, at least 95%, at least 96%, at least 97%, at least 99%, or 100% amino acid sequence identity to an AAV9 VP1 sequence according to SEQ ID NO: 1, except for the specified modifications. In some embodiments, the capsid protein comprises, consists essentially of, or consists of an amino acid sequence at least 85%, 90%, 95%, 96%, 97%, 98%, 99%, or 100% identical to any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof. In some embodiments, the capsid protein comprises, consists essentially of, or consists of a polypeptide sequence of any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774. In some embodiments, the rAAV virion transduces heart cells. In some embodiments, the rAAV virion transduces cardiomyocytes. In some embodiments, the rAAV virion traffics to at least one organ other than the liver. In some embodiments, the rAAV virion traffics to the heart. In some embodiments, the rAAV virion exhibits a higher heart transduction efficiency than an rAAV virion having an AAV9 VP1 capsid protein according to SEQ ID NO: 1. In some embodiments, the rAAV virion exhibits a higher heart-to-liver transduction ratio than an rAAV virion having an AAV9 VP1 capsid protein according to SEQ ID NO: 1, optionally at least 2, 3, 4, 5, 6, 7, 8, 9 or 10 times higher. In some embodiments, administration of the rAAV virion to a subject leads to a lower liver viral load than administration of an rAAV virion having an AAV9 VP1 capsid protein according to SEQ ID NO: 1, optionally at least 2, 3, 4, 5, 6, 7, 8, 9 or 10 times lower. In some embodiments, the rAAV virion exhibits a higher transduction efficiency, optionally higher heart transduction efficiency, than an rAAV virion having an AAV9 VP1 capsid protein according to SEQ ID NO: 1, assessed in a primate. In some embodiments, the rAAV virion exhibits a higher heart-to-liver transduction ratio than an rAAV virion having an AAV9 VP1 capsid protein according to SEQ ID NO: 1, assessed in a primate, optionally at least 2, 3, 4, 5, 6, 7, 8, 9 or 10 times higher. In some embodiments, administration of the rAAV virion to a subject leads to a lower liver viral load than administration of an rAAV virion having an AAV9 VP1 capsid protein according to SEQ ID NO: 1, assessed in a primate, optionally at least 2, 3, 4, 5, 6, 7, 8, 9 or 10 times lower. In some embodiments, the PKP2 expression cassette comprises a sequence having at least 95% identity to SEQ ID NO: 782 or SEQ ID NO: 783. In some embodiments, the PKP2 expression cassette comprises a nucleic acid sequence having at least 95% identity to SEQ ID NO: 786. In some embodiments, the PKP2 expression cassette comprises a cardiac specific promoter. In some embodiments, the cardiac specific promoter directs gene expression in the myocardium, the epicardium, or both. In some embodiments, the cardiac specific promoter is a troponin promoter, or an alpha-myosin heavy chain promoter. In some embodiments, the troponin promoter has a nucleic acid sequence having at least 95% identity to SEQ ID NO: 784. In some embodiments, the PKP2 expression cassette comprises a PKP2 promoter. In some embodiments, the PKP2 promoter has a nucleic acid sequence having at least 95% identity to SEQ ID NO: 785. In some embodiments, the PKP2 expression cassette comprises a constitutive promoter. In some embodiments, the constitutive promoter is a beta-actin promoter. In some embodiments, the PKP2 expression cassette comprises a cardiac specific enhancer. In some embodiments, the PKP2 expression cassette comprises a 3′ element. In some embodiments, the 3′ element comprises a Woodchuck Hepatitis Virus Posttranscriptional Regulatory Element (WPRE), a bovine growth hormone polyadenylation (bGH polyA) sequence, or a combination thereof.

In another aspect, provided herein are pharmaceutical compositions comprising an rAAV virion according to any embodiment provided herein and a pharmaceutically acceptable carrier.

In another aspect, provided herein are methods of transducing a cardiac cell, comprising contacting the cardiac cell with an rAAV virion according to any embodiment provided herein, wherein the rAAV virion transduces the cardiac cell. In some embodiments, the cardiac cell is a cardiomyocyte. In some embodiments, the rAAV virion exhibits higher transduction efficiency in the cell than an rAAV virion having an AAV9 VP1 capsid protein according to SEQ ID NO: 1.

In another aspect, provided herein are methods of delivering one or more gene products to a cardiac cell, comprising contacting the cardiac cell with an rAAV virion according to any embodiment provided herein. In some embodiments, the cardiac cell is a cardiomyocyte.

In another aspect, provided herein are methods of treating a heart disease or disorder in an individual in need thereof, comprising administering a therapeutically effective amount of an rAAV virion according to any embodiment provided herein to the subject, wherein the rAAV virion transduces cardiac tissue. In some embodiments, the heart disease or disorder is arrhythmogenic right ventricular cardiomyopathy (ARVC) or arrhythmogenic cardiomyopathy (ACM). In some embodiments, the AAV virion is administered intravenously, intracardially, pericardially, or intraarterially. In some embodiments, the method reverses, reduces, or prevents at least one of fibrofatty tissue replacement, myocardial atrophy, predominant right ventricular dilation, ventricular arrhythmias, sudden cardiac death, or exercise-triggered cardiac events. In some embodiments, the method reverses, reduces, or prevents fibrofatty tissue replacement in myocardium, epicardium, or both. In some embodiments, the method restores desmosome structure and/or function. In some embodiments, the method restores PKP2 protein and activity levels. In some embodiments, the method restores PKP2 induced gene expression. In some embodiments, the method restores expression of one or more of Ryanodine Receptor 2 (Ryr2), Ankyrin-B (Ank2), Cacnalc (CaV 1.2), triadin (Trdn), or calsequestrin-2 (Casq2). In some embodiments, the individual is identified as having at least one variation in a desmosome protein. In some embodiments, the desmosome protein is PKP2. In some embodiments, the variation comprises a deletion, an insertion, a single nucleotide variation, or a copy number variation.

In another aspect, provided herein are rAAV virions according to any embodiment provided herein for use in methods of treating a heart disease or disorder in an individual in need thereof, wherein the rAAV virion transduces cardiac tissue. In some embodiments, the heart disease or disorder is arrhythmogenic right ventricular cardiomyopathy (ARVC) or arrhythmogenic cardiomyopathy (ACM). In some embodiments, the AAV virion is administered intravenously, intracardially, pericardially, or intraarterially. In some embodiments, the method reverses, reduces, or prevents at least one of fibrofatty tissue replacement, myocardial atrophy, predominant right ventricular dilation, ventricular arrhythmias, sudden cardiac death, or exercise-triggered cardiac events. In some embodiments, the method reverses, reduces, or prevents fibrofatty tissue replacement in myocardium, epicardium, or both. In some embodiments, the method restores desmosome structure and/or function. In some embodiments, the method restores PKP2 protein and activity levels. In some embodiments, the method restores PKP2 induced gene expression. In some embodiments, the method restores expression of one or more of Ryanodine Receptor 2 (Ryr2), Ankyrin-.B (Ank2), Cacnalc (CaV1.2), triadin (Trdn), or calsequestrin-2 (Casq2). In some embodiments, the individual is identified as having at least one variation in a desmosome protein. In some embodiments, the desmosome protein is PKP2. In some embodiments, the variation comprises a deletion, an insertion, a single nucleotide variation, or a copy number variation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present disclosure will be obtained by reference to the following detailed description that sets forth illustrative embodiments, in which the principles of the disclosure are utilized, and the accompanying drawings of which:

FIG. 1 illustrates how cardiac desmosomes tie cells together.

FIG. 2 shows a summary of ARVC disease indications and possible disease mechanisms.

FIGS. 3A-3C show the results of acute silencing of PKP2 in iPSCM at day 8. FIG. 3A shows the disappearance of DSP from the cellular membrane. FIG. 3B shows a graph illustrating the reduction in sarcomere density. FIG. 3C shows the disarray of cell compaction in patterned iPSCM.

FIG. 4 shows a quantitative analysis of DSP membrane localization as determined by colocalization with PKG.

FIG. 5 shows an immunoblot which illustrates a reduced total amount of DSP protein, detected mainly in the insoluble fraction, in cells where PKP2 is silenced.

FIGS. 6A-6B show results of PKP2 transduction by AAV. FIG. 6A shows a vector map of the AAV construct. FIG. 6B shows an immunofluorescence image of restoration of DSP membrane localization. FIG. 6C shows a quantification of total DSP intensity post PKP2 silencing and AAV-PKP2 transgene rescue.

FIGS. 7A-7B show results of PKP2 transduction by AAV on contraction velocity. FIG. 7A shows the experimental timeline. FIG. 7B shows two contractility assays which demonstrate functional rescue of reduced velocity post PKP2 silencing.

FIG. 8 shows a second generation schematic of an AAV expression cassette of human and mouse PKP2a. The left panel shows all of the elements in the expression cassette. The right panel shows the arrangement of elements in the expression cassettes.

FIG. 9A and FIG. 9B show results of the second generation AAV-hPKP2α rescue of contraction velocity post PKP2 silencing in iPSC cardiomyocytes. FIG. 9A shows expression in soluble and insoluble fractions in cells transduced in different multiplicities of infection. FIG. 9B shows rescue of contraction velocity in cells post PKP2 silencing.

FIG. 10 shows expression of the second generation AAV-PKP2α in wildtype mice.

FIGS. 11A-11G show results of pilot expression safety studies of the second generation AAV9 human and mouse PKP2α in wildtype mice. FIG. 11A shows body weight before and after AAV9 injection. FIG. 11B shows ejection fraction in mice treated with the AAV9 human or mouse PKP2α. FIG. 11C and FIG. 11D show LV structure measured by internal diameters end diastole and systole. FIG. 11E, FIG. 11F, and FIG. 11G show electrophysiology activity by QRS (11E), QT interval (11F) and P/R amplitude (11G).

FIG. 12 shows a Kaplan-Meier survival curve of PKP2-cKO mice.

FIGS. 13A-13B show right ventricle (RV) dilated cardiomyopathy of PKP2-cKO mice. FIG. 13A (left panel) shows images that illustrate increased RV internal dimension at end-diastole (RVIDd) in PKP2-cKO mice. FIG. 13A (right panel) shows a graph of RVIDd over time in PKP2-cKO mice. FIG. 13B (left panel) shows images illustrating the increase in RV area in PKP2-cKO mice. FIG. 13B (right panel) shows a graph of RV area over time in PKP2-cKO mice.

FIGS. 14A-14B show development of left ventricle (LV) dilated cardiomyopathy of PKP2-cKO mice compared with control. FIG. 14A (left panel) shows images of increased LV internal dimension at end-systole (LVIDs) and end-diastole (LVIDd) in PKP2-cKO mice. FIG. 14A (right panel) shows a graph which shows the increase in LVIDs and LVIDd in PKP2-cKO mice over time. FIG. 14B shows a graph of LV performance as measured by percent ejection fraction over time.

FIG. 15 shows development of severe electrophysiological phenotypes of PKP2-cKO mice compared with control, specifically prolonged QRS interval and increased P/R amplitude ratio in PKP2-cKO mice. The top panel shows exemplary electrocardiogram of control and PKP2-cKO mice. The bottom panel shows graphs of the increase in QRS interval and increase in P/R amplitude in PKP2-cKO mice compared with control.

FIGS. 16A-16C show enhanced expression of fibrosis, tissue remodeling genes, and heart failure markers. FIG. 16A shows PKP2 RNA expression in RV and LV (top) and desmosome and Cx43 protein expression (bottom) of PKP2-cKO mice compared with control. FIG. 16B shows enhanced expression of fibrosis genes: TGFβ1, Col1a1, and Col3a1; and tissue remodeling genes: Timp1 and Mmp2 in PKP2-cKO mice compared with control. FIG. 16C shows enhanced expression of heart failure markers, NPPA and NPPB, in PKP2-cKO mice compared with control mice.

FIG. 17 shows the experimental design to evaluate PKP2 efficacy as gene therapy in the PKP2-cKO ARVC mouse model.

FIG. 18A shows a schematic of the AAV expression cassettes for human and mouse PKP2α.

FIG. 18B shows immunoblots of protein expression of mouse and human PKP2α from mice treated with AAV9:PKP2.

FIG. 19 shows a Kaplan-Meier survival curve of PKP2-cKO mice treated with AAV9:PKP2.

FIGS. 20A-20C show the efficacy of AAV9:PKP2 treatment of PKP2-cKO mice in reducing RV and LV dilation and maintaining cardiac function. FIG. 20A shows a graph illustrating improvement in ejection fraction in AAV9:PKP2 treated mice. FIG. 20B shows a graph illustrating reduction of RV dilation in AAV9:PKP2 treated mice. FIG. 20C shows graphs illustrating improvement in LVIDd (top) and LVIDs (bottom).

FIGS. 21A-21B show improvement in ECG parameters of PKP2-cKO mice treated with AAV:PKP2. FIG. 21A shows exemplary raw ECG traces of control and PKP2-cKO mice treated with AAV9:mPKP2 and buffer. FIG. 21B shows graphs illustrating improvement of P/R ratio, QT interval, and QRS interval in PKP2-cKO mice treated with AAV9:PKP2 compared with treatment with buffer.

FIGS. 22A-22B show AAV9:PKP2 treatment improvement in arrhythmias in PKP2-cKO mice. FIG. 22A (top) shows a table grading of severity of arrhythmias. FIG. 22A (bottom) shows a graph which summarizes improvement of arrhythmia scores of PKP2-cKO mice treated with AAV9:PKP2 compared with control. FIG. 22B shows a distribution graph showing improvement in severity of arrhythmias in PKP2-cKO mice treated with AAV9:PKP2 compared with control. Each dot represents an animal.

FIG. 23 shows the experimental design used to evaluate human PKP2 efficacy as a gene therapy using the PKP2-cKO ARVC mouse model.

FIGS. 24A-24D show results of AAV9:hPKP2 gene therapy treatment of PKP2-cKO mice. FIG. 24A shows results of ejection fraction. FIG. 24B show results of right ventricle size. FIG. 24C shows LV dilation as measured by LVIDd. FIG. 24D shows LV dilation as measured by LVIDs.

FIG. 25 shows results of AAV9:hPKP2 gene therapy treatment of PKP2-cKO mice for QT interval (top), PIR Ratio (middle), and Arrhythmia Score (bottom).

FIGS. 26A-26B show results of AAV9:hPKP2 treatment of PKP2-cKO mice in reducing expression of heart failure markers, fibrosis, and tissue remodeling markers in right ventricle (FIG. 26A) and left ventricle (FIG. 26B).

FIGS. 27A-27B shows results of AAV9:hPKP2 treatment of PKP2-cKO mice in reducing fibrosis development. FIG. 27A shows histological images of muscle from control and PKP2-cKO mice with and without AAV9:hPKP2 treatment. FIG. 27B shows a graph of collagen positive tissue from control and PKP2-cKO mice with and without AAV9:hPKP2 treatment.

FIGS. 28A-28B show expression of PKP2 and other desmosome proteins in soluble fraction (FIG. 28A) and insoluble fraction (FIG. 28B).

FIG. 29 depicts the AAV9 capsid highlighting amino acids in selected AAV9 variable regions (VR-IV and VR-VIII site).

FIG. 30 shows a schematic of directed evolution selection strategy and variant characterization. Following library generation, each library was subjected to two rounds of selection in primates.

FIG. 31 shows a vector map for the vector genomes used in screening for capsid protein variants.

FIG. 32 shows a plot of data from the second-round screening. Liver viral genome abundance is plotted against heart mRNA transcript abundance (“Heart transduction”) on a log, scale. In each case, the values are normalized against the values for a reference AAV9 virion.

FIGS. 33A-33C plot 102 variants selected as having the desired cell properties (high heart transduction relative to AAV9, high heart-to-liver ratio relative to AAV9, or both). FIG. 33A plots heart transduction measurements of the 102 selected variants on x-axis and heart-to-liver ratios on y-axis.

FIG. 33B shows the subset of variants from the sub-library no. 1 in Table 6 with both randomized VR-IV (amino acids 452 to 458 of AAV9 VP1) and substituted VR-VIII (amino acids 586 to 589 of AAV9 VP1). FIG. 33C shows novel variants with modified VR-VIII (amino acids 581 to 594 on AAV9 VP1).

FIG. 34 shows a schematic of re-testing rAAV virions having engineered capsid proteins in a mouse model.

FIGS. 35A-35C show heart transduction (FIG. 35A), liver viral load (FIG. 35B), and heart-to-liver ratio (FIG. 35C) measurements of the selected variants and AAV9 reference.

FIGS. 36A-36B show schematics of the modified viral capsids (FIG. 36A) and screening strategy for evaluating transduction efficiency in various organs and tissues of animal models transduced with barcoded modified viral capsids (FIG. 36B).

FIG. 37 shows graphs measuring transduction/viral load levels of novel capsids without an N452K mutation (ZC404, ZC470, ZC428, and ZC416) and with an N452K mutation (ZC373, ZC374, ZC375, and ZC376) in cynomolgus monkey heart and liver, mouse heart and liver, and human iPSCs.

FIG. 38 shows a schematic of a screening strategy for evaluating transduction efficiency in various organs and tissues of animal models transduced with modified viral capsids.

FIGS. 39A-39B show a heatmap of transduction efficiency of modified AAV capsids. Each column represents one capsid, and each row is one sample type. The average measurements of 4 animals, 3 animals, 6 animals, or 2 multiplicities of infection are shown for cynomolgus monkey, mouse, pig, and iPSC-CMs, respectively. The capsids are ordered in columns from left to right ranked by their heart-to-liver ratio in cynomolgus monkey. AAV9-1, AAV9-2, and AAV9-3 are all wildtype AAV9 capsid serve as control replicates.

FIG. 40 provides graphs showing transduction in heart, liver viral load, and the heart-to-liver transduction ratio in cynomolgus monkey, mouse, and pig using four novel AAV capsids. Results show fold change relative to wild-type AAV9 control.

FIG. 41 provides a graph showing heart-to-liver ratio, heart transduction, and liver viral load of four novel capsids compared to AAV9 wild-type control in Cynomolgus monkeys. Animals were administered 1E+13 vg/kg via intravenous bolus administration. Tissue was collected 4-weeks post injection. The figure shows fold change relative to wildtype AAV9 control.

FIG. 42 provides graphs showing heart-to-liver ratio, heart transduction, and liver viral load of ZC375, ZC401, ZC428, and ZC478 capsids compared to AAV9 wild-type control in CD-1 mice. Virus was administered at 2E+13 vg/kg for ZC375, ZC401, and ZC428, and 1.45E+13 vg % kg for ZC478 through retro-orbital injection. Dosage matched AAV9 controls were included. Tissue was collected 18 days post injection. Results show fold change relative to AAV9 control.

FIG. 43 provides graphs showing heart-to-liver ratio, heart transduction, and liver viral load of ZC401 capsid compared to AAV9 wild-type control in C57BL6NCrl mice. The viruses were administered at 2E+13 vg/kg through retro-orbital injection. Tissue was collected 18 days post injection. Results show fold change relative to AAV9.

FIG. 44 provides a graph showing heart and liver transduction by ZC401 capsid compared to AAV9 wild-type control in CD-1 mice. Viruses were administered at 2E+13 vg/kg (AAV9 and ZC401) or 1.2E+14 vg/kg (ZC401) through retro-orbital injection. Tissue was collected 18 days post injection. Results show fold change relative to AAV9.

FIG. 45 shows incorporation of N452K substitution into AAV9-based capsid variants. The figure provides an image of capsid structure illustrating the location of VR-VIII region and N452 (Asn452) on the wildtype AAV9 capsid and tables showing the names of sequences of parental capsids (on the left) and new N452K capsids (on the right) for AAV9-based VR-VIII substitution variants.

FIG. 46 shows testing N452K variants in multiple models. The figure shows a heatmap of transduction efficiency of modified AAV capsids from FIG. 45. Each column represents one capsid, and each row is one sample type. The N452K variants were tested in Cynomolgus monkeys, mice, and human iPSC-CMs using pooled barcode-based methodology. Heart transduction and iPSC-CM transduction were measured by NGS-based quantification of RNA samples. Liver viral load was measured by NGS-based quantification of DNA samples. Heart-to-liver ratio was calculated by dividing heart transduction by liver viral load. All the measurements were normalized to AAV9 control.

FIG. 47 is a graph showing iPSC-CM transduction efficiency improvements of N452K variants compared to matched parental capsids without the N452 substitution (in fold change). N452K substitution consistently enhances transduction efficiency.

FIG. 48 provides graphs showing heart-to-liver ratio, heart transduction, and liver viral load of select capsids from FIG. 46 compared to AAV9 wild-type control in Cynomolgus monkey (a non-human primate or “NHP”). All the values are relative to the performance of wildtype AAV9 control. ZC533, ZC536, and ZC538 show improved heart-to-liver ratio and/or improved heart transduction in NHPs relative to AAV9.

FIG. 49 shows a schematic of experiment comparing biodistribution and transduction of new capsids and AAV9 in NHPs. In this experiment, performance of top capsids was measured in NHPs injected individually (one test article per animal) at a therapeutic relevant dose. AAV9, ZC375, and ZC428 were administered at 6E+13 vg/kg systemically. This study was divided to two phases and in each phase, one novel capsid and AAV9 control were tested with 4 Cynomolgus Monkeys per test article. Animals were sacrificed at 28-day post injection. RNA and DNA were extracted from heart and liver tissues, followed by RT-qPCR based quantification of viral.

FIG. 50 is a graph showing viral transgene expression (“Heart RNA”) levels in the heart from the NHP biodistribution and transduction study depicted in FIG. 49. Viral transgene expression levels were measured by RT-qPCR analysis on RNA samples and normalized to the average of all AAV9 data points. Each dot on the figure represents one individual animal for which 4 heart biopsy samples were analyzed and averaged. Both ZC375 and ZC428 show comparable transgene expression in the heart compared to their matched AAV9 control.

FIGS. 51A-51B are graphs showing reduced liver tropism compared to AAV9. The figure shows viral transgene expression (“Liver RNA”; FIG. 51A) and viral genome load (“Liver DNA”; FIG. 51B) levels in the liver from the NHP biodistribution and transduction study in FIG. 49 (with animals systemically administered ZC375, ZC428, or wild-type control AAV9 at 6E+13 vg/kg). Viral transgene expression levels were measured by RT-PCR analysis on RNA samples and normalized to the average of all AAV9 data points. Viral genome load levels were measured by qPCR analysis on DNA samples and normalized to the average of all AAV9 data points. Each dot on the figure represents one individual animal for which 2 liver biopsy samples were analyzed and averaged. ZC375 and ZC428 show reduced transduction in the liver at both RNA and DNA levels compared to their matched AAV9 control.

FIGS. 52A-52B are graphs showing heart transduction to liver transduction ratios from the NHP biodistribution and transduction study depicted in FIG. 49, calculated by either heart RNA-based and liver RNA-based measurements (FIG. 52A), or heart RNA-based and liver DNA-based measurements (FIG. 52B). The ratios were individually calculated to each animal. ZC375 and ZC428 showed improved heart-to-liver ratio compared to their matched AAV9 control.

### DETAILED DESCRIPTION

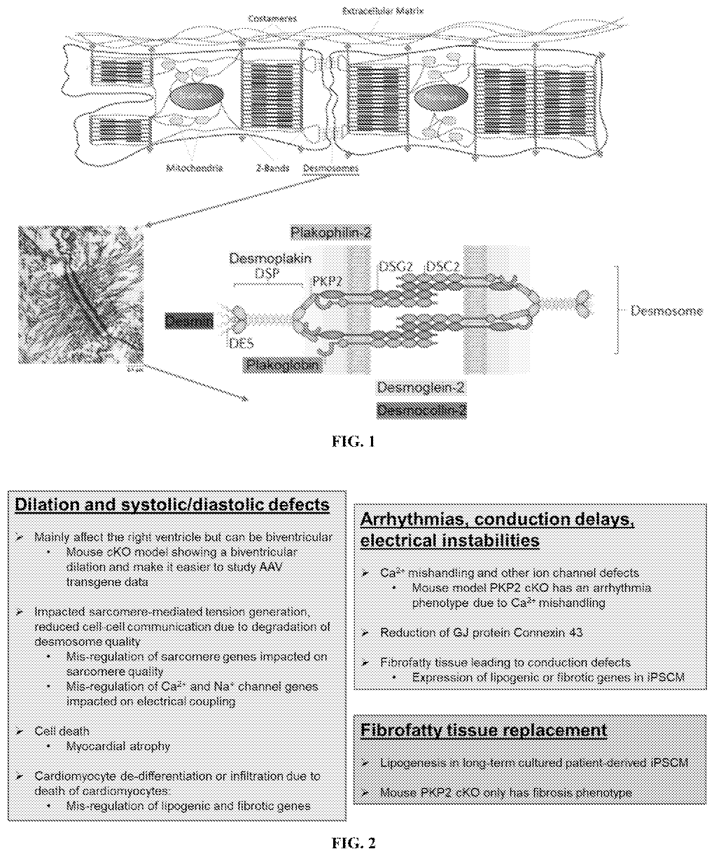

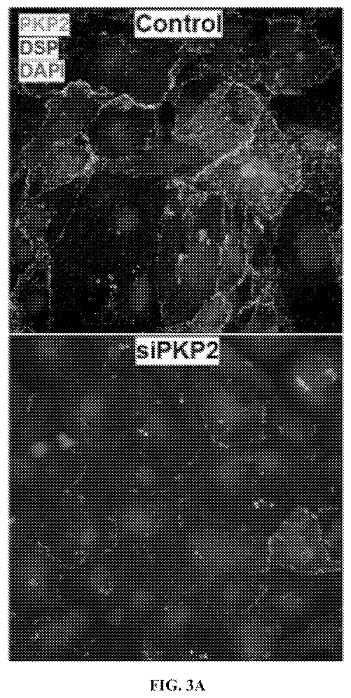

The most common genetic basis of arrhythmogenic right ventricular cardiomyopathy (ARVC) is mutations in genes encoding desmosomal proteins. Functionally, desmosomes are adhesive intercellular connections that hold intercalated cardiomyocytes together. Plakophillin-2 (PKP2), one of desmosomal genes, is most frequently identified as the causal factor for ARVC. Internal to the membrane-located complex, PKP2 interacts with desmosomal proteins, plakoglobin (PKG) and desmoplakin (DSP). DSP anchors the intermediate filaments, desmin, which form an interwoven network to stabilize the contractile units of cardiac cells, sarcomeres, and other organelles (FIG. 1, Brodehl et al., 2018; Moncayo-Arlandi and Brugada, 2017). It is believed that the loss of desmosome impacts cell-cell adhesion, signal transduction, and electrical coupling of cardiomyocytes (Wang et al., 2018). Furthermore, the lost signal transduction and electrical coupling are joint defective outcomes by additional collapse of connexin-containing Gap junctions (GJs). GJs are essential in electrically coupling cells and facilitate synchronous beating by allowing flow of small molecules between cells (Green et al., 2019) (FIG. 2 summary on ARVC disease indication and possible mechanisms). In addition, epicardial differentiation can contribute to fibrofatty remodeling that is observed in ARVC or ACM patients (Kohela et al., 2021).

To delineate the functionality of desmosomes, genetic mouse lines and patient-derived iPSCM models were generated. Cardiac knock-out mouse model of PKP2 (the Delmar mouse model, Cerrone et al., 2017) showed profound early development of biventricular dilation, fibrosis, and a significant reduction of genes regulating Ca²⁺ homeostasis, revealing underling mechanisms for arrhythmias possibly before overt structural changes. Several patient-derived iPSCM lines harboring PKP2 mutations showed reduction of PKP2 expression. Ca²⁺ handling defects, and lipid droplet accumulation induced by culturing in lipogenic induction media (Brodehl et al., 2019).

Reduction of PKP2 at both mRNA and protein level was reported in ARVC patient heart samples with PKP2 mutations (Akdis et al., 2016: Asimaki et al., 2009). Nonsense-mediated mRNA-decay (NMD) was proposed for some desmosomal gene mutations including PKP2 mutations, suggesting a much less known cellular mechanism in balancing expression of mutated transcripts and proteins (Gcerull and Brodehl, 2020; Mura et al., 2003). Those observations suggest a possibility of gene therapy-based intervention of ARVC by restoring expression level of WT PKP2 in heart.

In additional aspects, the disclosure provides recombinant adeno-associated virus (rAAV) virions comprising engineered capsid proteins. In particular, the disclosure provides engineered capsid proteins (including chimeric capsid proteins), methods of identifying them, and methods of using them. The methods of identifying new capsid proteins disclosed herein have wide applicability for any serotype of AAV, including chimeric capsid proteins. In addition, they can be applied to iteratively improve capsid proteins that have mutations from this or other methods, in general, the methods of the disclosure relate to preparation of randomized or semi-randomized libraries of AAV capsids in the form of cap gene polynucleotides, preparation of AAV virions comprising such capsids (either by incorporating the cap gene library into an AAV genome or providing it in trans such as on a plasmid transfected into the packaging line), positively or negatively selecting the AAV virions, and recovering the cap gene for sequencing. In some embodiments, the recovery and sequencing include nanopore sequencing. Other high-throughput or next-generation-sequencing (NGS) methods can be used.

In some embodiments, the present disclosure provides recombinant adeno-associated virus (rAAV) virions comprising:
a) a capsid protein as described herein; and
b) a heterologous nucleic acid comprising a nucleotide sequence encoding one or more gene products.

In another aspect, provided herein are recombinant adeno-associated virus (rAAV) virions, comprising a capsid protein and a plakophilin-2 (PKP2) expression cassette, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1: an amino acid insertion at position 584, or between positions 583 and 584, comprising one or more of an asparagine (N), a threonine (T), a tyrosine (Y), phenylalanine (F), and an alanine (A); an amino acid insertion at position 585, or between positions 584 and 585, comprising one or more of a histidine (H) and a methionine (M); an amino acid insertion at position 586, or between positions 585 and 586, comprising one or more of a histidine (H), a tyrosine (Y), a valine (V), a threonine (T), an alanine (A), an isoleucine (I), a tryptophan (W), a methionine (M), and a leucine (L): an amino acid insertion at position 587, or between positions 586 and 587, comprising one or more of an isoleucine (I) and a proline (P); an amino acid insertion at position 588, or between positions 587 and 588, comprising one or more of an isoleucine (I), a threonine (T), and a proline (P); an amino acid insertion at position 589, or between positions 588 and 589, comprising one or more of a glycine (G) and a glutamine (Q); one or more amino acid substitutions selected from the group consisting of N452K, N452A, N452V, N452I, G453A, G453N, S454T, S454D, G455N, Q456L, Q456K, N457L, N457V, Q458I, and Q458H; and/or one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some embodiments of rAAV virions disclosed herein the capsid protein comprises one, two, three, four or more substitutions or insertions in the VR-VIII site. In some embodiments, the capsid protein comprises, relative to reference SEQ ID NO:1, one, two, three, four or more substitutions or insertions at positions from 584 to 590 in the VR-VIII site, or one, two, three, four or more substitutions or insertions at positions from 585 to 590 in the VR-VIII site. In some embodiments, the capsid protein comprises, relative to reference sequence SEQ ID NO: 1: (i) one or more amino acid substitutions selected from the group consisting of T582D, T582E, N583V, H584Q, S586K, A587P, A587S, Q588G, Q588M, A589S, A591I, G594Q, and G594D; (ii) one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588r, and Q588R; (iii) one or more amino acid substitutions selected from the group consisting of Q585C, Q585S, S586I, A587V and A587G; or (iv) one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L. In some embodiments, the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence. In some embodiments, the capsid protein comprises, relative to reference sequence SEQ ID NO: 1, one or more amino acid substitutions selected from the group consisting of N452K, N452A, N452V, N452I, G453A, G453N, S454T, S454D, G455N, Q456L, Q456K, N457L, N457V, Q458I, and Q458H. In some embodiments, the capsid protein comprises, relative to reference sequence SEQ ID NO: 1, at position 452 an amino acid selected from the group consisting of: K and N. In some embodiments, the capsid protein comprises, relative to reference sequence SEQ ID NO: 1, an amino acid substitution N452K. In some embodiments, the capsid protein comprises, relative to reference sequence SEQ ID NO: 1: at position 584 an amino acid selected from the group consisting of: R and H; at position 585 an amino acid selected from the group consisting of: N, M, C, E, G, S, V, A, T, H, L and Q; at position 586 an amino acid selected from the group consisting of: M, D, N, G, A, T, R, I and S; at position 587 an amino acid selected from the group consisting of: T, N, V, L, I, S, R, P and A; at position 588 an amino acid selected from the group consisting of: Y, T, S, I, V, F, L, R, N, D, G and Q; at position 589 an amino acid selected from the group consisting of: L, L, R, S, G, N, T, V, Q, F, E, Y and A; and/or at position 590 an amino acid selected from the group consisting of: G, R, S, I, H, N, Y, L, M and Q. In some embodiments, the capsid protein comprises, relative to reference sequence SEQ ID NO: 1: at position 452 an amino acid selected from the group consisting of: K and N; at position 584 an amino acid selected from the group consisting of: R and H; at position 585 an amino acid selected from the group consisting of: N, M, C, E, G, S, V, A, T, H, L and Q; at position 586 an amino acid selected from the group consisting of: M, D, N, G, A, T, R, I and S; at position 587 an amino acid selected from the group consisting of: T, N, V, L, I, S, R, P and A; at position 588 an amino acid selected from the group consisting of: Y, T, S, I, V, F, L, R, N, D, G and Q; at position 589 an amino acid selected from the group consisting of: L, I, R, S, G, N, T, V, Q, F, E, Y and A; and at position 590 an amino acid selected from the group consisting of: G, R, S, I, H, N, Y, L, M and Q. In some embodiments, the capsid protein comprises, relative to reference sequence SEQ ID NO: 1: at position 584 amino acid R, at position 585 an amino acid selected from the group consisting of: N, M, C, E, G, S, V, A, T, H and, L; at position 586 an amino acid selected from the group consisting of: M, D, N, G, A, T, R, and I; at position 587 an amino acid selected from the group consisting of: T, N, V, L, I, S, R, and P; at position 588 an amino acid selected from the group consisting of: Y, T, S, I, V, F, L, R, N, D, and G; at position 589 an amino acid selected from the group consisting of: L, I, R, S, G, N, T, V, Q, F, E, and Y; and/or at position 590 an amino acid selected from the group consisting of: G, R, S, I, H, N, Y, L, and M. In some embodiments, the capsid protein comprises, relative to reference sequence SEQ ID NO: 1, at least two, three, four, five, six, seven or all eight of any of the following: (i) at position 452 amino acid K; (ii) at position 584 amino acid R; (iii) at position 585 an amino acid selected from the group consisting of: N, M, C, E, G, S, V, A, T, H, and L; (iv) at position 586 an amino acid selected from the group consisting of: M, D, N, G, A, T, R, and I: (v) at position 587 an amino acid selected from the group consisting of: T, N, V, L, I, S, R, and P; (vi) at position 588 an amino acid selected from the group consisting of: Y, T, S, I, V, F, L, R, N, D, and G; (vii) at position 589 an amino acid selected from the group consisting of: L, I, R, S, G, N, T, V, Q, F, E, and Y; and (viii) at position 590 an amino acid selected from the group consisting of: G, R, S, I, H, N, Y, L, and M. In some embodiments, the capsid protein comprises, relative to reference sequence SEQ ID NO: 1: at position 585 an amino acid selected from the group consisting of: E, N, G, M, C, V, T and Q; at position 586 an amino acid selected from the group consisting of: N, T, M, G, D, and S; at position 587 an amino acid selected from the group consisting of: T, L, I, K, S, N, V and A; at position 588 an amino acid selected from the group consisting of: V, F, Y, L, T, S, I, R and Q; at position 589 an amino acid selected from the group consisting of: S, N, L, T, I, R and A; and/or at position 590 an amino acid selected from the group consisting of: I, S, G, H, R and Q. In some embodiments, the capsid protein comprises, relative to reference sequence SEQ ID NO: 1: at position 452 an amino acid selected from the group consisting of: K and N; at position 585 an amino acid selected from the group consisting of: E, N, G, M, C, V, T and Q; at position 586 an amino acid selected from the group consisting of: N, T, M, G, D, and S; at position 587 an amino acid selected from the group consisting of: T, L, I, K, S, N, V and A; at position 588 an amino acid selected from the group consisting of: V, F, Y, L, T, S, I, R and Q; at position 589 an amino acid selected from the group consisting of: S, N, L, T, I, R and A; and at position 590 an amino acid selected from the group consisting of: I, S, G, H, R and Q. In some embodiments, the capsid protein comprises, relative to reference sequence SEQ ID NO: 1: at position 585 an amino acid selected from the group consisting of: E, N, G, M. C, V and T; at position 586 an amino acid selected from the group consisting of: N, T, M, G, and D; at position 587 an amino acid selected from the group consisting of: T, L, I, K, S, N and V; at position 588 an amino acid selected from the group consisting of: V, F, Y, L, T, S, I and R; at position 589 an amino acid selected from the group consisting of: S, N, L, T, I and R; and/or at position 590 an amino acid selected from the group consisting of: I, S, G, H and R. In some embodiments, the capsid protein comprises, relative to reference sequence SEQ ID NO: 1, at least two, three, four, five, six or all seven of any of the following: (i) at position 452 amino acid K; (ii) at position 585 an amino acid selected from the group consisting of: E, N, G, M, C, V and T; (iii) at position 586 an amino acid selected from the group consisting of: N, T, M, G, and D; (iv) at position 587 an amino acid selected from the group consisting of: T, L, I, K, S, N and V; (v) at position 588 an amino acid selected from the group consisting of: V, F, Y, L, T, S, I and R; (vi) at position 589 an amino acid selected from the group consisting of: S, N, L, T, I and R; and (vii) at position 590 an amino acid selected from the group consisting of: I, S, G, H and R. In some embodiments, the capsid protein comprises, relative to reference sequence SEQ ID NO: 1: at position 585 an amino acid selected from the group consisting of: E, N, M, C, and Q; at position 586 an amino acid selected from the group consisting of: A, M, G, D, N and S; at position 587 an amino acid selected from the group consisting of: T, N, V and A; at position 588 an amino acid selected from the group consisting of: V, Y, T, S, I and Q; at position 589 an amino acid selected from the group consisting of: S, G, L, I, R and A; and/or at position 590 an amino acid selected from the group consisting of: I, S, G, R and Q. In some embodiments, the capsid protein comprises, relative to reference sequence SEQ ID NO: 1: at position 452 an amino acid selected from the group consisting of: K and N; at position 585 an amino acid selected from the group consisting of: E, N, M, C, and Q; at position 586 an amino acid selected from the group consisting of: A, M, G, D, N and S; at position 587 an amino acid selected from the group consisting of: T, N, V and A; at position 588 an amino acid selected from the group consisting of: V, Y, T, S, I and Q; at position 589 an amino acid selected from the group consisting of: S, G, L, I, R and A; and at position 590 an amino acid selected from the group consisting of: I, S, G, R and Q. In some embodiments, the capsid protein comprises, relative to reference sequence SEQ ID NO: 1: at position 585 an amino acid selected from the group consisting of: E, N, M, and C: at position 586 an amino acid selected from the group consisting of: A, M, G, D, and N; at position 587 an amino acid selected from the group consisting of: T, N, and V; at position 588 an amino acid selected from the group consisting of: V, Y, T, S, and I; at position 589 an amino acid selected from the group consisting of: S, G, L, I and R; and/or at position 590 an amino acid selected from the group consisting of: I, S, G, and R. In some embodiments, the capsid protein comprises, relative to reference sequence SEQ ID NO: 1, at least two, three, four, five, six or all seven of any of the following: (i) at position 452 amino acid K; (ii) at position 585 an amino acid selected from the group consisting of: E, N, M, and C; (iii) at position 586 an amino acid selected from the group consisting of: A, M, G, D, and N; (iv) at position 587 an amino acid selected from the group consisting of: T, N, and V; (v) at position 588 an amino acid selected from the group consisting of: V, Y, T, S, and I; (vi) at position 589 an amino acid selected from the group consisting of: S, G, L, I and R; and (vii) at position 590 an amino acid selected from the group consisting of: I, S, G, and R. In some embodiments, the capsid protein comprises, relative to reference sequence SEQ ID NO: 1: at position 452 an amino acid selected from the group consisting of: K and N; and at position 587 amino acid substitution A587T; and optionally comprises amino acid N or R at one, two or more positions selected from the group consisting of: 584, 585, 586, 588, 589, and 590. In some embodiments, the capsid protein comprises, relative to reference sequence SEQ ID NO: 1: at position 452 an amino acid selected from the group consisting of: K and N; and amino acid N or R at one, two or more positions selected from the group consisting of: 584, 585, 586, 588, 589, and 590. In some embodiments, the capsid protein comprises, relative to reference sequence SEQ ID NO: 1: at position 452 an amino acid selected from the group consisting of: K and N; and amino acid S at two or more positions selected from the group consisting of: 585, 586, 587, 588, 589 and 590. In some embodiments, the capsid protein comprises, relative to reference sequence SEQ ID NO: 1: at position 452 an amino acid selected from the group consisting of: K and N; and at three, four, five or six positions in the region 585-590 of the VR-VIII site, amino acids selected from the group consisting of: N, S, T, R, and I. In some embodiments, the capsid protein comprises, relative to reference sequence SEQ ID NO: 1: at three, four, five or six positions in the region 585-590 of the VR-VIII site, amino acids selected from the group consisting of: N, S, T, and R. In some embodiments, the capsid protein comprises, relative to reference sequence SEQ ID NO: 1, amino acid substitutions Q585E, S586N, A587T, Q588V, A589S, Q590I, and N452K. In some embodiments, the capsid protein comprises, relative to reference sequence SEQ ID NO: 1, amino acid substitutions S586T, A587L, Q588F, A589N, Q590S, and N452K. In some embodiments, the capsid protein comprises, relative to reference sequence SEQ ID NO: 1, amino acid substitutions Q585N, A587T, Q588Y, A589L, Q590G, and N452K. In some embodiments, the capsid protein comprises, relative to reference sequence SEQ ID NO: 1, amino acid substitutions Q585G, A587I, Q588L, A589T, Q590H, and N452K. In some embodiments, the capsid protein comprises, relative to reference sequence SEQ ID NO: 1, amino acid substitutions Q585M, S586M, A587T, Q588T, and Q590R; and amino acid N at position 452. In some embodiments, the capsid protein comprises, relative to reference sequence SEQ ID NO: 1, amino acid substitutions Q585N, A587T, Q588Y, A589L, and Q590G; and amino acid N at position 452, in some embodiments, the capsid protein comprises, relative to reference sequence SEQ ID NO: 1, amino acid substitutions Q585C, A587T, Q588S, A589I, and Q590R; and amino acid N at position 452. In some embodiments, the capsid protein comprises, relative to reference sequence SEQ ID NO: 1, amino acid substitutions Q585E, S586D, A587N, Q588I, A589R, and Q590S; and amino acid N at position 452. In some embodiments, the capsid protein comprises, relative to reference sequence SEQ ID NO: 1, amino acid substitutions Q585E, S586D, A587N, Q588I, A589R, Q590S, and N452K. In some embodiments, the capsid protein comprises, relative to reference sequence SEQ ID NO: 1, amino acid substitutions Q585N, S586N, A587V, Q588I, A589S, Q590G, and N452K. In some embodiments, the capsid protein comprises, relative to reference sequence SEQ ID NO: 1, amino acid substitutions S586G and Q588Y; and amino acid N at position 452. In some embodiments, the capsid protein comprises, relative to reference sequence SEQ ID NO: 1, amino acid substitutions S586A, A587N, Q588Y, A589G, and N452K. In some embodiments, the capsid protein comprises, relative to reference sequence SEQ ID NO:1, amino acids ATN at positions 581-583, and amino acids AQTG at positions 591-594. In some embodiments, the capsid protein comprises, relative to reference sequence SEQ ID NO:1, amino acids ATNH at positions 581-584, and amino acids AQTG at positions 591-594. In some embodiments, the capsid protein comprises, relative to reference sequence SEQ ID NO:1: (i) amino acid sequence ATNHENTVSIAQTG at the VR-VIII positions 581-594, and amino acid K at the VR-IV position 452; (ii) amino acid sequence ATNHQTLFNSAQTG at the VR-VIII positions 581-594, and amino acid K at the VR-IV position 452; (iii) amino acid sequence ATNHNSTYLGAQTG at the VR-VIII positions 581-594, and amino acid K at the VR-IV position 452: (iv) amino acid sequence ATNHGSILTHAQTG at the VR-VIII positions 581-594, and amino acid K at the VR-IV position 452; (v) amino acid sequence ATNHMMTTARAQTG at the VR-VIII positions 581-594, and amino acid N at the VR-IV position 452; (vi) amino acid sequence ATNHNSTYLGAQTG at the VR-VIII positions 581-594, and amino acid N at the VR-IV position 452; (vii) amino acid sequence ATNHCSTSIRAQTG at the VR-VIII positions 581-594, and amino acid N at the VR-IV position 452: (viii) amino acid sequence ATNHEDNIRSAQTG at the VR-VIII positions 581-594, and amino acid N at the VR-IV position 452; (ix) amino acid sequence ATNHEDNIRSAQTG at the VR-VIII positions 581-594, and amino acid K at the VR-IV position 452; (x) amino acid sequence ATNHNNVISGAQTG at the VR-VIII positions 581-594, and amino acid K at the VR-IV position 452; (xi) amino acid sequence ATNHQGAYAQAQTG at the VR-VIII positions 581-594, and amino acid N at the VR-IV position 452; (xii) amino acid sequence ATNHQANYGQAQTG at the VR-VIII positions 581-594, and amino acid K at the VR-IV position 452; (xiii) amino acid sequence ATNHNMNRVNAQTG at the VR-VIII positions 581-594, and amino acid N at the VR-IV position 452; (xiv) amino acid sequence ATNHNNVISGAQTG at the VR-VIII positions 581-594, and amino acid N at the VR-IV position 452; (xv) amino acid sequence ATNHSNSVQSAQTG at the VR-VIII positions 581-594, and amino acid N at the VR-IV position 452; (xvi) amino acid sequence ATNHSSTFQGAQTG at the VR-VIII positions 581-594, and amino acid N at the VR-IV position 452; (xvii) amino acid sequence ATNHVSSFTSAQTG at the VR-VIII positions 581-594, and amino acid N at the VR-IV position 452; (xviii) amino acid sequence ATNHSTTNFRAQTG at the VR-VIII positions 581-594, and amino acid N at the VR-IV position 452; (xix) amino acid sequence ATNHSSIFNSAQTG at the VR-VIII positions 581-594, and amino acid N at the VR-IV position 452; (xx) amino acid sequence ATNHAGNYNNAQTG at the VR-VIII positions 581-594, and amino acid N at the VR-IV position 452; (xxi) amino acid sequence ATNHTSVISIAQTG at the VR-VIII positions 581-594, and amino acid N at the VR-IV position 452; (xxii) amino acid sequence ATNHHSRVEIAQTG at the VR-VIII positions 581-594, and amino acid N at the VR-IV position 452; (xxiii) amino acid sequence ATNHSSIIYSAQTG at the VR-VIII positions 581-594, and amino acid N at the VR-IV position 452; (xxiv) amino acid sequence ATNHSGRDSYAQTG at the VR-VIII positions 581-594, and amino acid N at the VR-IV position 452; (xxv) amino acid sequence ATNHSSSYNNAQTG at the VR-VIII positions 581-594, and amino acid N at the VR-IV position 452; (xxvi) amino acid sequence ATNIHHINPSINAQTG at the VR-VIII positions 581-594, and amino acid N at the VR-IV position 452; (xxvii) amino acid sequence ATNHNRNGLLAQTG at the VR-VIII positions 581-594, and amino acid N at the VR-IV position 452; (xxviii) amino acid sequence ATNHESTSVRAQTG at the VR-VIII positions 581-594, and amino acid N at the VR-IV position 452: (xxix) amino acid sequence ATNHNIRTEMAQTG at the VR-VIII positions 581-594, and amino acid N at the VR-IV position 452; (xxx) amino acid sequence ATNHQTLFNSAQTG at the VR-VIII positions 581-594, and amino acid N at the VR-IV position 452; (xxxi) amino acid sequence ATNHLSVSSIAQTG at the VR-VIII positions 581-594, and amino acid N at the VR-IV position 452; (xxxii) amino acid sequence ATNHEDIIRSAQTG at the VR-VIII positions 581-594, and amino acid N at the VR-IV position 452; (xxxiii) amino acid sequence ATNRQTAQAQAQTG at the VR-VIII positions 581-594, and amino acid N at the VR-IV position 452; or (xxxiv) amino acid sequence ATNRQIAQAQAQTG at the VR-VIII positions 581-594, and amino acid N at the VR-IV position 452. In some embodiments, the capsid protein comprises, relative to reference sequence SEQ ID NO: 1: (i) an amino acid insertion at position 584 comprising one or more of an asparagine (N), a threonine (T), a tyrosine (Y), phenylalanine (F), and an alanine (A); (ii) an amino acid insertion at position 585 comprising one or more of a histidine (H) and a methionine (M); (iii) an amino acid insertion at position 586 comprising one or more of a histidine (H), a tyrosine (Y), a valine (V), a threonine (T), an alanine (A), an isoleucine (I), a tryptophan (W), a methionine (M), and a leucine; (iv) an amino acid insertion at position 587 comprising one or more of an isoleucine (I) and a proline (P); (v) an amino acid insertion at position 588 comprising one or more of an isoleucine (I), a threonine (T), and a proline (P); and/or (vi) an amino acid insertion at position 589 comprising one or more of a glycine (G) and a glutamine (Q). In some embodiments, the capsid protein comprises, relative to reference sequence SEQ ID NO: 1: (i) an amino acid insertion at position 584 consisting of a TY, FN, or AT: (ii) an amino acid insertion at position 585 consisting of MH; (iii) an amino acid insertion at position 586 consisting of HY, VT, Al, WM, or ML; (iv) an amino acid insertion at position 587 consisting of PI; and/or (v) an amino acid insertion at position 588 consisting of IT or PT. In some embodiments, the capsid protein shares, or comprises a sequence sharing, at least 90%, at least 95%, at least 96%, at least 97%, at least 99% a, or 100% amino acid sequence identity to an AAV9 VP3 sequence according to SEQ ID NO: 487, except for the specified modifications. In some embodiments, the capsid protein shares, or comprises a sequence sharing, at least 90%”, at least 95%, at least 96%, at least 97%, at least 99%6, or 100% amino acid sequence identity to an AAV9 VP2 sequence according to SEQ ID NO: 486, except for the specified modifications. In some embodiments, the capsid protein shares, or comprises a sequence sharing, at least 90%, at least 95%, at least 96%, at least 97%, at least 99%, or 100% amino acid sequence identity to an AAV9 VP1 sequence according to SEQ ID NO: 1, except for the specified modifications. In some embodiments, the capsid protein comprises, consists essentially of, or consists of an amino acid sequence at least 85%, 90%, 95%, 96%, 97%, 98%, 99%, or 100% identical to any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof. In some embodiments, the capsid protein comprises, consists essentially of, or consists of a polypeptide sequence of any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706. 707, 708, 710, 772, and 774.

In some embodiments, the rAAV virion transduces heart cells. In some embodiments, the rAAV virion transduces cardiomyocytes. In some embodiments, the rAAV virion traffics to at least one organ other than the liver. In some embodiments, the rAAV virion traffics to the heart. In some embodiments, the rAAV virion exhibits a higher heart transduction efficiency than an rAAV virion having an AAV9 VP1 capsid protein according to SEQ ID NO: 1. In some embodiments, the rAAV virion exhibits a higher heart-to-liver transduction ratio than an rAAV virion having an AAV9 VP1 capsid protein according to SEQ ID NO: 1, optionally at least 2, 3, 4, 5, 6, 7, 8, 9 or 10 times higher. In some embodiments, administration of the rAAV virion to a subject leads to a lower liver viral load than administration of an rAAV virion having an AAV9 VP1 capsid protein according to SEQ ID NO: 1, optionally at least 2, 3, 4, 5, 6, 7, 8, 9 or 10 times lower. In some embodiments, the rAAV virion exhibits a higher transduction efficiency, optionally higher heart transduction efficiency, than an rAAV virion having an AAV9 VP1 capsid protein according to SEQ ID NO: 1, assessed in a primate. In some embodiments, the rAAV virion exhibits a higher heart-to-liver transduction ratio than an rAAV virion having an AAV9 VP1 capsid protein according to SEQ ID NO: 1, assessed in a primate, optionally at least 2, 3, 4, 5, 6, 7, 8, 9 or 10 times higher. In some embodiments, administration of the rAAV virion to a subject leads to a lower liver viral load than administration of an rAAV virion having an AAV9 VP1 capsid protein according to SEQ ID NO: 1, assessed in a primate, optionally at least 2, 3, 4, 5, 6, 7, 8, 9 or 10 times lower.

In some embodiments, the PKP2 expression cassette comprises a sequence having at least 95% identity to SEQ ID NO: 782 or SEQ ID NO: 783. In some embodiments, the PKP2 expression cassette comprises a nucleic acid sequence having at least 95% identity to SEQ ID NO: 786. In some embodiments, the PKP2 expression cassette comprises a cardiac specific promoter. In some embodiments, the cardiac specific promoter directs gene expression in the myocardium, the epicardium, or both. In some embodiments, the cardiac specific promoter is a troponin promoter, or an alpha-myosin heavy chain promoter. In some embodiments, the troponin promoter has a nucleic acid sequence having at least 95% identity to SEQ ID NO: 784. In some embodiments, the PKP2 expression cassette comprises a PKP2 promoter. In some embodiments, the PKP2 promoter has a nucleic acid sequence having at least 95% identity to SEQ ID NO: 785. In some embodiments, the PKP2 expression cassette comprises a constitutive promoter. In some embodiments, the constitutive promoter is a beta-actin promoter. In some embodiments, the PKP2 expression cassette comprises a cardiac specific enhancer. In some embodiments, the PKP2 expression cassette comprises a 3′ element. In some embodiments, the 3′ element comprises a Woodchuck Hepatitis Virus Posttranscriptional Regulatory Element (WPRE), a bovine growth hormone polyadenylation (bGH polyA) sequence, or a combination thereof.

In some embodiments, the rAAV virions disclosed herein comprise an AAV9 capsid protein as disclosed herein. In some embodiments, the rAAV virions disclosed herein comprise a chimeric AAV5/AAV9 capsid protein as disclosed herein. In some embodiments, the rAAV virions disclosed herein comprise a combinatory capsid protein as disclosed herein.

In some embodiments, the AAV9 capsid protein described herein comprises a sequence that shares at least about 80%, 85%, 90%, 95%, 96%, 97%, 98%, 99%, 99.5%, or 100% identity to SEQ ID NO: 1, as shown below. In some embodiments, the AAV9 capsid protein described herein comprises a sequence that shares at least about 80%, 85%, 90%, 95%, 96%, 97%, 98%, 99%, 99.5%, or 100% identity to SEQ ID NO: 487. The N-terminal residue of VP1, VP2, and VP3, as well as the VR sites (VR-IV, VR-V, VR-VII and VR-VIII), are indicated (in bold, and underlined) in the sequence of full-length VP1 (SEQ ID NO: 1) below. The wild type AAV9 VP1 has the amino acid sequence of SEQ ID NO:1. The wild type AAV9 VP2 has the amino acid sequence of SEQ ID NO:486. The wild type AAV9 VP3 has the amino acid sequence of SEQ ID NO:487.

```
VP1→
(SEQ ID NO: 1)
MAADGYLPDWLEDNISEGIREWWALKPGAPQPKANQQHQDNARGLVLPGYKYLGPGNGLDKGEP
VNAADAAALEHDKAYDQQLKAGDNPYLKYNHADAEFQERLKEDTSEGGNLGRAVFQAKKRLL
VP2→
(SEQ ID NO: 486)
EPLGIVEEAAKTAPGKKRPVEQSPQEPDSSAGIGKSGAQPAKKRLNFGQTGDTESVPDPQPIG
VP3→
(SEQ ID NO: 487)
EPPAAPSGVGSLTMASGGGAPVADNNEGADGVGSSSGNWHCDSQWLGDRVITTSTRTWALPTYN
NHLYKQISNSTSGGSSNDNAYFGYSTPWGYFDFNRFHCHFSPRDWQRLINNNWGFRPKRLNFKL
FNIQVKEVTDNNGVKTIANNLTSTVQVFTDSDYQLPYVLGSAHEGCLPPFPADVFMIPQYGYLT
LNDGSQAVGRSSFYCLEYFPSQMLRTGNNFQFSYEFENVPFHSSYASQSLDRLMNPLID
                      VR-IV VR-V
QYLYYISKTINGSGQNQQTLKFSVAGPSNMAVQGRNYIPGPSYRQQRVSTTVTQNNNSEFAWP
                                                                                        VR-VII
GASSWALNGRNSLMNPGPAMASHKEGEDRFPPLSGSLIFGKQGTGRDNVDADKVMITNEEEIK
                                  VR-VIII
TTNPVATESYGQVATNHQSAQAQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWELQKENSKRWNPEIQYTSNYYK
SNNVEFAVNTEGVYSEPRPIGTRYLTRNL.
```

### Methods of Treatment and Uses

PKP2 gene therapy vectors provided herein in various aspects are useful for treating an individual with a heart disease or condition. In additional aspects PKP2 gene therapy vectors provided herein are for use in treating an individual with a heart disease or condition. “Treating” or “treatment of a condition or subject in need thereof” refers to (I) taking steps to obtain beneficial or desired results, including clinical results such as the reduction of symptoms; (2) preventing the disease, for example, causing the clinical symptoms of the disease not to develop in a patient that is predisposed to the disease, for example a carrier of a genetic mutation in a desmosome gene such as PKP2, but does not yet experience or display symptoms of the disease; (3) inhibiting the disease, for example, arresting or reducing the development of the disease or its clinical symptoms; (4) relieving the disease, for example, causing regression of the disease or its clinical symptoms; or (5) delaying the disease. In one aspect, provided herein are methods for treating a heart disease or disorder in an individual in need thereof. In some cases, the method comprises administering a composition comprising a gene therapy vector comprising a nucleic acid encoding a plakophilin 2 (PKP2) polypeptide or a fragment thereof operatively linked to at least one promoter and a pharmaceutically acceptable carrier or excipient. In some cases, the heart disease or disorder is arrhythmogenic right ventricular cardiomyopathy (ARVC) or arrhythmogenic cardiomyopathy (ACM). In some cases, methods of treatment herein reduce at least one symptom of a arrhythmogenic cardiomyopathy, including but not limited to the method reverses, reduces, or prevents at least one of fibrofatty tissue replacement; myocardial atrophy; predominant right ventricular dilation; ventricular arrhythmias; sudden cardiac death; or exercise-triggered cardiac events; right ventricular cardiomyopathy, dilation, or heart failure; left ventricular cardiomyopathy, dilation, or heart failure; atrial arrhythmias; syncope; palpitations; shortness of breath; or chest pain. In some embodiments, the method reverses, reduces, or prevents fibrofatty tissue replacement in the myocardium, the epicardium, or both. In some cases, the method restores desmosome structure and/or function. In some cases, the method restores PKP2 mRNA expression and/or PKP2 protein and activity levels. In some cases, the method restores PKP2 induced gene expression. In some cases, PKP2 induced gene expression comprises expression of genes whose expression are direct or indirect causal factors leading to one or more disease phenotypes. In some embodiments, the method restores expression of one or more genes having a direct or indirect effect on one or more symptoms of the heart disease. In some cases, the method restores expression of one or more of Ryanodine Receptor 2 (Ryr2), Ankyrin-B (Ank2), Cacnalc (CaV1.2), triadin (Trdn), or calsequestrin-2 (Casq2).

In some embodiments of methods of treatment provided herein, the gene therapy vector comprises a viral vector. Any suitable viral vector is contemplated for use in methods herein including but not limited to a viral vector selected from the group consisting of an adeno-associated virus, an adenovirus, a lentivirus, a pox virus, a vaccinia virus, and a herpes virus. In some cases, the gene therapy vector is an adeno-associated virus. In some cases, the adeno-associated virus is selected from the group consisting of an AAV6, an AAV8, and an AAV9, or a derivative thereof. In some cases, the adeno-associated virus is an AAV9 or a derivative thereof. In some cases, the AAV9 has a nucleic acid sequence with at least 80%, 85%, 90%, 95%, or 99% identity to SEQ ID NO: 7. In some cases, the adeno-associated virus is modified to improve transduction of affected cells in the myocardium or the epicardium, such as cardiomyocytes, for example, in some cases, the adeno-associated virus is a derivative of an AAV6, an AAV8, or an AAV9. In some cases, the derivative is any AAV described in U.S. Patent Application No. 63/012,703, which is hereby incorporated by reference in its entirety.

In some embodiments or methods of treatment provided herein, the composition comprising a gene therapy vector is administered through any suitable route to reach the affected cells. For example, in some cases, the composition is administered intravenously, intracardially, pericardially, or intraarterially.

In some embodiments of methods of treatment provided herein, PKP2 is expressed by any promoter suitable for expression in the affected cells and tissues in the myocardium or the epicardium, for example cardiomyocytes. For example, in some cases, the promoter is a cardiac specific promoter. In some cases, the cardiac specific promoter is a troponin promoter or an alpha-myosin heavy chain promoter. In some cases, the promoter is a PKP2 promoter. In some cases, a cardiac specific enhancer is combined with the promoter. In some cases, the troponin promoter has a nucleic acid sequence having at least 80%, 85%, 90%, 95%, or 99% identity to SEQ ID NO: 784. In some cases, the PKP2 promoter has a nucleic acid sequence having at least 80%, 85%, 90%, 95%, or 99% identity to SEQ ID NO: 785. In some cases, the promoter is a constitutive promoter. In some cases, the constitutive promoter is a beta-actin promoter.

In some embodiments of methods of treatment provided herein the nucleic acid encoding the PKP2 gene has any suitable sequence encoding a PKP2 polypeptide for example, any nucleic acid encoding a polypeptide having a sequence of SEQ ID NO: 789. For example, in some cases, the PKP2 gene has a sequence having at least 80%, 85%, 90%, 95%, or 99% identity to SEQ ID NO: 782. In some cases, the PKP2 gene has a sequence having at least 80%, 85%, 90%, 95%, or 99% identity to SEQ ID NO: 783. In some cases, the nucleic acid sequence encoding the PKP2 gene is codon optimized.

In some embodiments of methods of treatment provided herein, the gene therapy vector has a gene expression cassette having a size of about 3 kb to about 5 kb. In some embodiments, the gene expression cassette has a size of about 4 kb to about 5 kb. In some embodiments, the gene expression cassette has a size of about 4.2 kb to about 4.8 kb. In some embodiments, the gene expression cassette has a size of about 4.5 kb. In some embodiments, the gene expression cassette has a size no larger than about 5 kb. In some embodiments, the gene expression cassette has a size no larger than about 4.9 kb. In some embodiments, the gene expression cassette has a size no larger than about 4.8 kb. In some embodiments, the gene expression cassette has a size no larger than about 4.7 kb. In some embodiments, the gene expression cassette has a size no larger than about 4.6 kb. In some embodiments, the gene expression cassette has a size no larger than about 4.5 kb. In some embodiments, the gene expression cassette has a size no larger than about 4.4 kb. In some embodiments, the gene expression cassette has a size no larger than about 4.3 kb. In some embodiments, the gene expression cassette has a size no larger than about 4.2 kb. In some embodiments, the gene expression cassette has a size no larger than about 4.1 kb. In some embodiments, the gene expression cassette has a size no larger than about 4 kb. In some embodiments, the gene expression cassette has a size no larger than about 3.9 kb. In some embodiments, the gene expression cassette has a size no larger than about 3.8 kb. In some embodiments, the gene expression cassette has a size no larger than about 3.7 kb. In some embodiments, the gene expression cassette has a size no larger than about 3.6 kb. In some embodiments, the gene expression cassette has a size no larger than about 3.5 kb. In some embodiments, the gene expression cassette has a size of at least about 3.1 kb. In some embodiments, the gene expression cassette has a size of at least about 3.3 kb. In some embodiments, the gene expression cassette has a size of at least about 3.5 kb. In some embodiments, the gene expression cassette has a size of at least about 3.7 kb. In some embodiments, the gene expression cassette has a size of at least about 3.9 kb. In some embodiments, the gene expression cassette has a size of at least about 4.1 kb. In some embodiments, the gene expression cassette has a size of at least about 4.2 kb. In some embodiments, the gene expression cassette has a size of at least about 4.3 kb. In some embodiments, the gene expression cassette has a size of at least about 4.4 kb. In some embodiments, the gene expression cassette has a size of at least about 4.5 kb. In some embodiments, the gene expression cassette has a size of at least about 4.6 kb. In some embodiments, the gene expression cassette has a size of at least about 4.7 kb. In some embodiments, the gene expression cassette has a size of at least about 4.8 kb. In some embodiments, the gene expression cassette has a size of at least about 4.9 kb. In some embodiments, the gene expression cassette has a size of at least about 5 kb.

In various embodiments of methods herein, the gene therapy vector comprising a PKP2 gene is formulated in a composition comprising a pharmaceutically acceptable carrier or excipient. For example, in some cases, the pharmaceutically acceptable carrier or excipient comprises a buffer, a polymer, a salt, or a combination thereof.

In some embodiments of methods of treatment provided herein, the individual is identified as having at least one variation in a desmosome protein. In some cases, the desmosome protein is PKP2. In some cases, the variation comprises a deletion, an insertion, a single nucleotide variation, or a copy number variation. In some cases, the individual is identified as having at least one variation in a desmosome protein via DNA sequencing, PCR, qPCR, in situ hybridization, or another other suitable method of identifying a gene variation in an individual.

### Gene Therapy Vectors

In another aspect, there are provided gene therapy vectors comprising a plakophilin 2 gene operatively linked to at least one promoter. In some cases, the gene therapy vector comprises a viral vector. In some cases, the viral vector is any suitable viral vector for treating a heart disease or condition. In some cases, the viral vector is suitable for delivering a gene to cells in the myocardium, the epicardium, or both. In some cases, the viral vector is selected from the group consisting of an adeno-associated virus, an adenovirus, a lentivirus, a pox virus, a vaccinia virus, and a herpes virus. In some cases, the gene therapy vector is an adeno-associated virus. In some cases, the adeno-associated virus is selected from the group consisting of an AAV6, an AAV8, and an AAV9, or a derivative thereof. In some cases, the adeno-associated virus is an AAV9 or a derivative thereof. In some cases, the AAV9 has a nucleic acid sequence with at least 95% identity SEQ ID NO: 711. In some cases, the adeno-associated virus is a derivative of AAV6, AAV8, or AAV9, optimized for transducing cells according to methods of treatment herein. In some cases, the derivative is any AAV described in U.S. Patent Application No. 63/012,703, which is hereby incorporated by reference in its entirety.

In some embodiments of gene therapy vectors provided herein, PKP2 is expressed by any promoter suitable for expression in the affected cells and tissues, for example cardiomyocytes. In some cases, PKP2 is expressed by a promoter that is active in cells of the myocardium, the epicardium, or both. For example, in some cases, the promoter is a cardiac specific promoter. In some cases, the cardiac specific promoter is a troponin promoter or an alpha-myosin heavy chain promoter. In some cases, the promoter is a PKP2 promoter. In some cases, a cardiac specific enhancer is combined with the promoter. In some cases, the troponin promoter has a nucleic acid sequence having at least 80%, 85%, 90%, 95%, or 99% identity to SEQ ID NO: 784. In some cases, the PKP2 promoter has a nucleic acid sequence having at least 80%, 85%, 90%, 95%, or 99% identity to SEQ ID NO: 785. In some cases, the promoter is a constitutive promoter. In some cases, the constitutive promoter is a beta-actin promoter.

In some embodiments of gene therapy vectors provided herein the nucleic acid encoding the PKP2 gene has any suitable sequence encoding a PKP2 polypeptide for example, any nucleic acid encoding a polypeptide having a sequence of SEQ ID NO: 712. For example, in some cases, the PKP2 gene has a sequence having at least 80%, 85%, 90%1, 95%, or 99% identity to SEQ ID NO: 782. In some cases, the PKP2 gene has a sequence having at least 80%, 85%, 90%, 95%, or 99% identity to SEQ ID NO: 783. In some cases, the nucleic acid sequence encoding the PKP2 gene is codon optimized.

In some embodiments of gene therapy vectors provided herein, the gene therapy vector comprises a 3′ element. In some embodiments, the 3′ element stabilizes the transcriptional product of the gene therapy vector (e.g., the PKP2 transcript). In some embodiments, the 3′ element comprises a bovine growth hormone (BGH) polyadenylation sequence. In some embodiments, the 3′ element comprises a woodchuck hepatitis virus posttranscriptional regulatory element (WPRE).

In some embodiments of gene therapy vectors provided herein, the gene therapy vector has a gene expression cassette having a size of about 3 kb to about 5 kb. In some embodiments, the gene expression cassette has a size of about 4 kb to about 5 kb. In some embodiments, the gene expression cassette has a size of about 4.2 kb to about 4.8 kb. In some embodiments, the gene expression cassette has a size of about 4.5 kb. In some embodiments, the gene expression cassette has a size no larger than about 5 kb. In some embodiments, the gene expression cassette has a size no larger than about 4.9 kb. In some embodiments, the gene expression cassette has a size no larger than about 4.8 kb. In some embodiments, the gene expression cassette has a size no larger than about 4.7 kb. In some embodiments, the gene expression cassette has a size no larger than about 4.6 kb. In some embodiments, the gene expression cassette has a size no larger than about 4.5 kb. In some embodiments, the gene expression cassette has a size no larger than about 4.4 kb. In some embodiments, the gene expression cassette has a size no larger than about 4.3 kb. In some embodiments, the gene expression cassette has a size no larger than about 4.2 kb. In some embodiments, the gene expression cassette has a size no larger than about 4.1 kb. In some embodiments, the gene expression cassette has a size no larger than about 4 kb. In some embodiments, the gene expression cassette has a size no larger than about 3.9 kb. In some embodiments, the gene expression cassette has a size no larger than about 3.8 kb. In some embodiments, the gene expression cassette has a size no larger than about 3.7 kb. In some embodiments, the gene expression cassette has a size no larger than about 3.6 kb. In some embodiments, the gene expression cassette has a size no larger than about 3.5 kb. In some embodiments, the gene expression cassette has a size of at least about 3.1 kb. In some embodiments, the gene expression cassette has a size of at least about 3.3 kb. In some embodiments, the gene expression cassette has a size of at least about 3.5 kb. In some embodiments, the gene expression cassette has a size of at least about 3.7 kb. In some embodiments, the gene expression cassette has a size of at least about 3.9 kb. In some embodiments, the gene expression cassette has a size of at least about 4.1 kb. In some embodiments, the gene expression cassette has a size of at least about 4.2 kb. In some embodiments, the gene expression cassette has a size of at least about 4.3 kb. In some embodiments, the gene expression cassette has a size of at least about 4.4 kb. In some embodiments, the gene expression cassette has a size of at least about 4.5 kb. In some embodiments, the gene expression cassette has a size of at least about 4.6 kb. In some embodiments, the gene expression cassette has a size of at least about 4.7 kb. In some embodiments, the gene expression cassette has a size of at least about 4.8 kb. In some embodiments, the gene expression cassette has a size of at least about 4.9 kb. In some embodiments, the gene expression cassette has a size of at least about 5 kb.

In various embodiments of gene therapy vectors provided herein, the gene therapy vector comprising a PKP2 gene is formulated in a composition comprising a pharmaceutically acceptable carrier or excipient. For example, in some cases, the pharmaceutically acceptable carrier or excipient comprises a buffer, a polymer, a salt, or a combination thereof.

In some embodiments, gene therapy vectors herein comprise nucleic acid sequences provided in Table 1 below.

TABLE 1
Sequences

| | | SEQ |
|---|---|---|
| | | ID |
| Name | Sequence | NO: |
| Human | ATGGCAGCCCCCGGCGCCCCAGCTGAGTACGGCTACATCGGGAC | 782 |
| PKP2 | CGTCCTGGGCCAGCAGATGCTGGGACAACTGGACAGCTCCAGCC | |
| | TGGCGCTGCCCTCCGAGGCCAAGCTGAAGCTGGCGGGGAGCAGC | |
| | GGCCGCGGCGGCCAGACAGTCAAGAGCCTGCGGATCCAGGAGC | |
| | AGGTGCAGCAGACCCTCGCCCGGAAGGGCCGCAGCTCCGTGGGC | |
| | AACGGAAATCTTCACCGAACCAGCAGTGTTCCTGAGTATGTCTA | |
| | CAACCTACACTTGGTTGAAAATGATTTTGTTGGAGGCCGTTCCCC | |
| | TGTTCCTAAAACCTATGACATGCTAAAGGCTGGCACAACTGCCA | |
| | CTTATGAAGGTCGCTGGGGAAGAGGAACAGCACAGTACAGCTCC | |
| | CAGAAGTCCGTGGAAGAAAGGTCCTTGAGGCATCCTCTGAGGAG | |
| | ACTGGAGATTTCTCCTGACAGCAGCCCGGAGAGGGCTCACTACA | |
| | CGCACAGCGATTACCAGTACAGCCAGAGAAGCCAGGCTGGGCA | |
| | CACCCTGCACCACCAAGAAAGCAGGCGGGCCGCCCTCCTAGTGC | |
| | CACCGAGATATGCTCGTTCCGAGATCGTGGGGGTCAGCCGTGCT | |
| | GGCACCACAAGCAGGCAGCGCCACTTTGACACATACCACAGACA | |
| | GTACCAGCATGGCTCTGTTAGCGACACCGTTTTTGACAGCATCCC | |
| | TGCCAACCCGGCCCTGCTCACGTACCCCAGGCCAGGGACCAGCC | |
| | GCAGCATGGGCAACCTCTTGGAGAAGGAGAACTACCTGACGGC | |
| | AGGGCTCACTGTCGGGCAGGTCAGGCCGCTGGTGCCCCTGCAGC | |
| | CCGTCACTCAGAACAGGGCTTCCAGGTCCTCCTGGCATCAGAGC | |
| | TCCTTCCACAGCACCCGCACGCTGAGGGAAGCTGGGCCCAGTGT | |
| | CGCCGTGGATTCCAGCGGGAGGAGAGCGCACTTGACTGTCGGCC | |
| | AGGCGGCCGCAGGGGGAAGTGGGAATCTGCTCACTGAGAGAAG | |
| | CACTTTCACTGACTCCCAGCTGGGGAATGCAGACATGGAGATGA | |
| | CTCTGGAGCGAGCAGTGAGTATGCTCGAGGCAGACCACATGCTG | |
| | CCATCCAGGATTTCTGCTGCAGCTACTTTCATACAGCACGAGTGC | |
| | TTCCAGAAATCTGAAGCTCGGAAGAGGGTTAACCAGCTTCGTGG | |
| | CATCCTCAAGCTTCTGCAGCTCCTAAAAGTTCAGAATGAAGACG | |
| | TTCAGCGAGCTGTGTGTGGGGCCTTGAGAAACTTAGTATTTGAA | |
| | GACAATGACAACAAATTGGAGGTGGCTGAACTAAATGGGGTAC | |
| | CTCGGCTGCTCCAGGTGCTGAAGCAAACCAGAGACTTGGAGACT | |
| | AAAAAACAAATAACAGGTTTGCTGTGGAATTTGTCATCTAATGA | |
| | CAAACTCAAGAATCTCATGATAACAGAAGCATTGCTTACGCTGA | |
| | CGGAGAATATCATCATCCCCTTTTCTGGGTGGCCTGAAGGAGAC | |
| | TACCCAAAAGCAAATGGTTTGCTCGATTTTGACATATTCTACAAC | |
| | GTCACTGGATGCCTAAGAAACATGAGTTCTGCTGGCGCTGATGG | |
| | GAGAAAAGCGATGAGAAGATGTGACGGACTCATTGACTCACTG | |
| | GTCCATTATGTCAGAGGAACCATTGCAGATTACCAGCCAGATGA | |
| | CAAGGCCACGGAGAATTGTGTGTGCATTCTTCATAACCTCTCCTA | |
| | CCAGCTGGAGGCAGAGCTCCCAGAGAAATATTCCCAGAATATCT | |
| | ATATTCAAAACCGGAATATCCAGACTGACAACAACAAAAGTATT | |
| | GGATGTTTTGGCAGTCGAAGCAGGAAAGTAAAAGAGCAATACC | |
| | AGGACGTGCCGATGCCGGAGGAAAAGAGCAACCCCAAGGGCGT | |
| | GGAGTGGCTGTGGCATTCCATTGTTATAAGGATGTATCTGTCCTT | |
| | GATCGCCAAAAGTGTCCGCAACTACACACAAGAAGCATCCTTAG | |
| | GAGCTCTGCAGAACCTCACGGCCGGAAGTGGACCAATGCCGACA | |
| | TCAGTGGCTCAGACAGTTGTCCAGAAGGAAAGTGGCCTGCAGCA | |
| | CACCCGAAAGATGCTGCATGTTGGTGACCCAAGTGTGAAAAAGA | |
| | CAGCCATCTCGCTGCTGAGGAATCTGTCCCGGAATCTTTCTCTGC | |
| | AGAATGAAATTGCCAAAGAAACTCTCCCTGATTTGGTTTCCATC | |
| | ATTCCTGACACAGTCCCGAGTACTGACCTTCTCATTGAAACTACA | |
| | GCCTCTGCCTGTTACACATTGAACAACATAATCCAAAACAGTTA | |
| | CCAGAATGCACGCGACCTTCTAAACACCGGGGGCATCCAGAAAA | |
| | TTATGGCCATTAGTGCAGGCGATGCCTATGCCTCCAACAAAGCA | |
| | AGTAAAGCTGCTTCCGTCCTTCTGTATTCTCTGTGGGCACACACG | |
| | GAACTGCATCATGCCTACAAGAAGGCTCAGTTTAAGAAGACAGA | |
| | TTTTGTCAACAGCCGGACTGCCAAAGCCTACCACTCCCTTAAAG | |
| | ACTGA | |
| Human | ATGGCTGCTCCTGGTGCTCCTGCCGAGTACGGCTACATCAGAAC | 783 |
| PKP2 | AGTGCTGGGCCAGCAGATCCTGGGACAGCTGGATTCTAGCTCTC | |
| (codon | TGGCCCTGCCTTCTGAGGCCAAGCTGAAACTGGCCGGCAGTTCT | |
| optimized) | GGAAGAGGCGGCCAGACAGTGAAGTCCCTGCGGATCCAAGAAC | |
| | AGGTGCAGCAGACCCTGGCCAGAAAGGGCAGATCTTCTGTCGGC | |
| | AACGGCAACCTGCACAGAACCAGCTCTGTGCCCGAGTACGTGTA | |
| | CAATCTGCACCTGGTGGAAAACGACTTCGTCGGCGGCAGATCCC | |
| | CTGTGCCTAAGACCTACGATATGCTGAAGGCCGGCACCACCGCC | |
| | ACCTATGAAGGCAGATGGGGAAGAGGCACAGCCCAGTACAGCA | |
| | GCCAGAAAAGCGTGGAAGAGAGAAGCCTGCGGCACCCTCTGCG | |
| | GAGACTGGAAATCAGCCCTGATAGCAGCCCAGAGAGAGCCCAC | |
| | TACACCCACAGCGACTACCAGTACTCCCAGAGATCTCAGGCCGG | |
| | CCACACACTGCACCACCAAGAGTCTAGAAGGGCCGCTCTGCTGG | |
| | TGCCTCCTAGATACGCCAGATCTGAGATCGTGGGCGTGTCCAGA | |
| | GCCGGCACAACAAGCAGACAGAGACACTTCGACACCTACCACC | |
| | GGCAGTATCAGCACGGCAGCGTGTCCGATACCGTGTTCGATAGC | |
| | ATCCCCGCCAATCCTGCTCTGCTGACATACCCTAGACCTGGCACC | |
| | TCCAGATGCATGGGCAATCTGCTGGAAAAAGAGAACTACCTGAC | |
| | CGCCGGACTGACCGTGGGACAAGTTCGACCTCTGGTTCCTCTGC | |
| | AGCCCGTGACACAGAACAGAGCCAGCAGAAGCAGCTGGCACCA | |
| | GTCCAGCTTCCACAGCACCAGAACACTGAGAGAAGCTGGCCCTA | |
| | GCGTGGCCGTGGATTCTTCTGGTAGAAGGGCTCACCTGACAGTT | |
| | GGCCAAGCAGCTGCAGGCGGAAGCGGAAATCTGCTGACCGAGA | |
| | GAAGCACCTTCACCGACAGCCAGCTGGGCAACGCCGACATGGA | |
| | AATGACACTGGAACGGGCCGTGTCCATGCTGGAAGCCGATCACA | |
| | TGCTGCCCAGCAGAATTAGCGCCGCTGCCACCTTTATCCAGCAC | |
| | GAGTGCTTCCAGAAGTCTGAGGCCCGGAAGAGAGTGAACCAGCT | |
| | GAGAGGCATCCTGAAGCTGCTGCAGCTCCTGAAGGTGCAGAACG | |
| | AGGATGTGCAGAGGGCTGTGTGTGGGGCCCTGAGAAATCTGGTG | |
| | TTCGAGGACAACGACAACAAGCTGGAAGTGGCCGAGCTGAACG | |
| | GCGTGCCAAGACTGCTGCAGGTTCTGAAACAGACCCGCGACCTG | |
| | GAAACAAAGAAGCAGATCACCGGCCTGCTCTGGAACCTGAGCA | |
| | GCAACGACAAGCTGAAGAACCTGATGATCACAGAGGCCCTGCTG | |
| | ACCCTGACAGAGAACATCATCATCCCTTTCAGCGGCTGGCCCGA | |
| | GGGCGATTACCCTAAAGCTAATGGCCTGCTGGACTTCGACATCT | |
| | TCTACAACGTGACCGGCTGCCTGAGAAACATGTCTAGCGCTGGC | |
| | GCCGATGGCAGAAAGGCCATGAGAAGATGTGACGGCCTGATCG | |
| | ACAGCCTGGTGCACTATGTGCGGGGCACAATCGCCGATTACCAG | |
| | CCTGATGATAAGGCCACCGAGAACTGCGTGTGCATCCTGCACAA | |
| | CCTGAGCTACCAGCTGGAAGCAGAGCTGCCCGAGAAGTACAGCC | |
| | AGAACATCTACATCCAGAACCGGAACATCCAGACCGACAACAA | |
| | CAAGAGCATCGGCTGCTTCGGCAGCCGCAGCCGGAAAGTGAAA | |
| | GAACAGTACCAGGACGTGCCCATGCCTGAGGAAAAGTCTAACCC | |
| | CAAAGGCGTGGAATGGCTGTGGCACAGCATCGTGATGCGGATGT | |
| | ACCTGAGCCTGATCGCCAAGAGCGTGCGGAATTACACGCAAGAG | |
| | GCATCTCTGGGCGCCCTGCAGAATCTGACAGCAGGATCTGGCCC | |
| | TATGCCTACCTCTGTGGCTCAGACCGTGGTGCAGAAAGAGTCTG | |
| | GCCTGCAGCACACCCGGAAGATGCTGCATGTGGGAGATGCCAGC | |
| | GTGAAGAAAACCGCCATCAGCCTGCTGAGAAACCTGAGCCGGA | |
| | ATCTGTCTCTGCAGAATGAGATCGCCAAAGAGACACTGCCCGAC | |
| | CTGGTGTCTATCATCCCTGACACCGTGCCTAGCACCGACCTGCTG | |
| | ATTGAGACAACAGCCAGCGCCTGCTACACCCTGAACAACATCAT | |
| | TCAGAACTCCTACCAGAACGCCCGCGATCTGCTGAACACAGGCG | |
| | GCATCCAGAAAATCATGGCCATCTCTGCCGGCGACGCCTACGCC | |
| | TCTAACAAGGCCTCTAAAGCCGCCAGCGTGCTGCTGTATTCTCTG | |
| | TGGGCCCATACCGAGCTGCACCATGCCTATAAGAAGGCCCAGTT | |
| | CAAAAAGACCGACTTCGTGAACAGCCGGACCGCCAAGGCCTACC | |
| | ACTCTCTGAAAGAT | |
| pcTNT | GTCATGGAGAAGACCCACCTTGCAGATGTCCTCACTGGGGCTGG | 784 |
| Promoter | CAGAGCCGGCAACCTGCCTAAGGCTGCTCAGTCCATTAGGAGCC | |
| | AGTAGCCTGGAAGATGTCTTTACCCCCAGCATCAGTTCAAGTGG | |
| | AGCAGCACATAACTCTTGCCCTCTGCCTTCCAAGATTCTGGTGCT | |
| | GAGACTTATGGAGTGTCTTGGAGGTTGCCTTCTGCCCCCCAACCC | |
| | TGCTCCCAGCTGGCCCTCCCAGGCCTGGGTTGCTGGCCTCTGCTT | |
| | TATCAGGATTCTCAAGAGGGACAGCTGGTTTATGTTGCATGACT | |
| | GTTCCCTGCATATCTGCTCTGGTTTTAAATAGCTTATCTGAGCAG | |
| | CTGGAGGACCACATGGGCTTATATGGCGTGGGGTACATGTTCCT | |
| | GTAGCCTTGTCCCTGGCACCTGCCAAAATAGCAGCCAACACCCC | |
| | CCACCCCCACCGCCATCCCCCTGCCCCACCCGTCCCCTGTCGCAC | |
| | ATTCCTCCCTCCGCAGGGCTGGCTCACCAGGCCCCAGCCCACAT | |
| | GCCTGCTTAAAGCCCTCTCCATCCTCTGCCTCACCCAGTGCCGGC | |
| | TGAGACTGAGCAGACGCCTCCA | |
| PKP2 | CATCTCAGCATCATGGTTGGATGTTTCCACCTGGCTACATAAGCA | 785 |
| promoter | AGCTTTACACAAGGTGTAATTTGCCTAAATAGTGGTCCATTCTAT | |
| | TGGGGTGGGAGCAATTGCTTCCAGGACTCACATCCATATGGCTC | |
| | CCACTTAGCCATGTGGCCTGCTGACAAAGGGTGGCGGAACTGTC | |
| | ACTACTCTGTTGTCCACGCTTTCAGTCCTTTGGTTTCCTCTTCACT | |
| | CCCTGGACGCTCATGTAAAAAGGGAGGCCATATACCTGTGCATT | |
| | GTGTGTCTAAGCATTCAGTGTGTGTCTAAAGGCAGAAGGGTGTG | |
| | GGTAGGAAAACAAAGACGAGGGAAGCTGCGTTCTCCAAACACT | |
| | TCAGACTTGAGTAAGTGGGGTTTTGCAGCAATTGAGTGATTTGA | |
| | GGGAAAGTGAACATACAAACCCAAGCAATCAAAGGGAATATTA | |
| | TCTTAATACCAGGGATACATGTTTTTCTTTCTGCCTCTTAAGTCC | |
| | AAAGAGGCAAATCAGGACAAGTGGCTTTGGTTGTAAACTTTAAG | |
| | GTCAAGGATCCTTTCTGTTGAGCTTAGCTCTCAAGTTCTCAGTAG | |
| | TCAACTGCGGTGAAACATAATTAATAGCACGATAAATACAAGTT | |
| | GTGGAAGATTCGATTGAAAGTTGGAGGCCCTCTCCGTGGATCTC | |
| | TCTACAAAGAGCCTGTAATAAAGAGGACTTAATCAACGTTAGCA | |
| | GGGCTATTTAAAAAGCATCGTCTATTAAAATTCATTTCTTCTCTA | |
| | GAGCCTCTTGTTGGAGTTTCTCTGTGTGGGTGTGTTCGTAAGAGA | |
| | GGAATGGGTTAGCAAGAGTACTGGGTACAATTTGTGTATCCAAG | |
| | AGAAAACAGAAGCTCTCAATGAGGAAGAACATATGTTTCTGGGA | |
| | CTGCATCTGTGCAAAAAGTACATAGTCCTGACGTTGTACTAAGA | |
| | AAAAAAACACTCTCTTTAGAAAGTCTTTTATTTCACACGTTATCT | |
| | TCTTGGCACATTTCCCTCATATTGCCCTTTCCGCCTGACCAAATA | |
| | GCCCTTTCTCACCCTCAGGTCCAGGAAAACCAGGAAACGTTTCC | |
| | AACAGTGCGACAAAGCCTGACTAACCAGACATACTACTCGCTCG | |
| | GGGATCCCGGAGGCAAGCCTCAGTCCAAGAACAGGAGTGACTCT | |
| | CGAGGGCTCACCTGCCTGCAGGGCAGCCCCTCCCTGCATCGAGC | |
| | GGAAATCCATCCTGTCCAGCGCGGGGCGTGGGCAGAGCGGGGC | |
| | GCGGCCCCGGCAGGCGGTATCCGCTGGGACTCCGACAACGTGCG | |
| | CGACCCCAGGCGAACCGCGCCCCTCTCCCCACCTCCCCGCGGGC | |
| | GGGTACAAGTCTCCAGGTGTCCGCGCGCTCAGCGGGTCCGGCCC | |
| | GCCCCCGCCCCCGCCCCCGGGCCCGACTGCGCGTGCCCGGCCGG | |
| | AGCCGCGCCCCCTGCTCAGGGAAGGCCGGGCGTCCGGCCCACGA | |
| | GGCCGAGCTCCCCCCCGGCCCGGGCCTCTCACCGGCGCGGGGGG | |
| | CGGGCCAGGGGGGGGGCCGGACTCGAGCGGGGCGGGGCTCGCG | |
| | CCAGCGCCCCCAGCTCCGTGGCGGCTTCGCCCGCGAGTCCAGAG | |
| | GCAGGCGAGCAGCTCGGTCGCCCCCACCGGCCCC | |
| AAV | ctgcgcgctcgctcgctcactgaggccgcccgggcaaagcccgggcgtcgggcgacctttggtcgcccg | 786 |
| Human | gcctcagtgagcgagcgagcgcgcagagagggagtggccaactccatcactaggggttccttgtagttaa | |
| PKP2a | tgattaacccgccatgctacttatctacgtagccatgctctaggaagatcggaattcGCCCTTAAGT | |
| Expression | CATGGAGAAGACCCACCTTGCAGATGTCCTCACTGGGGCTGGCA | |
| Cassette | GAGCCGGCAACCTGCCCAAGGCTGCTCAGTCCATTAGGAGCCAG | |
| (pcTnT | TAGCCTGGAAGATGTCTTTACCCCCAGCATCAGTTCAAGTGGAG | |
| promoter, | CAGCACATAACTCTTGCCCTCTGCCTTCCAAGATTCTGGTGCTGA | |
| codon | GACTTATGGAGTGTCTTGGAGGTTGCCTTCTGCCCCCCAACCCTG | |
| optimized) | CTCCCAGCTGGCCCTCCCAGGCCTGGGTTGCTGGCCTCTGCTTTA | |
| | TCAGGATTCTCAAGAGGGACAGCTGGTTTATGTTGCATGACTGTT | |
| | CCCTGCATATCTGCTCTGGTTTTAAATAGCTTATCTGAGCAGCTG | |
| | GAGGACCACATGGGCTTATATGGCGTGGGGTACATGTTCCTGTA | |
| | GCCTTGTCCCTGGCACCTGCCAAAATAGCAGCCAACACCCCCCA | |
| | CCCCCACCGCCATCCCCCTGCCCCACCCGTCCCCTGTGGCACATT | |
| | CCTCCCTCCGCAGGGCTGGCTCACCAGGCCCCAGCCCACATGCC | |
| | TGCTTAAAGCCCTCTCCATCCTCTGCCTCACCCAGTCCCCGCTGA | |
| | GACTGAGCAGACGCCTCCAGCCACCATGGCTGCTCCTGGTGCTC | |
| | CTGCCGAGTACGGCTACATCAGAACAGTGCTGGGCCAGCAGATC | |
| | CTGGGACAGCTGGATTCTAGCTCTCTGGCCCTGCCTTCTGAGGCC | |
| | AAGCTGAAACTGGCCGGCAGTTCTGGAAGAGGCGGCCAGACAG | |
| | TGAAGTCCCTGCGGATCCAAGAACAGGTGCAGCAGACCCTGGCC | |
| | AGAAAGGGCAGATCTTCTGTCGGCAACGGCAACCTGCACAGAAC | |
| | CAGCTCTGTGCCCGAGTACGTGTACAATCTGCACCTGGTGGAAA | |
| | ACGACTTCGTCGGCGGCAGATCCCCTGTGCCTAAGACCTACGAT | |
| | ATGCTGAAGGCCGGCACCACCGCCACCTATGAAGGCAGATGGG | |
| | GAAGAGGCACAGCCCAGTACAGCAGCCAGAAAAGCGTGGAAGA | |
| | GAGAAGCCTGCGGCACCCTCTGCGGAGACTGGAAATCAGCCCTG | |
| | ATAGCAGCCCAGAGAGAGCCCACTACACCCACAGCGACTACCA | |
| | GTACTCCCAGAGATCTCAGGCCGGCCACACACTGCACCACCAAG | |
| | AGTCTAGAAGGGCCGCTCTGCTGGTGCCTCCTAGATACGCCAGA | |
| | TCTGAGATCGTGGGCGTGTCCAGAGCCGGCACAACAAGCAGACA | |
| | GAGACACTTCGACACCTACCACCGGCAGTATCAGCACGGCAGCG | |
| | TGTCCGATACCGTGTTCGATAGCATCCCCGCCAATCCTGCTCTGC | |
| | TGACATACCCTAGACCTGGCACCTCCAGATCCATGGGCAATCTG | |
| | CTGGAAAAAGAGAACTACCTGACCGCCGGACTGACCGTGGGAC | |
| | AAGTTCGACCTCTGGTTCCTCTGCAGCCCGTGACACAGAACAGA | |
| | GCCAGCAGAAGCAGCTGGCACCAGTCCAGCTTCCACAGCACCAG | |
| | AACACTGAGAGAAGCTGGCCCTAGCGTGGCCGTGGATTCTTCTG | |
| | GTAGAAGGGCTCACCTGACAGTTGGCCAAGCAGCTGCAGGCGG | |
| | AAGCGGAAATCTGCTGACCGAGAGAAGCACCTTCACCGACAGCC | |
| | AGCTGGGCAACGCCGACATGGAAATGACACTGGAACGGGCCGT | |
| | GTCCATGCTGGAAGCCGATCACATGCTGCCCAGCAGAATTAGCG | |
| | CCGCTGCCACCTTTATCCAGCACGAGTGCTTCCAGAAGTCTGAG | |
| | GCCCGGAAGAGAGTGAACCAGCTGAGAGGCATCCTGAAGCTGC | |
| | TGCAGCTCCTGAAGGTGCAGAACGAGGATGTGCAGAGGGCTGTG | |
| | TGTGGGGCCCTGAGAAATCTGGTGTTCGAGGACAACGACAACAA | |
| | GCTGGAAGTGGCCGAGCTGAACGGCGTGCCAAGACTGCTGCAG | |
| | GTTCTGAAACAGACCCGCGACCTGGAAACAAAGAAGCAGATCA | |
| | CCGGCCTGCTCTGGAACCTGAGCAGCAACGACAAGCTGAAGAAC | |
| | CTGATGATCACAGAGGCCCTGCTGACCCTGACAGAGAACATCAT | |
| | CATCCCTTTCAGCGGCTGGCCCGAGGGCGATTACCCTAAAGCTA | |
| | ATGGCCTGCTGGACTTCGACATCTTCTACAACGTGACCGGCTGCC | |
| | TGAGAAACATGTCTAGCGCTGGCGCCGATGGCAGAAAGGCCATG | |
| | AGAAGATGTGACGGCCTGATCGACAGCCTGGTGCACTATGTGCG | |
| | GGGCACAATCGCCGATTACCAGCCTGATGATAAGGCCACCGAGA | |
| | ACTGCGTGTGCATCCTGCACAACCTGAGCTACCAGCTGGAAGCA | |
| | GAGCTGCCCGAGAAGTACAGCCAGAACATCTACATCCAGAACCG | |
| | GAACATCCAGACCGACAACAACAAGAGCATCGGCTGCTTCGGCA | |
| | GCCGCAGCCGGAAAGTGAAAGAACAGTACCAGGACGTGCCCAT | |
| | GCCTGAGGAAAAGTCTAACCCCAAAGGCGTGGAATGGCTGTGGC | |
| | ACAGCATCGTGATCCGGATGTACCTGAGCCTGATCGCCAAGAGC | |
| | GTGCGGAATTACACCCAAGAGGCATCTCTGGGCGCCCTGCAGAA | |
| | TCTGACAGCAGGATCTGGCCCTATGCCTACCTCTGTGGCTCAGAC | |
| | CGTGGTGCAGAAAGAGTCTGGCCTGCAGCACACCCGGAAGATGC | |
| | TGCATGTGGGAGATCCCAGCGTGAAGAAAACCGCCATCAGCCTG | |
| | CTGAGAAACCTGAGCCGGAATCTGTCTCTGCAGAATGAGATCGC | |
| | CAAAGAGACACTGCCCGACCTGGTGTCTATCATCCCTGACACCG | |
| | TGCCTAGCACCGACCTGCTGATTGAGACAACAGCCAGCGCCTGC | |
| | TACACCCTGAACAACATCATTCAGAACTCCTACCAGAACGCCCG | |
| | CGATCTGCTGAACACAGGCGGCATCCAGAAAATCATGGCCATCT | |
| | CTGCCGGCGACGCCTACGCCTCTAACAAGGCCTCTAAAGCCGCC | |
| | AGCGTGCTGCTGTATTCTCTGTGGGCCCATACCGAGCTGCACCAT | |
| | GCCTATAAGAAGGCCCAGTTCAAAAAGACCGACTTCGTGAACAG | |
| | CCGGACCGCCAAGGCCTACCACTCTCTGAAAGATTAAtaagcttggatc | |
| | caatcaacctctggattacaaaatttgtgaaagattgactggtattcttaactatgttgctccttttacgct | |
| | atgtggatacgctgctttaatgcctttgtatcatgctattgcttcccgtatggctttcattttctcctcctt | |
| | gtataaatcctggttgctgtctctttatgaggagttgtggcccgttgtcaggcaacgtggcgtggtgtgcac | |
| | tgtgtttgctgacgcaacccccactggttggggcattgccaccacctgtcagctcctttccgggactttcgc | |
| | tttccccctccctattgccacggcggaactcatcgccgcctgccttgcccgctgctggacaggggctcggct | |
| | gttgggcactgacaattccgtggtgttgtcggggaaATCATcgtcctttccTtggctgctcgcctgtgttgc | |
| | cacctggattctgcgcgggacgtccttctgctacgtcccttcggccctcaatccagcggaccttccttcccg | |
| | cggcctgctgccggctctgcggcctcttccgcgtcttcgagatctgcctcgactgtgccttctagttgccag | |
| | ccatctgttgtttgcccctcccccgtgccttccttgaccctggaaggtgccactcccactgtcctttcctaa | |
| | taaaatgaggaaattgcatcgcattgtctgagtagctctcattctattctggggggtggggtcgcgcaggac | |
| | agcaagggggaggattgggaagacaatagcaggcatgctggggaCTGGGGACTCGAGTTAAGGGCgaattcc | |
| | cgataaggatcttcctagagcatggctacgtagataagtagcatggcgggttaatcattaactacaaggaac | |
| | ccctagtgatggagttggccactccctctctgcgcgctcgctcgctcactgaggccgggcgaccaaaggtcg | |
| | cccgacgcccgggctttgcccgggcggcctcagtgagcgagcgagcgcgcag | |
| AAV | ctgcgcgctcgctcgctcactgaggccgcccgggcaaagcccgggcgtcgggcgacctttggtcgcccg | 787 |
| Human | gcctcagtgagcgagcgagcgcgcagagagggagtggccaactccatcactaggggttccttgtagttaa | |
| PKP2a | tgattaacccgccatgctacttatctacgtagccatgctctaggaagatcggaattcGCCCTTAACA | |
| Expression | TCTCAGCATCATGGTTGGATGTTTCCACCTGGCTACATAAGCAAG | |
| Cassette | CITTACACAAGGTGTAATTTGCCTAAATAGTGGTCCATTCTATTG | |
| (PKP2 | GGGTGGGAGCAATTGCTTCCAGGACTCACATCCATATGGCTCCC | |
| promoter, | ACTTAGCCATGTGGCCTGCTGACAAAGGGTGGCGGAACTGTCAC | |
| codon | TACTCTGTTGTCCACGCTTTCAGTCCTTTGGTTTCCTCTTCACTCC | |
| optimized) | CTGGACGCTCATGTAAAAAGGGAGGCCATATACCTGTGCATTGT | |
| | GTGTCTAAGCATTCAGTGTGTGTCTAAAGGCAGAAGGGTGTGGG | |
| | TAGGAAAACAAAGACGAGGGAAGCTGCGTTCTCCAAACACTTCA | |
| | GACTTGAGTAAGTGGGGTTTTGCAGCAATTGAGTGATTTGAGGG | |
| | AAAGTGAACATACAAACCCAAGCAATCAAAGGGAATATTATCTT | |
| | AATACCAGGGATACATGTTTTTCTTTCTGCCTCTTAAGTCCAAAG | |
| | AGGCAAATCAGGACAAGTGGCTTTGGTTGTAAACTTTAAGGTCA | |
| | AGGATCCTTTCTGTTGAGCTTAGCTCTCAAGTTCTCAGTAGTCAA | |
| | CTGCGGTGAAACATAATTAATAGCACGATAAATACAAGTIGTGG | |
| | AAGATTCGATTGAAAGTTGGAGGCCCTCTCCGTGGATCTCTCTAC | |
| | AAAGAGCCTGTAATAAAGAGGACTTAATCAACGTTAGCAGGGCT | |
| | ATTTAAAAAGCATCGTCTATTAAAATTCATTTCTTCTCTAGAGCC | |
| | TCTTGTTGGAGTTTCTCTGTGTGGGTGTGTTCGTAAGAGAGGAAT | |
| | GGGTTAGCAAGAGTACTGGGTACAATTTGTGTATCCAAGAGAAA | |
| | ACAGAAGCTCTCAATGAGGAAGAACATATGTTTCTGGGACTGCA | |
| | TCTGTGCAAAAAGTACATAGTCCTGACGTTGTACTAAGAAAAAA | |
| | AACACTCTCTTTAGAAAGTCTTTTATTTCACACGTTATCTTCTTGG | |
| | CACATTTCCCTCATATTGCCCTTTCCGCCTGACCAAATAGCCCTT | |
| | TCTCACCCTCAGGTCCAGGAAAACCAGGAAACGTTTCCAACAGT | |
| | GCGACAAAGCCTGACTAACCAGACATACTACTCGCTCGGGGATC | |
| | CCGGAGGCAAGCCTCAGTCCAAGAACAGGAGTGACTCTCGAGG | |
| | GCTCACCTGCCTGCAGGGCAGCCCCTCCCTGCATCGAGCGGAAA | |
| | TCCATCCTGTCCAGCGCGGGGCGTGGGCAGAGGGGGGCGCGGCC | |
| | CCGGCAGGCGGTATCCGCTGGGACTCCGACAACGTGCGCGACCC | |
| | CAGGCGAACCGCGCCCCTCTCCCCACCTCCCCGCGGGCGGGTAC | |
| | AAGTCTCCAGGTGTCCGCGCGCTCAGCGGGTCCGGCCCGCCCCC | |
| | GCCCCCGCCCCCGGGCCCGACTGCGCGTGCCCGGCCGGAGCCGC | |
| | GCCCCCTCCTCAGGGAAGGCCGGGCGTCCGGCCCACGAGGCCGA | |
| | GCTCCCCCCCGGCCCGGGCCTCTCACCGGCGCGGGGGGGGGCC | |
| | AGGGGCGGGGCCGGACTCGAGCGGGGCGGGGCTCGCGCCAGCG | |
| | CCCCCAGCTCCGTGGCGGCTTCGCCCGCGAGTCCAGAGGCAGGC | |
| | GAGCAGCTCGGTCGCCCCCACCGGCCCCATGGCTGCTCCTGGTG | |
| | CTCCTGCCGAGTACGGCTACATCAGAACAGTGCTGGGCCAGCAG | |
| | ATCCTGGGACAGCTGGATTCTAGCTCTCTGGCCCTGCCTTCTGAG | |
| | GCCAAGCTGAAACTGGCCGGCAGTTCTGGAAGAGGCGGCCAGA | |
| | CAGTGAAGTCCCTGCGGATCCAAGAACAGGTGCAGCAGACCCTG | |
| | GCCAGAAAGGGCAGATCTTCTGTCGGCAACGGCAACCTGCACAG | |
| | AACCAGCTCTGTGCCCGAGTACGTGTACAATCTGCACCTGGTGG | |
| | AAAACGACTTCGTCGGCGGCAGATCCCCTGTGCCTAAGACCTAC | |
| | GATATGCTGAAGGCCGGCACCACCGCCACCTATGAAGGCAGATG | |
| | GGGAAGAGGCACAGCCCAGTACAGCAGCCAGAAAAGCGTGGAA | |
| | GAGAGAAGCCTGCGGCACCCTCTGCGGAGACTGGAAATCAGCCC | |
| | TGATAGCAGCCCAGAGAGAGCCCACTACACCCACAGCGACTACC | |
| | AGTACTCCCAGAGATCTCAGGCCGGCCACACACTGCACCACCAA | |
| | GAGTCTAGAAGGGCCGCTCTGCTGGTGCCTCCTAGATACGCCAG | |
| | ATCTGAGATCGTGGGCGTGTCCAGAGCCGGCACAACAAGCAGAC | |
| | AGAGACACTTGGACACCTACCACCGGCAGTATCAGCACGGCAGC | |
| | GTGTCCGATACCGTGTTGGATAGCATCCCCGCCAATCCTGCTCTG | |
| | CTGACATACCCTAGACCTGGCACCTCCAGATCCATGGGCAATCT | |
| | GCTGGAAAAAGAGAACTACCTGACCGCCGGACTGACCGTGGGA | |
| | CAAGTTCGACCTCTGGTTCCTCTGCAGCCCGTGACACAGAACAG | |
| | AGCCAGCAGAAGCAGCTGGCACCAGTCCAGCTTCCACAGCACCA | |
| | GAACACTGAGAGAAGCTGGCCCTAGCGTGGCCGTGGATTCTTCT | |
| | GGTAGAAGGGCTCACCTGACAGTTGGCCAAGCAGCTGCAGGCG | |
| | GAAGCGGAAATCTGCTGACCGAGAGAAGCACCTTCACCGACAG | |
| | CCAGCTGGGCAACGCCGACATGGAAATGACACTGGAACGGGCC | |
| | GTGTCCATGCTGGAAGCCGATCACATGCTGCCCAGCAGAATTAG | |
| | CGCCGCTGCCACCTTTATCCAGCACGAGTGCTTCCAGAAGTCTG | |
| | AGGCCCGGAAGAGAGTGAACCAGCTGAGAGGCATCCTGAAGCT | |
| | GCTGCAGCTGCTGAAGGTGCAGAACGAGGATGTGCAGAGGGCT | |
| | GTGTGTGGGGCCCTGAGAAATCTGGTGTTCGAGGACAACGACAA | |
| | CAAGCTGGAAGTGGCCGAGCTGAACGGCGTGCCAAGACTGCTGC | |
| | AGGTTCTGAAACAGACCCGCGACCTGGAAACAAAGAAGCAGAT | |
| | CACCGGCCTGCTCTGGAACCTGAGCAGCAACGACAAGCTGAAGA | |
| | ACCTGATGATCACAGAGGCCCTGCTGACCCTGACAGAGAACATC | |
| | ATCATCCCTTTCAGCGGCTGGCCCGAGGGCGATTACCCTAAAGC | |
| | TAATGGCCTGCTGGACTTCGACATCTTCTACAACGTGACCGGCTG | |
| | CCTGAGAAACATGTCTAGCGCTGGCGCCGATGGCAGAAAGGCCA | |
| | TGAGAAGATGTGACGGCCTGATCGACAGCCTGGTGCACTATGTG | |
| | CGGGGCACAATCGCCGATTACCAGCCTGATGATAAGGCCACCGA | |
| | GAACTGCGTGTGCATCCTGCACAACCTGAGCTACCAGCTGGAAG | |
| | CAGAGCTGCCCGAGAAGTACAGCCAGAACATCTACATCCAGAAC | |
| | CGGAACATCCAGACCGACAACAACAAGAGCATCGGCTGCTTCGG | |
| | CAGCCGCAGCCGGAAAGTGAAAGAACAGTACCAGGACGTGCCC | |
| | ATGCCTGAGGAAAAGTCTAACCCCAAAGGCGTGGAATGGCTGTG | |
| | GCACAGCATCGTGATCCGGATGTACCTGAGCCTGATCGCCAAGA | |
| | GCGTGCGGAATTACACCCAAGAGGCATCTCTGGGCGCCCTGCAG | |
| | AATCTGACAGCAGGATCTGGCCCTATGCCTACCTCTGTGGCTCA | |
| | GACCGTGGTGCAGAAAGAGTCTGGCCTGCAGCACACCCGGAAG | |
| | ATGCTGCATGTGGGAGATCCCAGCGTGAAGAAAACCGCCATCAG | |
| | CCTGCTGAGAAACCTGAGCCGGAATCTGTCTCTGCAGAATGAGA | |
| | TCGCCAAAGAGACACTGCCCGACCTGGTGTCTATCATCCCTGAC | |
| | ACCGTGCCTAGCACCGACCTGCTGATTGAGACAACAGCCAGCGC | |
| | CTGCTACACCCTGAACAACATCATTCAGAACTCCTACCAGAACG | |
| | CCCGCGATCTGCTGAACACAGGCGGCATCCAGAAAATCATGGCC | |
| | ATCTCTGCCGGCGACGCCTACGCCTCTAACAAGGCCTCTAAAGC | |
| | CGCCAGCGTGCTGCTGTATTCTCTGTGGGCCCATACCGAGCTGCA | |
| | CCATGCCTATAAGAAGGCCCAGTTCAAAAAGACCGACTTCGTGA | |
| | ACAGCCGGACCGCCAAGGCCTACCACTCTCTGAAAGATGTCGAC | |
| | GGATCCGGTACCGATTACAAGGACGACGATGACAAGTGAAGCTT | |
| | AATAAAAGATCTTTATTTTCATTAGATCTGTGTGTTGGTTTTTTGT | |
| | GTGCTGGGGACTCGAGTTAAGGGCgaattcccgataaggatcttcctagagcatggc | |
| | tacgtagataagtagcatggcgggttaatcattaactacaaggaacccctagtgatggagttggccactcct | |
| | ctctgcgcgctcgctcgctcactgaggccgggcgaccaaaggtcgcccgacgcccgggctttgcccggg | |
| | cggcctcagtgagcgagcgagcgcgcag | |
| AAV9 | ACGGCGGGGTTTTACGAGATTGTGATTAAGGTCCCCAGCGACCT | 788 |
| genome | TGACGAGCATCTGCCCGGCATTTCTGACAGCTTTGTGAACTGGGT | |
| sequence | GGCCGAGAAGGAATGGGAGTTGCCGCCAGATTCTGACATGGATC | |
| | TGAATCTGATTGAGCAGGCACCCCTGACCGTGGCCGAGAAGCTG | |
| | CAGCGCGACTTTCTGACGGAATGGCGCCGTGTGAGTAAGGCCCC | |
| | GGAGGCCCTTTTCTTTGTGCAATTTGAGAAGGGAGAGAGCTACT | |
| | TCCACATGCACGTGCTCGTGGAAACCACCGGGGTGAAATCCATG | |
| | GTTTTGGGACGTTTCCTGAGTCAGATTCGCGAAAAACTGATTCA | |
| | GAGAATTTACCGCGGGATCGAGCCGACTTTGCCAAACTGGTTCG | |
| | CGGTCACAAAGACCAGAAATGGCGCCGGAGGGGGGAACAAGGT | |
| | GGTGGATGAGTGCTACATCCCCAATTACTTGCTCCCCAAAACCC | |
| | AGCCTGAGCTCCAGTGGGCGTGGACTAATATGGAACAGTATTTA | |
| | AGCGCCTGTTTGAATCTCACGGAGCGTAAACGGTTGGTGGCGCA | |
| | GCATCTGACGCACGTGTCGCAGACGCAGGAGCAGAACAAAGAG | |
| | AATCAGAATCCCAATTCTGATGCGCCGGTGATCAGATCAAAAAC | |
| | TTCAGCCAGGTACATGGAGCTGGTCGGGTGGCTCGTGGACAAGG | |
| | GGATTACCTCGGAGAAGCAGTGGATCCAGGAGGACCAGGCCTC | |
| | ATACATCTCCTTCAATGCGGCCTCCAACTCGCGGTCCCAAATCAA | |
| | GGCTGCCTTGGACAATGCGGGAAAGATTATGAGCCTGACTAAAA | |
| | CCGCCCCCGACTACCTGGTGGGCCAGCAGCCCGTGGAGGACATT | |
| | TCCAGCAATCGGATTTATAAAATTTTGGAACTAAACGGGTACGA | |
| | TCCCCAATATGCGGCTTCCGTCTTTCTGGGATGGGCCACGAAAA | |
| | AGTTCGGCAAGAGGAACACCATCTGGCTGTTTGGGCCTGCAACT | |
| | ACCGGGAAGACCAACATCGCGGAGGCCATAGCCCACACTGTGCC | |
| | CTTCTACGGGTGCGTAAACTGGACCAATGAGAACTTTCCCTTCA | |
| | ACGACTGTGTCGACAAGATGGTGATCTGGTGGGAGGAGGGGAA | |
| | GATGACCGCCAAGGTCGTGGAGTCGGCCAAAGCCATTCTCGGAG | |
| | GAAGCAAGGTGCGCGTGGACCAGAAATGCAAGTCCTCGGCCCA | |
| | GATAGACCCGACTCCCGTGATCGTCACCTCCAACACCAACATGT | |
| | GCGCCGTGATTGACGGGAACTCAACGACCTTCGAACACCAGCAG | |
| | CCGTTGCAAGACCGGATGTTCAAATTTGAACTCACCCGCCGTCT | |
| | GGATCATGACTTTGGGAAGGTCACCAAGCAGGAAGTCAAAGACT | |
| | TTTTCCGGTGGGCAAAGGATCACGTGGTTGAGGTGGAGCATGAA | |
| | TTCTACGTCAAAAAGGGTGGAGCCAAGAAAAGACCCGCCGCCA | |
| | GTGACGCAGATATAAGTGAGCCCAAACGGGTGCGCGAGTCAGTT | |
| | GCGCAGCCATCGACGTCAGACGCGGAAGCTTCGATCAACTACGC | |
| | AGACAGGTACCAAAACAAATGTTCTCGTCACGTGGGCATGAATC | |
| | TGATGCTGTTTCCCTGCAGACAATGCGAGAGAATGAATCAGAAT | |
| | TCAAATATCTGCTTCACTCACGGACAGAAAGACTGTTTAGAGTG | |
| | CTTTCCCGTGTCAGAATCTCAACCCGTTTCTGTCGTCAAAAAGGC | |
| | GTATCAGAAACTGTGCTACATTCATCATATCATGGGAAAGGTGC | |
| | CAGACGCTTGCACTGCCTGCGATCTGGTCAATGTGGATTTGGAT | |
| | GACTGCATCTTTGAACAATAAatgacttaaaccaggtATGGCTGCCGATG | |
| | GTTATCTTCCAGATTGGCTCGAGGACAACCTTAGTGAAGGAATT | |
| | CGCGAGTGGTGGGCTTTGAAACCTGGAGCCCCTCAACCCAAGGG | |
| | AAATCAACAACATCAAGACAACGCTCGAGGTCTTGTGCTTCCGG | |
| | GTTACAAATACCTTGGACCCGGCAACGGACTCGACAAGGGGGA | |
| | GCCGGTCAACGCAGCAGACGCGGCGGCCCTCGAGCACGACAAG | |
| | GCCTACGACCAGCAGCTCAAGGCCGGAGACAACCCGTACCTCAA | |
| | GTACAACCACGCCGACGCCGAGTTCCAGGAGCGGCTCAAAGAA | |
| | GATACGTCTTTTGGGGGCAACCTCGGGCGAGCAGTCTTCCAGGC | |
| | CAAAAAGAGGCTTCTTGAACCTCTTGGTCTGGTTGAGGAAGCGG | |
| | CTAAGACGGCTCCTGGAAAGAAGAGGCCTGTAGAGCAGTCTCCT | |
| | CAGGAACCGGACTCCTCCGCGGGTATTGGCAAATCGGGTGCACA | |
| | GCCCGCTAAAAAGAGACTCAATTTCGGTCAGACTGGCGACACAG | |
| | AGTCAGTCCCAGACCCTCAACCAATCGGAGAACCTCCCGCAGCC | |
| | CCCTCAGGTGTGGGATCTCTTACAATGGCTTCAGGTGGTGGCGC | |
| | ACCAGTGGCAGACAATAACGAAGGTGCCGATGGAGTGGGTAGT | |
| | TCCTCGGGAAATTGGCATTGCGATTCCCAATGGCTGGGGGACAG | |
| | AGTCATCACCACCAGCACCCGAACCTGGGCCCTGCCCACCTACA | |
| | ACAATCACCTCTACAAGCAAATCTCCAACAGCACATCTGGAGGA | |
| | TCTTCAAATGACAACGCCTACTTCGGCTACAGCACCCCCTGGGG | |
| | GTATTTTGACTTCAACAGATTCCACTGCCACTTCTCACCACGTGA | |
| | CTGGCAGCGACTCATCAACAACAACTGGGGATTCCGGCCTAAGC | |
| | GACTCAACTTCAAGCTCTICAACATTCAGGTCAAAGAGGTTACG | |
| | GACAACAATGGAGTCAAGACCATCGCCAATAACCTTACCAGCAC | |
| | GGTCCAGGTCTTCACGGACTCAGACTATCAGCTCCCGTACGTGCT | |
| | CGGGTCGGCTCACGAGGGCTGCCTCCCCCCGTTCCCAGCGGACG | |
| | TTTTCATGATTCCTCAGTACGGGTATCTGACGCTTAATGATGGAA | |
| | GCCAGGCCGTGGGTCGTTCGTCCTTTTACTGCCTGGAATATTTCC | |
| | CGTCGCAAATGCTAAGAACGGGTAACAACTTCCAGTTCAGCTAC | |
| | GAGTTIGAGAACGTACCTTTCCATAGCAGCTACGCTCACAGCCA | |
| | AAGCCTGGACCGACTAATGAATCCACTCATCGACCAATACTTGT | |
| | ACTATCTCTCAAAGACTATTAACGGTTCTGGACAGAATCAACAA | |
| | ACGCTAAAATTCAGTGTGGCCGGACCCAGCAACATGGCTGTCCA | |
| | GGGAAGAAACTACATACCTGGACCCAGCTACCGACAACAACGT | |
| | GTCTCAACCACTGTGACTCAAAACAACAACAGCGAATTTGCTTG | |
| | GCCTGGAGCTTCTTCTTGGGCTCTCAATGGACGTAATAGCTTGAT | |
| | GAATCCTGGACCTGCTATGGCCAGCCACAAAGAAGGAGAGGAC | |
| | CGTTTCTTTCCTTTGTCTGGATCTTTAATTTTTGGCAAACAAGGA | |
| | ACTGGAAGAGACAACGTGGATGCGGACAAAGTCATGATAACCA | |
| | ACGAAGAAGAAATTAAAACTACTAACCCGGTAGCAACGGAGTC | |
| | CTATGGACAAGTGGCCACAAACCACCAGAGTGCCCAAGCACAG | |
| | CCCCAGACCGGCTGGGTTCAAAACCAAGGAATACTTCCGGGTAT | |
| | GGTTTGGCAGGACAGAGATGTGTACCTGCAAGGACCCATTTGGG | |
| | CCAAAATTCCTCACACGGACGGCAACTTTCACCCTTCTCCGCTGA | |
| | TGGGAGGGTTTGGAATGAAGCACCCGCCTCCTCAGATCCTCATC | |
| | AAAAACACACCTGTACCTGGGGATCCTCCAACGGCCTTCAACAA | |
| | GGACAAGCTGAACTCTTTCATCACCCAGTATTCTACTGGCCAAGT | |
| | CAGCGTGGAGATCGAGTGGGAGCTGCAGAAGGAAAACAGCAAG | |
| | CGCTGGAACCCGGAGATCCAGTACACTTCCAACTATTACAAGTC | |
| | TAATAATGTTGAATTTGCTGTTAATACTGAAGGTGTATATAGTGA | |
| | ACCCCGCCCCATTGGCACCAGATACCTGACTCGTAATCTGTAA | |
| PKP2 | MAAPGAPAEYGYIRTVLGQQILGQLDSSSLALPSEAKLKLAGSSGR | 789 |
| Protein | GGQTVKSLRIQEQVQQTLARKGRSSVGNGNLHRTSSVPEYVYNLH | |
| | LVENDFVGGRSPVPKTYDMLKAGTTATYEGRWGRGTAQYSSQKS | |
| | VEERSLRHPLRRLEISPDSSPERAHYTHSDYQYSQRSQAGHTLHHQE | |
| | SRRAALLVPPRYARSEIVGVSRAGTTSRQRHFDTYHRQYQHGSVSD | |
| | TVFDSIPANPALLTYPRPGTSRSMGNLLEKENYLTAGLTVGQVRPL | |
| | VPLQPVTQNRASRSSWHQSSFHSTRTLREAGPSVAVDSSGRRAHLT | |
| | VGQAAAGGSGNLLTERSTFTDSQLGNADMEMTLERAVSMLEADH | |
| | MLPSRISAAATFIQHECFQKSEARKRVNQLRGILKLLQLLKVQNEDV | |
| | QRAVCGALRNLVFEDNDNKLEVAELNGVPRLLQVLKQTRDLETKK | |
| | QITGLLWNLSSNDKLKNLMITEALLTLTENIIIPFSGWPEGDYPKAN | |
| | GLLDFDIFYNVTGCLRNMSSAGADGRKAMRRCDGLIDSLVHYVRG | |
| | TIADYQPDDKATENCVCILHNLSYQLEAELPEKYSQNIYIQNRNIQT | |
| | DNNKSIGCFGSRSRKVKEQYQDVPMPEEKSNPKGVEWLWHSIVIR | |
| | MYLSLIAKSVRNYTQEASLGALQNLTAGSGPMPTSVAQTVVQKES | |
| | GLQHTRKMLHVGDPSVKKTAISLLRNLSRNLSLQNEIAKETLPDLVS | |
| | HIPDTVPSTDLLIETTASACYTLNNIIQNSYQNARDLLNTGGIQKIMAI | |
| | SAGDAYASNKASKAASVLLYSLWAHTELHHAYKKAQFKKTDFVN | |
| | SRTAKAYHSLKD | |
| WPRE | TCAACCTCTGGATTACAAAATTTGTGAAAGATTGACTGGTATTCT | 790 |
| | TAACTATGTTGCTCCTTTTACGCTATGTGGATACGCTGCTTTAAT | |
| | GCCTTTGTATCATGCTATTGCTTCCCGTATGGCTTTCATTTTCTCC | |
| | TCCTTGTATAAATCCTGGTTGCTGTCTCTTTATGAGGAGTTGTGG | |
| | CCCGTTGTCAGGCAACGTGGCGTGGTGTGCACTGTGTTTGCTGAC | |
| | GCAACCCCCACTGGTTGGGGCATTGCCACCACCTGTCAGCTCCTT | |
| | TCCGGGACTTTCGCTTTCCCCCTCCCTATTGCCACGGCGGAACTC | |
| | ATCGCCGCCTGCCTTGCCCGCTGCTGGACAGGGGCTCGGCTGTT | |
| | GGGCACTGACAATTCCGTGGTGTTGTCGGGGAAATCATCGTCCT | |
| | TTCCTTGGCTGCTCGCCTGTGTTGCCACCTGGATTCTGCGCGGGA | |
| | CGTCCTTCTGCTACGTCCCTTCGGCCCTCAATCCAGCGGACCTTC | |
| | CTTCCCGCGGCCTGCTGCCGGCTCTGCGGCCTCTTCCGCGTCTTC | |
| | G | |
| hGH | CCTCGACTGTGCCTTCTAGTTGCCAGCCATCTGTTGTTTGCCCCT | 791 |
| polyA | CCCCCGTGCCTTCCTTGACCCTGGAAGGTGCCACTCCCACTGTCC | |
| signal | TTTCCTAATAAAATGAGGAAATTGCATCGCATTGTCTGAGTAGG | |
| | TGTCATTCTATTCTGGGGGGTGGGGTGGGGCAGGACAGCAAGGG | |
| | GGAGGATTGGGAAGACAATAGCAGGCATGCTGGGGACTGGGGA | |
| WPRE- | TCAACCTCTGGATTACAAAATTTGTGAAAGATTGACTGGTATTCT | 792 |
| hGH | TAACTATGTTGCTCCTTTTACGCTATGTGGATACGCTGCTTTAAT | |
| polyA | GCCTTTGTATCATGCTATTGCTTCCCGTATGGCTTTCATTTTCTCC | |
| signal | TCCTTGTATAAATCCTGGTTGCTGTCTCTTTATGAGGAGTTGTGG | |
| cassette | CCCGTTGTCAGGCAACGTGGCGTGGTGTGCACTGTGTTTGCTGAC | |
| | GCAACCCCCACTGGTTGGGGCATTGCCACCACCTGTCAGCTGCTT | |
| | TCCGGGACTTTCGCTTTCCCCCTCCCTATTGCCACGGCGGAACTC | |
| | ATCGCCGCCTGCCTTGCCCGCTGCTGGACAGGGGCTCGGCTGTT | |
| | GGGCACTGACAATTCCGTGGTGTTGTCGGGGAAATCATCGTCCT | |
| | TTCCTTGGCTGCTCGCCTGTGTTGCCACCTGGATTCTGCGCGGGA | |
| | CGTCCTTCTGCTACGTCCCTTCGGCCCTCAATCCAGCGGACCTTC | |
| | CTTCCCGCGGCCTGCTGCCGGCTCTGCGGCCTCTTCCGCGTCTTC | |
| | GAGATCTGCCTCGACTGTGCCTTCTAGTTGCCAGCCATCTGTTGT | |
| | TTGCCCCTCCCCCGTGCCTTCCTTGACCCTGGAAGGTGCCACTCC | |
| | CACTGTCCTTTCCTAATAAAATGAGGAAATTGCATCGCATTGTCT | |
| | GAGTAGGTGTCATTCTATTCTGGGGGGTGGGGTGGGGCAGGACA | |
| | CCAAGGGGGAGGATTGGGAAGACAATAGCAGGCATGCTGGGGA | |
| | CTGGGGACTCGAGTTAAGGGCGAATTCCCGATAAGGATCTTCCT | |
| | AGAGCATGGCTACGTAGATAAGTAGCATGGCGGGTTAATCATTA | |
| | ACTACA | |
| AAV9 | MAADGYLPDWLEDNLSEGIREWWALKPGAPQPKANQQHQDNAR | 713 |
| capsid | GLVLPGYKYLGPGNGLDKGEPVNAADAAALEHDKAYDQQLKAGD | |
| amino acid | NPYLKYNHADAEFQERLKEDTSFGGNLGRAVFQAKKRLLEPLGLV | |
| sequence | EEAAKTAPGKKRPVEQSPQEPDSSAGIGKSGAQPAKKRLNFGQTGD | |
| | TESVPDPQPIGEPPAAPSGVGSLTMASGGGAPVADNNEGADGVGSS | |
| | SGNWHCDSQWLGDRVITTSTRTWALPTYNNHLYKQISNSTSGGSSN | |
| | DNAYFGYSTPWGYFDFNRFHCHESPRDWQRLINNNWGFRPKRLNF | |
| | KLFNIQVKEVTDNNGVKTIANNLTSTVQVFTDSDYQLPYVLGSAHE | |
| | GCLPPFPADVFMIPQYGYLTLNDGSQAVGRSSFYCLEYFPSQMLRT | |
| | GNNFQFSYEFENVPFHSSYAHSQSLDRLMNPLIDQYLYYLSKTINGS | |
| | GQNQQTLKFSVAGPSNMAVQGRNYIPGPSYRQQRVSTTVTQNNNS | |
| | EFAWPGASSWALNGRNSLMNPGPAMASHKEGEDRFFPLSGSLIFGK | |
| | QGTGRDNVDADKVMITNEEEIKTTNPVATESYGQVATNHQSAQAQ | |
| | AQTGWVQNQGILPGMVWQDRDVYLQGPIWAKIPHTDGNFHPSPL | |
| | MGGFGMKHPPPQILIKNTPVPADPPTAFNKDKLNSFITQYSTGQVSV | |
| | EIEWELQKENSKRWNPEIQYTSNYYKSNNVEFAVNTEGVYSEPRPI | |
| | GTRYLTRNL | |

Capsid Proteins with Variant Polypeptide Sequences

In one aspect, the present disclosure provides AAV9 capsid proteins, wherein the capsid protein comprises variant polypeptide sequences with respect to the parental sequence at one or more sites of the parental sequence. In some embodiments, the one or more sites of the parental sequence are selected from the group consisting of VR-IV site, VR-V site, VR-VIII site, and VR-VIII site. As labeled in the SEQ ID NO: 1 above, the VR-IV site is bx-tween residues 452 and 460) in the parental sequence (“NGSGQNQ”, SEQ ID NO: 2); the VR-V site is between residues 497 and 502 in the parental sequence (“NNSEFA”, SEQ ID NO: 3); the VR-VIII site is between residues 549 and 553 in the parental sequence (“GRDNV”, SEQ ID NO: 4); the VR-VIII site is between residues 581 and 594 in the parental sequence (“ATNHQSAQAQAQTG”, SEQ ID NO: 5). In some embodiments, the AAV9 capsid protein comprises a sequence that shares at least about 80%, 85%, 90%, 95%, 96%, 97%, 98%, 99%, 99.5%, or 100% identity to SEQ ID NO: 1, excluding the VR-IV site, VR-V site, VR-VIII site and/or the VR-VIII site. In some embodiments, the AAV9 capsid protein comprises a sequence that shares at least about 80%, 85%, 90%, 95%, 96%, 97%, 98%, 990, 99.5%, or 100% identity to SEQ ID NO: 1, excluding the VR-VIII site. In some embodiments, the AAV9 capsid protein comprises a sequence that shares at least about 80%, 85%, 90%, 95%, 96%, 97%, 98%, 99%, 99.5%, or 100% identity to SEQ ID NO: 487, excluding the VR-IV site, VR-V site, VR-VIII site and/or the VR-VIII site. In some embodiments, the AAV9 capsid protein comprises a sequence that shares at least about 80%, 85%, 90%, 95%, 96%, 97%, 98%, 99%, 99.5%, or 100% identity to SEQ ID NO: 487, excluding the VR-VIII site. In some embodiments, the AAV9 capsid protein comprises a variant polypeptide sequence at one or more of a VR-IV site, a VR-V site, a VR-VIII site, and a VR-VIII site of a parental sequence, wherein the parental sequence comprises a sequence at least 80%, at least 85%, at least 90%, at least 95%, at least 98%, at least 99%, or 100% identical to SEQ ID NO: 463. (In SEQ ID NO:463, the amino acids residues labeled “X” are excluded from sequence identity calculation.)

in some embodiments, a capsid protein described herein comprises an amino acid substitution or insertion in the VR-IV site (between residues 452 and 460 in SEQ ID NO:1 or in the sequence of SEQ ID NO:2 (NGSGQNQ)). In some embodiments, a capsid protein described herein comprises an amino acid substitution in the VR-IV site (between residues 452 and 460 in SEQ ID NO:1 or in the sequence of SEQ ID NO:2 (NGSGQNQ)). In some embodiments, the amino acid substitution or insertion in the VR-IV site is any amino acid substitution or insertion described herein. In some embodiments, a capsid protein described herein comprises an amino acid substitution at position 452 of SEQ ID NO:1 or the first amino acid of SEQ ID NO:2 (NGSGQNQ) in the VR-IV site. In some embodiments, a capsid protein described herein comprises an amino acid substitution N452K in SEQ ID NO:1 or comprises the sequence KGSGQNQ in the VR-IV site.

In some embodiments, a capsid protein described herein comprises an amino acid substitution or insertion in the VR-V site (between residues 497 and 502 in SEQ ID NO:1 or in the sequence of SEQ ID NO:3 (NNSEFA)). In some embodiments, a capsid protein described herein comprises an amino acid substitution in the VR-V site (between residues 497 and 502 in SEQ ID NO:1 or in the sequence of SEQ ID NO:3 (NNSEFA)). In some embodiments, the amino acid substitution or insertion in the VR-V site is any amino acid substitution or insertion described herein.

In some embodiments, a capsid protein described herein comprises an amino acid substitution or insertion in the VR-VIII site (between residues 549 and 553 in SEQ ID NO:1 or in the sequence of SEQ ID NO:4 (GRDNV)). In some embodiments, a capsid protein described herein comprises an amino acid substitution in the VR-VIII site (between residues 549 and 553 in SEQ ID NO:1 or in the sequence of SEQ ID NO:4 (GRDNV)). In some embodiments, the amino acid substitution or insertion in the VR-VII site is any amino acid substitution or insertion described herein.

In some embodiments, a capsid protein described herein comprises an amino acid substitution or insertion in the VR-VIII site (between residues 581 and 594 in SEQ ID NO:1 or in the sequence of SEQ ID NO:5 (ATNHQSAQAQAQTG)). In some embodiments, a capsid protein described herein comprises an amino acid substitution in the VR-VIII site (between residues 581 and 594 in SEQ ID NO:1 or in the sequence of SEQ ID NO:5 (ATNHQSAQAQAQTG)). In some embodiments, the amino acid substitution or insertion in the VR-VIII site is any amino acid substitution or insertion described herein.

In some embodiments, the AAV9 capsid protein comprises a variant polypeptide sequence that are either rationally designed; introduced by mutagenesis; or randomized through generating a library of sequences with random codon usage at one or more sites. The capsid proteins of the disclosure include any variant polypeptide sequences identified as enriched by directed evolution followed by sequencing, as shown in, but not limited to, the Examples. Without being limited to any particular substitution site, in some embodiments, one or more sites selected from the group consisting of the VR-IV site, the VR-V site, the VR-VIII site, and VR-VIII site have the amino acid substitutions as described herein.

Various amino acid substitutions, insertions, and deletion are provided herein. Any of these modifications may be combined with any of the others, though where the modifications overlap one must be selected or an insertion maybe used to put two modifications adjacent to or near one another. The combination of modifications may be tested, using methods described herein (e.g., in vitro test in iPSC-CMs, in vivo testing in model organisms individually, and in vivo re-screening of pooled rAAV virions) or other known methods to identify combinations having desired packing efficiency, tropism, or other desired properties. Similarly, modifications may be made at exactly the position desired herein, or the same modification may be may at any position near to the described position. Structural modeling of the capsid protein may be used to select modification for testing.

In some embodiments, the engineered capsid provided herein is any one of the capsids described herein. In some embodiments, the engineered capsid provided herein is any one of the VR-VIII-modified capsids described herein. In some embodiments, the engineered capsid provided herein is any one of the VR-IV-modified capsids described herein. In some embodiments, the engineered capsid provided herein is any one of the VR-VIII and VR-IV-modified capsids described herein. In some embodiments, the engineered capsid provided herein is any of the capsids described in any of the examples, tables or figures provided herein. In some embodiments, the engineered capsid provided herein is any of the capsids described in FIG. 45.

### Modifications Identified in Primate Screening

In one aspect, the present disclosure provides recombinant adeno-associated virus (rAAV) capsid proteins, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1, one or more of the modifications described herein.

Any of the modifications described herein may be used either alone or in combination with other modifications (e.g., in combination with other modifications described herein).

Solely for purposes of clarity and without limitation, it is noted that reference to amino acid position numbers at which modifications occur is relative to position of corresponding amino acids in SEQ ID NO:1. In some embodiments, the capsid protein does not comprise the full-length sequence corresponding to SEQ ID NO:1, but comprises a shorter variant of this sequence (e.g., comprises only a variant of SEQ ID NO:487, or a variant of SEQ ID NO:486). In such embodiments, the modifications described herein may not occur at the same numerical positions as in SEQ ID NO:1 but occur at the same site or consensus sequence relative to reference sequence SEQ ID NO: 1. In some embodiments, the capsid protein is a variant of SEQ ID NO:1, and the modifications described herein occur at the same numerical positions as in SEQ ID NO:1.

The capsid protein may comprise an amino acid insertion at position 584 comprising one or more of an asparagine (N), a threonine (T), a tyrosine (Y), phenylalanine (F), and an alanine (A).

The capsid protein may comprise an amino acid insertion at position 585 comprising one or more of a histidine (H) and a methionine (M).

The capsid protein may comprise an amino acid insertion at position 586 comprising one or more of a histidine (H), a tyrosine (Y), a valine (V), a threonine (T), an alanine (A), an isoleucine (I), a tryptophan (W), a methionine (M), and a leucine.

The capsid protein may comprise an amino acid insertion at position 587 comprising one or more of an isoleucine (I) and a proline (P).

The capsid protein may comprise an amino acid insertion at position 588 comprising one or more of an isoleucine (I), a threonine (T), and a proline (P).

The capsid protein may comprise one or more amino acid substitutions selected from the group consisting of N452K, N452A, N452V, G453A, G453N, S454T, S454D, G4S5N, Q456L, Q456K, N457L, N457V, Q458I, and Q458H.

The capsid protein may comprise an amino acid substitution N452K.

The capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W. H584E, H584D, Q585T, Q585C, Q585V, Q585L, Q585N, Q585S, Q585P, Q585A, Q585M, Q585E, Q585Y, Q585G, Q585H, Q585I, S586D, S586T, S586O, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

The capsid protein may comprise an amino acid insertion at position 584 consisting of a TY, FN, or AT.

The capsid protein may comprise an amino acid insertion at position 585 consisting of MH.

The capsid protein may comprise an amino acid insertion at position 586 consisting of HY, VT, Al, WM, or ML.

The capsid protein may comprise an amino acid insertion at position 587 consisting of Pl.

The capsid protein may comprise an amino acid insertion at position 588 consisting of IT or PT.

The capsid protein may comprise one or more amino acid substitutions selected from the group consisting of T582D, T582E, N583V, H584Q, S586K, A587P, A587S, Q5880, Q588M, A589S, A591I. G594Q, and G594D.

The capsid protein may comprise one or more amino acid substitutions selected from the group consisting of T582L, T582A, T582F, T582R. T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R.

The capsid protein may comprise one or more amino acid substitutions selected from the group consisting of Q585C, Q585S, and S586I.

The capsid protein may comprise one or more amino acid substitutions selected from the group consisting of Q585V, Q585T, Q585L, Q585C, Q585N, Q585S, Q585M, Q585E, Q585P, Q585A, Q585C, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q58811, Q588Y, Q588M, Q588K, Q588D, Q588G, A589R, A589[, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

The capsid protein may comprise one or more amino acid substitutions selected from the group consisting of A587V and A587G.

The capsid protein may comprise an amino acid sequence selected from SEQ ID NOs: 599-692 and wherein the capsid protein shares at least 80%, at least 90%, at least 95%, at least 98%, or 100% identity to SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, and 710.

The capsid protein may comprise an amino acid sequence selected from SEQ ID NOs: 599-692 and wherein the capsid protein shares at least 80%, at least 90%, at least 95%, at least 98%, or 100% identity to SEQ ID NOs: 496-589.

The capsid protein may comprise the amino acid sequence ANYG at positions 586-589 or at about positions 586-589.

The capsid protein may comprise two or more amino acid substitutions selected from the group consisting of N452K, N452A, N452V, G453A, G453N, S454T, S454D, G455N, Q456L, Q456K, N457L, N457V, Q458I, and Q458H.

The capsid protein may comprise the amino acid substitution N452K, N452A, or N452V.

The capsid protein may comprise the amino acid substitution N452K.

The capsid protein may comprise the amino acid substitution G453A or G453N.

The capsid protein may comprise the amino acid substitution S454T or S454D.

The capsid protein may comprise the amino acid substitution G455N.

The capsid protein may comprise the amino acid substitution Q456L or Q456K.

The capsid protein may comprise the amino acid substitution N457L or N457V.

The capsid protein may comprise the amino acid substitution Q458I or Q458H.

The capsid protein may comprise an amino acid sequence selected from KGSGQNQ (SEQ ID NO: 590), NASGQNQ (SEQ ID NO: 591). NGTGQNQ (SEQ ID NO: 592), NGSGLNQ (SEQ ID NO: 593), ANDNKLI (SEQ ID NO: 594), VNDNKVI (SEQ ID NO: 595). NGSGQNH (SEQ ID NO: 596), or ANDNKVI (SEQ ID NO: 597) at positions 452-458 or at about positions 452-458 and wherein the capsid protein shares at least 80%-4, at least 90%, at least 95%, at least 98%, or 100% identity to SEQ ID NOs: 488-495.

The capsid protein may comprise an amino acid sequence selected from NTVS (SEQ ID NO: 712), TLFN (SEQ ID NO: 713), STYL (SEQ ID NO: 714), SILT (SEQ ID NO: 715), MTTA (SEQ ID NO: 716), and STSI (SEQ ID NO: 717) at positions 586-589 or at about positions 586-589 relative to reference sequence SEQ ID NO: 1. In some of these embodiments, the capsid protein comprises N452K substitution relative to reference sequence SEQ ID NO: 1.

The capsid protein may comprise an amino acid sequence selected from GAYA (SEQ ID NO: 741), TKLA (SEQ ID NO: 742), SSFT (SEQ ID NO: 743), DNIR (SEQ ID NO: 744). NVIS (SEQ ID NO: 745), GTSI (SEQ ID NO: 746), ANYG (SEQ ID NO: 305) and DARA (SEQ ID NO: 747) at positions 586-589 or at about positions 586-589 relative to reference sequence SEQ ID NO: 1. In some of these embodiments, the capsid protein comprises N452K substitution relative to reference sequence SEQ ID NO: 1.

The capsid protein may comprise an amino acid sequence SAQA (SEQ ID NO: 748) at positions 586-589 or at about positions 586-589 relative to reference sequence SEQ ID NO: 1 or comprise the same sequence at the corresponding positions relative to reference sequence SEQ ID NO:1. In some of these embodiments, the capsid protein comprises N452K substitution relative to reference sequence SEQ ID NO: 1.

The capsid protein may comprise an amino acid sequence selected from ENTVSI (SEQ ID NO: 719), QTLFNS (SEQ ID NO: 720), NSTYLG (SEQ ID NO: 721), GSILTH (SEQ ID NO: 722). MMTTAR (SEQ ID NO: 723), and CSTSIR (SEQ ID NO: 724) at positions 585-590 or at about positions 585-590 relative to reference sequence SEQ ID NO: 1. In some of these embodiments, the capsid protein comprises N452K substitution relative to reference sequence SEQ ID NO: 1.

The capsid protein may comprise an amino acid sequence selected from QGAYAQ (SEQ ID NO: 749), NTKLAI (SEQ ID NO: 750), VSSFTS (SEQ ID NO: 751), EDNIRS (SEQ ID NO: 725), NNVISG (SEQ ID NO: 752), TGTSII (SEQ ID NO: 753), QANYGQ (SEQ ID NO: 754), and QDARAQ (SEQ ID NO: 755) at positions 585-590 or at about positions 585-590 relative to reference sequence SEQ ID NO: 1. In some of these embodiments, the capsid protein comprises N452K substitution relative to reference sequence SEQ ID NO: 1.

The capsid protein may comprise an amino acid sequence QSAQAQ (SEQ ID NO: 756) at positions 585-590 or at about positions 585-590 relative to reference sequence SEQ ID NO: 1 or comprise the same sequence at the corresponding positions relative to reference sequence SEQ ID NO:1. In some of these embodiments, the capsid protein comprises N452K substitution relative to reference sequence SEQ ID NO: 1.

The capsid protein may comprise AAV9 wild type amino acid sequence at positions 581-584 (i.e., ATNH) and/or at positions 591-594 (i.e., AQTG). The capsid protein may comprise AAV9 wild type amino acid sequence at positions 581-583 (i.e., ATN) and/or at positions 591-594 (i.e., AQTG).

### Modifications in VR-IV, VR-V and VR-VIII Sits

In some embodiments, the capsid protein of the present disclosure comprises a variant polypeptide sequence at the VR-IV site. In some embodiments, the entire VR-IV site (“NGSGQNQQT”, SEQ ID NO: 2) is substituted by a peptide of formula:

-(X)n-

wherein n is 7-11, and X represents any of the 20 standard amino acids (SEQ ID NO: 478).

In some embodiments, the variant polypeptide sequence at the VR-IV site is:

(SEQ ID NO: 478)
-X1-X2-X3-X4-X5-X6-X7-X8-X9-.

In some embodiments, the variant polypeptide sequence at the VR-TV site is:

-X1-X2-X3-X4-X5-X6-X7-X8-X9-

wherein X1 is G, S or V; X2 is Y, Q or I; X3 is H, W, V, or I; X4 is K or N; X5 is S, G or I; X6 is G or R; X7 is A, P or V; X8 is A or R; and/or X9 is Q or D (SEQ ID NO: 477).

In some embodiments, the variant polypeptide sequence at the VR-IV site is:

-X1-X2-X3-X4-X5-X6-X7-X8-X9-

wherein X1 is K, G, S or V; X2 is Y, Q or I; X3 is H, W, V, or I; X4 is K or N: X5 is S, G or I; X6 is G or R; X7 is A, P or V: X8 is A or R; and/or X9 is Q or D (SEQ ID NO: 729).

In some embodiments, the variant polypeptide sequence at the VR-IV site is:

-X1-X2-X3-X4-X5-X6-X7-X8-X9-

wherein X1 is K (SEQ ID NO: 730).

In some embodiments, the variant polypeptide sequence at the VR-IV site comprises or consists of the sequence KGSGQNQQT (SEQ ID NO:727).

In some embodiments, the capsid protein of the present disclosure comprises a variant polypeptide sequence with N452K substitution at the VR-IV site. In some embodiments, the capsid protein of the present disclosure comprises a variant polypeptide sequence with N452K substitution at the VR-IV site relative to reference SEQ ID NO:1 or comprises the sequence of KGSGQNQQT (SEQ ID NO:727). In some embodiments, such substitution is the only substitution in an AAV9 capsid protein. In some embodiments, such substitution is the only substitution in the capsid protein of the present disclosure relative to reference SEQ ID NO:1. In some embodiments, the capsid protein comprises amino acid substitution N452K as the only substitution in a wild type AAV9 capsid protein (such as in the parental sequence of SEQ ID NO:487 or SED ID NO:1). In some embodiments, such substitution is the only substitution in the AAV9 capsid protein's VR-IV and/or VR-III sites. In some embodiments, the capsid protein of the present disclosure (such as an AAV9 capsid protein) comprises amino acid substitution N452K at the VR-IV site in addition to any other substitution or insertion described herein or known in the art (including, but not limited to, any other substitution or insertion at the VR-IV site. VR-V site, VR-VII site and/or VR-VIII site). In some embodiments, the capsid protein of the present disclosure comprises amino acid substitution N452K at the VR-IV site relative to reference SEQ ID NO:1 or the sequence KGSGQNQQT (SEQ ID NO:727) in addition to any other substitution, insertion, or chimeric modification described herein or known in the art. In some embodiments, the capsid protein of the present disclosure comprises the sequence KGSGQNQQT (SEQ ID NO:727) in addition to any chimeric modification described herein or known in the art. In some embodiments, N452K substitution is combined with any other substitution(s) or insertion(s) described herein (e.g., in the VR-IV site and/or the VR-VIII site), and/or any chimeric modification(s) described herein. In some embodiments, such substitution is combined with any substitution(s) or insertion(s) in the VR-IV site described herein or known in the art. In some embodiments, such substitution is combined with any substitution(s) or insertion(s) in the VR-V site described herein or known in the art. In some embodiments, such substitution is combined with any substitution(s) or insertion(s) in the VR-VIII site described herein or known in the art. In some embodiments, such substitution is combined with any substitution(s) or insertion(s) in the VR-VIII site described herein or known in the art. In some embodiments, the capsid protein of the present disclosure comprises amino acid substitution N452K at the VR-IV site in addition to any one, two, three or more substitutions or insertions at the VR-VIII site. In some embodiments, the capsid protein of the present disclosure comprises amino acid substitution N452K, relative to reference sequence SEQ ID NO: 1, in addition to one, two, three or more substitutions or insertions at the VR-VIII site described herein. In some embodiments, the capsid protein, such as the capsid protein with N452K substitution at the VR-IV site relative to reference SEQ 10 NO:1, increases transduction efficiency (e.g., of any tissue, such as muscle, heart, skeletal muscle, brain, etc.). In some embodiments, the capsid protein of the present disclosure, such as the capsid protein with N452K substitution at the VR-IV site relative to reference SEQ ID NO:1, increases transduction efficiency of the heart.

In some embodiments, the capsid protein of the present disclosure comprises wild type AAV9 amino acid (which is N) at position 452 of the VR-IV site relative to reference SEQ ID NO:1.

In some embodiments, the engineered capsid protein of the present disclosure comprises N or K at position 452 of the VR-IV site relative to reference SEQ ID NO:1.

In some embodiments, the variant polypeptide sequence at the VR-IV site comprises or consists of a sequence selected from GYHKSGAAQ (SEQ ID NO: 6), VIIKSGAAQ (SEQ ID NO: 7), GYHKIGAAQ (SEQ ID NO: 8), GYHKSGVAQ (SEQ ID NO: 9), VYHKSGAAQ (SEQ ID NO: 10), GYHKJSAAQ (SEQ ID NO: 11), TTVPSSSRY (SEQ ID NO: 12), VIIRVVRLS (SEQ ID NO: 13). TVLGQNQQT (SEQ ID NO: 14), IYHKSGAAQ (SEQ ID NO: 15), TVLDKNQQT (SEQ ID NO: 16), YSGTDVRYK (SEQ ID NO: 17), VTASGKEHR (SEQ ID NO: 18), GYRKSGAAQ (SEQ ID NO: 19), NRTVSNGSE (SEQ ID NO: 20), TVLDRINKT (SEQ ID NO: 21), TGVGHLTSA (SEQ ID NO: 22), GYHKGGAAQ (SEQ ID NO: 23), VIAKSGAAQ (SEQ ID NO: 24), GYHKSGAAH (SEQ ID NO: 25), FIIKSGAAQ (SEQ ID NO: 26), GYHKVVRLS (SEQ ID NO: 27), GATRSAVES (SEQ ID NO: 28), TVSGQNQQT (SEQ ID NO: 29), LSHKSOAAQ (SEQ ID NO: 30), SSSGQNQQT (SEQ ID NO: 31), SGSGQNQQT (SEQ ID NO: 32), SQVNGRPRD (SEQ ID NO: 33), GYHKEWCGS (SEQ ID NO: 34), VVSSKSLNS (SEQ ID NO: 35), GYHKSGAAP (SEQ ID NO: 36), DASSREKVR (SEQ ID NO: 37), SYHKSGAAQ (SEQ ID NO: 38), TANGSQKYL (SEQ ID NO: 39), VURVGAAQ (SEQ ID NO: 40), SSTNKISTA (SEQ ID NO: 41), TVLDRIQQT (SEQ ID NO: 42), GYHKSGAVQ (SEQ ID NO: 43), TVLDQNQQT (SEQ ID NO: 44), VNMSSPIKT (SEQ ID NO: 45), AAYNSNSAF (SEQ ID NO: 46), GYHKSGAAR (SEQ ID NO: 47), VIIRVVRLQ (SEQ ID NO: 48), RFWTQNQQT (SEQ ID NO: 49), SSPRASSAL (SEQ ID NO: 50), IIIRVVRLS (SEQ ID NO: 51), KSSNLTAMP (SEQ ID NO: 52), NLNSDRHSA (SEQ ID NO: 53), LSLKSGAAQ (SEQ ID NO: 54), TVLDRNQQT (SEQ ID NO: 55), GSERVSNSG (SEQ ID NO: 56), VIAKIGAAQ (SEQ ID NO: 57), VYHKIGAAQ (SEQ ID NO: 58), LSYKSGAAQ (SEQ ID NO: 59), STVSQPVRT (SEQ ID NO: 60), GHHKSGAAQ (SEQ ID NO: 61), YAGIDPRYH (SEQ ID NO: 62), DRSRKSMCD (SEQ ID NO: 63), VIIRSGAAQ (SEQ ID NO: 64), GYHKSGGSA (SEQ ID NO: 65), VIIKIGAAQ (SEQ ID NO: 66), GYHKVVQLS (SEQ ID NO: 67), VIIKLVAAQ (SEQ ID NO: 68), KVSSHSVCD (SEQ ID NO: 69), GYHKRVRLS (SEQ ID NO: 70), GYHKSSAAQ (SEQ ID NO: 71), GYRKIGAAQ (SEQ ID NO: 72), GYHKSGAAC (SEQ ID NO: 73). GYRQSGAAQ (SEQ ID NO: 74), VIIKLIAAQ (SEQ ID NO: 75), VIIRVVRAQ (SEQ ID NO: 76), GYHKSGAAW (SEQ ID NO: 77), GYHKSGAVS (SEQ ID NO: 78), GYHKEWCSS (SEQ ID NO: 79), SSSSNRLAD (SEQ ID NO: 80), SNNSSSAKF (SEQ ID NO: 81), VKLSSTSSS (SEQ ID NO: 82), GYHKEWCAQ (SEQ ID NO: 83), AGSGQNQQT (SEQ ID NO: 84), NPHGTATYL (SEQ ID NO: 85), NGSGQNQHT (SEQ ID NO: 86), GYHKVGAAQ (SEQ ID NO: 87), VIIRVVRLK (SEQ ID NO: 88), NSIPSTSKW (SEQ ID NO: 89), VIIRVVQLQ (SEQ ID NO: 90), SQVNGRPQD (SEQ ID NO: 91), NGSGQDQQT (SEQ ID NO: 92), GLNSSDRRL (SEQ ID NO: 93), IYHKIGAAQ (SEQ ID NO: 94), YIKSGAAQL (SEQ ID NO: 95), YSGTDVQYK (SEQ ID NO: 96), LOSGQNQQT (SEQ ID NO: 97), PVSSGADRR (SEQ ID NO: 98), EHSTKLNAC (SEQ ID NO: 99), NGSDRINKR (SEQ ID NO: 100), VIIKGGAAQ (SEQ ID NO: 101), GYHRVVRLS (SEQ ID NO: 102), VIIRVVRLL (SEQ ID NO: 103), and VILKSGAAQ (SEQ ID NO: 104). In some of any of these embodiments, the first amino acid is substituted with K instead of any other amino acid at this position (or has N452K substitution relative to reference sequence SEQ ID NO:1).

In some embodiments, the variant polypeptide sequence at the VR-IV site comprises, consists essentially of, or consists of a polypeptide sequence at least about 60%”, 70%, 80%, 90%, or 100% identical to one of SEQ ID NOs: 6-104.

In some embodiments, the variant polypeptide sequence at the VR-IV site comprises, consists essentially of, or consists of a sequence at least about 60%, 70%, 77%, 80%, 88%, 90%, or 100% identical to KGSGQNQQT (SEQ ID NO:727). In some embodiments, the variant polypeptide sequence at the VR-IV site comprises, consists essentially of, or consists of a sequence consisting of at most 1, 2, 3, or 4 amino-acid substitutions relative to KGSGQNQQT (SEQ ID NO:727). In some embodiments, the variant polypeptide sequence at the VR-IV site comprises, consists essentially of, or consists of a sequence consisting of at most 1, 2, 3, or 4 conservative amino-acid substitutions relative KGSGQNQQT (SEQ ID NO:727). In some embodiments, the variant polypeptide sequence at the VR-IV site is KGSGQNQQT (SEQ ID NO:727).

In some embodiments, the variant polypeptide sequence at the VR-IV site comprises, consists essentially of, or consists of a sequence at least about 60%, 70%, 77%, 80%, 88%, 90%, or 100% identical to GYHKSGAAQ (SEQ ID NO: 6). In some embodiments, the variant polypeptide sequence at the VR-IV site comprises, consists essentially of, or consists of a sequence consisting of at most 1, 2, 3, or 4 amino-acid substitutions relative to GYHKSGAAQ (SEQ ID NO: 6). In some embodiments, the variant polypeptide sequence at the VR-IV site comprises, consists essentially of, or consists of a sequence consisting of at most 1, 2, 3, or 4 conservative amino-acid substitutions relative GYHKSGAAQ (SEQ ID NO: 6). In some embodiments, the variant polypeptide sequence at the VR-IV site is GYHKSGAAQ (SEQ ID NO: 6). In some of any of these embodiments, the first amino acid is substituted with K (KYHKSGAAQ; SEQ ID NO: 757).

In some embodiments, the variant polypeptide sequence at the VR-IV site comprises, consists essentially of, or consists of a sequence at least about 60%, 70%, 77%, 80%, 88%, 90%, or 100% identical to SQVNGRPRD (SEQ ID NO: 33). In some embodiments, the variant polypeptide sequence at the VR-IV site comprises, consists essentially of, or consists of a sequence consisting of at most 1, 2, 3, or 4 amino-acid substitutions relative to SQVNGRPRD (SEQ ID NO: 33). In some embodiments, the variant polypeptide sequence at the VR-IV site comprises, consists essentially of, or consists of a sequence consisting of at most 1, 2, 3, or 4 conservative amino-acid substitutions relative SQVNGRPRD (SEQ ID NO: 33). In some embodiments, the variant polypeptide sequence at the VR-IV site is SQVNGRPRD (SEQ ID NO: 33). In some of any of these embodiments, the first amino acid is substituted with K (KQVNGRPRD; SEQ ID NO: 758).

In some embodiments, the capsid protein of the present disclosure comprises a variant polypeptide sequence at the VR-V site. In some embodiments, the entire VR-V site (“NNSEFA”, SEQ ID NO: 3) is substituted by a peptide of formula:

-(X)n-

wherein n is 4-8, and X represents any of the 20 standard amino acids (SEQ ID NO: 479).

In some embodiments, the variant polypeptide sequence at the VR-V site is:

(SEQ ID NO: 479)
-X1-X2-X3-X4-X5-X6-

In some embodiments, the variant polypeptide sequence at the VR-V site is:

-X1-X2-X3-X4-X5-X6-

wherein X1 is S, L, H, N, or A; X2 is T, M, K, G, or N; X3 is S, T, M or I; X4 is S, P, F, M, or N; X5 is F, S, P or L; and X6 is I, V, or T (SEQ ID NO: 474).

In some embodiments, the variant polypeptide sequence at the VR-V site comprises or consists of a sequence selected from LNSMLI (SEQ ID NO: 105), NGMSFT (SEQ ID NO: 106), HKTFSI (SEQ ID NO: 107), SMSNFV (SEQ ID NO: 108), ATIPPI (SEQ ID NO: 109), SSTHFD (SEQ ID NO: 110). NNQFSY (SEQ ID NO: 111), NMGHYS (SEQ ID NO: 112), SKQMFQ (SEQ ID NO: 113), WPSAGV (SEQ ID NO: 114), NGGYQC (SEQ ID NO: 115), STSPIV (SEQ ID NO: 116), SQSGLW (SEQ ID NO: 117), VNSQFS (SEQ ID NO: 118), SGIEFR (SEQ ID NO: 119), SASKFT (SEQ ID NO: 120), QLNWTS (SEQ ID NO: 121), SMGFPV (SEQ ID NO: 122), SSFMGL (SEQ ID NO: 123), GSNFHV (SEQ ID NO: 124), DMTLYA (SEQ ID NO: 125), MGCLFT (SEQ ID NO: 126), ALAFNS (SEQ ID NO: 127), SKFLFA (SEQ ID NO: 128), QDAGLL (SEQ ID NO: 129), QDASLL (SEQ ID NO: 130). RDDMFS (SEQ ID NO: 131), LSRCFQ (SEQ ID NO: 132), LSRDFQ (SEQ ID NO: 133), QGLTPV (SEQ ID NO: 134), QWDVFT (SEQ ID NO: 135), PRVSFA (SEQ ID NO: 136), QSYYNP (SEQ ID NO: 137), RASHLG (SEQ ID NO: 138), IILFVP (SEQ ID NO: 139), IISFSY (SEQ ID NO: 140), LDSMLI (SEQ ID NO: 141), NIGHYS (SEQ ID NO: 142), NRMSFT (SEQ ID NO: 143), NGMSFA (SEQ ID NO: 144), IILLLP (SEQ ID NO: 145), RMRSLL (SEQ ID NO: 146), RRRCRF (SEQ ID NO: 147), PKQMFQ (SEQ ID NO: 148), LMSNFV (SEQ ID NO: 149), GASHLG (SEQ ID NO: 150), CASISW (SEQ ID NO: 151), SMTTFR (SEQ ID NO: 152), AAIPPI (SEQ ID NO: 153), PGCESL (SEQ ID NO: 154), SMGFAC (SEQ ID NO: 155), FLPSLM (SEQ ID NO: 156), NGISFT (SEQ ID NO: 157), ESSRWA (SEQ ID NO: 158), QLYFVP (SEQ ID NO: 159), SSNFHV (SEQ ID NO: 160), LEFMLI (SEQ ID NO: 161), QFDSFD (SEQ ID NO: 162), SPVFAC (SEQ ID NO: 163), VRLIFD (SEQ ID NO: 164), NGMSFI (SEQ ID NO: 165), LLFPPI (SEQ ID NO: 166), GAGVTG (SEQ ID NO: 167), QWMSFT (SEQ ID NO: 168), SIGFPV (SEQ ID NO: 169), RMQSLL (SEQ ID NO: 170), TSALQV (SEQ ID NO: 171), SLTHFD (SEQ ID NO: 172), QELPFL (SEQ ID NO: 173), LYFLLP (SEQ ID NO: 174), LSFFFA (SEQ ID NO: 175), LSRJFQ (SEQ ID NO: 176), DEVILF (SEQ ID NO: 177), RAGVAG (SEQ ID NO: 178), NGMSLP (SEQ ID NO: 179), PFEDFQ (SEQ ID NO: 180), QYGSLF (SEQ ID NO: 181), NYTFVL (SEQ ID NO: 182), MSGYQC (SEQ ID NO: 183), NYAFVP (SEQ ID NO: 184), RAGVTG (SEQ ID NO: 185), WNSMLI (SEQ ID NO: 186), IRRFSI (SEQ ID NO: 187), NGMSFY (SEQ ID NO: 188), IIQFSY (SEQ ID NO: 189), NGCLFT (SEQ ID NO: 190), RDASLL (SEQ ID NO: 191), ADSMLI (SEQ ID NO: 192), VDSQFS (SEQ ID NO: 193), SIGNFV (SEQ ID NO: 194), NGMSLL (SEQ ID NO: 195), NYTFVP (SEQ ID NO: 196), IRRLVF (SEQ ID NO: 197), PMSNFV (SEQ ID NO: 198), LWVFPV (SEQ ID NO: 199), VRLHFD (SEQ ID NO: 200), SMSNLF (SEQ ID NO: 201), STSLIV (SEQ ID NO: 202), and HKTFGI (SEQ ID NO: 203).

In some embodiments, the variant polypeptide sequence at the VR-V site comprises, consists essentially of, or consists of a polypeptide sequence at least about 60%, 70%, 80%, 90%,95%, or 100% identical to one of SEQ ID NOs: 105-203.

In some embodiments, the variant polypeptide sequence at the VR-V site comprises, consists essentially of, or consists of a sequence at least about 60%, 70%, 80%, 83%, 90%, or 100% identical to LNSMLI (SEQ ID NO: 105). In some embodiments, the variant polypeptide sequence at the VR-V site comprises, consists essentially of, or consists of a sequence consisting of at most 1, 2, 3, or 4 amino-acid substitutions relative to LNSMLI (SEQ ID NO: 105). In some embodiments, the variant polypeptide sequence at the VR-V site comprises, consists essentially of, or consists of a sequence consisting of at most 1, 2, 3, or 4 conservative amino-acid substitutions relative LNSMLI (SEQ ID NO: 105). In some embodiments, the variant polypeptide sequence at the VR-V site is LNSMLI (SEQ ID NO: 105).

In some embodiments, the capsid protein of the present disclosure comprises a variant polypeptide sequence at the VR-VIII site. In some embodiments, the entire VR-VIII site (“GRDNV”, SEQ ID NO: 4) is substituted by a peptide of formula:

-(X)n-

wherein n is 3-7, and X represents any of the 20 standard amino acids (SEQ ID NO: 480).

In some embodiments, the variant polypeptide sequence at the VR-VII site is:

(SEQ ID NO: 480)
-X1-X2-X3-X4-X5-

In some embodiments, the variant polypeptide sequence at the VR-VIII site is:

-X1-X2-X3-X4-X5-

wherein X1 is V, L, Q, C, or R; X2 is S, H, G, C, or D; X3 is Y, S, L, G, or N; X4 is S, L, H, Q, or N; and X5 is V, I, or R (SEQ ID NO: 475).

In some embodiments, the variant polypeptide sequence at the VR-VIII site comprises or consists of a sequence selected from RGNQV (SEQ ID NO: 204), VSLNR (SEQ ID NO: 205), CDYSV (SEQ ID NO: 206), QHGHI (SEQ ID NO: 207), LCSLV (SEQ ID NO: 208), PTIYV (SEQ ID NO: 209), DVIHI (SEQ ID NO: 210), AEFYA (SEQ ID NO: 211), NSVVC (SEQ ID NO: 212), VRSNC (SEQ ID NO: 213), LANNI (SEQ ID NO: 214), NLQFM (SEQ ID NO: 215), EFRDL (SEQ ID NO: 216), DFGSL (SEQ ID NO: 217), VTNYC (SEQ ID NO: 218), WNTNA (SEQ ID NO: 219), TESTC (SEQ ID NO: 220), SGAAV (SEQ ID NO: 221), GGCDI (SEQ ID NO: 222), SGSVV (SEQ ID NO: 223), SSNAC (SEQ ID NO: 224), YNTTV (SEQ ID NO: 225), SKCLA (SEQ ID NO: 226), SAYTV (SEQ ID NO: 227), VRDTV (SEQ ID NO: 228), WRSMV (SEQ ID NO: 229), AYHGV (SEQ ID NO: 230), GMNTI (SEQ ID NO: 231), AETSL (SEQ ID NO: 232), TLVYV (SEQ ID NO: 233), NHDWI (SEQ ID NO: 234), TVGIV (SEQ ID NO: 235), SLPTV (SEQ ID NO: 236), TGILC (SEQ ID NO: 237), TDTYI (SEQ ID NO: 238), LPVTY (SEQ ID NO: 239), GDVYI (SEQ ID NO: 240), LYGTV (SEQ ID NO: 241), GCEF1 (SEQ ID NO: 242), SAGLL (SEQ ID NO: 243), IKSNI (SEQ ID NO: 244), VTTSL (SEQ ID NO: 245), AVTSV (SEQ ID NO: 246), RDIHI (SEQ ID NO: 247), SAISL (SEQ ID NO: 248), VASTC (SEQ ID NO: 249), IKGLL (SEQ ID NO: 250), GSYHT (SEQ ID NO: 251), RIGFV (SEQ ID NO: 252), NDIYI (SEQ ID NO: 253), AVSCV (SEQ ID NO: 254), QINLL (SEQ ID NO: 255), VSSCV (SEQ ID NO: 256), LNLDV (SEQ ID NO: 257), LGATI (SEQ ID NO: 258), PVLCV (SEQ ID NO: 259), SARHI (SEQ ID NO: 260), RATLI (SEQ ID NO: 261), PYNHA (SEQ ID NO: 262), IGDSI (SEQ ID NO: 263), SPMLC (SEQ ID NO: 264), YDSTL (SEQ ID NO: 265), ALKHV (SEQ ID NO: 266), ADLLT (SEQ ID NO: 267), NNGHL (SEQ ID NO: 268), INSEV (SEQ ID NO: 269), SNKTT (SEQ ID NO: 270), GSTGL (SEQ ID NO: 271), DSDMI (SEQ ID NO: 272), TSNFI (SEQ ID NO: 273), RNFTT (SEQ ID NO: 274), SHKYS (SEQ ID NO: 275), VSDIV (SEQ ID NO: 276), RVVQA (SEQ ID NO: 277), AACAV (SEQ ID NO: 278), RGRQI (SEQ ID NO: 279), AVANI (SEQ ID NO: 280), AGYDL (SEQ ID NO: 281), LSEAA (SEQ ID NO: 282), MSNYL (SEQ ID NO: 283), NFSDN (SEQ ID NO: 284), SCCDV (SEQ ID NO: 285), LASSV (SEQ ID NO: 286), PDHAV (SEQ ID NO: 287), KFDII (SEQ ID NO: 288), NSSSA (SEQ ID NO: 289), HTMHV (SEQ ID NO: 290), TLSYC (SEQ ID NO: 291), ADTHR (SEQ ID NO: 292), SMYSV (SEQ ID NO: 293), SVNLV (SEQ ID NO: 294), MSGHI, (SEQ ID NO: 295), KISDT (SEQ ID NO: 296), TGLLA (SEQ ID NO: 297), AWTTS (SEQ ID NO: 298), GGALI (SEQ ID NO: 299), SCIEV (SEQ ID NO: 300), PPVIC (SEQ ID NO: 301), and GTYNL (SEQ ID NO: 302).

In some embodiments, the variant polypeptide sequence at the VR-VIII site comprises, consists essentially of, or consists of a polypeptide sequence at least about 60%, 70%, 80%, 90%, or 100% identical to one of SEQ ID NOs: 204-302.

In some embodiments, the variant polypeptide sequence at the VR-VII site comprises, consists essentially of, or consists of a sequence at least about 60%, 70%, 80%, 90%, or 100% identical to RGNQV (SEQ ID NO: 204). In some embodiments, the variant polypeptide sequence at the VR-VIII site comprises, consists essentially of, or consists of a sequence consisting of at most 1, 2, 3, or 4 amino-acid substitutions relative to RGNQV (SEQ ID NO: 204). In some embodiments, the variant polypeptide sequence at the VR-VIII site comprises, consists essentially of, or consists of a sequence consisting of at most 1, 2, 3, or 4 conservative amino-acid substitutions relative RGNQV (SEQ ID NO: 204). In some embodiments, the variant polypeptide sequence at the VR-VIII site is RGNQV (SEQ ID NO: 204).

### Modifications in VR-VIII Site

In some embodiments, the capsid protein of the present disclosure comprises a variant polypeptide sequence at the VR-VIII site.

In some embodiments, the amino acids at positions 586 to 589 (relative to reference sequence SEQ ID NO:1) of the VR-VIII site (“SAQA”) are substituted by a peptide of formula:

-(X)n-

wherein n is 2-6, and X represents any of the 20 standard amino acids (SEQ ID NO: 481).

In some embodiments, the variant polypeptide sequence at the VR-VIII site is:

(SEQ ID NO: 481)
-X1-X2-X3-X4-

In some embodiments, the variant polypeptide sequence at the VR-VIII site is:

-X1-X2-X3-X4-

wherein X1 is S, N, or A; X2 is V, M, N, or A; X3 is Y, V, S, or O; and X4 is Y, T, M, G, or N (SEQ ID NO: 476).

In some embodiments, the variant polypeptide sequence at the VR-VIII site comprises:

-X1-X2-X3-X4-

wherein X1 is S, N, T, M, G, or D; X2 is A, T, L, I, K, S, N or V; X3 is Q, V, F, Y, L, T, S, I, R, or Q; and X4 is A, S, N, L, T, I, or R (SEQ ID NO: 731).

In some embodiments, the variant polypeptide sequence at the VR-VIII site comprises:

-X1-X2-X3-X4-

wherein X1 is S, N, T, M, G, or D; X2 is T, L, I, K, S, N or V; X3 is V, F, Y, L, T, S, I, R, or Q; and X4 is A, S, N, L, T, I, or R (SEQ ID NO: 732).

In some embodiments, the variant polypeptide sequence at the VR-VIII site comprises:

-X1-X2-X3-X4-

wherein X1 is S, N, M, or T; X2 is A, T, L, or I; X3 is Q, V, F, Y, T, S, or L; and X4 is A, S, N, L, I, or T (SEQ ID NO: 733).

In some embodiments, the variant polypeptide sequence at the VR-VIII site comprises:

-X1-X2-X3-X4-

wherein X1 is S, N, M, or T; X2 is T, L, or I; X3 is V, F, Y, T, S, or L; and X4 is A, S, N, L, I, or T (SEQ ID NO: 734).

In some embodiments, the variant polypeptide sequence at the VR-VIII site comprises:

-X1-X2-X3-X4-

wherein X1 is S, M, D, N, G, A, T, R, or I; X2 is T, N, V, A, L, I, S, R, or P; X3 is Y, T, S, I, V, F, L, R, N, D, G, or Q; and X4 is L, A, I, R, S, G, N, T, V, Q, F, E, or Y (SEQ ID NO: 760).

In some embodiments, the variant polypeptide sequence at the VR-VIII site comprises:

-X1-X2-X3-X4-

wherein X1 is S, M, D, N, G, or A; X2 is T, N, V, or A; X3 is Y, T, S, I, or V; and X4 is L, A, I, R, S, or G (SEQ ID NO: 761).

In some embodiments, the variant polypeptide sequence at the VR-VIII site comprises or consists of a sequence selected from NVSY (SEQ ID NO: 303), SMVN (SEQ ID NO: 304), ANYG (SEQ ID NO: 305), NVGT (SEQ ID NO: 306), SAYM (SEQ ID NO: 307), EKVT (SEQ ID NO: 308), TTPG (SEQ ID NO: 309), GVYS (SEQ ID NO: 310), SYVG (SEQ ID NO: 311), LQYN (SEQ ID NO: 312), DPAK (SEQ ID NO: 313), THFS (SEQ ID NO: 314), IGGV (SEQ ID NO: 315), SSWN (SEQ ID NO: 316), SVYV (SEQ ID NO: 317), TLNG (SEQ ID NO: 318), NTSN (SEQ ID NO: 319), VQYA (SEQ ID NO: 320), DQYR (SEQ ID NO: 321), MPVS (SEQ ID NO: 322), SAQA (SEQ ID NO: 323), MTVA (SEQ ID NO: 324), TVMG (SEQ ID NO: 325), FSSI (SEQ ID NO: 326), SLRL (SEQ ID NO: 327), SAMG (SEQ ID NO: 328), YIKL (SEQ ID NO: 329), LMTM (SEQ ID NO: 330), QVHL (SEQ I) NO: 331), YNSV (SEQ ID NO: 332), CVIS (SEQ ID NO: 333), RLDG (SEQ ID NO: 334), AIMV (SEQ ID NO: 335), OTTO (SEQ ID NO: 336), ASYT (SEQ ID NO: 337), LHVG (SEQ ID NO: 338), LQFA (SEQ ID NO: 339), VRGD (SEQ ID NO: 340), NVMI (SEQ ID NO: 341), SLYG (SEQ ID NO: 342), GTVG (SEQ ID NO: 343), FNSV (SEQ ID NO: 344), TRLG (SEQ ID NO: 345), LKVL (SEQ ID NO: 346), SIRV (SEQ ID NO: 347), KIQG (SEQ ID NO: 348), QILG (SEQ ID NO: 349), QRDA (SEQ ID NO: 350), EAVR (SEQ ID NO: 351), AITV (SEQ ID NO: 352), KESI (SEQ ID NO: 353), LMVN (SEQ ID NO: 354), INLS (SEQ ID NO: 355), GQVS (SEQ ID NO: 356), TSLL (SEQ ID NO: 357), SSTL (SEQ ID NO: 358), YEKF (SEQ ID NO: 359), DGKL (SEQ ID NO: 360), QVYS (SEQ ID NO: 361), QKEG (SEQ ID NO: 362), ARDM (SEQ ID NO: 363), DNFR (SEQ ID NO: 364), SHGL, (SEQ ID NO: 365), VSVN (SEQ ID NO: 366), GLKD (SEQ ID NO: 367), QPVF (SEQ ID NO: 368), VYSM (SEQ ID NO: 369), VMAQ (SEQ ID NO: 370), FVGM (SEQ ID NO: 371), WSTP (SEQ ID NO: 372), SYPV (SEQ ID NO: 373), TTYS (SEQ ID NO: 374), TVTT (SEQ ID NO: 375), KDKT (SEQ ID NO: 376), YREL (SEQ ID NO: 377), LSHF (SEQ ID NO: 378), SPOT (SEQ ID NO: 379), LMGT (SEQ ID NO: 380), AASL (SEQ ID NO: 381), FSNN (SEQ ID NO: 382), QARL (SEQ ID NO: 383), YHIA (SEQ ID NO: 384), ARQD (SEQ ID NO: 385), VAYT (SEQ ID NO: 386), TPSY (SEQ ID NO: 387), MILH (SEQ ID NO: 388), LGNV (SEQ ID NO: 389), TSIS (SEQ ID NO: 390), TMVY (SEQ ID NO: 391), LVVG (SEQ ID NO: 392), SPLY (SEQ ID NO: 393), YKSE (SEQ ID NO: 394), FTRIL (SEQ ID NO: 395), VSYN (SEQ ID NO: 396), ERTP (SEQ ID NO: 397), FRSE (SEQ ID NO: 398), NYTE (SEQ ID NO: 399), QTIN (SEQ ID NO: 400), and DVHR (SEQ ID NO: 401). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises or consists of a sequence selected from NTVS (SEQ ID NO: 712), TLFN (SEQ ID NO: 713), STYL (SEQ ID NO: 714), SILT (SEQ ID NO: 715), MTTA (SEQ ID NO: 716), and STSI (SEQ ID NO: 717). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence STYL (SEQ ID NO: 714). In some embodiments, a capsid described herein comprises the variant polypeptide sequence at the VR-VIII site comprising the sequence STYL (SEQ ID NO: 714), and further comprises N452K substitution (relative to reference sequence SEQ ID NO:1) in the VR-IV site. In some embodiments, a capsid described herein comprises the variant polypeptide sequence at the VR-VIII site comprising the sequence STYL (SEQ ID NO: 714) and does not comprise N452K substitution (relative to reference sequence SEQ ID NO:1) in the VR-IV site. In some embodiments, the variant polypeptide sequence at the VR-VIII site comprises the sequence STYL (SEQ ID NO: 714). In some embodiments, a capsid described herein comprises the variant polypeptide sequence at the VR-VIII site comprising the sequence NSTYLG (SEQ ID NO: 721), and further comprises N452K substitution (relative to reference sequence SEQ ID NO:1) in the VR-IV site. In some embodiments, a capsid described herein comprises the variant polypeptide sequence at the VR-VIII site comprising the sequence NSTYLG (SEQ ID NO: 721) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence MTTA (SEQ ID NO: 716). In some embodiments, a capsid described herein comprises the variant polypeptide sequence at the VR-VIII site comprising the sequence MTTA (SEQ ID NO: 716), and further comprises N452K substitution (relative to reference sequence SEQ ID NO:1) in the VR-IV site. In some embodiments, a capsid described herein comprises the variant polypeptide sequence at the VR-VIII site comprising the sequence MTTA (SEQ ID NO: 716) and does not comprise N452K substitution (relative to reference sequence SEQ ID NO:1) in the VR-IV site. In some embodiments, the variant polypeptide sequence at the VR-VIII site comprises the sequence MMTTAR (SEQ ID NO: 723). In some embodiments, a capsid described herein comprises the variant polypeptide sequence at the VR-VIII site comprising the sequence MMTTAR (SEQ ID NO: 723), and further comprises N452K substitution (relative to reference sequence SEQ ID NO:1) in the VR-IV site. In some embodiments, a capsid described herein comprises the variant polypeptide sequence at the VR-VIII site comprising the sequence MMTTAR (SEQ ID NO: 723) and does not comprise N452K substitution (relative to reference sequence SEQ TID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence STSI (SEQ ID NO: 717). In some embodiments, a capsid described herein comprises the variant polypeptide sequence at the VR-VIII site comprising the sequence STSI (SEQ ID NO: 717), and further comprises N452K substitution (relative to reference sequence SEQ ID NO:1) in the VR-IV site. In some embodiments, a capsid described herein comprises the variant polypeptide sequence at the VR-VIII site comprising the sequence STSI (SEQ ID NO: 717) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence NVIS (SEQ ID NO: 745). In some embodiments, a capsid described herein comprises the variant polypeptide sequence at the VR-VIII site comprising the sequence NVIS (SEQ ID NO: 745), and further comprises N452K substitution (relative to reference sequence SEQ ID NO:1) in the VR-IV site. In some embodiments, a capsid described herein comprises the variant polypeptide sequence at the VR-VIII site comprising the sequence NVIS (SEQ ID NO: 745) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence DNIR (SEQ ID NO: 744). In some embodiments, a capsid described herein comprises the variant polypeptide sequence at the VR-VIII site comprising the sequence DNIR (SEQ ID NO: 744), and further comprises N452K substitution (relative to reference sequence SEQ ID NO:1) in the VR-IV site. In some embodiments, a capsid described herein comprises the variant polypeptide sequence at the VR-VIII site comprising the sequence DNIR (SEQ ID NO: 744) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a polypeptide sequence at least about 60%, 70%, 80%, 90%, 95%, or 100% identical to one of SEQ ID NOs: 303-401.

In some embodiments, the variant polypeptide sequence at the VR-VIII site comprises, consists essentially of, or consists of a sequence at least about 60%, 70%, 80%, 90%, or 100% identical to ANYG (SEQ ID NO: 305). In some embodiments, the variant polypeptide sequence at the VR-VIII site comprises, consists essentially of, or consists of a sequence consisting of at most 1, 2, or 3 amino-acid substitutions relative to ANYG (SEQ ID NO: 305). In some embodiments, the variant polypeptide sequence at the VR-VIII site comprises, consists essentially of, or consists of a sequence consisting of at most 1, 2, or 3 conservative amino-acid substitutions relative ANYG (SEQ ID NO: 305). In some embodiments, the variant polypeptide sequence at the VR-VIII site is ANYG (SEQ ID NO: 305).

In some embodiments, the variant polypeptide sequence at the VR-VIII site comprises, consists essentially of, or consists of a sequence at least about 60%, 70%, 80%, 90%, or 100% identical to NVSY (SEQ ID NO: 303). In some embodiments, the variant polypeptide sequence at the VR-VIII site comprises, consists essentially of, or consists of a sequence consisting of at most 1, 2, or 3 amino-acid substitutions relative to NVSY (SEQ ID NO: 303). In some embodiments, the variant polypeptide sequence at the VR-VIII site comprises, consists essentially of, or consists of a sequence consisting of at most 1, 2, or 3 conservative amino-acid substitutions relative NVSY (SEQ ID NO: 303). In some embodiments, the variant polypeptide sequence at the VR-VIII site is NVSY (SEQ ID NO: 303).

In some embodiments, the variant polypeptide sequence at the VR-VIII site comprises, consists essentially of, or consists of a polypeptide sequence at least about 60%, 70%, 80%, 90%, 95%, or 100% identical to one of SEQ ID NOs: 712-717.

In some embodiments, the variant polypeptide sequence at the VR-VIII site comprises, consists essentially of, or consists of a sequence at least about 60%, 70%, 80%, 90%, or 100% identical to NTVS (SEQ ID NO: 712). In some embodiments, the variant polypeptide sequence at the VR-VIII site comprises, consists essentially of, or consists of a sequence consisting of at most 1, 2, or 3 amino-acid substitutions relative to NTVS (SEQ ID NO: 712). In some embodiments, the variant polypeptide sequence at the VR-VIII site comprises, consists essentially of, or consists of a sequence consisting of at most 1, 2, or 3 conservative amino-acid substitutions relative NTVS (SEQ ID NO: 712). In some embodiments, the variant polypeptide sequence at the VR-VIII site is NTVS (SEQ ID NO: 712).

In some embodiments, the variant polypeptide sequence at the VR-VIII site comprises, consists essentially of, or consists of a sequence at least about 60%, 70%, 80%, 90%, or 100% identical to TLFN (SEQ ID NO: 713). In some embodiments, the variant polypeptide sequence at the VR-VIII site comprises, consists essentially of, or consists of a sequence consisting of at most 1, 2, or 3 amino-acid substitutions relative to TLFN (SEQ ID NO: 713). In some embodiments, the variant polypeptide sequence at the VR-VIII site comprises, consists essentially of, or consists of a sequence consisting of at most 1, 2, or 3 conservative amino-acid substitutions relative TLFN (SEQ ID NO: 713). In some embodiments, the variant polypeptide sequence at the VR-VIII site is TLFN (SEQ ID NO: 713).

In some embodiments, the variant polypeptide sequence at the VR-VIII site comprises, consists essentially of, or consists of a sequence at least about 60%, 70%, 80%, 90%, or 100% identical to STYL (SEQ ID NO: 714). In some embodiments, the variant polypeptide sequence at the VR-VIII site comprises, consists essentially of, or consists of a sequence consisting of at most 1, 2, or 3 amino-acid substitutions relative to STYL (SEQ ID NO: 714). In some embodiments, the variant polypeptide sequence at the VR-VIII site comprises, consists essentially of, or consists of a sequence consisting of at most 1, 2, or 3 conservative amino-acid substitutions relative STYL (SEQ ID NO: 714). In some embodiments, the variant polypeptide sequence at the VR-VIII site is STYL (SEQ ID NO: 714).

In some embodiments, the variant polypeptide sequence at the VR-VIII site comprises, consists essentially of, or consists of a sequence at least about 60%, 70%, 80%, 90%, or 100% identical to SILT (SEQ ID NO: 715). In some embodiments, the variant polypeptide sequence at the VR-VIII site comprises, consists essentially of, or consists of a sequence consisting of at most 1, 2, or 3 amino-acid substitutions relative to SILT (SEQ ID NO: 715). In some embodiments, the variant polypeptide sequence at the VR-VIII site comprises, consists essentially of, or consists of a sequence consisting of at most 1, 2, or 3 conservative amino-acid substitutions relative SILT (SEQ ID NO: 715). In some embodiments, the variant polypeptide sequence at the VR-VIII site is SILT (SEQ ID NO: 715).

In some embodiments, the variant polypeptide sequence at the VR-VIII site comprises, consists essentially of, or consists of a sequence at least about 60%, 70%, 80%, 90%, or 1000% identical to MTTA (SEQ ID NO: 716). In some embodiments, the variant polypeptide sequence at the VR-VIII site comprises, consists essentially of, or consists of a sequence consisting of at most 1, 2, or 3 amino-acid substitutions relative to MTTA (SEQ ID NO: 716). In some embodiments, the variant polypeptide sequence at the VR-VIII site comprises, consists essentially of, or consists of a sequence consisting of at most 1, 2, or 3 conservative amino-acid substitutions relative MTTA (SEQ ID NO: 716). In some embodiments, the variant polypeptide sequence at the VR-VIII site is MTTA (SEQ ID NO: 716).

In some embodiments, the variant polypeptide sequence at the VR-VIII site comprises, consists essentially of, or consists of a sequence at least about 60%, 70%, 80%, 90%, or 100% identical to STSI (SEQ ID NO: 717). In some embodiments, the variant polypeptide sequence at the VR-VIII site comprises, consists essentially of, or consists of a sequence consisting of at most 1, 2, or 3 amino-acid substitutions relative to STSI (SEQ ID NO: 717). In some embodiments, the variant polypeptide sequence at the VR-VIII site comprises, consists essentially of, or consists of a sequence consisting of at most 1, 2, or 3 conservative amino-acid substitutions relative STSI (SEQ ID NO: 717). In some embodiments, the variant polypeptide sequence at the VR-VIII site is STSI (SEQ ID NO: 717).

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 712-717, or a functional fragment thereof.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 402-410 and 464-468, or a functional fragment thereof.

TABLE 1
Capsid Protein Sequences

| Name/Alternate Name | SEQ ID NO: |
|---|---|
| CR9-01/TN1 | 402 |
| CR9-07 | 403 |
| CR9-07-A/TN5 | 482 |
| CR9-07-E/TN6 | 483 |
| CR9-08 | 464 |
| CR9-09 | 465 |
| CR9-10/TN3 | 404 |
| CR9-11 | 466 |
| CR9-13 | 405 |
| CR9-14/TN4 | 406 |
| CR9-15 | 467 |
| CR9-16 | 468 |
| CR9-17 | 407 |
| CR9-20 | 408 |
| CR9-21 | 409 |
| CR9-22 | 410 |
| HV1/TN7 | 484 |
| HV2/TN11 | 485 |

In some embodiments, the capsid protein of the present disclosure comprises a variant polypeptide sequence at the VR-VIII site. In some embodiments, the entire VR-VIII site comprises or consists of amino acids ATNHQSAQAQAQTG (SEQ ID NO: 5), wherein amino acids QSAQAQ (SEQ ID NO: 756) are substituted by a peptide of formula:

-(X)ₙ-

wherein n is 4-8, and X represents any of the 20 standard amino acids (SEQ ID NO: 481).

In some embodiments, the variant polypeptide sequence at the VR-VIII site is or comprises:

(SEQ ID NO: 481)
-X1-X2-X3-X4-X5-X6-

In some embodiments, the variant polypeptide sequence at the VR-VIII site is or comprises:
-X₁-X₂-X₃-X₄-X₅-X₆
   wherein X₁ is N, M, C, E, G, S, V, A, T, H, L, or Q; X₂ is M, D, N, G, A, T, R, I, or S; X₃ is T, N, V, L, I, S, R, P, or A; X₄ is Y, T, S, I, V, F, L, R, N, D, G, or Q; X₅ is L, I, R, S, G, N, T, V, Q, F, E, Y, or A, and X₆ is G, R, S, I, H, N, Y, L, M, or Q (SEQ ID NO: 762).

In some embodiments, the variant polypeptide sequence at the VR-VIII site is or comprises:
-X₁-X₂-X₃-X₄-X₅-X₆-X₇-
   wherein X₁ is R or H; X₂ is N, M, C, E, G, S, V, A, T, H, L, or Q; X₃ is M, D, N, G, A, T, R, I, or S; X₄ is T, N, V, L, I, S, R, P, or A; X₅ is Y, T, S, I, V, F, L, R, N, D, G, or Q; X₆ is L, I, R, S, G, N, T, V, Q, F, E, Y, or A, and X₇ is G, R, S, I, H, N, Y, L, M, or Q (SEQ ID NO: 781).

In some embodiments, the variant polypeptide sequence at the VR-VIII site is or comprises:
-X₁-X₂-X₃-X₄-X₅-X₆
   wherein X₁ is N, M, C, E, G, S, V, A, T, H, or L; X₂ is M, D, N, G, A, T, R, or I; X₃ is T, N, V, L, I, S, R, or P; X₄ is Y, T, S, I, V, F, L, R, N, D, or G; X₅ is L, I, R, S, G, N, T, V, Q, F, E, or Y, and X₆ is G, R, S, I, H, N, Y, L, or M (SEQ ID NO: 763).

In some embodiments, the variant polypeptide sequence at the VR-VIII site is or comprises:
-X₁-X₂-X₃-X₄-X₅-X₆
   wherein X₁ is Q, E, N, G, M, C, V, or T; X₂ is S, N, T, M, G, or D; X₃ is A, T, L, I, K, S, N or V; X₄ is Q, V, F, Y, L, T, S, I, or R; X₅ is A, S, N, L, T, I, or R, and X₆ is Q, I, S, G, H or R (SEQ ID NO: 735).

In some embodiments, the variant polypeptide sequence at the VR-VIII site is or comprises:
-X₁-X₂-X₃-X₄-X₅-X₆
   wherein X₁ is Q, F, N, G, M, C, V, or T; X₂ is S, N, T, M, G, or D; X₃ is T, L, I, K, S, N or V: X₄ is V, F, Y, L, T, S, I, R, or Q; X₅ is A, S, N, L, T, I, or R, and X₆ is I, S, G, H or R (SEQ ID NO: 736).

In some embodiments, the variant polypeptide sequence at the VR-VIII site is or comprises:
-X₁-X₂-X₃-X₄-X₅-X₆
   wherein X₁ is Q, E, N, M, C, or G; X₂ is S, N, M, or T; X₃ is A, T, L, or I; X₄ is Q, V, F, Y, T, S, or L; X₅ is A, S, N, L, I, or T; and X₆ is I, S, G, R, or H (SEQ ID NO: 737).

In some embodiments, the variant polypeptide sequence at the VR-VIII site is or comprises:
-X₁-X₂-X₃-X₄-X₅-X₆
   wherein X₁ is E, N, M, C, or G; X₂ is S, N, M, or T; X₃ is T, L, or I; X₄ is V, F, Y, T, S, or L; X₅ is A, S, N, L, I, or T; and X₆ is I, S, G, R, or H (SEQ ID NO: 738).

In some embodiments, the variant polypeptide sequence at the VR-VIII site is or comprises:
-X₁-X₂-X₃-X₄-X₅-X₆
   wherein X₁ is Q, E, N, G, M, or C; X₂ is S, N, T, or M; X₃ is A, T, L, T, or S; X₄ is Q, V, F, Y, L, or I; X₅ is A, S, N, L, T, or I; and X₆ is I, S, Q, G, H, or R (SEQ ID NO: 718).

In some embodiments, the variant polypeptide sequence at the VR-VIII site is or comprises:
-X₁-X₂-X₃-X₄-X₅-X₆
   wherein X₁ is E, N, G, M, C, V, or T; X₂ is N, T, M, G, or D; X₃ is T, L, I, K, S, N or V; X₄ is V, F, Y, L, T, S, I, R; X₅ is S, N, L, T, I, or R, and X₆ is I, S, G, H or R (SEQ ID NO: 764).

In some embodiments, the variant polypeptide sequence at the VR-VIII site is or comprises:
-X₁-X₂-X₃-X₄-X₅-X₆
   wherein X₁ is E, N, M, C, or Q; X₂ is A, M, G, D, N, or S; X₃ is T, N, V, or A; X₄ is V, Y, T, S, I, or Q; X₅ is S, G, L, I, R, or A; and X₆ is I, S, G, R, or Q (SEQ ID NO: 765).

In some embodiments, the variant polypeptide sequence at the VR-VIII site is or comprises:
-X₁-X₂-X₃-X₄-X₅-X₆
   wherein X₁ is E, N, M, or C; X₂ is A, M, G, D, or N; X₃ is T, N, or V; X₄ is V, Y, T, S, or I; X₅ is S, G, L, I, or R; and X₆ is I, S, G, or R (SEQ ID NO: 766).

In some embodiments, the capsid protein of the present disclosure comprises a variant polypeptide sequence at the VR-VIII site. In some embodiments, the entire VR-VIII site comprises the following peptide of formula:

ATNH-(X)ₙ-AQTG

wherein n is 4-8, and X represents any of the 20 standard amino acids (SEQ ID NO: 740).

In some embodiments, the entire VR-VIII site comprises the following peptide of formula:

ATNH-X₁-X₂-X₃-X₄-X₅-X₆-AQTG (SEQ ID NO: 740).

In some embodiments, X₁-X₂-X₃-X₄-X₅-X₆ are as described above. For example, in some embodiments, X₁ is Q, E, N, G, M, C, V, or T; X₂ is S, N, T, M, G, or D; X₃ is A, T, L, I, K, S, N or V; X₄ is Q, V, F, Y, L, T, S, I, R, or Q; X₅ is A, S, N, L, T, I, or R, and X₆ is Q, I, S, G, H or R (SEQ ID NO: 728). In some embodiments, X₁ is Q, E, N, G, M, or C; X₂ is S, N, T, or M; X₃ is A, T, L, I, or S; X₄ is Q, V, F, Y, L, or I; X₅ is A, S, N, L, T, or I; and X₆ is I, S, Q, G, H, or R (SEQ ID NO: 739).

In some of these embodiments, the capsid protein comprises N or K at position 452 relative to reference sequence SEQ ID NO: 1 (in addition to the variant polypeptide sequence described herein).

In some of these embodiments, the capsid protein may further comprise N452K substitution relative to reference sequence SEQ ID NO: 1 (in addition to the variant polypeptide sequence described herein).

In some embodiments, the variant polypeptide sequence at the VR-VIII site comprises or consists of a sequence selected from ENTVSI (SEQ ID NO: 719), QTLFNS (SEQ ID NO: 720), NSTYLG (SEQ ID NO: 721), GSILTH (SEQ ID NO: 722), MMTTAR (SEQ ID NO: 723), and CSTSIR (SEQ ID NO: 724). In some of these embodiments, the capsid protein may further comprise N452K substitution relative to reference sequence SEQ ID NO: 1 (in addition to the variant polypeptide sequence). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises or consists of a sequence selected from NSTYLG (SEQ ID NO: 721), MMTTAR (SEQ ID NO: 723), CSTSIR (SEQ ID NO: 724), EDNIRS (SEQ ID NO: 725), NNVISG (SEQ ID NO: 752), QGAYAQ (SEQ ID NO: 749), VSSFTS (SEQ ID NO: 751), TGTSII (SEQ ID NO: 753), and QHYSAQAQ (SEQ ID NO: 759), In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises or consists of a sequence selected from NSTYLG (SEQ ID NO: 721), MMTTAR (SEQ ID NO: 723), CSTSIR (SEQ ID NO: 724), EDNIRS (SEQ ID NO: 725), and NNVISG (SEQ ID NO: 752). In some of these embodiments, the capsid protein may further comprise N452K substitution relative to reference sequence SEQ ID NO: 1 (in addition to the variant polypeptide sequence described herein). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence NSTYLG (SEQ ID NO: 721). In some embodiments, the variant polypeptide sequence at the VR-VIII site comprises, consists essentially of, or consists of a sequence at least about 60%, 70%, 80%, 83%, 90%, or 100% identical to NSTYLG (SEQ ID NO: 721). In some embodiments, a capsid described herein comprises the variant polypeptide sequence at the VR-VIII site comprising the sequence NSTYLG (SEQ ID NO: 721), and further comprises N452K substitution (relative to reference sequence SEQ ID NO:1) in the VR-IV site. In some embodiments, a capsid described herein comprises the variant polypeptide sequence at the VR-VIII site comprising the sequence NSTYLG (SEQ ID NO: 721) and does not comprise N452K substitution (relative to reference sequence SEQ ID NO:1) in the VR-IV site. In some of these embodiments, the capsid protein comprises the sequence at least 85%, 90%, 95%, 98%, 99% or 10(0% identical to VP3 of SEQ ID NO:487 except for the specific substitutions at the VR-VIII site and, optionally, position 452 described herein.

In some embodiments, the variant polypeptide sequence at the VR-VIII site comprises the sequence MMTTAR (SEQ ID NO: 723). In some embodiments, the variant polypeptide sequence at the VR-VIII site comprises, consists essentially of, or consists of a sequence at least about 60%, 70%, 80%, 83%, 90%, or 100% identical to MMTTAR (SEQ ID NO: 723). In some embodiments, a capsid described herein comprises the variant polypeptide sequence at the VR-VIII site comprising the sequence MMTTAR (SEQ ID NO: 723), and further comprises N452K substitution (relative to reference sequence SEQ ID NO:1) in the VR-IV site. In some embodiments, a capsid described herein comprises the variant polypeptide sequence at the VR-VIII site comprising the sequence MMTTAR (SEQ ID NO: 723) and does not comprise N452K substitution (relative to reference sequence SEQ ID NO:1) in the VR-TV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence CSTSIR (SEQ ID NO: 724), in some embodiments, the variant polypeptide sequence at the VR-VIII site comprises, consists essentially of, or consists of a sequence at least about 60%, 70%, 80%, 83%, 90%, or 100% identical to CSTSIR (SEQ ID NO: 724). In some embodiments, a capsid described herein comprises the variant polypeptide sequence at the VR-VIII site comprising the sequence CSTSIR (SEQ ID NO: 724), and further comprises N452K substitution (relative to reference sequence SEQ ID NO:1) in the VR-IV site. In some embodiments, a capsid described herein comprises the variant polypeptide sequence at the VR-VIII site comprising the sequence CSTSIR (SEQ ID NO: 724) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence NNVISG (SEQ ID NO: 752). In some embodiments, the variant polypeptide sequence at the VR-VIII site comprises, consists essentially of, or consists of a sequence at least about 60%, 70%, 80%, 83%, 90%, or 100% identical to NNVISG (SEQ ID NO: 752). In some embodiments, a capsid described herein comprises the variant polypeptide sequence at the VR-VIII site comprising the sequence NNVISG (SEQ ID NO: 752), and further comprises N452K substitution (relative to reference sequence SEQ ID NO:1) in the VR-IV site. In some embodiments, a capsid described herein comprises the variant polypeptide sequence at the VR-VIII site comprising the sequence NNVISG (SEQ ID NO: 752) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises the sequence EDNIRS (SEQ ID NO: 725). In some embodiments, the variant polypeptide sequence at the VR-VIII site comprises, consists essentially of, or consists of a sequence at least about 60%, 70%, 80%, 83%, 90%, or 100% identical to EDNIRS (SEQ ID NO: 725). In some embodiments, a capsid described herein comprises the variant polypeptide sequence at the VR-VIII site comprising the sequence EDNIRS (SEQ ID NO: 725), and further comprises N452K substitution (relative to reference sequence SEQ ID NO:1) in the VR-IV site. In some embodiments, a capsid described herein comprises the variant polypeptide sequence at the VR-VIII site comprising the sequence EDNIRS (SEQ ID NO: 725) and does not comprise N452K substitution (relative to reference sequence SEQ ID NO:1) in the VR-IV site. In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a polypeptide sequence at least about 60%, 70%, 80%, 90%, 95% a, or 100% identical to one of SEQ ID NOs: 719-724.

In some embodiments, the variant polypeptide sequence at the VR-VITI site comprises, consists essentially of, or consists of a sequence at least about 60%, 70%, 80%, 83%, 90o, or 100% identical to ENTVSI (SEQ ID NO: 719). In some embodiments, the variant polypeptide sequence at the VR-VIII site comprises, consists essentially of, or consists of a sequence consisting of at most 1, 2, or 3 amino-acid substitutions relative to ENTVSI (SEQ ID NO: 719). In some embodiments, the variant polypeptide sequence at the VR-VIII site comprises, consists essentially of, or consists of a sequence consisting of at most 1, 2, or 3 conservative amino-acid substitutions ENTVSI (SEQ ID NO: 719). In some embodiments, the variant polypeptide sequence at the VR-VIII site is NTVS ENTVSI (SEQ ID NO: 719). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to QTLFNS (SEQ ID NO: 720). In some embodiments, the variant polypeptide sequence at the VR-VIII site comprises, consists essentially of, or consists of a sequence consisting of at most 1, 2, or 3 amino-acid substitutions relative to QTLFNS (SEQ ID NO: 720). In some embodiments, the variant polypeptide sequence at the VR-VIII site comprises, consists essentially of, or consists of a sequence consisting of at most 1, 2, or 3 conservative amino-acid substitutions relative QTLFNS (SEQ I) NO: 720). In some embodiments, the variant polypeptide sequence at the VR-VIII site is QTLFNS (SEQ ID NO: 720), In some of these embodiments, the capsid protein comprises the sequence at least 85%, 90′, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to NSTYLG (SEQ ID NO: 721). In some embodiments, the variant polypeptide sequence at the VR-VIII site comprises, consists essentially of, or consists of a sequence consisting of at most 1, 2, or 3 amino-acid substitutions relative to NSTYLG (SEQ ID NO: 721). In some embodiments, the variant polypeptide sequence at the VR-VIII site comprises, consists essentially of, or consists of a sequence consisting of at most 1, 2, or 3 conservative amino-acid substitutions relative NSTYLG (SEQ ID NO: 721). In some embodiments, the variant polypeptide sequence at the VR-VIII site is NSTYLG (SEQ ID NO: 721), In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to GSILTH (SEQ ID NO: 722). In some embodiments, the variant polypeptide sequence at the VR-VIII site comprises, consists essentially of, or consists of a sequence consisting of at most 1, 2, or 3 amino-acid substitutions relative to GSILTH (SEQ ID NO: 722). In some embodiments, the variant polypeptide sequence at the VR-VIII site comprises, consists essentially of, or consists of a sequence consisting of at most 1, 2, or 3 conservative amino-acid substitutions relative GSILTH (SEQ ID NO: 722). In some embodiments, the variant polypeptide sequence at the VR-VIII site is GSILTH (SEQ ID NO: 722), In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VITI site comprises, consists essentially of, or consists of a sequence at least about 60%, 70%, 80%, 83%, 90%, or 100% identical to MMTTAR (SEQ ID NO: 723). In some embodiments, the variant polypeptide sequence at the VR-VIII site comprises, consists essentially of, or consists of a sequence consisting of at most 1, 2, or 3 amino-acid substitutions relative to MMTTAR (SEQ ID NO: 723). In some embodiments, the variant polypeptide sequence at the VR-VIII site comprises, consists essentially of, or consists of a sequence consisting of at most 1, 2, or 3 conservative amino-acid substitutions relative MMTTAR (SEQ ID NO: 723). In some embodiments, the variant polypeptide sequence at the VR-VIII site is MMTTAR (SEQ ID NO: 723). In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the variant polypeptide sequence at the VR-VIII site comprises, consists essentially of, or consists of a sequence at least about 60%, 70%, 80%, 83%, 90%, or 100% identical to CSTSIR (SEQ ID NO: 724). In some embodiments, the variant polypeptide sequence at the VR-VIII site comprises, consists essentially of, or consists of a sequence consisting of at most 1, 2, or 3 amino-acid substitutions relative to CSTSIR (SEQ ID NO: 724). In some embodiments, the variant polypeptide sequence at the VR-VIII site comprises, consists essentially of, or consists of a sequence consisting of at most 1, 2, or 3 conservative amino-acid substitutions relative CSTSIR (SEQ ID NO: 724). In some embodiments, the variant polypeptide sequence at the VR-VIII site is CSTSIR (SEQ ID NO: 724), In some of these embodiments, the capsid protein comprises the sequence at least 85%, 90%, 95%, 98%, 99% or 100% identical to VP3 of SEQ ID NO:487 except for the specific substitutions at the VR-VIII site and, optionally, position 452 described herein.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 719-724, or a functional fragment thereof.

In some embodiments, the variant polypeptide sequence at the VR-VIII site comprises amino acid R or H at position 584 relative to reference sequence SEQ ID NO: 1. In some embodiments, the variant polypeptide sequence at the VR-VIII site comprises R at position 584.

In some embodiments, the variant polypeptide sequence at the VR-VIII site comprises A587T substitution (i.e., T at position 587) relative to reference sequence SEQ ID NO: 1.

In some embodiments, the variant polypeptide sequence at the VR-VIII site comprises amino acid N or R at one, two, three or more positions selected from the group consisting of:584, 585, 586, 588, 589, and 590 (or amino acid N or R within −3 to +3 positions from position 587), relative to reference sequence SEQ ID NO: 1. In some embodiments, the variant polypeptide sequence at the VR-VIII site comprises amino acid N or R at two, three or more positions selected from the group consisting of:584, 585, 586, 588, 589, and 590 (or amino acid N or R within −3 to +3 positions from position 587), relative to reference sequence SEQ ID NO: 1.

In some embodiments, the variant polypeptide sequence at the VR-VIII site comprises A587T substitution (i.e., T at position 587), and comprises amino acid N or R at one, two, three or more positions selected from the group consisting of:584, 585, 586, 588, 589, and 590 (or amino acid N or R within −3 to +3 positions from position 587), relative to reference sequence SEQ ID NO: 1.

In some embodiments, the variant polypeptide sequence at the VR-VIII site comprises amino acid S at two, three or more positions selected from the group consisting of: 585, 586, 587, 588, 589 and 590 (or two or more amino acids S at positions in the region 585-590), relative to reference sequence SEQ ID NO: 1.

In some embodiments, the variant polypeptide sequence at the VR-VIII site comprises, at three, four or more positions in the region 585-590, relative to reference sequence SEQ ID NO: 1, one, two or more amino acids (in any combination) selected from the group consisting of: N, S, T, R, and I. In some embodiments, the variant polypeptide sequence at the VR-VIII site comprises, at three, four or more positions in the region 585-590, relative to reference sequence SEQ ID NO: 1, one, two or more amino acids (in any combination) selected from the group consisting of: N, S, T, and R.

In some embodiments, the variant polypeptide sequence at the VR-VIII site comprises any one or more amino acids (e.g., any 2, 3, 4 or more, in any combination) selected from the group consisting of: N, S, T, R and I, at three, four or more positions in the region 585-590 (i.e., position 585, 586, 587, 588, 589, and/or 590), relative to reference sequence SEQ ID NO: 1. In some embodiments, the variant polypeptide sequence at the VR-VIII site comprises any one or more amino acids (e.g., any 2, 3, 4 or more, in any combination) selected from the group consisting of: N, S, T and R, at three, four or more positions in the region 585-590 (i.e., positions 585, 586, 587, 588, 589, and 590), relative to reference sequence SEQ ID NO: 1. For example, and without limitation, in the region 585-590, there may be three or more N, three or more S, three or more T, etc., or all three of N, S and T, or two or three of one referenced amino acid (e.g., N) and one or more of any other referenced amino acid (e.g., T), or one of each of these amino acids (i.e., all five of N, S, T, R and 1), and so on, in any combination.

In some of these embodiments, the capsid protein comprises N or K at position 452 relative to reference sequence SEQ ID NO: 1 (in addition to the variant polypeptide sequence described herein).

In some embodiments, the capsid protein may comprise N452K substitution relative to reference sequence SEQ ID NO: 1 (either by itself, or in addition to the variant polypeptide having one or more substitutions described herein, such as any substitution or substitution pattern at the VR-VIII site described herein).

In some embodiments, the capsid protein comprises N452K substitution relative to reference sequence SEQ ID NO: 1 (and, optionally, comprises 80%, 85%, 90%, 95%, 98%, 99% or 100% identity to VP3 of SEQ ID NO:487 and/or VP1 of SEQ ID NO:1 at positions other than 452).

In some embodiments, the variant VP1 capsid protein of SEQ ID NO:1 comprises one of the substitution patterns at the VR-VIII site positions 581-594 or 585-590 and/or position 452 of AAV9 VP1 presented in the below tables. In some embodiments, the variant VP1 capsid protein of SEQ ID NO:1 comprises a substitution pattern at the VR-VIII site positions 581-594 of AAV9 VP1 that has at least about 75%, 78.5%, 80%, 85%, 90%, 93% or 100% sequence identity to that presented in the below tables.

| | VR-VIII |
|---|---|
| Position 452 | Alignment (581-594) |
| N or K | ATNHENTVSIAQTG |
| N or K | ATNHQTLFNSAQTG |
| N or K | ATNHNSTYLGAQTG |
| N or K | ATNHGSILTHAQTG |
| N or K | ATNHMMTTARAQTG |
| N or K | ATNHCSTSIRAQTG |
| N or K | ATNHQGAYAQAQTG |
| N or K | ATNHNTKLAIAQTG |
| N or K | ATNHVSSFTSAQTG |
| N or K | ATNHEDNIRSAQTG |
| K | ATNHQSAQAQAQTG |
| N or K | ATNHNNVISGAQTG |
| N or K | ATNHTGTSIIAQTG |
| N or K | ATNHQWMSAQAQAQTG |
| N or K | ATNHQDARAQAQTG |
| N orK | ATNHQHYSAQAQAQTG |
| N or K | ATNHQSAQAQAQTG |
| N or K | ATNHNIRTEMAQTG |
| N or K | ATNHSTTNFRAQTG |

| Position | Position | Position | Position | Position | Position | Position |
|---|---|---|---|---|---|---|
| 585 | 586 | 587 | 588 | 589 | 590 | 452 |
| Q585E | S586N | A587T | Q588V | A589S | Q590I | N or N452K |
| Q | S586T | A587L | Q588F | A589N | Q590S | N or N452K |
| Q585N | S | A587T | Q588Y | A589L | Q590G | N or N452K |
| Q585G | S | A587I | Q588L | A589T | Q590H | N or N452K |
| Q585E | S586N | A587T | Q588V | A589S | Q590I | N or N452K |
| Q | S586T | A587L | Q588F | A589N | Q590S | N or N452K |
| Q585N | S | A587T | Q588Y | A589L | Q590G | N or N452K |
| Q585G | S | A587I | QS88L | A589T | QS90H | N or N452K |
| Q585M | S586M | A587T | Q588T | A | Q590R | N or N452K |
| Q585C | S | A587T | Q588S | A589I | Q590R | N or N452K |
| Q | S586G | A | Q588Y | A | Q | N or N452K |
| QS85N | S586T | A587K | Q588L | A | Q590I | N or N452K |
| Q585V | S | A587S | Q588F | A589T | Q590S | N or N452K |
| Q585E | S586D | A587N | Q588I | A589R | Q590S | N or N452K |
| Q | S | A | Q | A | Q | N or N452K |
| Q585N | S586N | A587V | Q588I | A589S | QS90G | N or N452K |
| Q585T | S586G | A587T | QS88S | A589I | Q590I | N or N452K |
| Q | S586D | A | Q588R | A | Q | N or N452K |
| Q585N | S586I | A587R | Q588T | A589E | Q590M | N or N452K |
| Q585S | S586T | A587T | Q588N | A589F | Q590R | N or N452K |
| C | S | A | Q | A | Q | N or N452K |

In some embodiments, the capsids in the above table have: (i) ATNH at positions 581, 582, 583 and 584, respectively, and/or (ii) AQTG at positions 591, 592, 593 and 594, respectively.

In some embodiments, the variant VP1 capsid protein of SEQ ID NO:1 comprises one of the following amino acids at the VR-VIII site positions 581-594 or 585-590:

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, N, | N, T, | T, L, | V, F, | S, N, | I, S, | A | Q | T | G |
| | | | | G, M, | M, G, | I, K, | Y, L, | L, T, | G, H, | | | | |
| | | | | C, V, | D, S, | S, N, | T, S, | I, R, | R, Q, | | | | |
| | | | | T, Q, | I | V, A, | I, R, | A, E, | M | | | | |
| | | | | S | | R | Q, N | F | | | | | |

In some embodiments, the variant VP1 capsid protein of SEQ ID NO:1 comprises one of the substitution patterns at the VR-VIII site positions 581-594 or 585-590 and/or position 452 of AAV9 VP1 presented in the below tables. In some embodiments, the variant VP1 capsid protein of SEQ. ID NO:1 comprises a substitution pattern at the VR-VIII site positions 581-594 of AAV9 VP1 that has at least about 75%, 78.5%, 80%, 85%, 90%, 93% or 100% sequence identity to that presented in the below tables.

| | VR-VIII |
|---|---|
| Position 452 | Alignment (581-594) |
| N or K | ATNHNSTYLGAQTG |
| N or K | ATNHMMTTARAQTG |
| N or K | ATNHCSTSIRAQTG |
| N or K | ATNHQGAYAQAQTG |
| N or K | ATNHVSSFTSAQTG |
| N or K | ATNHEDNIRSAQTG |
| N or K | ATNHNNVISGAQTG |
| N or K | ATNHTGTSIIAQTG |
| N or K | ATNHQHYSAQAQAQTG |

| Position | Position | Position | Position | Position | Position | Position |
|---|---|---|---|---|---|---|
| 585 | 586 | 587 | 588 | 589 | 590 | 452 |
| Q585N | S | A587T | Q588Y | A589L | Q590G | N or N452K |
| Q585M | S586M | A587T | Q588T | A | Q590R | N or N452K |
| Q585C | S | A587T | Q588S | A589I | Q590R | N or N452K |
| Q | S586G | A | Q588Y | A | Q | N or N452K |
| Q585V | S | A587S | Q588F | A589T | Q590S | N or N452K |
| Q585E | S586D | A587N | Q588I | A589R | Q590S | N or N452K |
| Q585N | S586N | A587V | Q588I | A589S | Q590G | N or N452K |
| Q585T | S586G | A587T | Q588S | A589I | Q590I | N or N452K |

In some embodiments, the capsids in the above table have: (i) ATNH at positions 581, 582, 583 and 584, respectively, and/or (ii) AQTG at positions 591, 592, 593 and 594, respectively.

In some embodiments, the variant VP1 capsid protein of SEQ ID NO:1 comprises one of the following amino acids at the VR-VIII site positions 581-594 or 585-590:

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, N, | N, M, | T, S, | F, Y, | S, L, | I, S, | A | Q | T | G |
| | | | | M, C, | G, D, | N, V, | T, S, | T, I, | G, R, | | | | |
| | | | | V, T, | S | A | I, | R, A | Q | | | | |
| | | | | Q | | | | | | | | | |

In some embodiments, the disclosure provides a recombinant adeno-associated virus (rAAV) capsid protein, wherein the capsid protein comprises, relative to reference sequence SEQ ID NO: 1, amino acid substitutions Q585E, S586N, A587T, Q588V, A589S, Q590I, and N452K.

In some embodiments, the disclosure provides a recombinant adeno-associated virus (rAAV) capsid protein, wherein the capsid protein comprises, relative to reference sequence SEQ ID NO: 1, amino acid substitutions S586T, A587L, Q588F, A589N, Q590S, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585N, A587T, Q588Y, A589L, Q590O, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585N, A587T, Q588Y, A589L, and Q590O.

In some embodiments, the disclosure provides a recombinant adeno-associated virus (rAAV) capsid protein, wherein the capsid protein comprises, relative to reference sequence SEQ ID NO: 1, amino acid substitutions Q585G, A587I, Q588L, A589T, Q590H, and N452K.

In some embodiments, the disclosure provides a recombinant adeno-associated virus (rAAV) capsid protein, wherein the capsid protein comprises, relative to reference sequence SEQ ID NO: 1, amino acid substitutions Q585M, S586M, A587T, Q588T, A589A, and Q590R.

In some embodiments, the disclosure provides a recombinant adeno-associated virus (rAAV) capsid protein, wherein the capsid protein comprises, relative to reference sequence SEQ ID NO: 1, amino acid substitutions Q585C, A587T, Q588S, A589I, and Q590R.

In some embodiments, the disclosure provides a recombinant adeno-associated virus (rAAV) capsid protein, wherein the capsid protein comprises the amino acid sequence of SEQ ID NO: 488. In some embodiments, the disclosure provides a recombinant adeno-associated virus (rAAV) capsid protein, wherein the capsid protein comprises the amino acid sequence of SEQ ID NO: 499. In some embodiments, the disclosure provides a recombinant adeno-associated virus (rAAV) capsid protein, wherein the capsid protein comprises the amino acid sequence of SEQ ID NO: 504. In some embodiments, the disclosure provides a recombinant adeno-associated virus (rAAV) capsid protein, wherein the capsid protein comprises the amino acid sequence of SEQ ID NO: 505. In some embodiments, the disclosure provides a recombinant adeno-associated virus (rAAV) capsid protein, wherein the capsid protein comprises the amino acid sequence of SEQ ID NO: 506. In some embodiments, the disclosure provides a recombinant adeno-associated virus (rAAV) capsid protein, wherein the capsid protein comprises the amino acid sequence of SEQ ID NO: 510. In some embodiments, the disclosure provides a recombinant adeno-associated virus (rAAV) capsid protein, wherein the capsid protein comprises the amino acid sequence of SEQ ID NO: 512. In some embodiments, the disclosure provides a recombinant adeno-associated virus (rAAV) capsid protein, wherein the capsid protein comprises the amino acid sequence of SEQ ID NO: 513. In some embodiments, the disclosure provides a recombinant adeno-associated virus (rAAV) capsid protein, wherein the capsid protein comprises the amino acid sequence of SEQ ID NO: 516. In some embodiments, the disclosure provides a recombinant adeno-associated virus (rAAV) capsid protein, wherein the capsid protein comprises the amino acid sequence of SEQ ID NO: 518. In some embodiments, the disclosure provides a recombinant adeno-associated virus (rAAV) capsid protein, wherein the capsid protein comprises the amino acid sequence of SEQ ID NO: 521. In some embodiments, the disclosure provides a recombinant adeno-associated virus (rAAV) capsid protein, wherein the capsid protein comprises the amino acid sequence of SEQ ID NO: 522, in some embodiments, the disclosure provides a recombinant adeno-associated virus (rAAV) capsid protein, wherein the capsid protein comprises the amino acid sequence of SEQ ID NO: 533. In some embodiments, the disclosure provides a recombinant adeno-associated virus (rAAV) capsid protein, wherein the capsid protein comprises the amino acid sequence of SEQ ID NO: 536. In some embodiments, the disclosure provides a recombinant adeno-associated virus (rAAV) capsid protein, wherein the capsid protein comprises the amino acid sequence of SEQ ID NO: 539. In some embodiments, the disclosure provides a recombinant adeno-associated virus (rAAV) capsid protein, wherein the capsid protein comprises the amino acid sequence of SEQ ID NO: 558. In some embodiments, the disclosure provides a recombinant adeno-associated virus (rAAV) capsid protein, wherein the capsid protein comprises the amino acid sequence of SEQ ID NO: 562. In some embodiments, the disclosure provides a recombinant adeno-associated virus (rAAV) capsid protein, wherein the capsid protein comprises the amino acid sequence of SEQ ID NO: 566. In some embodiments, the disclosure provides a recombinant adeno-associated virus (rAAV) capsid protein, wherein the capsid protein comprises the amino acid sequence of SEQ ID NO: 571. In some embodiments, the disclosure provides a recombinant adeno-associated virus (rAAV) capsid protein, wherein the capsid protein comprises the amino acid sequence of SEQ ID NO: 576. In some embodiments, the disclosure provides a recombinant adeno-associated virus (rAAV) capsid protein, wherein the capsid protein comprises the amino acid sequence of SEQ ID NO: 578. In some embodiments, the disclosure provides a recombinant adeno-associated virus (rAAV) capsid protein, wherein the capsid protein comprises the amino acid sequence of SEQ ID NO: 579. In some embodiments, the disclosure provides a recombinant adeno-associated virus (rAAV) capsid protein, wherein the capsid protein comprises the amino acid sequence of SEQ ID NO: 580. In some embodiments, the disclosure provides a recombinant adeno-associated virus (rAAV) capsid protein, wherein the capsid protein comprises the amino acid sequence of SEQ ID NO: 581. In some embodiments, the disclosure provides a recombinant adeno-associated virus (rAAV) capsid protein, wherein the capsid protein comprises the amino acid sequence of SEQ ID NO: 585. In some embodiments, the disclosure provides a recombinant adeno-associated virus (rAAV) capsid protein, wherein the capsid protein comprises the amino acid sequence of SEQ ID NO: 588. In some embodiments, the disclosure provides a recombinant adeno-associated virus (rAAV) capsid protein, wherein the capsid protein comprises the amino acid sequence of SEQ ID NO: 589.

In some embodiments, the disclosure provides a recombinant adeno-associated virus (rAAV) capsid protein, wherein the capsid protein comprises the amino acid sequence of SEQ ID NO: 705. In some embodiments, the disclosure provides a recombinant adeno-associated virus (rAAV) capsid protein, wherein the capsid protein comprises the amino acid sequence of SEQ ID NO: 706. In some embodiments, the disclosure provides a recombinant adeno-associated virus (rAAV) capsid protein, wherein the capsid protein comprises the amino acid sequence of SEQ ID NO: 707. In some embodiments, the disclosure provides a recombinant adeno-associated virus (rAAV) capsid protein, wherein the capsid protein comprises the amino acid sequence of SEQ ID NO: 708. In some embodiments, the disclosure provides a recombinant adeno-associated virus (rAAV) capsid protein, wherein the capsid protein comprises the amino acid sequence of SEQ ID NO: 710. In some embodiments, the disclosure provides a recombinant adeno-associated virus (rAAV) capsid protein, wherein the capsid protein comprises the amino acid sequence of SEQ ID NO: 767. In some embodiments, the disclosure provides a recombinant adeno-associated virus (rAAV) capsid protein, wherein the capsid protein comprises the amino acid sequence of SEQ ID NO: 768. In some embodiments, the disclosure provides a recombinant adeno-associated virus (rAAV) capsid protein, wherein the capsid protein comprises the amino acid sequence of SEQ ID NO: 769. In some embodiments, the disclosure provides a recombinant adeno-associated virus (rAAV) capsid protein, wherein the capsid protein comprises the amino acid sequence of SEQ ID NO: 770. In some embodiments, the disclosure provides a recombinant adeno-associated virus (rAAV) capsid protein, wherein the capsid protein comprises the amino acid sequence of SEQ ID NO: 771. In some embodiments, the disclosure provides a recombinant adeno-associated virus (rAAV) capsid protein, wherein the capsid protein comprises the amino acid sequence of SEQ ID NO: 772. In some embodiments, the disclosure provides a recombinant adeno-associated virus (rAAV) capsid protein, wherein the capsid protein comprises the amino acid sequence of SEQ ID NO: 773. In some embodiments, the disclosure provides a recombinant adeno-associated virus (rAAV) capsid protein, wherein the capsid protein comprises the amino acid sequence of SEQ ID NO: 774. In some embodiments, the disclosure provides a recombinant adeno-associated virus (rAAV) capsid protein, wherein the capsid protein comprises the amino acid sequence of SEQ ID NO: 775. In some embodiments, the disclosure provides a recombinant adeno-associated virus (rAAV) capsid protein, wherein the capsid protein comprises the amino acid sequence of SEQ ID NO: 776. In some embodiments, the disclosure provides a recombinant adeno-associated virus (rAAV) capsid protein, wherein the capsid protein comprises the amino acid sequence of SEQ ID NO: 777. In some embodiments, the disclosure provides a recombinant adeno-associated virus (rAAV) capsid protein, wherein the capsid protein comprises the amino acid sequence of SEQ ID NO: 778.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

In some embodiments, the capsid protein comprises, consists essentially of, or consists of a polypeptide sequence at least 80%, 85%, 90%, 95%, 96%, 97%, 98%, 99%, 99.5%, or 100% identical to one of SEQ ID NOs: 767, 768, 769, 770, 771, 772, 773, 774, 775, 776, 777, 778, or a functional fragment thereof, in some embodiments, the capsid protein comprises, consists essentially of, or consists of a polypeptide sequence at least 80%, 85%, 90%, 95%, 96%, 97%, 98%, 99%.0, 99.5%, or 100% identical to one of SEQ ID NOs: 705-708, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 705, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 706, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 707, or a functional fragment thereof.

In some embodiments, the capsid protein comprises, a polypeptide sequence at least 80%, 85%, 90%, 95%, 960, 97%, 98%, 99%, 99.5%, or 100% identical to SEQ ID NO: 708, or a functional fragment thereof.

In some embodiments, the capsid protein comprises, a polypeptide sequence at least 80%, 85%, 90%, 95%, %%, 97%, 98%, 99%, 99.5%, or 100% identical to SEQ ID NO: 710, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 772, or a functional fragment thereof.

In some embodiments, the capsid protein comprises, a polypeptide sequence at least 80%, 85%, 90%, 95%, %%, 97%, 98%, 99%, 99.5%, or 100% identical to SEQ ID NO: 774, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 488, or a functional fragment thereof.

In some embodiments, the capsid protein comprises, a polypeptide sequence at least 80%, 85%, 90%, 95%, 96%, 97%, 98%, 99%0, 99.5%, or I00% identical to SEQ ID NO: 512, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 513, or a functional fragment thereof.

In some embodiments, the capsid protein comprises, a polypeptide sequence at least 80%, 85%, 90%, 95%, 96%, 97%, 98%, 99%, 99.5%, or 100% identical to SEQ ID NO: 539, or a functional fragment thereof.

In some embodiments, the capsid protein comprises, a polypeptide sequence at least 80%, 85%, 90%, 95%, 96%, 97%, 98%, 99%, 99.5%, or 100o identical to SEQ ID NO: 589, or a functional fragment thereof.

In some embodiments, the capsid protein comprises, at amino acid positions 581-594 relative to reference sequence SEQ ID NO:1, the amino acid sequence of any one of SEQ ID NOs: 618, 684, 642, 630, 615, 692, 616, 668, 726, 608, 603, 657, 675, and 622, and optimally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 618, and optionally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 684, and optionally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 642, and optionally wherein the capsid protein further comprises an amino acid substitution of N452K. In some embodiments, the capsid protein comprises, at amino acid positions 581-594 relative to reference sequence SEQ ID NO:1, the amino acid sequence of SEQ ID NO: 630, and optionally wherein the capsid protein further comprises an amino acid substitution of N452K.

In some embodiments, the capsid protein comprises, at amino acid positions 581-594 relative to reference sequence SEQ ID NO:1, the amino acid sequence of any one of SEQ ID NOs: 598, 602, 607, 608, 609, 613, 615, 616, 618, 619, 621, 624, 625, 630, 636, 639, 642, 661, 665, 669, 674, 679, 681, 682, 683, 684, 688, 691, 692, and 726. In some of these embodiments, the capsid comprises at amino acid position 452, relative to reference sequence SEQ ID NO:1, amino acid N or K. In some of these embodiments, the capsid protein comprises an amino acid substitution N452K.

In some embodiments, the capsid protein comprises, at amino acid positions 581-594 relative to reference sequence SEQ ID NO:1, the amino acid sequence of any one of SEQ ID NOs: 598, 608, 615, 616, 618, 642, 692, and 726. In some of these embodiments, the capsid comprises at amino acid position 452, relative to reference sequence SEQ ID NO:1, amino acid N or K. In some of these embodiments, the capsid protein comprises an amino acid substitution N452K.

In some embodiments, the capsid protein of the present disclosure comprises a variant polypeptide sequence at the VR-VIII site, wherein the VR-VIII site (e.g., the entire VR-VIII site) comprises, consists essentially of, or consists of, a sequence having at least about 60%, 65%, 70%, 71%, 74%, 75%, 78%, 78.5%, 79%, 80%, 83%, 85%, 86%, 90%, 92%, 93% or 100% identity to any one of the following sequences:

VR-VIII Alignment (581-594)

ATNHENTYSIAQTG
ATNHQTLFNSAQTG
ATNHNSTYLGAQTG
ATNHGSILTHAQTG
ATNHMMTTARAQTG
ATNHCSTSIRAQTG
ATNHQGAYAQAQTG
ATNHNTKLAIAQTG
ATNHVSSFTSAQTG
ATNHEDNIRSAQTG
ATNHQSAQAQAQTG
ATNHNNVISGAQTG
ATNHTGTSIIAQTG
ATNHQWMSAQAQAQTG
ATNHQDARAQAQTG
ATNHQHYSAQAQAQTG
ATNHQSAQAQAQTG
ATNHNIRTEMAQTG
ATNHSTTNFRAQTG
ATNHQANYGQAQTG
ATNHNMNRVNAQTG
ATNHSNSVQSAQTG
ATNHSSTFQGAQTG
ATNHSTTNFRAQTG
ATNHSSIFNSAQTG
ATNHAGNYNNAQTG
ATNHTSVISIAQTG
ATNHHSRVEYAQTG
ATNHSSIIYSAQTG
ATNHSGRDSYAQTG
ATNHSSSYNNAQTG
ATNHHNPSINAQTG
ATNHNRNGLLAQTG
ATNHESTSVRAQTG
ATNHLSVSSIAQTG
ATNHEDIIRSAQTG
ATNRQTAQAQAQTG
ATNRQIAQAQAQTG

In some embodiments, the capsid protein of the present disclosure comprises a variant polypeptide sequence at the VR-VIII site, wherein the entire VR-VIII site comprises amino acids ATNHQSAQAQAQTG (SEQ ID NO: 5), and wherein there is an insertion of one, two or more amino acids in this site. In some embodiments, the insertion is within the variant polypeptide of sequence QSAQAQ (SEQ ID NO: 756), within SEQ ID NO:5. In some embodiments, the insertion is between amino acids ATNHQ and amino acids SAQAQAQTG of SEQ ID NO:5. In other words, in some embodiments, the insertion at the VR-VIII site is between position 585 and position 586 relative to reference sequence SEQ ID NO:1. In some embodiments, the insertion is insertion of amino acids WM (e.g., between positions 585 and 586 relative to reference sequence SEQ ID NO:1). In some embodiments, the insertion is insertion of amino acids HY (e.g., between positions 585 and 586 relative to reference sequence SEQ ID NO:1). In some of these embodiments, the capsid protein may further comprise N452K substitution relative to reference sequence SEQ ID NO: 1 (in addition to the variant polypeptide at VR-VIII site described herein).

### Chimeric AAV5/AAV9 Capsid

The present disclosure also provides recombinant adeno-associated virus (rAAV) capsid proteins comprising a sequence at least 80%, at least 85%, at least 90%, at least 95%, at least 98%, at least 99%, or 100% identical to SEQ I) NO: 463. (in SEQ ID NO:463, the amino acids residues labeled “X” are excluded from sequence identity calculation.) In some embodiments, the capsid protein is an AAV5/AAV9 chimeric capsid protein. In some embodiments, the AAV5/AAV9 chimeric capsid protein sequence is more than about 50%, 55%, 60%, 65%, 70%, 75%, 80%, 85%, 90% a, 95%, 96%, 97%, 98%, 99%, or 99.5% identical to the AAV9 capsid protein sequence (SEQ ID NO: 1). In some embodiments, the C-terminal 500 residues of the AAV5/AAV9 chimeric capsid protein sequence is at least about 80%, 85%, 90%, 95%, 96%, 97%, 98%, 99%, 99.5%, or 100% identical to the C-terminal 500 residues of the AAV9 capsid protein sequence (SEQ ID NO: 1). In some embodiments, the residue at the position equivalent to Q688 of the AAV9 capsid protein sequence (SEQ ID NO: 1) is a lysine (K) in the chimeric capsid protein.

In some embodiments, the chimeric capsid protein comprises at least 1, 2, 3, 4, 5 or more polypeptide segments that are derived from AAV5 capsid protein. In some embodiments, the chimeric capsid protein comprises at least 1, 2, 3, 4, 5 or more polypeptide segments that are derived from AAV9 capsid protein. In some embodiments, at least one polypeptide segment is derived from the AAV5 capsid protein and at least one polypeptide segment is derived from the AAV9 capsid protein.

In some embodiments, the first 250 residues at the N-terminus of the chimeric capsid protein comprise one or more AAV5 capsid derived polypeptide segments. In some embodiments, the first 225 residues at the N-terminus of the chimeric capsid protein comprise one or more AAV5 capsid derived polypeptide segments. In some embodiments, the first 200 residues at the N-terminus of the chimeric capsid protein comprise one or more AAV5 capsid derived polypeptide segments. In some embodiments, the first 150 residues at the N-terminus of the chimeric capsid protein comprise one or more AAV5 capsid derived polypeptide segments. In some embodiments, the first 100 residues at the N-terminus of the chimeric capsid protein comprise one or more AAV5 capsid derived polypeptide segments. In some embodiments, the first 50 residues at the N-terminus of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% sequence identity to the corresponding AAV5 capsid sequence.

In some embodiments, residues 50-250 of the chimeric capsid protein comprise one or more AAV5 capsid derived polypeptide segments. In some embodiments, residues 50-200 of the chimeric capsid protein comprise one or more AAV5 capsid derived polypeptide segments. In some embodiments, residues 50-150 of the chimeric capsid protein comprise one or more AAV5 capsid derived polypeptide segments. In some embodiments, residues 100-250 of the chimeric capsid protein comprise one or more AAV5 capsid derived polypeptide segments. In some embodiments, residues 100-200 of the chimeric capsid protein comprise one or more AAV5 capsid derived polypeptide segments. In some embodiments, residues 150-250 of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or IOW % sequence identity to the corresponding AAV5 capsid sequence.

In some embodiments, the last 100 residues at the C-terminus of the chimeric capsid protein comprise one or more AAV5 capsid derived polypeptide segments. In some embodiments, the last 50 residues at the C-terminus of the chimeric capsid protein comprise one or more AAV5 capsid derived polypeptide segments. In some embodiments, each of the one or more AAV5 capsid derived polypeptide segments has at least about 80%, 85%, 90%, 95%, 96%, 97%, 98%, 99%, 99.5%, or 100% sequence identity to the corresponding AAV5 capsid sequence. In some embodiments, the chimeric capsid protein comprises one or more AAV5 capsid derived polypeptide segments at or near the N-terminus of the chimeric capsid protein, as described above, and one or more AAV5 capsid derived polypeptide segments at or near the C-terminus of the chimeric capsid protein, as described in this paragraph.

In some embodiments, the chimeric capsid protein comprises, in N-terminal to C-terminal order, a first polypeptide segment having sequence at least about 80%, 85%, 90%, 95%, 96%, 97%, 98%, 99%, 99.5%, or 100% identical to SEQ ID NO: 411 or at least about 80%, 85%, 90%, 95%, 96%, 97%, 98%, 99%, 99.5%, or 100% identical to SEQ ID NO: 412; a second polypeptide segment having sequence at least about 80%, 85%, 90%, 95%, 96%, 97%, 98%, 99%, 99.5%, or 100% identical to SEQ ID NO: 413 or at least about 80%, 85%, 90%, 95%, 96%, 97%, 98%, 99%, 99.5%, or 100% identical to SEQ ID NO: 414; a third polypeptide segment having sequence at least about 80%, 85%, 90%, 95%, %%⁰′, 97%, 98%, 99%, 99.5%, or 100% identical to SEQ ID NO: 415 or at least about 80%, 85%, 90%, 95%, 96%, 97%, 98%, 99%, 99.5%, or 100% identical to SEQ ID NO: 416; a fourth polypeptide segment having sequence at least about 80%, 85%, 90%, 95%, 96%, 97%, 98%, 99%, 99.5%, or 100% identical to SEQ ID NO: 417 or at least about 80%, 85%, 90%, 95%, 96%, 97%, 98%, 99%, 99.5%, or 100% identical to SEQ ID NO: 418; and a fifth polypeptide segment having sequence at least about 80%, 85%, 90%, 95%, 96%, 97%, 98%, 99%, 99.5%, or 100% identical to SEQ ID NO: 419 or at least about 80%, 85%, 90%, 95%, 96%, 97%0, 98%, 99%, 99.5%, or 100% identical to SEQ ID NO: 420. In some embodiments, at least one polypeptide segment is derived from the AAV5 capsid protein and at least one polypeptide segment is derived from the AAV9 capsid protein.

AAV9 derived polypeptide segment 1:
(SEQ ID NO: 411)
MAADGYLPDWLEDNLSEGIREWWALKPGAPQPKANQQHQDNARGLVLPGY
Sequence of AAV5 derived polypeptide segment 1:
(SEQ ID NO: 412)
MSFVDHPPDWLEEVGEGLREFLGLEAGPPKPKPNQQHQDQARGLVLPGY
Sequence of AAV9 derived polypeptide segment 2:
(SEQ ID NO: 413)
KYLGPGNGLDKGEPVNAADAAALEHDKAYDQQLK
Sequence of AAV5 derived polypeptide segment 2:
(SEQ ID NO: 414)
NYLGPGNGLDRGEPVNRADEVAREHDISYNEQLE
Sequence of AAV9 derived polypeptide segment 3:
(SEQ ID NO: 415)
AGDNPYLKYNHADAEFQERLKEDTSFGGNLGRAVFQAKKRLLEP
Sequence of AAV5 derived polypeptide segment 3:
(SEQ ID NO: 416)
AGDNPYLKYNHADAEFQEKLADDTSFGGNLGKAVFQAKKRVLEP
Sequence of AAV9 derived polypeptide segment 4:
(SEQ ID NO: 417)
LGLVEEAAKTAPGKKRPVEQSPQEPDSSAGIGKSGAQPAKKRLNFGQTGDTESVPDPQPI
GEPPAAPSGVGSLTMASGGGAPVA
Sequence of AAVS derived polypeptide segment 4:
(SEQ ID NO: 418)
FGLVEEGAKTAPTGKRIDDHFPKRKKARTEEDSKPSTSSDAEAGPSGSQQLQIPAQPASS
LGADTMSAGGGGPLG
Sequence of AAV9 derived polypeptide segment 5:
(SEQ ID NO: 419)
DNNEGADGVGSSSGNWHCDSQWLGDRVITTSTRTWALPTYNNHLYKQISNSTSGGSSN
DNAYFGYSTPWGYFDFNRFHCHFSPRDWQRLINNNWGFRPKRLNFKLFNIQVKEVTDNNGVKT
IANNLTSTVQVFTDSDYQLPYVLGSAHEGCLPPFPADVFMIPQYGYLTLNDGSQAVGRSSFYCLE
YFPSQMLRTGNNFQFSYEFENVPFHSSYAHSQSLDRLMNPLIDQYLYYLSKTINGSGQNQQTLKF
SVAGPSNMAVQGRNYIPGPSYRQQRVSTTVTQNNNSEFAWPGASSWALNGRNSLMNPGPAMA
SHKEGEDRFFPLSGSLIFGKQGTgrdnvDADKVMITNEEEIKTTNPVATESYGQVATNHQSAQAQA
QTGWVQNQGILPGMVWQDRDVYLQGPIWAKIPHTDGNFHPSPLMGGFGMKHPPPQILIKNTPV
PADPPTAFNKDKLNSFITQYSTGQVSVEIEWELQKENSKRWNPEIQYTSNYYKSNNVEFAVNTE
GVYSEPRPIGTRYLTRNL
Sequence of AAV9 derived polypeptide segment 5 with Q688K mutation:
(SEQ ID NO: 420)
DNNEGADGVGSSSGNWHCDSQWLGDRVITTSTRTWALPTYNNHLYKQISNSTSGGSSN
DNAYFGYSTPWGYFDFNRFHCHFSPRDWQRLINNNWGFRPKRLNFKLFNIQVKEVTDNNGVKT
IANNLTSTVQVFTDSDYQLPYVLGSAHEGCLPPFPADVFMIPQYGYLTLNDGSQAVGRSSFYCLE
YFPSQMLRTGNNFQFSYEFENVPFHSSYAHSQSLDRLMNPLIDQYLYYLSKTINGSGQNQQTLKF
SVAGPSNMAVQGRNYIPGPSYRQQRVSTTVTQNNNSEFAWPGASSWALNGRNSLMNPGPAMA
SHKEGEDRFFPLSGSLIFGKQGTgrdnvDADKVMITNEEEIKTTNPVATESYGQVATNHQSAQAQA
QTGWVQNQGILPGMVWQDRDVYLQGPIWAKIPHTDGNFHPSPLMGGFGMKHPPPQILIKNTPV
PADPPTAFNKDKLNSFITQYSTGQVSVEIEWELKKENSKRWNPEIQYTSNYYKSNNVEFAVNTE
GVYSEPRPIGTRYLTRNL

In some embodiments, the chimeric capsid protein comprises, consists essentially of, or consists of a polypeptide sequence at least about 80%, 85%, 90%, 95%, 96%, 97%, 98%, 99%, 99.5%, or 100% identical to one of SEQ ID NOs: 421-444, or a functional fragment thereof.

TABLE 2
Capsid Protein Sequences

| Name/Alternate Name | SEQ ID NO: |
|---|---|
| ZC23 | 421 |
| ZC24 | 422 |
| ZC25 | 423 |
| ZC26 | 424 |
| ZC27 | 425 |
| ZC28 | 426 |
| ZC29 | 427 |
| ZC30 | 428 |
| ZC31 | 429 |
| ZC32 | 430 |
| ZC33 | 431 |
| ZC34 | 432 |
| ZC35 | 433 |
| ZC40/TN8 | 434 |
| ZC41 | 435 |
| ZC42 | 436 |
| ZC43 | 437 |
| ZC44/TN10 | 438 |
| ZC45 | 439 |
| ZC46 | 440 |
| ZC47/TN14 | 441 |
| ZC48 | 442 |
| ZC49 | 443 |
| ZC50 | 444 |

### Combinatory Capsid Protein

In one aspect, the present disclosure provides combinatory capsid proteins. As used herein, “combinatory capsid protein” refers to a AAV5/AAV9 chimeric capsid protein as described in the present disclosure, which further comprises amino acid variations with respect to the chimeric parental sequence at one or more sites. In some embodiments, the one or more sites of the chimeric parental sequence are selected from those equivalent to the VR-IV site, the VR-V site, the VR-VIII site, and the VR-VIII site of the AAV9 capsid protein.

The combinatory capsid proteins of the present disclosure include any variant polypeptide sequences identified as shown in, but not limited to, the Examples. Without being limited to any particular example, in some embodiments, the combinatory capsid protein comprises a chimeric AAV5/AAV9 capsid protein backbone, and further comprises the variant polypeptide sequence at one or more sites selected from the group consisting of those equivalent to the VR-IV site, the VR-V site, the VR-VII site, and the VR-VIII site of the AAV9 capsid protein as described herein.

In some embodiments, the combinatory capsid protein comprises, in N-terminal to C-terminal order, a first polypeptide segment having sequence at least about 80%, 85%, 90%, 95%, 96%, 97%, 98%, 99%, 99.5%, or 100% identical to SEQ ID NO: 411 or at least about 80%, 85%, 90%, 95%, 96%, 97%, 98%, 99%, 99.5%, or 100% identical to SEQ ID NO: 412; a second polypeptide segment having sequence at least about 80%, 85%, 90%, 95%, 96%, 97%, 98%, 99%, 99.5%, or 100% identical to SEQ ID NO: 413 or at least about 80%, 85%, 90%, 95%, 96%, 97%, 98%, 99%, 99.5%, or 100% identical to SEQ ID NO: 414; a third polypeptide segment having sequence at least about 80%, 85%, 90%, 95%, 96%, 97%, 98%, 99%, 99.5%, or 100% identical to SEQ ID NO: 415 or at least about 80%, 85%, 90%, 95%, 96%, 97%, 98%, 99%, 99.5%, or 100% identical to SEQ ID NO: 416; a fourth polypeptide segment having sequence at least about 80%, 85%, 90%, 95%, 96%, 97%, 98%, 99%, 99.5%, or 100% identical to SEQ ID NO: 417 or at least about 80%, 85%, 90%, 95%, 9%, 97%, 98%, 99%, 99.5%, or 100% identical to SEQ ID NO: 418; and a fifth polypeptide segment having sequence at least about 80%, 85%, 90%, 95%, 96%, 97%, 98%, 99%, 99.5%, or 100% identical to SEQ ID NO: 419 or at least about 80%, 85%, 90%, 95%, 96%, 97%, 98%, 99%, 99.5%, or 100% identical to SEQ ID NO: 420 (here, regions equivalent to the VR-IV site, the VR-V site, the VR-VII site and the VR-VIII site of the AAV9 capsid protein are excluded in the sequence identity calculation of the fifth polypeptide segment). In some embodiments, the combinatory capsid protein comprises a variant polypeptide sequence at one or more of a VR-IV site, a VR-V site, a VR-VII site, and a VR-VIII site of a parental sequence, wherein the parental sequence comprises a sequence at least 80%, at least 85%, at least 90%, at least 95%, at least 98%, at least 99%, or 100% identical to SEQ ID NO: 463. (in SEQ ID NO:463, the amino acids residues labeled “X” are excluded from sequence identity calculation.)

In some embodiments, at least one polypeptide segment is derived from the AAV5 capsid protein and at least one polypeptide segment is derived from the AAV9 capsid protein.

In some embodiments, the combinatory capsid protein further comprises variant polypeptide sequence at one or more sites selected from those equivalent to the VR-IV site, the VR-V site, the VR-VII site, and the VR-VIII site of the AAV9 capsid protein.

In some embodiments, the combinatory capsid protein has a variant polypeptide sequence at the site equivalent to the VR-IV site of the AAV9 capsid protein, which comprises, consists essentially of, or consists of a sequence at least about 60%, 70%, 80%, 90%, or 100% identical to GYHKSGAAQ (SEQ ID NO: 6). In some embodiments, the variant polypeptide sequence at the site equivalent to the VR-IV site of the AAV9 capsid protein comprises, consists essentially of, or consists of a sequence consisting of at most 1, 2, 3, or 4 conservative amino-acid substitutions relative GYHKSGAAQ (SEQ ID NO: 6).

In some embodiments, the combinatory capsid protein has a variant polypeptide sequence at the site equivalent to the VR-V site of the AAV9 capsid protein, which comprises, consists essentially of, or consists of a sequence at least about 60%, 70%, 80%, 90%, or 100% identical to LNSMLI (SEQ ID NO: 105). In some embodiments, the variant polypeptide sequence at the site equivalent to the VR-V site of the AAV9 capsid protein comprises, consists essentially of, or consists of a sequence consisting of at most 1, 2, 3, or 4 conservative amino-acid substitutions relative LNSMLI (SEQ ID NO: 105).

In some embodiments, the combinatory capsid protein has a variant polypeptide sequence at the site equivalent to the VR-VIII site of the AAV9 capsid protein, which comprises, consists essentially of, or consists of a sequence at least about 60%, 70%, 80%, 90%, or 100% identical to ANYG (SEQ ID NO: 305) or NVSY (SEQ ID NO: 303). In some embodiments, the variant polypeptide sequence at the site equivalent to the VR-VIII site of the AAV9 capsid protein comprises, consists essentially of, or consists of a sequence consisting of at most 1, 2, 3, or 4 conservative amino-acid substitutions relative ANYG (SEQ ID NO: 305) or NVSY (SEQ ID NO: 303).

In some embodiments, the residue at the position equivalent to Q688 of the AAV9 capsid protein sequence (SEQ ID NO: 1) is a lysine (K) in the combinatory capsid protein.

In some embodiments, the combinatory capsid protein comprises, consists essentially of, or consists of a polypeptide sequence at least about 80%, 85%, 90%, 95%, 96%, 97%, 98%, 99%, 99.5%, or 100% identical to one of SEQ ID NOs: 445-462, or a functional fragment thereof.

TABLE 3
Capsid Protein Sequences

| Name/Alternate Name | SEQ ID NO: |
|---|---|
| TN47-07 | 445 |
| TN47-10/TN12 | 446 |
| TN47-13 | 447 |
| TN47-14 | 448 |
| TN47-17 | 449 |
| TN47-22 | 450 |
| TN40-07 | 451 |
| TN40-10 | 452 |
| TN40-13 | 453 |
| TN40-14 | 454 |
| TN40-17 | 455 |
| TN40-22 | 456 |
| TN44-07/TN13 | 457 |
| TN44-10 | 458 |
| TN44-13 | 459 |
| TN44-14 | 460 |
| TN44-17 | 461 |
| TN44-22 | 462 |

### Additional Mutations

Additional amino acid substitutions may be incorporated, for example, to further improve transduction efficiency or tissue selectivity. Exemplary non-limiting substitutions include, but are not limited to, S651A, T378A or T582A relative to the sequence of AAV5, in either an AAV5 or AAV9-based capsid.

In some embodiments, the capsid protein comprises a mutation selected from S651A, T578A, T582A, K251R, Y709F, Y693F, or S485A relative to the sequence of AAV5, in either an AAV5 or AAV9-based capsid. In some embodiments, the capsid protein comprises a mutation selected from K251 R, Y709F, Y693F, or S485A relative to the sequence of AAV5, in either an AAV5 or AAV9-based capsid.

### Transduction Efficiency, Tropism, and NAb Evasion

Transduction efficiency can be determined using methods known in the art or those described in the Examples. In some embodiments, the rAAV virion with engineered capsid protein exhibits increased transduction efficiency in cardiac cells compared to an AAV virion comprising the parental sequence. The rAAV virion referenced in this section is any rAAV virion with modified or engineered capsid protein described herein.

In some embodiments, the rAAV virion exhibits increased transduction efficiency in human cardiac fibroblast (hCF) cells compared to an AAV virion comprising the parental sequence. In some embodiments, the human cardiac fibroblasts are located in the left ventricle of the heart.

In some embodiments, the rAAV virion exhibits at least 2-, 3-, 4-, 5-, 6, 7-, 8-, 9-, 10-, 11-, 12-, 13-, 14, or 15-fold increased transduction efficiency in hCF cells at a multiplicity of infection (MOI) of 100,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in hCF cells at a multiplicity of infection (MOI) of 100,000. In some embodiments, the rAAV virion exhibits at least 2-, 3-, 4-, 5-, 6, 7-, 8-, 9-, 10, 11-, 12-, 13-, 14, or 15-fold increased transduction efficiency in hCF cells at a multiplicity of infection (MOI) of 100,000. In some embodiments, the rAAV virion exhibits about 20% to 30%, about 30% to 40%, about 40% to 50% c, about 50% to 80%, about 80% to 100%, about 100% to 125%, about 125% to 150%, about 150% to 175%, or about 175% to 200% increased transduction efficiency in hCF cells at a multiplicity of infection (MOI) of 100,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in hCF cells at a multiplicity of infection (MOI) of 1,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in hCF cells at a multiplicity of infection (MOI) of 1,000. In some embodiments, the rAAV virion exhibits about 20% to 30%, about 30% to 40%, about 40%- to 50%, about 50% to 80%, about 80% to 100%, about 100% to 125%, about 125% to 150%, about 150% to 175%, or about 175% to 200% increased transduction efficiency in hCF cells at a multiplicity of infection (MOI) of 1,000.

In some embodiments, the rAAV virion exhibits increased transduction efficiency in induced pluripotent stem cell-derived cardiomyocyte (iPS-CM) cells compared to an AAV virion comprising the parental sequence. Accordingly, the fold improvement discussed in this section is as compared to an AAV virion comprising the parental sequence (e.g., AAV9).

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 100,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 100,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 100,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 75.000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 75,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 75,000.

In some embodiments, the rAAV virion exhibits at least 2-, 3-, 4-, 5-, 6, 7-, 8-, 9-, 10-, 11-, 12-, 13-, 14, or 15-fold increased transduction efficiency in iPS-CM cells at a multiplicity of infection (MOI) of 1,000. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in iPS-CM cells at a multiplicity of infection (MOI) of 1,000. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in iPS-CM cells at a multiplicity of infection (MOI) of 1,000.

In some embodiments, the rAAV virion comprising the engineered capsid protein of the present disclosure exhibits increased transduction efficiency in heart compared to an AAV virion comprising the parental sequence. In some embodiments, transduction efficiency in heart is monitored by injecting C57BL′6J mice with either AAV9:CAG-GFP or CAG-GFP encapsulated by the engineered capsid protein of the present disclosure. In some embodiments, the injection dosage is 2.5E+11 vg/mouse. In some embodiments, the injection dosage is 2E+11 vg/mouse. In some embodiments, the injection dosage is 1E+11 vg/mouse. In some embodiments, the rAAV virion exhibits at least 2-, 3-,4-, 5-, 6, 7-, 8-, 9-, 10-, 11-, 12-, 13-, 14, or 15-fold increased transduction efficiency in heart. In some embodiments, the rAAV virion exhibits at least 2-, 3-, 4-, 5-, 6, 7-, 8-, 9-, 10-, 11-, 12-, 13-, 14, or 15-fold increased transduction efficiency in heart relative to wild-type AAV9. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart relative to wild-type AAV9. In some embodiments, the rAAV virion exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart. In some embodiments, the rAAV virion exhibits about 20% to 30%, about 30% to 40%, about 40′ to 50%, about 50% to 80%, about 80% to 100%, about 100% to 125%, about 125% to 150%, about 150% to 175%, or about 175% to 200% increased transduction efficiency in heart relative to wild-type AAV9.

In some embodiments, the rAAV virion comprising the engineered capsid protein of the present disclosure exhibits decreased transduction efficiency in liver cells compared to an AAV virion comprising the parental sequence. In some embodiments, liver transduction efficiency is monitored by injecting C57BL/6J mice with either AAV9:CAG-GFP or CAG-GFP encapsulated by the engineered capsid protein of the present disclosure. In some embodiments, the injection dosage is 2.5E+11 vg/mouse. In some embodiments, the injection dosage is 2E+11 vg/mouse. In some embodiments, the injection dosage is 1E+11 vg/mouse. In some embodiments, the rAAV virion exhibits at least 2-, 3-, 4-, 5-, 6, 7-, 8-, 9-, 10-, 11-, 12-, 13-, 14, or 15-fold decreased transduction efficiency in liver. In some embodiments, the injection dosage is 1E+11 vg/mouse. In some embodiments, the rAAV virion exhibits at least 2-, 3-, 4-, 5-, 6, 7-, 8-, 9-, 10-, 11-, 12-, 13-, 14, or 15-fold decreased transduction efficiency in liver relative to wild-type AAV9. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver. In some embodiments, the rAAV virion exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver relative to wild-type AAV9. In some embodiments, the rAAV virion exhibits about 20% to 30%, about 30% to 40%, about 40% to 50%, about 50% to 80%, or about 80% to 100 decreased transduction efficiency in liver. In some embodiments, the rAAV virion exhibits about 20% to 30%, about 30% to 40%, about 40% to 50%, about 50% to 80%, or about 80% to 100 decreased transduction efficiency in liver relative to wild-type AAV9.

Selectivity for a cell type and/or a tissue/organ type is increased when the ratio of the transduction efficiencies for one cell/tissue/organ type over another is increased for rAAV virions comprising the engineered capsid protein of the present disclosure compared to an AAV virion comprising the parental sequence. In some embodiments, the rAAV virion comprising the engineered capsid protein exhibits increased selectivity for iPS-CM cells over liver cells. In some embodiments, the rAAV virion comprising the engineered capsid protein exhibits increased selectivity for heart over liver when injected in vivo, in some embodiments, the rAAV virion comprising the engineered capsid protein exhibits increased selectivity for the left ventricle of the heart over liver when injected in vivo.

In some embodiments, the rAAV virion comprising the engineered capsid protein exhibits at least 2-, 3-, 4-, 5-, 6, 7-, 8-, 9-, 10-, 11-, 12-, 13-, 14, or 15-fold increased selectivity of iPS-CM cells over liver cells and/or heart over liver. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased selectivity of iPS-CM cells over liver cells and/or heart over liver. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% increased selectivity of iPS-CM cells over liver cells and/or heart over liver.

In some embodiments, the rAAV virion comprising the engineered capsid protein exhibits at least 2-, 3-, 4-, 5-, 6, 7-, 8-, 9-, 10-, 11-, 12-, 13-, 14, or 15-fold increased selectivity of heart tissue over liver tissue. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased selectivity of heart tissue over liver tissue. In some embodiments, the rAAV virion comprising the engineered capsid protein exhibits at least or more than 30%, 40%, 50%, 80%, 100%, 125%, 150%, 175%, 200%, 250%, 300%, 400%, 500%, 600%, 700%, 800% or 1000% increased selectivity of heart tissue over liver tissue. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% increased selectivity of heart tissue over liver tissue.

In some embodiments, the rAAV virion comprising the engineered capsid protein of the present disclosure exhibits improved ability to evade human NAb (neutralizing antibodies) compared to an AAV virion comprising the parental sequence. In some embodiments, the ability to evade human NAb is measured via an NAb inhibition assay. Non-limiting examples of NAb inhibition assays are described in the Example section of the present disclosure. In some embodiments, NAb inhibition assays are performed by incubating AAV virions with pooled human NAb (e.g., IgG) before treating a target cell at a pre-determined MOI and measure the decrease of transduction efficiency compared to AAV virions not incubated with pooled human NAb. Less NAb inhibition indicates improved ability of the AAV virion to evade human NAb. In some embodiments, the rAAV virion comprising the engineered capsid protein exhibits at least 2-, 3-, 4-, 5-, 6, 7-, 8-, 9-, 10, 11-, 12-, 13-, 14, or 15-fold improved ability to evade human NAb. In some embodiments, the rAAV virion comprising the engineered capsid protein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold improved ability to evade human NAb. In some embodiments, the rAAV virion comprising the engineered capsid protein exhibits about 20% to 30%, about 30% to 40%, about 40% to 50%, about 50% to 80%, about 80% to 100%, about 100% to 125%, about 125% to 150%, about 150% to 175%, or about 175% to 200% improved ability to evade human NAb.

The polynucleotide encoding the capsid protein can comprise a sequence comprising either the native codons of the wild-type cap gene, or alternative codons selected to encode the same protein. The codon usage of the insertion can be varied. It is within the skill of those in the art to select appropriate nucleotide sequences and to derive alternative nucleotide sequences to encode any capsid protein of the disclosure. Reverse translation of the protein sequence can be performed using the codon usage table of the host organism, i.e., Eukaryotic codon usage for humans.

In some embodiments, the disclosure provides a polynucleotide encoding an AAV9 derived capsid protein comprising a sequence at least 80%, 85%, 90%, 95%, 99%, or 100% identical to any one of SEQ ID NOs: 402-410 and 464-468.

In some embodiments, the disclosure provides a polynucleotide encoding an AAV5/AAV9 chimeric capsid protein comprising a sequence at least 80%, 85%, 90%, 95%, 99%, or 100% identical to any one of SEQ ID NOs: 421-444.

In some embodiments, the disclosure provides a polynucleotide encoding a combinatory capsid protein comprising a sequence at least 80%, 85%, 90%, 95%, 99%, or 100% identical to any one of SEQ ID NO: 445-462.

In some embodiments, the disclosure provides a polynucleotide encoding an AAV9 derived capsid protein comprising a sequence having at least or more than 80%, 85%, 90%, 95%, 99%, or 100% sequence identity to any one of SEQ ID NOs: 705-708.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 515, 581, 539 and 527.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 707, 512, 539 and 589.

In some embodiments, the disclosure provides a polynucleotide encoding an AAV9 derived capsid protein comprising a sequence having at least 80%, 85%, 90%, 95%, 99%, or 100% sequence identity to any one of SEQ ID NOs: 707, 512, 539 and 589. In some embodiments, the disclosure provides a polynucleotide encoding an AAV9 derived capsid protein comprising a sequence having at least 80%, 85%, 90%, 95%, 99%, or 100% sequence identity to SEQ ID NO: 707. In some embodiments, the disclosure provides a polynucleotide encoding an AAV9 derived capsid protein comprising a sequence having at least 80%, 85%, 90%, 95%, 99%, or 100% sequence identity to SEQ ID NO: 512. In some embodiments, the disclosure provides a polynucleotide encoding an AAV9 derived capsid protein comprising a sequence having at least 80%, 85%, 90%, 95%, 99%, or 100% sequence identity to SEQ ID NO: 539. In some embodiments, the disclosure provides a polynucleotide encoding an AAV9 derived capsid protein comprising a sequence having at least 80%, 85%, 90%, 95%, 99%, or 100% sequence identity to SEQ ID NO: 589.

In some embodiments, the disclosure provides a polynucleotide encoding an AAV9 derived capsid protein comprising a sequence at least 80%, 85%, 90%, 95%, 97%, 98%, 99%, or 100% identical to any one of SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, and 710.

In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 488. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 499. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 504. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 505. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 506. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 510. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 512. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 513. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 516. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 518. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 521. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 522. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 533. In some embodiments, the disclosure provides a polynucleotide encoding an AAV9 derived capsid protein comprising a sequence at least 80%, 85%, 90%, 95%, 97%, 98%, 99′0, or 100% identical to SEQ ID NOs: 536. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 539. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 558. In some embodiments, the disclosure provides a polynucleotide encoding an AAV9 derived capsid protein comprising a sequence at least 80%, 85%, 90%, 95%, 97% N, 98%, 99%, or 100% identical to SEQ ID NOs: 562. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 566. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 571. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 576. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 578. In some embodiments, the disclosure provides a polynucleotide encoding an AAV9 derived capsid protein comprising a sequence at least 80%, 85%, 90%-, 95%, 970%, 98%, 99%, or 100% identical to SEQ ID NOs: 579. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 580. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 581. In some embodiments, the disclosure provides a polynucleotide encoding an AAV9 derived capsid protein comprising a sequence at least 80%, 85%, 90%, 95%, 97%, 98%, 99%, or 100%.0 identical to SEQ ID NOs: 585. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 588. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 589. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 705. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 706. In some embodiments, the disclosure provides a polynucleotide encoding an AAV9 derived capsid protein comprising a sequence at least 80%, 85%, 90%, 95%, 97%, 98%, 99%, or 100%.o identical to SEQ ID NOs: 707. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 708. In some embodiments, the disclosure provides a polynucleotide encoding an AAV9 derived capsid protein comprising a sequence at least 80%, 85%, 90%, 95%, 97%, 98%, 99%, or 100% identical to SEQ ID NOs: 710.

In some embodiments, the disclosure provides an AAV9, AAV5/AAV9 chimeric, or combinatory capsid protein comprising a sequence at least 80%, 85%, 90%, 95%, 99%, or 100% identical to a modified capsid selected from SEQ ID NOs: 402-410, 421-462, 464-468, wherein the amino acid substitutions, optionally conservative substitutions, with the specified percent identity level are tolerated.

In some embodiments, any rAAV comprising N452K mutation as described herein exhibits at least 2-, 3-, 4-, 5-, 6, 7-, 8-, 9-, 10-, 11-, 12-, 13-, 14, or 15-fold increased transduction efficiency in heart relative to wild-type AAV9 and/or relative to transduction of liver. In some embodiments, any rAAV comprising N452K mutation as described herein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold increased transduction efficiency in heart relative to wild-type AAV9 and/or relative to transduction of liver. In some embodiments, any rAAV virion comprising N452K mutation as described herein exhibits at least or more than 30%, 40%, 50%, 80%, 100%, 125%, 150%, 175%, 260%, 250%, 300%,400%, 500%, 600%, 700%, 800% or 1000% increased transduction efficiency in heart relative to wild-type AAV9 and/or relative to transduction of liver. In some embodiments, any rAAV comprising N452K mutation as described herein exhibits about 20% to 30%, about 30% to 40%, about 40% to 50%, about 50% to 80%, about 80% to 100%, about 100% to 125%, about 125% to 150%, about 150% to 175%, or about 175% to 200% increased transduction efficiency in heart relative to wild-type AAV9 and/or relative to transduction of liver.

In some embodiments, any rAAV comprising N452K mutation as described herein exhibits at least 2-, 3-, 4-, 5-, 6, 7-, 8-, 9-, 10-, 11-, 12-, 13-, 14, or 15-fold decreased transduction efficiency in liver relative to wild-type AAV9. In some embodiments, any rAAV comprising N452K mutation as described herein exhibits about 2- to about 16-fold, about 2- to about 14-fold, about 2- to about 12-fold, about 2- to about 10-fold, about 2- to about 8-fold, about 2- to about 6-fold, about 2- to about 4-fold, or about 2- to about 3-fold decreased transduction efficiency in liver relative to wild-type AAV9. In some embodiments, any rAAV virion comprising N452K mutation as described herein exhibits at least or more than 30%, 40%, 50%, 80%, 100%, 125%, 150%, 175%, 200%, 250%, 300%, 400%, 500%, 600%, 700%, 800% or 1000% decreased transduction efficiency in liver relative to wild-type AAV9. In some embodiments, any rAAV comprising N452K mutation as described herein exhibits about 20% to 30%, about 30% to 40%, about 40% to 50%, about 50% to 80%, or about 80% to 100 decreased transduction efficiency in liver relative to wild-type AAV9.

### Gene Products/Transgenes

The transgenes and gene products described herein are non-limiting. Any transgene encoding any gene product may be used in the rAAV virions described herein.

In some embodiments, the rAAV virion of the present disclosure comprises a viral vector comprising a transgene.

A transgene can be a gene or nucleotide sequence that encodes a product, or a functional fragment thereof. A product can be, for example, a polypeptide or a non-coding nucleotide. By non-coding nucleotide, it is meant that the sequence transcribed from the transgene or nucleotide sequence is not translated into a polypeptide. In some embodiments, the product encoded by the transgene or nucleotide operably linked to an enhancer described herein is a non-coding polynucleotide. A non-coding polynucleotide can be an RNA, such as for example a microRNA (miRNA or mIR), short hairpin RNA (shRNA), long non-coding RNA (lnRNA), and/or a short interfering RNA (siRNA). In some embodiments, the transgene encodes a product natively expressed by a cardiac cell, e.g., a cardiomyocyte.

In some embodiments, the transgene encodes a polypeptide. In some embodiments, the transgene encodes a non-coding polynucleotide such as, for example, a microRNA (miRNA or mIR).

In some embodiments, the transgene comprises a nucleotide sequence encoding a human protein. In some embodiments, the transgene comprises a human nucleotide sequence (a human DNA sequence). In some embodiments, the transgene comprises a DNA sequence that has been codon-optimized. In some embodiments, the transgene comprises a nucleotide sequence encoding a wild-type protein, or a functionally active fragment thereof. In some embodiments, the transgene comprises a nucleotide sequence encoding a variant of a wild-type protein, such as a functionally active variant thereof.

In some embodiments, the transgene comprises a sequence encoding a product selected from vascular endothelial growth factor (VEGF), a VEGF isoform, VEGF-A, VEGF-B, VEGF-C, VEGF-D, VEGF-DdNdC, VEGF-Al 16A, VEGF-A165, VEGF-A121, VEGF-2, placenta growth factor (PIGF), fibroblast growth factor 4 (FGF-4), human growth factor (HGF), human granulocyte colony-stimulating factor (hGCSF), and hypoxia inducible factor 1α (HIF-1α).

In some embodiments, the transgene comprises a sequence encoding a product selected from SERCA2a, stromal cell-derived factor-1 (SDF-1), adenylyl cyclase type 6, S100A1, miRNA-17-92, miR-302-367, anti-miR-29a, anti-miR-30a, antimiR-141, cyclin A2, cyclin-dependent kinase 2, Tbx20, miRNA-590, miRNA-199, anti-sense oligonucleotide against Lp(a), interfering RNA against PCSK9, anti-sense oligonucleotide against apolipoprotein C-111, lipoprotein lipaseS447X, anti-sense oligonucleotide against apolipoprotein B, anti-sense oligonucleotide against c-myc, and E2F oligonucleotide decoy.

In some embodiments, the transgene encodes a gene product whose expression complements a defect in a gene responsible for a genetic disorder. In some embodiments, the disclosure provides, without limitation, polynucleotides encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOS1s (Noonan); SCN5A (Brugada); KCNQ1 and KCNH2s (Long QT Syndrome); DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, a polynucleotide encodes one or more of: cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3): titin (TTN); myosin, light chain 2 (MYL2); actin, alpha cardiac muscle 1 (ACTC1); potassium voltage-gated channel, KQT-like subfamily, member I (KCNQ1); myocyte enhancer factor 2c (MEF2C); and cardiac LIM protein (CSRP3).

In some embodiments, the transgene comprises a nucleotide sequence encoding a protein selected from DWORF, junctophilin (e.g., JPH2), BAG family molecular chaperone regulator 3 (BAG3), phospholamban (PLN), alpha-crystallin B chain (CRYAB), LMNA (such as Lamin A and Lamin C isoforms), troponin I type 3 (TNNI3), lysosomal-associated membrane protein 2 (LAMP2, such as LAMP2a, LAMP2b and LAMP2c isoforms), desmoplakin (DSP, such as DPI and DPII isoforms), desmoglein 2 (DSG2), junction plakoglobin (JUP), and plakophilin-2 (PKP2). In some embodiments, the transgene comprises a nucleotide sequence encoding a matrix metallopeptidase 11 (MMP11) protein, a synaptopodin 2 like (SYNPO2L) protein (e.g., SYNPO2LA or SYNPO2LA), or an RNA binding motif protein 20 (RBM20). In some embodiments, the transgene comprises a nucleotide sequence encoding an inhibitory oligonucleotide targeting metastasis suppressor protein 1 (MTSS1).

In some embodiments, the transgene in the viral vector (such as that in the rAAV virion of the present disclosure) is selected from DWORF, JPH2, BAG3, CRYAB, LMNA (e.g., Lamin A isoform of LMNA, or Lamin C isoform of LMNA), TNNI3, PLN, LAMP2 (e.g., LAMP2a, LAMP2b, or LAMP2c), DSP (e.g., DPI isoform of DSP or DPII isoform of DSP), DSG2 and JUP.

In some embodiments, the transgene comprises a polynucleotide sequence encoding a MYBPC3 polypeptide.

In some embodiments, the transgene comprises a polynucleotide sequence encoding a DWORF polypeptide.

In some embodiments, the transgene comprises a polynucleotide sequence encoding a junctophilin 2 (JPH2) polypeptide. In some embodiments, the transgene comprises a polynucleotide sequence encoding a full-length JPH2 polypeptide. In some embodiments, the transgene comprises a polynucleotide sequence encoding an N-terminal fragment of the JPH2 polypeptide. In some embodiments, the transgene comprises a polynucleotide sequence encoding an N-terminal fragment of the JPH2 polypeptide, which retains the JPH2 activity.

In some embodiments, the transgene comprises a polynucleotide sequence encoding a BAG3 polypeptide. In some embodiments, the transgene comprises a polynucleotide sequence encoding a C151R mutant form of BAG3 polypeptide.

In some embodiments, the transgene comprises a polynucleotide sequence encoding a CRYAB polypeptide.

In some embodiments, the transgene comprises a polynucleotide sequence encoding a LMNA polypeptide. In some embodiments, the transgene comprises a polynucleotide sequence encoding the LaminA isoform of LMNA. In some embodiments, the transgene comprises a polynucleotide sequence encoding the LaminC isoform of LMNA.

In some embodiments, the transgene comprises a polynucleotide sequence encoding a TNNI3 polypeptide.

In some embodiments, the transgene comprises a polynucleotide sequence encoding a PLN polypeptide.

In some embodiments, the transgene comprises a polynucleotide sequence encoding a LAMP2 polypeptide. In some embodiments, the transgene comprises a polynucleotide sequence encoding the LAMP2a isoform. In some embodiments, the transgene comprises a polynucleotide sequence encoding the LAMP2b isoform. In some embodiments, the transgene comprises a polynucleotide sequence encoding the LAMP2c isoform.

In some embodiments, the transgene comprises a polynucleotide sequence encoding a DSP polypeptide. In some embodiments, the transgene comprises a polynucleotide sequence encoding the DPI isoform of DSP. In some embodiments, the transgene comprises a polynucleotide sequence encoding the DPII isoform of DSP.

In some embodiments, the transgene comprises a polynucleotide sequence encoding a DSG2 polypeptide.

In some embodiments, the transgene comprises a polynucleotide sequence encoding a JUP polypeptide.

In some embodiments, the rAAV virion of the present disclosure comprises a heterologous nucleic acid comprising a nucleotide sequence that encodes one or more gene products selected from MYBPC3, KCNH2, TRPM4, DSG2, ATP2A2, CACNA1C, DMD, DMPK, EPGS, EVC, EVC2, FBN1, NF1, SCN5A, SOS1, NPR1, ERBB4, VIP, MYH6, MYH7, or a mutant, variant, or fragment thereof. In some embodiments, the rAAV virion of the present disclosure comprises a heterologous nucleic acid comprising a nucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAF1, BAG3, KCNQ1, MYLK3, CRYAB, ALPK3 and ACTN2. In some embodiments, the rAAV virion of the present disclosure comprises a heterologous nucleic acid comprising a nucleotide sequence that encodes one or more gene products selected from MYBPC3, DWORF, JPH2, BAG3, CRYAB, Lamin A isoform of LMNA, Lamin C isoform of LMNA. TNNI3, PLN, LAMP2a, LAMP2b. LAMP2c, DPI isoform of DSP, DPII isoform of DSP, DSG2. MYH6, MYH7, RBM20, and JUP.

In some embodiments, the rAAV virion of the present disclosure comprises a heterologous nucleic acid comprising a nucleotide sequence that encodes one or more gene products selected from ASCL1, MYOCD, MEF2C, and TBX5. In some embodiments, the rAAV virion of the present disclosure comprises a heterologous nucleic acid comprising a nucleotide sequence that encodes one or more gene products selected from ASCL1, MYOCD, MEF2C, AND TBX5, CCNB 1, CCND1, CDK1, CDK4, AURKB, OCT4, BAF60C, ESRRG, GATA4, GATA6, HAND2, IRX4, ISLL, MESP1, MESP2, NKX2.5, SRF, TBX20, ZFPM2, and MIR-133.

In some embodiments, the rAAV virion of the present disclosure comprises a heterologous nucleic acid comprising a nucleotide sequence that encodes one or more gene products selected from MYBPC3, DWORF, KCNH2, TRPM4, DSG2, and ATP2A2.

In some embodiments, the rAAV virion of the present disclosure comprises a heterologous nucleic acid comprising a nucleotide sequence that encodes one or more gene products selected from TGFBR2, TGFBR1, EMD, KCNQ1, TAZ, COL3A1, JUP, CASQ2, MLRP44, DNAJC19, LMNA, TNNI3, DSP, DSG2, RAF1, SOS1, FBN1, LAMP2, FXN, RAFI, BAG3, KCNQ1, MYLK3, CRYAB, ALPK3 and ACTN2.

In some embodiments, the rAAV virion of the present disclosure comprises a heterologous nucleic acid comprising a nucleotide sequence that encodes one or more gene products selected from CACNA1C, DMD, DMPK, EPG5, EVC, EVC2, FBN1, NF1, SCN5A, SOSI, NPR1, ERBB4, VIP, MYH6, MYH7, and Cas9. In some embodiments, the rAAV virion of the present disclosure comprises a heterologous nucleic acid comprising a nucleotide sequence that encodes saCas9.

In some embodiments, the rAAV virion of the present disclosure comprises a heterologous nucleic acid comprising a nucleotide sequence that encodes one or more gene products selected from MYOCD, ASCL1, GATA4, MEF2C, TBX5, miR-133, and MESP1.

In some embodiments, the transgene in the rAAV virion of the present disclosure encodes any of the above-identified gene products.

In some embodiments, the capsids described herein improve heart transduction efficiency, liver viral load, and/or heart-to-liver transduction ratio of the rAAV virions carrying any of the transgenes described herein (and encoding, and resulting in the expression of, any of the gene products described herein).

### Compostions

Efforts to identify capsid variants with properties useful for gene therapy have included shuffling the DNA of AAV2 and AAV5 cap genes as described in U.S. Pat. No. 9,233,131; as well as directed evolution as described in Int'l Pat. Appl. Nos. WO2012/145601A2 and WO2018/222503A 1. The disclosures of these documents are incorporated here for all purposes, and particularly for the methods of making and using AAV virions and for the polynucleotide sequences and gene products therein disclosed, as well as for the combinations of transcription factors useful in treating cardiac diseases or disorders.

The AAV capsid is encoded by the cap gene of AAV, which is also termed the right open-reading frame (ORF) (in contrast to the left ORF, rep). The structures of representative AAV capsids are described in various publications including Xie et al. (2002) Proc. Natl. Acad. Sci USA 99:10405-1040 (AAV2); Govindasamy et al. (2006) J. Virol. 80:11556-11570 (AAV4); Nam et al. (2007) J. Virol. 81:12260-12271 (AAV8) and Govindasamy et al. (2013) J. Virol. 87:11187-11199 (AAV5).

The AAV capsid contain 60 copies (in total) of three viral proteins (VPs), VP1. VP2, and VP3, in a predicted ratio of 1:1:10, arranged with T=1 icosahedral symmetry. The three VPs are translated from the same mRNA, with VP1 containing a unique N-terminal domain in addition to the entire VP2 sequence at its C-terminal region. VP2 contains an extra N-terminal sequence in addition to VP3 at its C terminus. In most crystal structures, only the C-terminal polypeptide sequence common to all the capsid proteins (˜530 amino acids) is observed. The N-terminal unique region of VP1, the VP1-VP2 overlapping region, and the first 14 to 16 N-terminal residues of VP3 are thought to be primarily disordered. Cryo-electron microscopy and image reconstruction data suggest that in intact AAV capsids, the N-terminal regions of the VP1 and VP2 proteins are located inside the capsid and are inaccessible for receptor and antibody binding. Thus, receptor attachment and transduction phenotypes are, generally, determined by the amino acid sequences within the common C-terminal domain of VP1, VP2 and VP3

In some embodiments, the one or more amino acid insertions, substitutions, or deletions is/are in the GH loop, or loop IV, of the AAV capsid protein, e.g., in a solvent-accessible portion of the GH loop, or loop IV, of the AAV capsid protein. For the GH loop/loop IV of AAV capsid, see, e.g., van Vliet et al. (2006) Mol. Ther. 14:809; Padron et al. (2005) Virol. 79:5047; and Shen et al. (2007) Mol. Ther. 15: 1955. In some embodiments, a “parental” AAV capsid protein is a wild-type AAV9 capsid protein. In some embodiments, a “parental” AAV capsid protein is a wild-type AAV5 capsid protein. In some embodiments, a “parental” AAV capsid protein is a chimeric AAV capsid protein. Amino acid sequences of various AAV capsid proteins are known in the art. See. e.g., GenBank Accession No. NP_049542 for AAV1; GenBank Accession No. NP 044927 for AAV4; GenBank Accession No. AAD13756 for AAV5; GenBank Accession No. AAB95450 for AAV6; GenBank Accession No. YP_077178 for AAV7; GenBank Accession No. YP_077180 for AAV 8; GenBank Accession No. AAS99264 for AAV9 and GenBank Accession No. AAT46337 for AAV10. See. e.g., Santiago-Ortiz et al. (2015) Gene Ther. 22:934 for a predicted ancestral AAV capsid.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the AAV5 genome is provided in GenBank Accession No. AF085716. The life cycle and genetics of AAV are reviewed in Muzyczka, Current Topics in Microbiology and Immunology, 158: 97-129 (1992), Production of pseudotyped rAAV is disclosed in, for example, WO 01/83692. Other types of rAAV variants, for example rAAV with capsid mutations, are also contemplated. See, for example. Marsic et al., Molecular Therapy, 22(11): 1900-1909 (2014), Illustrative AAV vectors are provided in U.S. Pat. No. 7,105,345: U.S. Ser. No. 15/782,980; U.S. Pat. Nos. 7,259,151; 6,962,815; 7,718,424; 6,984,517; 7,718,424; 6,156,303; 8,524,446; 7,790,449; 7,906,111; 9,737,618; U.S. application Ser. No. 15/433,322; U.S. Pat. No. 7,198,951, each of which is incorporated by reference in its entirety for all purposes.

The rAAV virions of the disclosure comprise a heterologous nucleic acid comprising a nucleotide sequence encoding one or more gene product. The gene product(s) may be either a polypeptide or an RNA, or both. When the gene product is a polypeptide, the nucleotide sequence encodes a messenger RNA, optionally with one or more introns, which is translated into the gene product polypeptide. The nucleotide sequence may encode one, two, three, or more gene products (though the number is limited by the packaging capacity of the rAAV virion, typically about 5.2 kb). The gene products may be operatively linked to one promoter (for a single transcriptional unit) or more than one. Multiple gene products may also be produced using internal ribosome entry signal (IRES) or a self-cleaving peptide (e.g., a 2A peptide).

In some embodiments, the gene product is a polypeptide. In some embodiments, the polypeptide gene product is a polypeptide that induces reprogramming of a cardiac fibroblast, to generate an induced cardiomyocyte-like cell (iCM). In some embodiments, the polypeptide gene product is a polypeptide that enhances the function of a cardiac cell. In some embodiments, the polypeptide gene product is a polypeptide that provides a function that is missing or defective in the cardiac cell. In some embodiments, the polypeptide gene product is a genome-editing endonuclease.

In some embodiments, the gene product comprises a fusion protein that is fused to a heterologous polypeptide. In some embodiments, the gene product comprises a genome editing nuclease fused to an amino acid sequence that provides for subcellular localization, i.e., the fusion partner is a subcellular localization sequence (e.g., one or more nuclear localization signals (NLSs) for targeting to the nucleus, two or more NLSs, three or more NLSs, etc.).

In general, a viral vector is produced by introducing a viral DNA or RNA construct into a “producer cell” or “packaging cell” line. Packaging cell lines include but are not limited to any easily-transfectable cell line. Packaging cell lines can be based on HEK291, 293T cells, NIH3T3, COS, HeLa or Sf9 cell lines. Examples of packaging cell lines include but are not limited to: Sf9 (ATCC® CRL-1711™). Exemplary packing cell lines and methods for generating rAAV virions are provided by Int'l Pat. Pub. Nos. WO2017075627, WO2015/031686, WO2013/063379, WO2011/020710. WO2009/104964, WO2008/024998, WO20031042361, and WO1995/013392; U.S. Pat. Nos. 9,441,206B2, 8,679,837, and 7,091,029B2.

In some embodiments, the gene product is a functional cardiac protein. In some embodiments, the gene product is a genome-editing endonuclease (optionally with a guide RNA, single-guide RNA, and/or repair template) that replaces or repairs a non-functional cardiac protein into a functional cardiac protein. Functional cardiac proteins include, but are not limited to cardiac troponin T; a cardiac sarcomeric protein; β-myosin heavy chain; myosin ventricular essential light chain 1; myosin ventricular regulatory light chain 2; cardiac a-actin; a-tropomyosin; cardiac troponin 1; cardiac myosin binding protein C; four-and-a-half LIM protein 1; titin; 5′-AMP-activated protein kinase subunit gamma-2; troponin 1 type 3, myosin light chain 2, actin alpha cardiac muscle 1; cardiac LIM protein; caveolin 3 (CAV3): galactosidase alpha (GLA); lysosomal-associated membrane protein 2 (LAMP2); mitochondrial transfer RNA glycine (MTTG); mitochondrial transfer RNA isoleucine (MTTI); mitochondrial transfer RNA lysine (MTTK); mitochondrial transfer RNA glutamine (MTTQ): myosin light chain 3 (MYL3); troponin C (TNNC1); transthyretin (TTR); sarcoendoplasmic reticulum calcium-ATPase 2a (SERCA2a); stromal-derived factor-1 (SDF-1); adenylate cyclase-6 (AC6); beta-ARKet (β-adrenergic receptor kinase C terminus); fibroblast growth factor (FGF); platelet-derived growth factor (PDGF); vascular endothelial growth factor (VEGF); hepatocyte growth factor; hypoxia inducible growth factor; thymosin beta 4 (TMSB4X); nitric oxide synthase-3 (NOS3); unocartin 3 (UCN3); melusin; apoplipoprotein-E (Apo); superoxide dismutase (SOD); and S100A1 (a small calcium binding protein; see, e.g., Ritterhoff and Most (2012) Gene Ther. 19:613; Kraus et al. (2009) Mol. Cell. Cardiol. 47:445).

In some embodiments, the gene product is a gene product whose expression complements a defect in a gene responsible for a genetic disorder. The disclosure provides rAAV virions comprising a polynucleotide encoding one or more of the following—e.g., for use, without limitation, in the disorder indicated in parentheses, or for other disorders caused by each: TAZ (Barth syndrome); FXN (Freidrich's Ataxia); CASQ2 (CPVT); FBN1 (Marfan); RAF1 and SOS1s (Noonan); SCN5A (Brugada); KCNQ1 and KCNH2s (Long QT Syndrome): DMPK (Myotonic Dystrophy 1); LMNA (Limb Girdle Dystrophy Type 1B); JUP (Naxos); TGFBR2 (Loeys-Dietz); EMD (X-Linked EDMD); and ELN (SV Aortic Stenosis). In some embodiments, the rAAV virion comprises a polynucleotide encoding one or more of cardiac troponin T (TNNT2); BAG family molecular chaperone regulator 3 (BAG3); myosin heavy chain (MYH7); tropomyosin 1 (TPM1); myosin binding protein C (MYBPC3); 5′-AMP-activated protein kinase subunit gamma-2 (PRKAG2); troponin 1 type 3 (TNNI3); titin (TTN); myosin, light chain 2 (MYL2); actin, alpha cardiac muscle 1 (ACTC1); potassium voltage-gated channel, KQT-like subfamily, member 1 (KCNQ1); plakophilin 2 (PKP2); myocyte enhancer factor 2c (MEF2C); and cardiac LIM protein (CSRP3).

In some embodiments, the gene products of the disclosure are polypeptide reprogramming factors. Reprogramming factors are desirable as means to convert one cell type into another. Non-cardiomyocytes cells can be differentiated into cardiomyocytes cells in vitro or in vivo using any method available to one of skill in the art. For example, see methods described in Ieda et al. (2010) Cell 142:375-386; Christoforou et al. (2013) PLoS ONE 8:e63577; Addis et al. (2013) J. Mol. Cell Cardiol. 60:97-106; Jayawardena et al. (2012) Circ. Res. 110: 1465-1473; Nam Y et al. (2003) PNAS USA 110:5588-5593; Wada R et al. (2013) PNAS USA 110: 12667-12672; and Fu J et al. (2013). Stem Cell Reports 1:235-247.

In cardiac context, the reprogramming factors may be capable of converting a cardiac fibroblast to a cardiac myocyte either directly or through an intermediate cell type. In particular, direct reprogramming is possible, or reprogramming by first converting the fibroblast to a pluripotent or totipotent stem cell. Such a pluripotent stem cell is termed an induced pluripotent stem (iPS) cell. An iPS cell that is subsequently converted to a cardiac myocyte (CM) cell is termed an iPS-CM cell. In the examples, iPS-CM derived in vitro from cardiac fibroblasts are used in vivo to select capsid proteins of interest. The disclosure also envisions using the capsid proteins disclosure to in turn generate iPS-CM cells in vitro but, particular, in vivo, as part of a therapeutic gene therapy regimen. Induced cardiomyocyte-like (iCM) cells refer to cells directly reprogrammed into cardiomyocytes.

Induced cardiomyocytes express one or more cardiomyocyte-specific markers, where cardiomyocyte-specific markers include, but are not limited to, cardiac troponin 1, cardiac troponin-C, tropomyosin, caveolin-3, myosin heavy chain, myosin light chain-2a, myosin light chain-2v, ryanodine receptor, sarcomeric a-actinin, Nkx2.5, connexin 43, and atrial natriuretic factor. Induced cardiomyocytes can also exhibit sarcomeric structures. Induced cardiomyocytes exhibit increased expression of cardiomyocyte-specific genes ACTC1 (cardiac a-actin), ACTN2 (actinin a2), MYH6 (a-myosin heavy chain), RYR2 (ryanodine receptor 2), MYL2 (myosin regulatory light chain 2, ventricular isoform), MYL7 (myosin regulatory light chain, atrial isoform), TNNT2 (troponin T type 2, cardiac), and NPPA (natriuretic peptide precursor type A), PLN (phospholamban). Expression of fibroblasts markers such as Colla2 (collagen 1a2) is downregulated in induced cardiomyocytes, compared to fibroblasts from which the iCM is derived.

Reprogramming methods involving polypeptide reprogramming factors (in some cases supplemented by small-molecule reprogramming factors supplied in conjunction with the rAAV) include those described in US2018/0112282A1, WO2018/005546, WO2017/173137, US2016/0186141, US2016/0251624, US2014/0301991, and US2013/0216503A1, which are incorporated in their entirety, particularly for the reprogramming methods and factors disclosed.

In some embodiments, cardiac cells are reprogrammed into induced cardiomyocyte-like (iCM) cells using one or more reprogramming factors that modulate the expression of one or more polynucleotides or proteins of interest, such as Achaete-scute homolog 1 (ASCL1), Myocardin (MYOCD), myocyte-specific enhancer factor 2C (MEF2C), and/or T-box transcription factor 5 (TBX5). In some embodiments, the one or more reprogramming factors are provided as a polynucleotide (e.g., an RNA, an mRNA, or a DNA polynucleotide) that encode one or more polynucleotides or proteins of interest. In some embodiments, the one or more reprogramming factors are provided as a protein.

In some embodiments, the reprogramming factors are microRNAs or microRNA antagonists, siRNAs, or small molecules that are capable of increasing the expression of one or more polynucleotides or proteins of interest. In some embodiments, expression of a polynucleotides or proteins of interest is increased by expression of a microRNA or a microRNA antagonist. For example, endogenous expression of an Oct polypeptide can be increased by introduction of microRNA-302 (miR-302), or by increased expression of miR-302. See, e.g., Hu et al., Stem Cells 31(2): 259-68 (2013), which is incorporated herein by reference in its entirety. Hence, miRNA-302 can be an inducer of endogenous Oct polypeptide expression. The miRNA-302 can be introduced alone or with a nucleic acid that encodes the Oct polypeptide. In some embodiments, a suitable nucleic acid gene product is a microRNA. Suitable microRNAs include, e.g., mir-1, mir-133, mir-208, mir-143, mir-145, and mir-499.

In some embodiments, the methods of the disclosure comprise administering an rAAV virion of the disclosure before, during, or after administration of the small-molecule reprogramming factor. In some embodiments, the small-molecule reprogramming factor is a small molecule selected from the group consisting of SB431542, LDN-193189, dexamethasone, LY364947, D4476, myricetin, IWR1, XAV939, docosahexaenoic acid (DHA), S-Nitroso-TV- acetylpenicillamine (SNAP), Hh-Ag1.5, alprostadil, cromakalim, MNITMT, A769662, retinoic acid p-hydoxyanlide, decamethonium dibromide, nifedipine, piroxicam, bacitracin, aztreonam, harmalol hydrochloride, amide-C2 (A7), Ph-C12 (CIO), mCF3-C-7 (J5), G856-7272 (A473), 3475707, or any combination thereof.

In some embodiments, the gene products comprise reprogramming factors that modulate the expression of one or more proteins of interest selected from ASCL1, MYOCD, MEF2C, and TBX5. In some embodiments, the gene products comprise one or more reprogramming factors selected from ASCL1, MYOCD, MEF2C, AND TBX5, CCNB1, CCND1, CDKI, CDK4, AURKB, OCT4, BAF60C, ESRRG, GATA4, GATA6, HAND2, IRX4, ISLL, MESP1, MESP2, NKX2.5, SRF, TBX20, ZFPM2, and miR-133.

In some embodiments, the gene products comprise GATA4, MEF2C, and TBX5 (i.e., GMT). In some embodiments, the gene products comprise MYOCD, MEF2C, and TBX5 (i.e., MyMT). In some embodiments, the gene products comprise MYOCD, ASCL1, MEF2C, and TBX5 (i.e., MyAMT). In some embodiments, the gene products comprise MYOCD and ASCL1 (i.e., MyA). In some embodiments, the gene products comprise GATA4, MEF2C, TBX5, and MYOCD (i.e., 4F). In other embodiments, the gene products comprise GATA4, MEF2C, TBX5, ESSRG, MYOCD, ZFPM2, and MESP1 (i.e., 7F). In some embodiments, the gene products comprise one or more of ASCL1, MEF2C, GATA4, TBX5, MYOCD, ESRRG, AND MESPL.

In some embodiments, the rAAV virions generate cardiac myocytes in vitro or in vivo. Cardiomyocytes or cardiac myocytes are the muscle cells that make up the cardiac muscle. Each myocardial cell contains myofibrils, which are long chains of sarcomeres, the contractile units of muscle cells. Cardiomyocytes show striations similar to those on skeletal muscle cells, but unlike multinucleated skeletal cells, they contain only one nucleus. Cardiomyocytes have a high mitochondrial density, which allows them to produce ATP quickly, making them highly resistant to fatigue. Mature cardiomyocytes can express one or more of the following cardiac markers: α-Actinin, MLC2v, MY20, cMHC, NKX2-5, GATA4, cTNT, cTNI, MEF2C, MLC2a, or any combination thereof. In some embodiments, the mature cardiomyocytes express NKX2-5, MEF2C or a combination thereof. In some embodiments, cardiac progenitor cells express early stage cardiac progenitor markers such as GATA4, ISL1 or a combination thereof.

In some embodiments, the gene product is a polynucleotide. In some embodiments, as described below, the gene product is a guide RNA capable of binding to an RNA-guided endonuclease. In some embodiments, the gene product is an inhibitory nucleic acid capable of reducing the level of an mRNA and/or a polypeptide gene product, e.g., in a cardiac cell. For example, in some embodiments, the polynucleotide gene product is an interfering RNA capable of selectively inactivating a transcript encoded by an allele that causes a cardiac disease or disorder. As an example, the allele is a myosin heavy chain 7, cardiac muscle, beta (MYH7) allele that comprises a hypertrophic cardiomyopathy-causing mutation. Other examples include, e.g., interfering RNAs that selectively inactivate a transcript encoded by an allele that causes hypertrophic cardiomyopathy (HCM), dilated cardiomyopathy (DCM) or Left Ventricular Non-Compaction (LVNC), where the allele is a MYL3 (myosin light chain 3, alkali, ventricular, skeletal slow), MYH7, TNNI3 (troponin I type 3 (cardiac)), TNNT2 (troponin T type 2 (cardiac)), TPMI (tropomyosin 1 (alpha)) or ACTC1 allele comprising an HCM-causing, a DCM-causing or a LVNC-causing mutation. See. e.g., U.S. Pat. Pub. No. 2016/0237430 for examples of cardiac disease-causing mutations.

In some embodiments, the gene product is a polypeptide-encoding RNA. In some embodiments, the gene product is an interfering RNA. In some embodiments, the gene product is an aptamer. In some embodiments, the gene product is a polypeptide. In some embodiments, the gene product is a therapeutic polypeptide, e.g., a polypeptide that provides clinical benefit. In some embodiments, the gene product is a site-specific nuclease that provide for site-specific knock-down of gene function. In some embodiments, the gene product is an RNA-guided endonuclease that provides for modification of a target nucleic acid. In some embodiments, the gene products are: i an RNA-guided endonuclease that provides for modification of a target nucleic acid; and ii) a guide RNA that comprises a first segment that binds to a target sequence in a target nucleic acid and a second segment that binds to the RNA-guided endonuclease. In some embodiments, the gene products are: i) an RNA-guided endonuclease that provides for modification of a target nucleic acid; ii) a first guide RNA that comprises a first segment that binds to a first target sequence in a target nucleic acid and a second segment that binds to the RNA-guided endonuclease; and iii) a first guide RNA that comprises a first segment that binds to a second target sequence in the target nucleic acid and a second segment that binds to the RNA-guided endonuclease.

A nucleotide sequence encoding a heterologous gene product in an rAAV virion of the present disclosure can be operably linked to a promoter. For example, a nucleotide sequence encoding a heterologous gene product in an rAAV virion of the present disclosure can be operably linked to a constitutive promoter, a regulatable promoter, or a cardiac cell-specific promoter. Suitable constitutive promoters include a human elongation factor 1α subunit (EF1α) promoter, a β-actin promoter, an α-actin promoter, a β-glucuronidase promoter, CAG promoter, super core promoter, and a ubiquitin promoter. In some embodiments, a nucleotide sequence encoding a heterologous gene product in an rAAV virion of the present disclosure is operably linked to a cardiac-specific transcriptional regulator element (TRE), where cardiac-specific TREs include promoters and enhancers. Suitable cardiac-specific TREs include, but are not limited to, TREs derived from the following genes: myosin light chain-2 (MLC-2), a- myosin heavy chain (a-MHC), desmin, AE3, cardiac troponin C (cTnC), and cardiac actin. Franz et al. (1997) Cardiovasc. Res. 35:360-566; Robbins et al. (1995) Ann. NY. Acad. Sci. 752:492-505; Linn et al. (1995) Circ. Res. 76:584-591; Parmacek et al. (1994) Mol. Cell. Biol. 14: 1870-1885; Hunter et al. (1993) Hypertension 22:608-617; and Sartorelli et al. (1992) Proc. Natl. Acad. Sci. USA 89:4047-4051. See also, Pacak et al. (2008) Genet Vaccines Ther. 6:13. In some embodiments, the promoter is an a-MHC promoter, an MLC-2 promoter, or cTnT promoter.

The polynucleotide encoding a gene product is operably linked to a promoter and/or enhancer to facilitate expression of the gene product. Depending on the host/vector system utilized, any of a number of suitable transcription and translation control elements, including constitutive and inducible promoters, transcription enhancer elements, transcription terminators, etc. may be used in the rAAV virion (e.g., Bitter et al. (1987) Methods in Enzymology, 153:516-544).

Separate promoters and/or enhancers can be employed for each of the polynucleotides. In some embodiments, the same promoter and/or enhance is used for two or more polynucleotides in a single open reading frame. Vectors employing this configuration of genetic elements are termed “polycistronic.” An illustrative example of a polycistronic vector comprises an enhancer and a promoter operatively linked to a single open-reading frame comprising two or more polynucleotides linked by 2A region(s), whereby expression of the open-reading frame result in multiple polypeptides being generated co-translationally. The 2A region is believed to mediate generation of multiple polypeptide sequences through codon skipping; however, the present disclosure relates also to polycistronic vectors that employ post-translational cleavage to generate two or more proteins of interest from the same polynucleotide. Illustrative 2A sequences, vectors, and associated methods are provided in US20040265955A1, which is incorporated herein by reference.

Non-limiting examples of suitable eukaryotic promoters (promoters functional in a eukaryotic cell) include CMV. CMV immediate early, HSV thymidine kinase, early and late SV40, long terminal repeats (LTRs) from retrovirus, and mouse metallothionein-1. In some embodiments, promoters that are capable of conferring cardiac specific expression will be used. Non-limiting examples of suitable cardiac specific promoters include desmin (Des), alpha-myosin heavy chain (a-MHC), myosin light chain 2 (MLC-2), cardiac troponin T (cTnT) and cardiac troponin C (cTnC). Non-limiting examples of suitable neuron specific promoters include synapsin I (SYN), calcium/calmodulin-dependent protein kinase H, tubulin alpha 1, neuron-specific enolase and platelet-derived growth factor beta chain promoters and hybrid promoters by fusing cytomegalovirus enhancer (E) to those neuron-specific promoters.

Examples of suitable promoters for driving expression reprogramming factors include, but are not limited to, retroviral long terminal repeat (LTR) elements; constitutive promoters such as CMV, HSV1-TK, SV40, EF-1a, β-actin, phosphoglycerol kinase (PGK); inducible promoters, such as those containing Tet- operator elements; cardiac specific promoters, such as desmin (DES), alpha-myosin heavy chain (a-MHC), myosin light chain 2 (MLC-2), cardiac troponin T (cTnT) and cardiac troponin C (cTnC); neural specific promoters, such as nestin, neuronal nuclei (NeuN), microtubule-associate protein 2 (MAP2), beta III tubulin, neuron specific enolase (NSE), oligodendrocyte lineage (Olig1/2), and glial fibrillary acidic protein (GFAP); and pancreatic specific promoters, such as Pax4, Nkx2.2, Ngn3, insulin, glucagon, and somatostatin.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, a-myosin heavy chain. AE3, cardiac troponin C, and cardiac actin. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Cell. Biol. 14:1870-1885: Hunter et al. (1993) Hypertension 22:608-617; and Sartorelli et al. (1992) PNAS USA 89:4047-4051.

The promoter can be one naturally associated with a gene or nucleic acid segment. Similarly, for RNAs (e.g., microRNAs), the promoter can be one naturally associated with a microRNA gene (e.g., a miRNA-302 gene). Such a naturally associated promoter can be referred to as the “natural promoter” and may be obtained by isolating the 5′ non-coding sequences located upstream of the coding segment and/or exon. Similarly, an enhancer may be one naturally associated with a nucleic acid sequence. However, the enhancer can be located either downstream or upstream of that sequence.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers can include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference).

The promoters employed may be constitutive, inducible, developmentally-specific, tissue-specific, and/or useful under the appropriate conditions to direct high level expression of the nucleic acid segment. For example, the promoter can be a constitutive promoter such as, a CMV promoter, a CMV cytomegalovirus immediate early promoter, a CAG promoter, an EF-1α promoter, a HSV1-TK promoter, an SV40 promoter, a β-actin promoter, a PGK promoter, or a combination thereof. Examples of eukaryotic promoters that can be used include, but are not limited to, constitutive promoters, e.g., viral promoters such as CMV. SV40 and RSV promoters, as well as regulatable promoters, e.g., an inducible or repressible promoter such as the tot promoter, the hsp70 promoter and a synthetic promoter regulated by CRE. In certain embodiments, cell type-specific promoters are used to drive expression of reprogramming factors in specific cell types. Examples of suitable cell type-specific promoters useful for the methods described herein include, but are not limited to, the synthetic macrophage-specific promoter described in He et al (2006), Human Gene Therapy 17:949-959; the granulocyte and macrophage-specific lysozyme M promoter (see, e.g., Faust et al (2000), Blood 96(2):719-726); and the myeloid-specific CD11b promoter (see, e.g., Dziennis et al (1995), Blood 85(2):319-329), Other examples of promoters that can be employed include a human EF1α elongation factor promoter, a CMV cytomegalovirus immediate early promoter, a CAG chicken albumin promoter, a viral promoter associated with any of the viral vectors described herein, or a promoter that is homologous to any of the promoters described herein (e.g., from another species). Examples of prokaryotic promoters that can be used include, but are not limited to, SP6, T7, T5, tac, bla, trp, gal, lac, or maltose promoters.

In some embodiments, an internal ribosome entry sites IRES) element can be used to create multigene, or polycistronic, messages. IRES elements are able to bypass the ribosome scanning model of 5′-methylated Cap dependent translation and begin translation at internal sites (Pelletier and Sonenberg, Nature 334(6180):320-325 (1988)). IRES elements from two members of the picornavirus family (polio and encephalomyocarditis) have been described (Pelletier and Sonenberg, Nature 334(6180):320-325 (1988)), as well an IRES from a mammalian message (Macejak & Samow, Nature 353:90-94 (1991)). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

In some embodiments, a nucleotide sequence is operably linked to a polyadenylation sequence. Suitable polyadenylation sequences include bovine growth hormone polyA signal (bGHpolyA) and short poly A signal. Optionally the rAAV vectors of the disclosure comprise the Woodchuck Post-transcriptional Regulatory Element (WPRE). In some embodiments, the polynucleotide encoding gene products are join by sequences include so-called self-cleaving peptide, e.g., P2A peptides.

In some embodiments, the gene product comprises a site-specific endonuclease that provides for site-specific knock-down of gene function, e.g., where the endonuclease knocks out an allele associated with a cardiac disease or disorder. For example, where a dominant allele encodes a defective copy of a gene that, when wild-type, is a cardiac structural protein and/or provides for normal cardiac function, a site-specific endonuclease can be targeted to the defective allele and knock out the defective allele. In some embodiments, a site-specific endonuclease is an RNA-guided endonuclease.

In addition to knocking out a defective allele, a site-specific nuclease can also be used to stimulate homologous recombination with a donor DNA that encodes a functional copy of the protein encoded by the defective allele. For example, a subject rAAV virion can be used to deliver both a site-specific endonuclease that knocks out a defective allele a functional copy of the defective allele (or fragment thereof), resulting in repair of the defective allele, thereby providing for production of a functional cardiac protein (e.g., functional troponin, etc.). In some embodiments, a subject rAAV virion comprises a heterologous nucleotide sequence that encodes a site-specific endonuclease and a heterologous nucleotide sequence that encodes a functional copy of a defective allele, where the functional copy encodes a functional cardiac protein. Functional cardiac proteins include, e.g., troponin, a chloride ion channel, and the like.

Site-specific endonucleases that are suitable for use include, e.g., zinc finger nucleases (ZFNs); meganucleases; and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Pat. Pub. No. 2011/0301073. Suitable site-specific endonucleases include engineered meganuclease re-engineered homing endonucleases. Suitable endonucleases include an I-Tevl nuclease. Suitable meganucleases include I-Scel (see, e.g., Bellaiche et al. (1999) Genetics 152: 1037); and I-Crel (see, e.g., Heath et al. (1997) Nature Sructural Biology 4:468), Site-specific endonucleases that are suitable for use include CRISPRi systems and the Cas9-based SAM system.

In some embodiments, the gene product is an RNA-guided endonuclease. In some embodiments, the gene product comprises an RNA comprising a nucleotide sequence encoding an RNA-guided endonuclease. In some embodiments, the gene product is a guide RNA, e.g., a single-guide RNA. In some embodiments, the gene products are: I) a guide RNA; and 2) an RNA-guided endonuclease. The guide RNA can comprise: a) a protein-binding region that binds to the RNA-guided endonuclease; and b) a region that binds to a target nucleic acid. An RNA-guided endonuclease is also referred to herein as a “genome editing nuclease.”

Examples of suitable genome editing nucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some embodiments, the gene product comprises a class 2 CRISPR/Cas endonuclease. In some embodiments, the gene product comprises a class 2 type II CRISPR/Cas endonuclease (e.g., a Cas9 protein). In some embodiments, the gene product comprises a class 2 type V CRISPR/Cas endonuclease (e.g., a Cpf1 protein, a C2c1 protein, or a C2c3 protein). In some embodiments, the gene product comprises a class 2 type VI CRISPR/Cas endonuclease (e.g., a C2c2 protein; also referred to as a “Cas13a” protein). In some embodiments, the gene product comprises a CasX protein. In some embodiments, the gene product comprises a CasY protein.

Nucleic Acids, Vector, Cells, and Generation of rAAV Virions

In some embodiments, the disclosure provides nucleic acids encoding any AAV capsid protein described herein (such as AAV capsid proteins comprising one or more of the modifications described herein).

In some embodiments, the disclosure provides a vector or a plasmid comprising a nucleic acid encoding any AAV capsid protein described herein. In some embodiments, the vector or plasmid further comprises a promoter operably linked to the nucleic acid encoding the AAV capsid proteins. In some embodiments, the promoter is any promoter active in a cell to be used for expressing the capsid protein (e.g., a producer or host cell). In some embodiments, the promoter is P40 promoter. In some embodiments, the promoter is a polyhedrin promoter.

In some embodiments, the vector or plasmid comprising a nucleic acid encoding any AAV capsid protein described herein further comprises a nucleic acid encoding a replication (Rep) protein. In some embodiments, the Rep protein is a Rep protein from the same serotype of AAV as the inverted terminal repeats (ITRs) used to flank the transgene (to be packaged into virions using any of the AAV capsid proteins described herein). In some embodiments, the Rep protein is an AAV2 Rep protein. In some embodiments, the Rep protein is an AAV8 Rep protein. In some embodiments, the vector or plasmid comprising a nucleic acid encoding any AAV capsid protein described herein does not further comprise a nucleic acid encoding a Rep protein.

In some embodiments, the disclosure provides a cell comprising a nucleic acid encoding any AAV capsid protein described herein. In some embodiments, the disclosure provides a cell comprising a vector or a plasmid comprising a nucleic acid encoding any AAV capsid protein described herein. In some embodiments, the cell further comprises a vector or plasmid comprising a nucleic acid encoding a Rep protein, wherein the Rep protein may be expressed by the same or different vector or plasmid as the AAV capsid protein described herein.

In some embodiments, the disclosure provides a host cell comprising a nucleic acid encoding any AAV capsid protein described herein. In some embodiments, the disclosure provides a host cell comprising a vector or a plasmid comprising a nucleic acid encoding any AAV capsid protein described herein.

In some embodiments, a host cell comprising a nucleic acid encoding any AAV capsid protein described herein is for producing an rAAV virion described herein (such as an rAAV virion comprising a modified AAV capsid protein as described herein). In some embodiments, the nucleic acid encoding any AAV capsid protein is transiently transfected into a cell. In some embodiments, the nucleic acid encoding any AAV capsid protein is stably inserted into the cell genome.

In some embodiments, the host cell is a mammalian cell. In some embodiments, the host cell is selected from the group consisting of: are HEK293, HEK293T, HeLa, Vero, MDCK, MRC-5, PER.C6, BHK21 and CHO. In some embodiments, the host cell is HEK293 cell.

In some embodiments, the host cell is an insect cell. In some embodiments, the host cell is Sf9 insect cell. In some embodiments where the insect cells are used as host cells, the vectors or plasmids described herein are first introduced into a recombinant baculovirus and then carried into insect cells by baculovirus infection.

In some embodiments, the host cells are further transfected with one or more vectors or phasmids comprising helper functions and/or viral structural proteins necessary for replication and/or encapsidation of the vector(s) carrying the transgene.

In some embodiments, the host cells are further transfected with a viral vector carrying a transgene (such as any transgene described herein). In some embodiments, the transgene is flanked by inverted terminal repeats (ITRs). In some embodiments, the ITRs are of the same serotype as the Rep protein expressed in the host cells. In some embodiments, the ITRs are AAV2 ITRs. In some embodiments, the ITRs are AAV8 ITRs. Any combinations of Rep proteins and ITRs known in the art can be used in the cells and methods described herein.

In some embodiments, a host cell (e.g., a mammalian or an insect cell) further comprises a helper plasmid expression Adenovirus helper genes.

In some embodiments, a host cell comprises one or more packaging factors stably integrated into cell genome. In some embodiments, the host cell comprises a nucleic acid encoding any of the AAV capsid proteins described herein stably integrated into its genome. In some embodiments, the host cell comprises a nucleic acid encoding a Rep protein stably integrated into its genome. In some embodiments, the host cell comprises an Adenovirus helper gene stably integrated into its genome. In some embodiments, the host cell comprises a nucleic acid encoding an AAV capsid protein described herein, a nucleic acid encoding a Rep protein, and an Adenovirus helper gene(s) stably integrated into its genome.

The methods of production of rAAV virions are known in the art. In some embodiments, an rAAV virion can be generated using the host cells as described herein.

In some embodiments, the method of producing an rAAV virion in cell comprises:
i. introducing (e.g., by transient transfection or stable integration techniques) a nucleic acid encoding any of the AAV capsid proteins described herein, a nucleic acid encoding a Rep protein (such as any AAV Rep protein known in the art or described herein), an Adenovirus helper gene(s) (such as any Adenovirus helper genes known in the art), and/or a transgene cassette comprising a transgene flanked by ITRs (e.g., wherein the transgene expresses a therapeutic protein) into the cell (e.g., via DNA transfection, viral infection, and/or stable integration), wherein each of the introduced nucleic acids or genes is operably linked to a promoter active in the cell;
ii culturing the cell (e.g., using a suspension cell culture or an adherent cell culture) under conditions suitable for production of an rAAV virion (e.g., suitable for packaging protein expression and/or suitable for viral packaging), and
iii. collecting the produced rAAV virion (e.g., from media supernatant and/or from cell lysate following cell lysis), and
iv. optionally further purifying the rAAV virion, e.g., by density gradient ultracentrifugation and/or chromatography-based methods.

In some embodiments, the vectors, promoters, packaging factors, packaging systems, host cells, and/or methods of rAAV virion production are any of those known in the art.

### Methods of Use

In some embodiments, the disclosure provides methods of identifying AAV capsid proteins that confer on rAAV virions increased transduction efficiency in target cells. The methods comprise providing a population of rAAV virions whose rAAV genomes comprise a library of cap polynucleotides encoding variant AAV capsid proteins; optionally contacting the population with non-target cells for a time sufficient to permit attachment of undesired rAAV virions to the non-target cells; contacting the population with target cells for a time sufficient to permit transduction of the cap polynucleotide into the target cells by the rAAV virions; and sequencing the cap polynucleotides from the target cells, thereby identifying AAV capsid proteins that confer increased transduction efficiency in the target cells. In some embodiments, the method further comprises depleting the population of rAAV virions by contacting the population with non-target cells for time sufficient to permit attachment of the rAAV virions to the non-target cells. Non-limiting examples of such identification methods are provided in the Examples.

The disclosure provides methods for generating cardiomyocytes and/or cardiomyocyte-like cells in vitro using an rAAV virion. Selected starting cells are transduced with an rAAV and optionally exposed to small-molecule reprogramming factors (before, during, or after transduction) for a time and under conditions sufficient to convert the starting cells across lineage and/or differentiation boundaries to form cardiac progenitor cells and/or cardiomyocytes. In some embodiments, the starting cells are fibroblast cells. In some embodiments, the starting cells express one or more markers indicative of a differentiated phenotype. The time for conversion of starting cells into cardiac progenitor and cardiomyocyte cells can vary. For example, the starting cells can be incubated after treatment with one or more polynucleotides or proteins of interest until cardiac or cardiomyocyte cell markers are expressed. Such cardiac or cardiomyocyte cell markers can include any of the following markers: α-GATA4, TNNT2, MYH6, RYR2, NKX2-5, MEF2C, ANP, Actinin, MLC2v, MY20, cMHC, ISL1, cTNT, cTNI, and MLC2a, or any combination thereof. In some embodiments, the induced cardiomyocyte cells are negative for one or more neuronal cells markers. Such neuronal cell markers can include any of the following markers: DCX, TUBB3, MAP2, and ENO2.

Incubation can proceed until cardiac progenitor markers are expressed by the starting cells. Such cardiac progenitor markers include GATA4, TNNT2, MYH6, RYR2, or a combination thereof. The cardiac progenitor markers such as GATA4, TNNT2, MY16, RYR2, or a combination thereof can be expressed by about 8 days, or by about 9 days, or by about 10 days, or by about 11 days, or by about 12 days, or by about 14 days, or by about 15 days, or by about 16 days, or by about 17 days, or by about 18 days, or by about 19 days, or by about 20 days after starting incubation of cells in the compositions described herein. Further incubation of the cells can be performed until expression of late stage cardiac progenitor markers such as NKX2-5, MEF2C or a combination thereof occurs.

Reprogramming efficiency may be measured as a function of cardiomyocyte markers. Such pluripotency markers include, but are not limited to, the expression of cardiomyocyte marker proteins and mRNA, cardiomyocyte morphology and electrophysiological phenotype. Non-limiting examples of cardiomyocyte markers include, a-sarcoglycan, atrial natriuretic peptide (ANP), bone morphogenetic protein 4 (BMP4), connexin 37, connexin 40, crypto, desmin, GATA4, GATA6, MEF2C, MYH6, myosin heavy chain. NKX2.5, TBX5, and Troponin T.

The expression of various markers specific to cardiomyocytes may be detected by conventional biochemical or immunochemical methods (e.g., enzyme- linked immunosorbent assay, immunohistochemical assay, and the like). Alternatively, expression of a nucleic acid encoding a cardiomyocyte- specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting, and analyzing mRNA coding for any marker proteins. Nucleic acid sequences coding for markers specific to cardiomyocytes are known and are available through public databases such as GenBank. Thus, marker-specific sequences needed for use as primers or probes are easily determined.

Cardiomyocytes exhibit some cardiac-specific electrophysiological properties. One electrical characteristic is an action potential, which is a short-lasting event in which the difference of potential between the interior and the exterior of each cardiac cell rises and falls following a consistent trajectory. Another electrophysiological characteristic of cardiomyocytes is the cyclic variations in the cytosolic-free Ca²⁺ concentration, named as Ca²⁺ transients, which are employed in the regulation of the contraction and relaxation of cardiomyocytes. These characteristics can be detected and evaluated to assess whether a population of cells has been reprogrammed into cardiomyocytes.

The present disclosure provides a method of delivering a gene product to a cardiac cell, e.g., a cardiac fibroblast. The methods generally involve infecting a cardiac cell (e.g., a cardiac fibroblast) with an rAAV virion, where the gene product(s) encoded by the heterologous nucleic acid present in the rAAV virion is/are produced in the cardiac cell (e.g., cardiac fibroblast). Delivery of gene product(s) to a cardiac cell (e.g., cardiac fibroblast) can provide for treatment of a cardiac disease or disorder. Delivery of gene product(s) to a cardiac cell (e.g., cardiac fibroblast) can provide for generation of an induced cardiomyocyte-like (iCM) cell from the cardiac fibroblast. Delivery of gene product(s) to a cardiac cell (e.g., cardiac fibroblast) can provide for editing of the genome of the cardiac cell (e.g., cardiac fibroblast).

In some embodiments, infecting or transducing a cardiac cell (e.g., cardiac fibroblast) is carried out in vitro. In some embodiments, infecting or transducing a cardiac cell (e.g., cardiac fibroblast) is carried out in vitro; and the infected/transduced cardiac cell (e.g., cardiac fibroblast) is introduced into (e.g., transfused into or implanted into) an individual in need thereof, e.g., directly into cardiac tissue of an individual in need thereof. For in vitro transduction, an effective amount of rAAV virions to be delivered to cells is from about 10⁵ to about 10¹³ of the rAAV virions. Other effective dosages can be readily established by one of ordinary skill in the art through routine trials establishing dose response curves.

In some embodiments, infecting a cardiac cell (e.g., cardiac fibroblast) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in vivo injection, i.e., injection directly into cardiac tissue, a therapeutically effective dose will be on the order of from about 10⁶ to about 10¹⁵ of the rAAV virions, e.g., from about 10⁵ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁴ to about 10⁵, from about 10⁵ to about 10⁶, from about 10⁶ to about 10⁷, from about 10⁶ to about 10⁷, from about 10⁷ to about 10⁸, from about 10⁸ to about 10⁹, from about 10⁹ to about 10¹⁰, from about 10¹⁰ to about 10¹¹, to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual, or are administered via another route. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, the rAAV virion is administered at, at least at, or at no more than, 10² vg/kg, 10³ vg/kg, 10⁴ vg/kg, 10⁵ vg/kg, 10⁶ vg/kg, 10⁸ vg/kg, 10⁹ vg/kg, 10¹⁰ vg/kg, 10¹¹ vg/kg, 10¹² vg/kg, 10¹³ vg/kg, 2×10¹³ vg/kg, 3×10¹³ vg/kg, 4×10¹³ vg/kg, 5×10¹³ vg/kg, 6×10¹³ vg/kg, 7+10¹³ vg/kg, 8×10¹³ vg/kg, 9×10¹³ vg/kg, 10¹⁴ vg/kg, 2×10¹⁴ vg/kg, 3×10¹⁴ vg/kg, 4×10¹⁴ vg/kg, 5×10¹⁴ vg/kg, 6×10¹⁴ vg/kg, 7×10¹⁴ vg/kg, 8×10¹⁴ vg/kg, 9×10¹⁴ vg/kg, 10¹⁵ vg/kg, 10¹⁶ vg/kg, 10¹⁷ vg/kg, or 10¹⁸ vg/kg (or at any range of amounts in between these values). In some embodiments, the rAAV virion is administered at 2×10¹³ vg/kg. In some embodiments, the rAAV virion is administered at 1.43×10¹³ vg/kg. In some embodiments, the rAAV virion is administered at 1.2×10¹⁴ vg/kg.

In some embodiments, an effective amount of an rAAV virion of the present disclosure is administered locally to the heart. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery, e.g., intravenously. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, more than one administration (e.g., two, three, four or more administrations) may be employed to achieve the desired level of gene expression. In some embodiments, the mom than one administration is administered at various intervals, e.g., daily, weekly, twice monthly, monthly, every 3 months, every 6 months, yearly, etc. In some embodiments, multiple administrations are administered over a period of time of from I month to 2 months, from 2 months to 4 months, from 4 months to 8 months, from 8 months to 12 months, from 1 year to 2 years, from 2 years to 5 years, or more than 5 years.

The present disclosure provides a method of reprogramming a cardiac fibroblast to generate an induced cardiomyocyte-like cell (iCM). The method generally involves infecting a cardiac fibroblast with an rAAV virion of the present disclosure, wherein the rAAV virion comprises a heterologous nucleic acid comprising a nucleotide sequence encoding one or more reprogramming factors.

The expression of various markers specific to cardiomyocytes is detected by conventional biochemical or immunochemical methods (e.g., enzyme-linked immunosorbent assay; immunohistochemical assay; and the like). Alternatively, expression of nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting, and analyzing mRNA coding for any marker proteins. Nucleic acid sequences coding for markers specific to cardiomyocytes are known and are available through public data bases such as GenBank; thus, marker-specific sequences needed for use as primers or probes is easily determined.

Induced cardiomyocytes can also exhibit spontaneous contraction. Whether an induced cardiomyocyte exhibits spontaneous contraction can be determined using standard electrophysiological methods (e.g., patch clamp).

In some embodiments, induced cardiomyocytes can exhibit spontaneous Ca²⁺ oscillations. Ca²⁺ oscillations can be detected using standard methods, e.g., using any of a variety of calcium-sensitive dyes, intracellular Ca²⁺ ion-detecting dyes include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, rhod-3, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange. Calcium Crimson, Calcium Green-SN, Magnesium Green, Oregon Green 488 BAPTA-1, Oregon Green 488 BAPTA-2, X-rhod-1, Fura Red. Rhod-5F, Rhod-5N, X-Rhod-5N, Mag-Rhod-2, Mag-X- Rhod-1, Fluo-5N, Fluo-SF, Fluo-4FF, Mag-Fluo-4, Aequorin, dextran conjugates or any other derivatives of any of these dyes, and others (see, e.g., the catalog or Internet site for Molecular Probes, Eugene, see, also, Nuccitelli, ed., Methods in Cell Biology, Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed, Academic Press (1999); Calcium Signaling Protocols (Methods in Molecular Biology), 2005, D.G. Lamber, ed., Humana Press.).

In some embodiments, an iCM is generated in vitro; and the iCM is introduced into an individual, e.g., the iCM is implanted into a cardiac tissue of an individual in need thereof. A method of the present disclosure can comprise infecting a population of cardiac fibroblasts in vitro, to generate a population of iCMs; and the population of iCMs is implanted into a cardiac tissue of an individual in need thereof.

In some embodiments, an iCM is generated in vivo. For example, in some embodiments, an rAAV virion of the present disclosure that comprises a heterologous nucleic acid comprising a nucleotide sequence encoding one or more reprograming factors is administered to an individual. In some embodiments, the rAAV virion is administered directly into cardiac tissue of an individual in need thereof. In some embodiments, from about 10⁶ to about 10⁵, from about 10⁵ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure that comprises a heterologous nucleic acid comprising a nucleotide sequence encoding one or more reprogramming factors are administered to an individual, e.g., are administered directly into cardiac tissue in the individual or via another route of administration. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁴ vg/kg, from about 10¹⁴ vg/kg to about 10¹⁶ vg/kg, or more than 10¹⁶ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

The present disclosure provides a method of modifying (“editing”) the genome of a cardiac cell. The present disclosure provides a method of modifying (“editing”) the genome of a cardiac fibroblast. The present disclosure provides a method of modifying (“editing”) the genome of a cardiomyocyte. The methods generally involve infecting a cardiac cell (e.g., a cardiac fibroblast or a cardiomyocyte) with an rAAV virion of the present disclosure, wherein the rAAV virion comprises a heterologous nucleic acid comprising a nucleotide sequence encoding a genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding an RNA-guided genome-editing endonuclease. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; and ii) one or more guide RNAs. In some embodiments, the method comprises infecting a cardiac fibroblast or a cardiomyocyte with an rAAV virion of the present disclosure, wherein the rAAV virion comprises a heterologous nucleic acid comprising a nucleotide sequence encoding: i) an RNA-guided genome-editing endonuclease; ii) a guide RNAs; and iii) a donor template DNA. Suitable RNA-guided genome-editing endonucleases are described above.

In some embodiments, infecting a cardiac cell (e.g., cardiac fibroblast; a cardiomyocyte) is carried out in vitro. In some embodiments, infecting a cardiac cell (e.g., cardiac fibroblast; a cardiomyocyte) is carried out in vitro; and the infected cardiac cell (e.g., cardiac fibroblast) is introduced into (e.g., implanted into) an individual in need thereof, e.g., directly into cardiac tissue of an individual in need thereof. For in vitro transduction, an effective amount of rAAV virions to be delivered to cells will be on the order of from about 10⁸ to about 10¹³ of the rAAV virions. Other effective dosages can be readily established by one of ordinary skill in the art through routine trials establishing dose response curves.

In some embodiments, infecting a cardiac cell (e.g., cardiac fibroblast; a cardiomyocyte) is carried out in vivo. For example, in some embodiments, an effective amount of an rAAV virion of the present disclosure is administered directly into cardiac tissue of an individual in need thereof. An “effective amount” will fall in a relatively broad range that can be determined through experimentation and/or clinical trials. For example, for in viva injection, i.e., injection directly into cardiac tissue, a therapeutically effective dose will be on the order of from about 10⁶ to about 10¹⁵ of the rAAV virions, e.g., from about 10¹¹ to 10¹² rAAV virions, of the present disclosure. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

For example, from about 10⁶ to about 10⁷, from about 10⁷ to about 10⁸, from about 10⁸ to about 10⁹, from about 10⁹ to about 10¹⁰, from about 10¹⁰ to about 10¹¹, from about 10¹¹ to about 10¹², from about 10¹² to about 10¹³, from about 10¹³ to about 10¹⁴, from about 10¹⁴ to about 10¹⁵ genome copies, or more than 10¹⁵ genome copies, of an rAAV virion of the present disclosure are administered to an individual, e.g., are administered directly into cardiac tissue in the individual. The number of rAAV virions administered to an individual can be expressed in viral genomes (vg) per kilogram (kg) body weight of the individual. In some embodiments, and effective amount of an rAAV virion of the present disclosure is from about 10² vg/kg to 10⁴ vg/kg, from about 10⁴ vg/kg to about 10⁶ vg/kg, from about 10⁶ vg/kg to about 10⁸ vg/kg, from about 10⁸ vg/kg to about 10¹⁰ vg/kg, from about 10¹⁰ vg/kg to about 10¹² vg/kg, from about 10¹² vg/kg to about 10¹⁴ vg/kg, from about 10¹⁴ vg/kg to about 10¹⁶ vg/kg, from about 10¹⁶ vg/kg to about 10¹⁸ vg/kg, or more than 10¹⁸ vg/kg. In some embodiments, an effective amount of an rAAV virion of the present disclosure is administered via intramyocardial injection through the epicardium. In some embodiments, an effective amount of an rAAV virion of the present disclosure is administered via vascular delivery through the coronary artery. In some embodiments, an effective amount of an rAAV virion of the present disclosure is administered via systemic delivery through the superior vena cava. In some embodiments, an effective amount of an rAAV virion of the present disclosure is administered via systemic delivery through a peripheral vein.

In some embodiments, the genome editing comprises homology-directed repair (HDR). In some embodiments, the HDR corrects a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

In some embodiments, the genome editing comprises non-homologous end joining (NHEJ). In some embodiments, the NHEJ deletes a defect in an endogenous target nucleic acid in the cardiac fibroblast or the cardiomyocyte, wherein the defect is associated with, or leads to, a defect in structure and/or function of the cardiac fibroblast or the cardiomyocyte, or a component of the cardiac fibroblast or the cardiomyocyte.

A method of the present disclosure for editing the genome of a cardiac cell can be used to correct any of a variety of genetic defects that give rise to a cardiac disease or disorder. Mutations of interest include mutations in one or more of the following genes: cardiac troponin T(TNNT2); myosin heavy chain (MYH7); tropomyosin 1 (TPM1); myosin binding protein C (MYBPC3); 5′-AMP-activated protein kinase subunit gamma-2 (PRKAG2); troponin I type 3 (TNNI3); titin (TTN); myosin, light chain 2 (MYL2); actin, alpha cardiac muscle 1 (ACTC1); potassium voltage-gated channel, KQT- like subfamily, member 1 (KCNQ1); plakophilin 2 (PKP2); myocyte enhancer factor 2c (MEF2C); and cardiac LIM protein (CSRP3), Specific mutations of interest include, without limitation, MYH7 R663H mutation; TNNT2 R173W; PKP2 2013delC mutation; PKP2 Q617X mutation; and KCNQ1 0269S missense mutation. Mutations of interest include mutations in one or more of the following genes: MYH6, ACTN2, SERCA2. GATA4, TBX5, MYOCD. NKX2-5, NOTCH1, MEF2C, HAND2, and HAND1. In some embodiments, the mutations of interest include mutations in the following genes: MEF2C, TBX5, and MYOCD. Cardiac diseases and disorders that can be treated with a method of the present disclosure include coronary heart disease, cardiomyopathy, endocarditis, congenital cardiovascular defects, and congestive heart failure. Cardiac diseases and disorders that can be treated with a method of the present disclosure include hypertrophic cardiomyopathy; a valvular heart disease; myocardial infarction; congestive heart failure; long QT syndrome; atrial arrhythmia; ventricular arrhythmia; diastolic heart failure; systolic heart failure; cardiac valve disease; cardiac valve calcification; left ventricular non-compaction; ventricular septal defect; and ischemia.

In some embodiments, the disclosure provides a method of transducing a cardiac cell. In some embodiments, the disclosure provides a method of transducing a cardiac cell, comprising contacting the cardiac cell with an rAAV virion described herein, wherein the rAAV virion transduces the cardiac cell. In some embodiments, the cardiac cell is a cardiomyocyte.

In some embodiments, the disclosure provides a method of transducing a cardiac cell, comprising contacting the cardiac cell with an rAAV virion, wherein the rAAV virion comprises a capsid protein, wherein the capsid protein is any capsid protein described herein.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
an amino acid insertion at position 584 comprising one or more of an asparagine (N), a threonine (T), a tyrosine (Y), phenylalanine (F), and an alanine (A);
an amino acid insertion at position 585 comprising one or more of a histidine (H) and a methionine (M);
an amino acid insertion at position 586 comprising one or more of a histidine (H), a tyrosine (Y), a valine (V), a threonine (T), an alanine (A), an isoleucine (I), a tryptophan (W), a methionine (M), and a leucine;
an amino acid insertion between positions 585 and 586 comprising one or two of histidine (H), tyrosine (Y), tryptophan (W), and methionine (M) (e.g., an insertion of WM or HY);
an amino acid insertion at position 587 comprising one or more of an isoleucine (I) and a proline (P);
an amino acid insertion at position 588 comprising one or more of an isoleucine (I), a threonine (T), and a proline (P);
an amino acid insertion at position 589 comprising one or more of a glycine (G) and a glutamine (Q);
one or more amino acid substitutions selected from the group consisting of N452K, N452A, N452V, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q58811, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585E, S586N, A587T, Q588V, A589S, Q590I, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions S586T, A587L, Q588F, A589N, Q590S, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585N, A587T, Q588Y, A589L, Q590G, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585G, A587I, Q588L, A589T, Q590H, and N452K.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585M, S586M, A587T, Q588T, A589A, and Q590R.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ TID NO: 1: amino acid substitutions Q585C, A587T, Q588S, A589I, and Q590R.

In some embodiments, the disclosure provides a method of transducing a cardiac cell, comprising contacting the cardiac cell with an rAAV virion, wherein the rAAV virion comprises a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1: amino acid substitutions Q585N, A587T, Q588Y, A589L, and Q590G.

In some embodiments, the disclosure provides a method of delivering one or more gene products to a cardiac cell. In some embodiments, the method of delivering one or more gene products to a cardiac cell comprises contacting the cardiac cell with an rAAV virion described herein. In some embodiments, the cardiac cell is a cardiomyocyte.

In some embodiments, the disclosure provides a method of delivering one or more gene products to a cardiac cell with an rAAV virion comprising a capsid protein, wherein the capsid protein is any capsid protein described herein.

In some embodiments, the disclosure provides a method of delivering one or more gene products to a cardiac cell with an rAAV virion comprising a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
(a) amino acid substitutions Q585E, S586N, A587T, Q588V, A589S, Q590I, and N452K;
(b) amino acid substitutions S586T, A587L, Q588F, A589N, Q590S, and N452K;
(c) amino acid substitutions Q585N, A587T, Q588Y, A589L, Q590G, and N452K;
(d) amino acid substitutions Q585G, A587I, Q588L, A589T, Q59011, and N452K;
(e) amino acid substitutions Q585M, S586M, A587T, Q588T, A589A, and Q590R;
(f) amino acid substitutions Q585C, A587T, Q588S, A589I, and Q590R, or
(g) amino acid substitutions Q585N, A587T, Q588Y, A589L, and Q590G.

### Methods of Treatment

The disclosure provides a methods of treating a cardiac pathology in a subject in need thereof, comprising administering a therapeutically effective amount of a pharmaceutical composition comprising an rAAV virion to the subject, wherein the rAAV virion transduces cardiac tissue.

Subjects in need of treatment using compositions and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Subjects suitable for treatment using the compositions, cells and methods of the present disclosure include individuals (e.g., mammalian subjects, such as humans, non-human primates, domestic mammals, experimental non- human mammalian subjects such as mice, rats, etc.) having a cardiac condition including but limited to a condition that results in ischemic heart tissue (e.g., individuals with coronary artery disease) and the like.

In some examples, an individual suitable for treatment suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method include individuals who have a degenerative muscle disease. e.g., familial cardiomyopathy, dilated cardiomyopathy, hypertrophic cardiomyopathy, restrictive cardiomyopathy, or coronary artery disease with resultant ischemic cardiomyopathy.

For example, the cardiac pathology can be selected from the group consisting of congestive heart failure, myocardial infarction, cardiac ischemia, myocarditis, and arrhythmia. In some embodiments, the subject is diabetic. In some embodiments, the subject is non-diabetic. In some embodiments, the subject suffers from diabetic cardiomyopathy.

For therapy, the rAAV virions of the disclosure and/or pharmaceutical compositions thereof can be administered locally or systemically. An rAAV virion can be introduced by injection, catheter, implantable device, or the like. An rAAV virion can be administered in any physiologically acceptable excipient or carrier that does not adversely affect the cells. For example, rAAV virions of the disclosure and/or pharmaceutical compositions thereof can be administered intravenously or through an intracardiac route (e.g., epicardially or intramyocardially). Methods of administering rAAV virions of the disclosure and/or pharmaceutical compositions thereof to subjects, particularly human subjects include injection or infusion of the pharmaceutical compositions (e.g., compositions comprising rAAV virions). Injection may include direct muscle injection and infusion may include intravascular infusion. The rAAV virions or pharmaceutical compositions can be inserted into a delivery device which facilitates introduction by injection into the subjects. Such delivery devices include tubes, e.g., catheters, for injecting cells and fluids into the body of a recipient subject. The tubes can additionally include a needle, e.g., a syringe, through which the cells of the invention can be introduced into the subject at a desired location.

In some embodiments, the rAAV virion is administered by subcutaneous, intravenous, intramuscular, intraperitoneal, or intracardiac injection or by intracardiac catheterization. In some embodiments, the rAAV virion is administered by direct intramyocardial injection or transvascular administration. In some embodiments, the rAAV virion is administered by direct intramyocardial injection, antegrade intracoronary injection, retrograde injection, transendomyocardial injection, or molecular cardiac surgery with recirculating delivery (MCARD).

The rAAV virions can be inserted into such a delivery device, e.g., a syringe, in different forms. The rAAV virion can be supplied in the form of a pharmaceutical composition. Such a composition can include an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The choice of the excipient and any accompanying constituents of the composition can be adapted to optimize administration by the route and/or device employed.

Recombinant AAV may be administered locally or systemically. Recombinant AAV may be engineered to target specific cell types by selecting the appropriate capsid protein of the disclosure. To determine the suitability of various therapeutic administration regimens and dosages of AAV virion compositions, the rAAV virions can first be tested in a suitable animal model. At one level, recombinant AAV are assessed for their ability to infect target cells in vivo. Recombinant AAV can also be assessed to ascertain whether it migrates to target tissues, whether they induce an immune response in the host, or to determine an appropriate number, or dosage, of rAAV virions to be administered. It may be desirable or undesirable for the recombinant AA V to generate an immune response, depending on the disease to be treated. Generally, if repeated administration of a virion is required, it will be advantageous if the virion is not immunogenic. For testing purposes, rAAV virion compositions can be administered to immunodeficient animals (such as nude mice, or animals rendered immunodeficient chemically or by irradiation). Target tissues or cells can be harvested after a period of infection and assessed to determine if the tissues or cells have been infected and if the desired phenotype (e.g., induced cardiomyocyte) has been induced in the target tissue or cells.

Recombinant AAV virions can be administered by various routes, including without limitation direct injection into the heart or cardiac catheterization. Alternatively, the rAAV virions can be administered systemically such as by intravenous infusion. When direct injection is used, it may be performed either by open-heart surgery or by minimally invasive surgery. In some embodiments, the recombinant viruses are delivered to the pericardial space by injection or infusion. Injected or infused recombinant viruses can be traced by a variety of methods. For example, recombinant AAV labeled with or expressing a detectable label (such as green fluorescent protein, or beta-galactosidase) can readily be detected. The recombinant AAV may be engineered to cause the target cell to express a marker protein, such as a surface-expressed protein or a fluorescent protein. Alternatively, the infection of target cells with recombinant AAV can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

In some embodiments, the disclosure provides a method of treating a cardiac pathology in a subject in need thereof, comprising administering a therapeutically effective amount of an rAAV virion described herein.

In some embodiments, the disclosure provides a method of treating a cardiac pathology in a subject in need thereof, comprising administering a therapeutically effective amount of an rAAV virion comprising a capsid protein, wherein the capsid protein is any capsid protein described herein.

In some embodiments, the disclosure provides a method of treating a cardiac pathology in a subject in need thereof, comprising administering a therapeutically effective amount of an rAAV virion comprising a capsid protein, wherein the capsid protein shares at least 80% polypeptide sequence identity to an AAV9 VP3 reference sequence according to SEQ ID NO: 487, and wherein the capsid protein comprises, relative to reference sequence SEQ ID NO: 1:
(a) amino acid substitutions Q585E, S586N, A587T, Q588V, A589S, Q590I, and N452K;
(b) amino acid substitutions S586T, A587L, Q588F, A589N, Q590S, and N452K;
(c) amino acid substitutions Q585N, A587T, Q588Y, A589L, Q590G, and N452K;
(d) amino acid substitutions Q585G, A587I, Q588L, A589T, Q590I, and N452K;
(e) amino acid substitutions Q585M, S586M, A587T, Q588T, A589A, and Q590R;
(f) amino acid substitutions Q585C, A587T, Q588S, A589L, and Q590R, or
(g) amino acid substitutions Q585N, A587T, Q588Y, A589L, and Q590G.

### Pharmaceutical Compositions

The present disclosure provides pharmaceutical composition comprising an rAAV virion of the disclosure. The pharmaceutical composition may include one or more of a pharmaceutically acceptable carrier, diluent, excipient, and buffer. In some embodiments, the pharmaceutically acceptable carrier, diluent, excipient, or buffer is suitable for use in a human. Such excipients, carriers, diluents, and buffers include any pharmaceutical agent that can be administered without undue toxicity. Pharmaceutically acceptable excipients include, but are not limited to, liquids such as water, saline, glycerol, and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as pH buffering substances may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A.H. Kibbe et al., eds., 3rd ed. Amer. Pharmaceutical Assoc.

To prepare the composition, rAAV virion is generated and purified as necessary or desired. The rAAV can be mixed with or suspended in a pharmaceutically acceptable carrier. These rAAV can be adjusted to an appropriate concentration, and optionally combined with other agents. The concentration of rAAV virion and/or other agent included in a unit dose can vary widely. The dose and the number of administrations can be optimized by those skilled in the art. For example, about 10²-10¹⁰ vector genomes (vg) may be administered. In some embodiments, the dose be at least about 10² vg, about 10³ vg, about 10⁴ vg, about 10⁵ vg, about 10⁶ vg about 10⁷ vg, about 10⁸ vg, about 10⁹ vg, about 10¹⁰ vg, or more vector genomes. Daily doses of the compounds can vary as well. Such daily doses can range, for example, from at least about 10² vg/day, about 10³ vg/day, about 10⁴ vg/day, to about 10⁵ vg/day, about 10⁶ vg/day, about 10⁷ vg/day, about 10⁸ vg/day, about 10⁹ vg/day, about 10¹⁰ vg/day, or more vector genomes per day.

In certain embodiments, the method of treatment is enhanced by the administration of one or more anti-inflammatory agents, e.g., an anti-inflammatory steroid or a nonsteroidal anti-inflammatory drug (NSAID).

Anti-inflammatory steroids for use in the invention include the corticosteroids, and in particular those with glucocorticoid activity, e.g., dexamethasone and prednisone. Nonsteroidal anti-inflammatory drugs (NSAIDs) for use in the invention generally act by blocking the production of prostaglandins that cause inflammation and pain, cyclooxygenase-1 (COX-1) and/or cyclooxygenase-2 (COX-2). Traditional NSAIDs work by blocking both COX-1 and COX-2. The COX-2 selective inhibitors block only the COX-2 enzyme. In certain embodiment, the NSAID is a COX-2 selective inhibitor, e.g., celecoxib (Celebrex®), rofecoxib (Vioxx), and valdecoxib (B extra). In certain embodiments, the anti-inflammatory is an NSAID prostaglandin inhibitor, e.g., Piroxicam.

The amount of rAAV virion for use in treatment will vary not only with the particular carrier selected but also with the route of administration, the nature of the condition being treated and the age and condition of the patient. Ultimately, the attendant health care provider may determine proper dosage. A pharmaceutical composition may be formulated with the appropriate ratio of each compound in a single unit dosage form fir administration with or without cells. Cells or vectors can be separately provided and either mixed with a liquid solution of the compound composition or administered separately.

Recombinant AAV can be formulated for parenteral administration (e.g., by injection, for example, bolus injection or continuous infusion) and may be presented in unit dosage form in ampoules, prefilled syringes, small volume infusion containers or multi-dose containers with an added preservative. The pharmaceutical compositions can take the form of suspensions, solutions, or emulsions in oily or aqueous vehicles, and can contain formulatory agents such as suspending, stabilizing and/or dispersing agents. Suitable carriers include saline solution, phosphate buffered saline, and other materials commonly used in the art.

The compositions can also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univase), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g, Amiloride (Midamor), Bumetanide (Bumex), Chlorothiazide (Diuril), Chlothalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof.

Additional agents can also be included such as antibacterial agents, antimicrobial agents, anti-viral agents, biological response modifiers, growth factors; immune modulators, monoclonal antibodies and/or preservatives. The compositions of the invention may also be used in conjunction with other forms of therapy.

The rAAV virions described herein can be administered to a subject to treat a disease or disorder. Such a composition may be in a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compounds and compositions of the invention may be essentially continuous over a preselected period of time or may be in a series of spaced doses. Both local and systemic administration is contemplated. In some embodiments, localized delivery of rAAV virion is achieved. In some embodiments, localized delivery of rAAV virions is used to generate a population of cells within the heart. In some embodiments, such a localized population operates as “pacemaker cells” for the heart. In some embodiments, the rAAV virions are used to generate, regenerate, repair, replace, and/or rejuvenate one or more of a sinoatrial (SA) node, an atrioventricular (AV) node, a bindle of His, and/or Purkinje fibers.

To control tonicity, an aqueous pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g., between 6.5 and 7.5, or between 7.0 and 7.8.

The composition is preferably sterile. The composition is preferably gluten free. The composition is preferably non-pyrogenic.

In some embodiments, a composition comprising cells may include a cryoprotectant agent. Non-limiting examples of cryoprotectant agents include a glycol (e.g., ethylene glycol, propylene glycol, and glycerol), dimethyl sulfoxide (DMSO), formamide, sucrose, trehalose, dextrose, and any combinations thereof.

One or more of the following types of compounds can also be present in the composition with the rAAV virions: a WNT agonist, a GSK3 inhibitor, a TGF-beta signaling inhibitor, an epigenetic modifier, LSD1 inhibitor, an adenylyl cyclase agonist, or any combination thereof. Kits

A variety of kits are described herein that include any of composition (e.g., rAAV virions) described herein. The kit can include any of compositions described herein, either mixed together or individually packaged, and in dry or hydrated form. The rAAV virions and/or other agents described herein can be packaged separately into discrete vials, bottles, or other containers. Alternatively, any of the rAAV virions and/or agents described herein can be packaged together as a single composition, or as two or more compositions that can be used together or separately. The compounds and/or agents described herein can be packaged in appropriate ratios and/or amounts to facilitate conversion of selected cells across differentiation boundaries to form cardiac progenitor cells and/or cardiomyocytes.

The kit can include instructions for administering those compositions, compounds and/or agents. Such instructions can provide the information described throughout this application. The rAAV virion or pharmaceutical composition can be provided within any of the kits in the form of a delivery device. Alternatively, a delivery device can be separately included in the kits, and the instructions can describe how to assemble the delivery device prior to administration to a subject.

Any of the kits can also include syringes, catheters, scalpels, sterile containers for sample or cell collection, diluents, pharmaceutically acceptable carriers, and the like. The kits can provide other factors such as any of the supplementary factors or drugs described herein for the compositions in the preceding section or other parts of the application.

### Viral Vectors

Suitable viral vectors for methods and gene therapy vectors provided herein include, but are not limited to, viral vectors (e.g. viral vectors based on vaccinia virus; poliovirus; adenovirus (e.g., Li et al. (1994) Invest Opthalmol Vis Sci 35:2543-2549; Borras et al. (1999) Gene Ther 6:515-524; Li and Davidson, (1995) Proc. Natl. Acad. Sci. 92:7700-7704; Sakamoto et al. (1999) Hum Gene Ther 5: 1088-1097; WO 94/12649; WO 93/03769; WO 93/19191; WO 94:28938; WO 95/11984 and WO 95/00655); adeno-associated virus (e.g., Ali et al. (1998) Hum Gene Ther 9(l):81-86, 1998, Flannery et al. (1997) Proc. Natl. Acad. Sci. 94:6916-6921; Bennett et al. (1997) Invest Opthalmol Vis Sci 38:2857-2863; Jomary et al. (1997) Gene Ther 4:683-690; Rolling et al. (1999), Hum Gene Ther 10:641-648; Ali et al. (1996) Hum Mol Genet. 5:591-594; WO 93/09239, Samulski et al. (1989) J. Vir. 63: 3822-3828; Mendelson et al. (1988) Virol. 166: 154-165; and Flotte et al. (1993) Proc. Natl. Acad. Sci. 90: 10613-10617; SV40; herpes simplex virus; human immunodeficiency virus (e.g., Miyoshi et al. (1997) Proc. Natl. Acad. Sci. 94: 10319-10323; Takahashi et al. (1999) J Virol 73:7812-7816); a retroviral vector (e.g., Murine-Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available. The following vectors are provided by way of example; for eukaryotic cells: pXTI, pSGS (Stratagene), pSVK3, pBPV, pMSG, pSVLSV40 (Pharmacia), and pAd (Life Technologies). However, any other vector is contemplated for use so long as it is compatible with the methods of the present disclosure.

The ability of certain viruses to infect cells or enter cells via receptor-mediated endocytosis, and express viral genes stably and efficiently have made them attractive candidates for the transfer of foreign nucleic acids into cells (e.g., mammalian cells). Viral vectors are contemplated to include control sequences such as promoters for expression of the polypeptide of interest. Although many viral vectors integrate into the host cell genome, if desired, the segments that allow such integration can be removed or altered to prevent such integration. Moreover, in some embodiments, the vectors do not contain a mammalian origin of replication. Non-limiting examples of virus vectors are described below that are contemplated for use in delivering nucleic acids encoding PKP2 into a selected cell. In some embodiments, the viral vector is derived from a replication-deficient virus.

In general, other useful viral vectors are based on non-cytopathic eukaryotic viruses in which non-essential genes have been replaced with the polypeptide of interest. Non-cytopathic viruses include certain retroviruses, the life cycle of which involves reverse transcription of genomic viral RNA into DNA with subsequent proviral integration into host cellular DNA. In general, the retroviruses are replication-deficient (e.g., capable of directing synthesis of the desired transcripts, but incapable of manufacturing an infectious particle). Such genetically altered retroviral expression vectors have general utility for the high-efficiency transduction of polynucleotide in vivo.

In some embodiments, a polynucleotide encoding PKP2 is housed within an infective virus that has been engineered to express a specific binding ligand. The virus particle will thus bind with specificity to the cognate receptors of the target cell and deliver the contents to the cell. In some embodiments, the virus is modified to impart particular viral tropism, e.g., the virus preferentially infects fibroblasts, heart cells, or more particularly cardiac fibroblasts (CFs). For AAV, in some cases, capsid proteins are mutated to alter the tropism of the viral vector. For example, lentivirus tropism is often modified by using different envelope proteins; this is known as “pseudotyping.”

In some embodiments, the viral vector is a retroviral vector. Retroviruses often integrate their genes into the host genome, transfer a large amount of foreign genetic material, infect a broad spectrum of species and cell types, and are often packaged in special cell-lines (Miller et al., Am. J. Clin. Oncol., 15(3):216-221, 1992). In some embodiments, a retroviral vector is altered so that it does not integrate into the host cell genome.

In some embodiments, the recombinant retrovirus comprises a viral polypeptide (e.g., retroviral env) to aid entry into the target cell. Such viral polypeptides are well-established in the art, for example, U.S. Pat. No. 5,449,614. In some embodiments, the viral polypeptide is an amphotropic viral polypeptide, for example, amphotropic env, which aids entry into cells derived from multiple species, including cells outside of the original host species. In some embodiments, the viral polypeptide is a xenotropic viral polypeptide that aids entry into cells outside of the original host species. In some embodiments, the viral polypeptide is an ecotropic viral polypeptide, for example, ecotropic env, which aids entry into cells of the original host species.

Examples of viral polypeptides capable of aiding entry of retroviruses into cells include, but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env, RD114, FeLV-C, FeLV-B, MLV IOA 1 env gene, and variants thereof, including chimeras. Yee et al. (1994) Methods Cell Biol, Pt A:99-1 12 (VSV-G); U.S. Pat. No. 5,449,614. In some cases, the viral polypeptide is genetically modified to promote expression or enhanced binding to a receptor.

In embodiments, the retroviral construct is derived from a range of retroviruses, e.g., MMLV, HIV-1, SIV, FIV, or other retrovirus described herein. In some embodiments, the retroviral construct encodes all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides often help facilitate viral entry. In an exemplary embodiment, the recombinant retrovirus is HIV-1 virus comprising a VSV-g polypeptide, but not comprising a HIV 1 env polypeptide.

In some embodiments, the retroviral construct comprises: a promoter, a multi-cloning site, and/or a resistance gene. Examples of promoters include but are not limited to CMV, SV40, EF1a, β-actin; retroviral LTR promoters, and inducible promoters. In some embodiments, the retroviral construct comprises a packaging signal (e.g., a packaging signal derived from the MFG vector; a psi packaging signal). Examples of some retroviral constructs known in the art include but are not limited to: pMX, pBabeX or derivatives thereof. Onishi et al. (1996) Experimental Hematology, 24:324-329. In some cases, the retroviral construct is a self-inactivating lentiviral vector (SIN) vector. Miyoshi et al. (1998) J. Virol 72(10):8150-8157. In some cases, the retroviral construct is LL-CG, LS-CG, CL-CG, CS-CG, CLG or MFG. Miyoshi et al. (1998) J. Virol 72(10):8150-8157; Onishi et al. (1996) Experimental Hematology, 24:324-329; Riviere et al. (1995) Proc. Natl. Acad. Sci., 92:6733-6737.

In some embodiments, a retroviral vector is constructed by inserting a nucleic acid (e.g., one encoding a polypeptide of interest or an RNA) into the viral genome in the place of some viral sequences to produce a virus that is replication-defective. To produce virions, a packaging cell line containing the gag, pol, and env genes, but without the LTR and packaging components, is constructed (Mann et al., Cell 33:153-159, 1983), When a recombinant plasmid containing a cDNA, together with the retroviral LTR and packaging sequences is introduced into a special cell line (e.g., by calcium phosphate precipitation or lipid transfection), the packaging sequence allows the RNA transcript of the recombinant plasmid to be packaged into viral particles, which are then secreted into the culture media (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986; Mann et al., Cell, 33:153-159, 1983), The media containing the recombinant retroviruses is then collected, optionally concentrated, and used for gene transfer. Retroviral vectors are able to infect a broad variety of cell types. However, integration and stable expression typically involves the division of host cells (Paskind et al., Virology, 67:242-248, 1975).

In some embodiments, the viral vector is a lentiviral vector. Lentiviruses are complex retroviruses, which, in addition to the common retroviral genes gag, pol, and env, contain other genes with regulatory or structural function. Information on lentiviral vectors is available, for example, in Naldini et al., Science 272(5259):263-267, 1996; Zufferey et al., Nat Biotechnol 15(9):871-875, 1997; Blomer et al., J Virol. 71(9):6641-6649, 1997; U.S. Pat. Nos. 6,013,516 and 5,994,136, each of which is incorporated herein by reference in its entirety. Some examples of lentivirus include the Human Immunodeficiency Viruses: HIV-1, HIV-2, and the Simian Immunodeficiency Virus: SIV. Lentiviral vectors have been generated by attenuating the HIV virulence genes, for example, the genes env, vif, vpr, vpu and nef are deleted to make the vector biologically safe. The lentivirus employed is sometimes replication and/or integration defective.

Recombinant lentiviral vectors are capable of infecting non-dividing cells and are sometimes used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, which is incorporated herein by reference in its entirety. In some embodiments, the recombinant virus is targeted by linkage of the envelope protein with an antibody or a particular ligand for targeting to a receptor of a particular cell type. For example, a target-specific vector is sometimes generated by inserting a nucleic acid segment (including a regulatory region) of interest into the viral vector, along with another gene that encodes a ligand for a receptor on a specific target cell type.

Lentiviral vectors are known in the art, see Naldini et al., (1996 and 1998): Zufferey et al., (1997); Dull et al., 1998, U.S. Pat. Nos. 6,013,516; and 5,994,136 all incorporated herein by reference. In general, these vectors are plasmid-based or virus-based and are configured to carry the essential sequences for incorporating foreign nucleic acid, for selection and for transfer of the nucleic acid into a host cell. In some cases, a lentiviral vector is introduced into a cell concurrently with one or more lentiviral packaging plasmids, which include, without limitation, pMD2.G, pRSV-rev, pMDLG-pRRE, and pRRL-GOI. Introduction of a lentiviral vector alone or in combination with lentiviral packaging plasmids into a cell, in some embodiments causes the lentiviral vector to be packaged into a lentiviral particle. In some embodiments, the lentiviral vector is a non-integrating lentiviral (NIL) vector. Illustrative methods for generating NIL vectors, such as the D64V substitution in the integrase gene, are provided in U.S. Pat. No. 8,119,119.

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200(2):535-546, 1992)). In some cases, PKP2 is introduced into the cell using adenovirus assisted transfection. Increased transfection efficiencies have been reported in cell systems using adenovirus coupled systems (Kelleher and Vos, Biotechniques, 17(6):1110-7, 1994: Cotten et al., Proc Natl Acad Sci USA, 89(13):6094-6098, 1992; Curiel, Nat Immun, 13(2-3):141-64, 1994.).

In some embodiments, the viral vector is an adeno-associated virus (AAV) vector. AAV is an attractive vector system as it has a low frequency of integration and it can infect non-dividing cells, thus making it useful for delivery of polynucleotides into mammalian cells, for example, in tissue culture (Muzyczka, Curr Top Microbiol Immunol, 158:97-129, 1992) or in vivo. Details concerning the generation and use of rAAV vectors are described in U.S. Pat. Nos. 5,139,941 and 4,797,368, each incorporated herein by reference in its entirety.

AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC. 001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(l): 67-76 (2006); and the AAV-11 genome is provided in Virology, 330(2): 375-383 (2004), The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and often persists essentially for the lifetime of those cells as a transcriptionally active nuclear episome (extrachromosomal element). Of particular importance to the present disclosure, AAV, and AAV9 in particular, are capable of infecting cells of the heart, such as myocardium, epicardium, or both (Prasad et al, 2011; Piras et al, 2016; Ambrosi et al., 2019), The AAV proviral genome is inserted as cloned DNA in plasmids, which makes construction of recombinant genomes feasible. Furthermore, because the signals directing AAV replication and genome encapsidation are contained within the ITRs of the AAV genome, in some cases, some or all of the internal approximately 4.3 kb of the genome (encoding replication and structural capsid proteins, rep-cap) is replaced with foreign DNA, To generate AAV vectors, in some cases, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. In some cases, AAV is even be lyophilized. Finally, AAV-infected cells are not resistant to superinfection. The AAV vectors of the disclosure include self-complementary, duplexed AAV vectors, synthetic ITRs, and/or AAV vectors with increased packaging compacity. Illustrative methods are provided in U.S. Pat. Nos. 8,784,799; 8,999,678; 9,169,494; 9,447,433; and U.S. Pat. No. 9,783,824, each of which is incorporated by reference in its entirety.

AAV DNA in the rAAV genomes is contemplated to be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV- 10, AAV-11, AAV- 12, AAV-13 and AAV rh74. Production of pseudotyped rAAV is disclosed in, for example, WO 01/83692. Other types of rAAV variants, for example rAAV with capsid mutations, are also contemplated. See, for example. Marsic et al., Mol. Therapy. 22):1900-09 (2014), The nucleotide sequences of the genomes of various AAV serotypes are known in the art. AAV vectors of the present disclosure include AAV vectors of serotypes AAV1, AAV2, AAV4, AAV5, AAV6, AAV7, AAV8, AAV9, AAV10, AAV39, AAV43, AAV.rh74, and AAV.rh8. Illustrative AAV vectors are provided in U.S. 63/012,703; U.S. Pat. No. 7,105,345; U.S. Ser. No. 15/782,980; U.S. Pat. Nos. 7,259,151; 6,962,815; 7,718,424; 6,984,517; 7,718,424; 6,156,303; 8,524,446; 7,790,449: U.S. Pat. Nos. 7,906,111; 9,737,618; U.S. application Ser. No. 15/433,322; U.S. Pat. No. 7,198,951, each of which is incorporated by reference in its entirety.

In some embodiments, the AAV expression vector is pseudotyped to enhance targeting. To promote gene transfer and sustain expression in cardiomyocytes, AAV6, AAV8, and AAV9, are contemplated for use. In some cases, the AAV2 genome is packaged into the capsid of producing pseudotyped vectors AAV2′5. AAV2/7, and AAV2′8 respectively, as described in Balaji et al. J Surg Res. 184:691-98 (2013). In some embodiments, an AAV9 is used to target expression in myofibroblast-like lineages, as described in Piras et al. Gene Therapy 23:469-478 (2016). In some embodiments, AAV I, AAV6, or AAV9 is used, and in some embodiments, the AAV is engineered, as described in Asokari et al. Hum Gene Ther. 24:906-13 (2013); Pozsgai et al. Mol Ther. 25:855-69 (2017); Kotterman et al. Nature Reviews Genetics 15:445-51 (2014); and US20160340393A 1 to Schaffer et al. In some embodiments, the viral vector is AAV engineered to increase target cell infectivity as described in US20180066285A1.

In some embodiments, the AAV vectors of the disclosure comprise a modified capsid, in particular as capsid engineered to enhance or promote in vivo or ex vivo transduction of cardiac cells, or more particularly cardiomyocytes: or that evade the subject's immune system; or that have improved biodistribution. Illustrative AAV capsids are provided in U.S. Pat. Nos. 7,867,484; 9,233,131; 10,046,016; WO 2016/133917; WO 2018/222503; and WO 20019/060454, each of which is incorporated by reference in its entirety. In an AAV capsid (or in particular an AAV9 capsid), one or more substitutions are contemplated to increase infectivity towards cells in the myocardium, epicardium, or both. More particularly, in some embodiments, the AAV vectors of the disclosure, optionally AAV9-based vectors, comprise in their capsid proteins one or more substitutions. In some embodiments, the AAV vectors of the disclosure comprise the AAV-A9 capsid and/or serotype. It will be appreciated that these substitutions and insertions are contemplated to be combined together to generate various capsid proteins useful in the present disclosure.

### Methods of Producing Viral Vectors

In general, a viral vector is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. In some embodiments, the retroviral packaging cell comprises a gene encoding a viral polypeptide, e.g., VSV-g, that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as El A or El B or other adenoviral proteins. For example, in some cases, proteins supplied by packaging cells are retrovirus-derived proteins such as gag, pol, and env; lentivirus-derived proteins such as gag, poi, env, vpr, vpu, vpx, vif, tat, rev, and nef; and adenovirus-derived proteins such as El A and El B. In many examples, the packaging cells supply proteins derived from a virus that differs from the virus from which the viral vector is derived. Methods of producing recombinant viruses from packaging cells and their uses are well established: see, e.g., U.S. Pat. Nos. 5,834,256; 6,910,434; 5,591,624; 5,817,491; 7,070,994; and 6,995.009.

Packaging cell lines include but are not limited to any easily-transfectable cell line. Packaging cell lines are often based on 293T cells, NIH3T3, COS or HeLa cell lines. Packaging cells are often used to package virus vector plasmids deficient in at least one gene encoding a protein required for virus packaging. Any cells that supply a protein or polypeptide lacking from the proteins encoded by such viral vectors or plasmids are contemplated for use as packaging cells. Examples of packaging cell lines include but are not limited to: Platinum-E (Plat-E), Platinum-A (Plat- A), BOSC 23 (ATCC CRL 11554) and Bing (ATCC CRL 11270), Morita et al. (2000) Gene Therapy 7(12): 1063-1066; Onishi et al. (1996) Experimental Hematology, 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

Virus vector plasmids (or constructs), include: pMXs, pMxs-IB, pMXs-puro, pMXs-neo (pMXs-IB is a vector carrying the blasticidin-resistant gene instead of the puromycin-resistant gene of pMXs-puro) Kimatura et al. (2003) Experimental Hematology 31: 1007-1014; MFG Riviere et al. (1995) Proc. Natl. Acad. Sci., 92:6733-6737: pBabePuro; Morgenstern et al. (1990) Nucleic Acids Research 18:3587-3596; LL-CG, CL-CG, CS-CG, CLG Miyoshi et al. (1998) J. Vir. 72:8150-8157 and the like as the retrovirus system, and pAdexl Kanegae et al. (1995) Nucleic Acids Research 23:3816-3821 and the like as the adenovirus system. In exemplary embodiments, the retroviral construct comprises blasticidin (e.g., pMXs-IB), puromycin (e.g., pMXs-puro, pBabePuro), or neomycin (e.g., pMXs-neo). Morgenstem et al. (1990) Nucleic Acids Research 18:3587-3596.

### Promoters and Enhancers

In some embodiments, a nucleic acid encoding a PKP2 is operably linked to a promoter and/or enhancer to facilitate expression of PKP2. Depending on the host/vector system utilized, any of a number of suitable transcription and translation control elements, including constitutive, tissue specific, and inducible promoters, transcription enhancer elements, transcription terminators, etc. are suitable for use in the expression vector (e.g., Bitter et al. (1987) Methods in Enzymology, 153:516-544).

Non-limiting examples of suitable eukaryotic promoters (promoters functional in a eukaryotic cell) include CMV, CMV immediate early, HSV thymidine kinase, early and late SV40, long terminal repeats (LTRs) from retrovirus, and mouse metallothionein-I. In some embodiments, promoters that are capable of conferring cardiac-specific expression will be used, including but not limited to promoters that confer expression in the myocardium, the epicardium, or both (Prasad et al., 2011), Non-limiting examples of suitable cardiac-specific promoters include alpha-myosin heavy chain (a-MHC), myosin light chain 2 (MLC-2), cardiac troponin T (cTnT), and cardiac troponin C (cTnC). In some embodiments, a PKP2 or a desmin promoter is used. In some cases, a chimeric promoter with cardiac specific expression is used. In some cases, a cardiac specific enhancer is combined with the promoter.

Examples of suitable promoters for driving expression PKP2 include, but are not limited to, retroviral long terminal repeat (LTR) elements; constitutive promoters such as CMV, HSVI-TK, SV40, EF-1a, β-actin, phosphoglycerol kinase (PGK): inducible promoters, such as those containing Tet-operator elements; and cardiac-specific promoters, such as alpha-myosin heavy chain (a-MHC), myosin light chain 2 (MLC-2), cardiac troponin T (cTnT), and cardiac troponin C (cTnC). In some embodiments, a PKP2 or a desmin promoter is used. In some embodiments, a chimeric promoter with cardiac specific expression is used. In some cases, a cardiac specific enhancer is combined with the promoter.

In some embodiments, a polynucleotide is operably linked to a cell type-specific transcriptional regulator element (TRE), where TREs include promoters and enhancers. Suitable TREs include, but are not limited to, TREs derived from the following genes: myosin light chain-2, a-myosin heavy chain, AE3, cardiac troponin C, and cardiac actin. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Cell. Biol. 14: 1870-1885; Hunter et al. (1993) Hypertension 22:608-617; and Sartorelli et al. (1992) Proc. Natl. Acad. Sci. USA 89:4047-4051.

Alternatively, certain advantages will be gained by positioning the coding nucleic acid segment under the control of a recombinant or heterologous promoter, which refers to a promoter that is not normally associated with a nucleic acid in its natural environment. A recombinant or heterologous enhancer refers also to an enhancer not normally associated with a nucleic acid sequence in its natural environment. Such promoters or enhancers often include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences are sometimes produced using recombinant cloning and/or nucleic acid amplification technology, including PCR, in connection with the compositions disclosed herein (see U.S. Pat. Nos. 4,683,202, 5,928,906, each incorporated herein by reference).

In some embodiments, the vectors of the disclosure include one or more polyA signals. Illustrative polyA signals useful in the vectors of the disclosure include the short polyA signal and the bGH polyA signal. In some embodiments, the vectors of the disclosure include one or more 3′ elements. Illustrative 3′ elements include the woodchuck hepatitis virus posttranscriptional regulatory element (WPRE).

### Gene Therapy Vector Compositions

To prepare the composition, the vectors and/or the cells are generated, and the vectors or cells are purified as necessary or desired. The vectors, and/or other agents are sometimes suspended in a pharmaceutically acceptable carrier. In some embodiments, the composition is lyophilized. These compounds and cells are often adjusted to an appropriate concentration, and optionally combined with other agents. The absolute weight of a given compound and/or other agent included in a unit dose varies widely. The dose and the number of administrations are contemplated to be optimized by those skilled in the art.

For example, in some embodiments, about 10²-10¹⁰ vector genomes (vg) are be administered. In some embodiments, the dose be at least about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes. In some embodiments, the dose be about 10² vg, about 10³ vg, about 10⁴ vg, about 10⁵ vg, about 10⁶ vg, about 10⁷ vg, about 10⁸ vg, about 10⁹ vg, about 10¹⁰ vg, or more vector genomes.

Daily doses of the compounds vary as well. Such daily doses often range, for example, from at least about 10² vg/day, about 10³ vg/day, about 10⁴ vg/day, about 10⁵ vg/day, about 10⁶ vg/day, about 10⁷ vg/day, about 10⁸ vg/day, about 10⁹ vg/day, about 10¹⁰ vg/day, or more vector genomes per day.

In some embodiments, the method of the disclosure comprises administering a vector or vector system of the disclosure (e.g. an rAAV vector) by intracardiac injection, intramyocardiac injection, endocardial injection, intracardiac catheterization, or systemic administration. In some embodiments, the subject (e.g., a human) is treated by administering between about 1×10⁸ and about 1×10¹⁵ GC of a vector (e.g., an AAV vector or lentiviral vector) by intracardiac injection, intramyocardiac injection, endocardial injection, intracardiac catheterization, or systemic administration. In some embodiments, the subject is treated by administering between about 1×10⁸ and about 1×10¹⁵ GC, between about 1×10⁸ and about 1×10¹⁵ GC, between about 1×10⁹ and about 1×10¹⁴ GC, between about 1×10¹⁰ and about 1×10¹³ GC, between about 1×10¹¹ and about 1×10¹² GC, or between about 1×10¹² and about 1×10¹³ GC of vector. In some embodiments, the subject is treated by administering between about 1×10⁸ and about 1×10¹⁰ GC, between about 1×10⁹ and about 1×10¹¹ GC, between about 1×10¹⁰ and about 1×10¹² GC, between about 1×10¹¹ and about 1×10¹³ GC, between about 1×10¹² and about 1×10¹³ GC, or between about 1×10¹³ and about 1×10¹⁵ GC of vector. In some embodiments, the subject is treated by administering at least 1×10⁸, at least about 1×10⁹, at least about 1×10¹⁰, at least about 1×10¹¹, at least about 1×10¹², at least about 1×10¹³, or at least about 1×10¹⁵ GC of vector. In some embodiments, the subject is treated by administering at most 1×10⁸, at most about 1×10⁹, at most about 1×10¹⁰, at most about 1×10¹¹, at most about 1×10¹², at most about 1×10¹³, or at most about 1×10¹⁵ GC of vector. In some embodiments, the subject (e.g., a human) is treated by administering between about 1×10⁸ and about 1×10¹⁵ GC/kg of a vector(e.g., an AAV vector or lentiviral vector) by intracardiac injection or systemically. In some embodiments, the subject is treated by administering between about 1×10⁸ and about 1×10¹⁵ GC/kg, between about 1×10⁸ and about 1×10¹⁵ GC/kg, between about 1×10⁹ and about 1×10¹⁴ GC/kg, between about 1×10¹⁰ and about 1×10¹³ GC/kg, between about 1×10¹¹ and about 1×10¹² GC/kg, or between about 1×10¹² and about 1×10¹³ GC/kg of vector. In some embodiments, the subject is treated by administering between about 1×10⁸ and about 1×10¹⁰ GC/kg, between about 1×10⁹ and about 1×10¹¹ GC/kg, between about 1×10¹⁰ and about 1×10¹² GC/kg, between about 1×10¹¹ and about 1×10¹³ GC/kg, between about 1×10¹² and about 1×10¹⁴ GC/kg, or between about 1×10¹³ and about 1×10¹⁵ GC/kg of vector. In some embodiments, the subject is treated by administering at least 1×10⁸, at least about 1×10⁹, at least about 1×10¹⁰, at least about 1×10¹¹, at least about 1×10¹², at least about 1×10¹³, or at least about 1×10¹⁵ GC/kg of vector. In some embodiments, the subject is treated by administering at most 1×10⁸, at most about 1×10⁹, at most about 1×10¹⁰, at most about 1×10¹¹, at most about 1×10¹², at most about 1×10¹³, or at most about 1×10¹⁵ GC/kg of vector. It will be appreciated that the amount of vectors and for use in treatment will vary not only with the particular carrier selected but also with the route of administration, the nature of the condition being treated and the age and condition of the patient. Ultimately, in some embodiments, the attendant health care provider will determine proper dosage. A pharmaceutical composition is contemplated to be formulated with the appropriate ratio of each compound in a single unit dosage form for administration.

The compositions are sometimes formulated for sustained release (for example, using microencapsulation, see WO 94/07529, and/or U.S. Pat. No. 4,962,091), The formulations, where appropriate, are conveniently presented in discrete unit dosage forms and, in some embodiments, are prepared by any of the methods well known to the pharmaceutical arts. Such methods often include the step of mixing the therapeutic agent with liquid carriers, solid matrices, semi-solid carriers, finely divided solid carriers or combinations thereof, and then, if necessary, introducing or shaping the product into the desired delivery system.

One or more suitable unit dosage forms containing the compounds, in some embodiments, are administered by a variety of routes including parenteral (including subcutaneous, intravenous, intramuscular, and intraperitoneal), intracardially, pericardially, oral, rectal, dermal, transdermal, intrathoracic, intrapulmonary, and intranasal (respiratory) routes.

The gene therapy vectors provided herein are prepared in many forms that include aqueous solutions, suspensions, tablets, hard or soft gelatin capsules, and liposomes and other slow-release formulations, such as shaped polymeric gels. Administration of gene therapy vectors often involves parenteral or local administration in an aqueous solution. Similarly, compositions containing gene therapy vectors are sometimes administered in a device, scaffold, or as a sustained release formulation. Different types of formulating procedures are described in U.S. Pat. No. 6,306,434 and in the references contained therein.

Vectors, in some embodiments, are formulated for parenteral administration (e.g., by injection, for example, bolus injection or continuous infusion) and are often presented in unit dosage form in ampoules, prefilled syringes, small volume infusion containers or multi-dose containers with an added preservative. The pharmaceutical compositions often take the form of suspensions, solutions, or emulsions in oily or aqueous vehicles, and sometimes contain formulatory agents such as suspending, stabilizing and/or dispersing agents. Suitable carriers include saline solution, phosphate buffered saline, and other materials commonly used in the art.

The compositions sometimes also contain other ingredients such as agents useful for treatment of cardiac diseases, conditions and injuries, such as, for example, an anticoagulant (e.g., dalteparin (fragmin), danaparoid (orgaran), enoxaparin (lovenox), heparin, tinzaparin (innohep), and/or warfarin (coumadin)), an antiplatelet agent (e.g., aspirin, ticlopidine, clopidogrel, or dipyridamole), an angiotensin-converting enzyme inhibitor (e.g., Benazepril (Lotensin), Captopril (Capoten), Enalapril (Vasotec), Fosinopril (Monopril), Lisinopril (Prinivil, Zestril), Moexipril (Univase), Perindopril (Aceon), Quinapril (Accupril), Ramipril (Altace), and/or Trandolapril (Mavik)), angiotensin II receptor blockers (e.g., Candesartan (Atacand), Eprosartan (Teveten), Irbesartan (Avapro), Losartan (Cozaar), Telmisartan (Micardis), and/or Valsartan (Diovan)), a beta blocker (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL). Nadolol (Corgard), Propranolol (Inderal), Sotalol (Betapace), and/or Timolol (Blocadren)), Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), diuretics (e.g., Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and/or Spironolactone (Aldactone)), vasodilators (e.g., Isosorbide dinitrate (Isordil), Nesiritide (Natrecor), Hydralazine (Apresoline), Nitrates and/or Minoxidil), statins, nicotinic acid, gemfibrozil, clofibrate, Digoxin, Digitoxin, Lanoxin, or any combination thereof.

Additional agents are sometimes included such as antibacterial agents, antimicrobial agents, anti-viral agents, biological response modifiers, growth factors; immune modulators, monoclonal antibodies and/or preservatives. The compositions provided herein are contemplated to also be used in conjunction with other forms of therapy.

The viral vectors described herein are suitable for administration to a subject to treat a disease or disorder. In some embodiments, such a composition is in a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compounds and compositions of provided herein, in some embodiments, are administered continuously over a preselected period of time or alternatively are administered in a series of spaced doses. Both local and systemic administration is contemplated. In some embodiments, localized delivery of a viral or non-viral vector is achieved. In some embodiments, localized delivery of cells and/or vectors is used to generate a population of cells within the heart. In some embodiments, such a localized population operates as “pacemaker cells” for the heart.

### Definitions

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder is, in some cases, inflammatory, metabolic, toxic, infiltrative, fibroplastic, hematological, genetic, or unknown in origin. There are two general types of cardiomyopathies: ischemic (resulting from a lack of oxygen) and non-ischemic. In some cases, a cardiomyopathy is arrhythmogenic right ventricular cardiomyopathy (ARVC) or arrhythmogenic cardiomyopathy (ACM).

“Heart failure” (HF) is a complex clinical syndrome that often result from any structural or functional cardiovascular disorder causing systemic perfusion inadequate to meet the body's metabolic demands without excessively increasing left ventricular filling pressures. It is characterized by specific symptoms, such as dyspnea and fatigue, and signs, such as fluid retention. As used herein, “chronic heart failure” or “congestive heart failure” or “CHF” refer, interchangeably, to an ongoing or persistent forms of heart failure. Common risk factors for CHF include old age, diabetes, high blood pressure and being overweight. CHF is broadly classified according to the systolic function of the left ventricle as HF with reduced or preserved ejection fraction (HFrEF and HFpEF). The term “heart failure” does not mean that the heart has stopped or is failing completely, but that it is weaker than is normal in a healthy person. In some cases, the condition is mild, causing symptoms that are noticeable when exercising, in others, the condition is more severe, causing symptoms that are, in some cases, life-threatening, even while at rest. The most common symptoms of chronic heart failure include shortness of breath, tiredness, swelling of the legs and ankles, chest pain and a cough. In some embodiments, the methods of the disclosure decrease, prevent, or ameliorate one or more symptoms of CHF (e.g., HFrEF) in a subject suffering from or at risk for CHF (e.g., HFrEF). In some embodiments, the disclosure provides methods of treating CHF and conditions that sometimes lead to CHF.

As used herein “acute heart failure” or “decompensated heart failure” refer, interchangeably, to a syndrome of the worsening of signs and symptoms reflecting an inability of the heart to pump blood at a rate commensurate to the needs of the body at normal filling pressure. AHF typically develops gradually over the course of days to weeks and then decompensates requiring urgent or emergent therapy due to the severity of these signs or symptoms. In some cases, AHF is the result of a primary disturbance in the systolic or diastolic function of the heart or of abnormal venous or arterial vasoconstriction, but generally represents an interaction of multiple factors, including volume overload. The majority of patients with AHF have decompensation of chronic heart failure (CHF) and consequently much of the discussion of the pathophysiology, presentation, and diagnosis of CHF is directly relevant to an understanding of AHF. In other cases. AHF results from an insult to the heart or an event that impairs heart function, such as an acute myocardial infarction, severe hypertension, damage to a heart valve, abnormal heart rhythms, inflammation or infection of the head, toxins, and medications. In some embodiments, the methods of the disclosure decrease, prevent, or ameliorate one or more symptoms of AHF in a subject suffering from or at risk for AHF. In some embodiments, the disclosure provides methods of treating A-F and conditions that sometimes lead to AHF. In some cases, ARF is the result of ischemia associated with myocardial infarction.

As used herein, the terms “subject” or “individual” refers to any animal, such as a domesticated animal, a zoo animal, or a human. In some cases, the “subject” or “individual” is a mammal like a dog, cat, horse, livestock, a zoo animal, or a human. Alternatively, or in combination, the subject or individual is a domesticated animal such as a bird, a pet, or a farm animal. Specific examples of “subjects” and “individuals” include, but are not limited to, individuals with a cardiac disease or disorder, and individuals with cardiac disorder-related characteristics or symptoms, such as arrhythmogenic right ventricular cardiomyopathy (ARVC) or arrhythmogenic cardiomyopathy (ACM).

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Calos eds. (1987) Gene Transfer Vectors for Mammalian Cells (Cold Spring Harbor Laboratory): Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental immunology; Manipulating the Mouse Embryo: A Laboratory Manual, 3rd edition (2002) Cold Spring Harbor Laboratory Press; Sohail (2004) Gene Silencing by RNA Interference: Technology and Application (CRC Press); Sell (2013) Stem Cells Handbook.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A. B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cardiomyocyte” includes a plurality of cardiomyocytes.

Also as used herein, “and/or” refers to and encompasses any and all possible combinations of one or more of the associated listed items, as well as the lack of combinations when interpreted in the alternative (“or”).

“Administration,” “administering” and the like, when used in connection with a gene therapy vector or composition thereof as provided herein refer both to direct administration, which, in some cases includes administration to non-cardiomyocytes in vitro, administration to non-cardiomyocytes in vivo, administration to a subject by a medical professional or by self-administration by the subject and/or to indirect administration, which, in some cases, is the act of prescribing a composition comprising a gene therapy vector provided herein. When used herein in reference to a cell, it refers to introducing a composition to the cell. Typically, an effective amount is administered, which amount is often to be determined by one of skill in the art. Any suitable method of administration is contemplated to be used. In some cases, a gene therapy vector is administered to the cells by, for example, by addition of the gene therapy vector to the cell culture media or injection in vivo to the site of cardiac injury. In some cases, administration to a subject is achieved by, for example, intravascular injection, intramyocardial delivery, and the like.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium, or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. In some cases, cardiac cells are derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac a-actinin. The term “cardiomyocyte” as used herein is an umbrella term comprising any cardiomyocyte subpopulation or cardiomyocyte subtype, e.g., atrial, ventricular and pacemaker cardiomyocytes.

The term “culture” or “cell culture” means the maintenance of cells in an artificial, in vitro environment. A “cell culture system” is used herein to refer to culture conditions in which a population of cells are grown as monolayers or in suspension. “Culture medium” is used herein to refer to a nutrient solution for the culturing, growth, or proliferation of cells. Culture medium is characterized, in some cases, by functional properties such as, but not limited to, the ability to maintain cells in a particular state (e.g., a pluripotent state, a quiescent state, etc.), or to mature cells, such as, in some embodiments, to promote the differentiation of progenitor cells into cells of a particular lineage (e.g., a cardiomyocyte).

As used herein, the term “expression” or “express” refers to the process by which nucleic acids or polynucleotides are transcribed into mRNA and/or the process by which the transcribed mRNA is subsequently being translated into peptides, polypeptides, or proteins. If the polynucleotide or nucleic acid is derived from genomic DNA, in some cases, expression includes splicing of the mRNA in a eukaryotic cell. In some cases, the expression level of a gene is determined by measuring the amount of mRNA or protein in a cell or tissue sample.

As used herein, an “expression cassette” is a DNA polynucleotide comprising one or more polynucleotides or nucleic acids encoding protein(s) or nucleic acid(s) that is configured to express the polynucleotide in a host cell. Typically, expression of the polynucleotide(s) is placed under the control of certain regulatory elements, including constitutive or inducible promoters, tissue-specific regulatory elements, and enhancers. Such polynucleotides are said to be “operably linked to” or “operatively linked to” the regulatory elements (e.g., a promoter).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Treatment,” “treating,” and “treat” are defined as acting upon a disease, disorder, or condition with an agent to reduce or ameliorate harmful or any other undesired effects of the disease, disorder, condition and/or their symptoms.

As used herein, the term “effective amount” and the like refers to an amount that is sufficient to induce a desired physiologic outcome (e.g., treatment of a disease). An effective amount is sometimes administered in one or more administrations, applications, or dosages. Such delivery is dependent on a number of variables including the time period which the individual dosage unit is to be used, the bioavailability of the composition, the route of administration, etc. It is understood, however, that specific amounts of the compositions (e.g., gene therapy vectors) for any particular subject depends upon a variety of factors including the activity of the specific agent employed, the age, body weight, general health, sex, and diet of the subject, the time of administration, the rate of excretion, the composition combination, severity of the particular disease being treated and form of administration.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant, in some cases, alters the amino acid sequence of a polypeptide encoded by the reference polynucleotide. In some cases, nucleotide changes result in amino acid substitutions, deletions, additions, fusions, and truncations in the polypeptide encoded by the reference sequence. Generally, differences are limited so that the sequences of the reference polypeptide and the variant are closely similar overall and, in many regions, identical.

As used herein, the term “nucleic acid” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers. As used herein, the word “polynucleotide” or “nucleic acid” preceded by a gene name (for example, “PKP2 nucleic acid”) refers to a polynucleotide sequence encoding the corresponding protein (for example, a “PKP2 protein”).

The terms “polypeptide,” “peptide,” and “protein,” are used interchangeably herein and refer to a polymeric form of amino acids of any length, which sometimes include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues, immunologically tagged proteins, and the like. As used herein, the word “protein” preceded by a gene name (for example, “PKP2 protein”) refers to either the native protein or a functional variant thereof. A “native protein” is a protein encoded by a genomic copy of a gene of an organism, preferably the organism for which the vector is intended (e.g., a human, a rodent, a primate, or an animal of veterinary interest), in any of the gene's functional isoforms or functional allelic variations.

As used herein, a “functional variant” or “variant” of a protein is a variant with any number of amino acid substitutions, insertions, truncations, or internal deletions that retains the functional attributes of the protein, including, e.g., the protein's ability to induce, in combination with other factors, organization of desmosomes. In some cases, functional variants are identified computationally, such as variants having only conservative substitutions, or experimentally using in vitro or in vivo assays.

As used herein, a “codon variant” of a polynucleotide sequence is polynucleotide sequence that encodes the same protein as a reference polynucleotide sequence having one or more synonymous codon substitutions. Selection of synonymous codons is within the skill of those in the art, the coding as the genetic code being known. In some cases, codon optimization is performed using a variety of computational tools (such the GENSMART™ Codon Optimization tool available at www.genscript.com). Generally, codon optimization is used to increase the expression of protein in a heterologous system, for instance when a human coding sequence is expressed in a bacterial system. The term “codon variant” is intended to encompass both sequences that are optimized in this manner and sequences that are optimized for other purposes, such as removal of CpG islands and/or cryptic start sites.

The term “vector” refers to a macromolecule or complex of molecules comprising a polynucleotide or protein to be delivered to a host cell, either in vitro or in vivo. A vector is sometimes a modified RNA, a lipid nanoparticle (encapsulating either DNA or RNA), a transposon, an adeno-associated virus (AAV) vector, an adenovirus, a retrovirus, an integrating lentiviral vector (LVV), or a non-integrating LVV. Thus, as used herein “vectors” include naked polynucleotides used for transformation (e.g. plasmids) as well as any other composition used to deliver a polynucleotide to a cell, included vectors capable of transducing cells and vectors useful for transfection of cells.

As used herein, the term “viral vector” refers either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also cell components in addition to nucleic acid(s).

The term “genetic modification” refers to a permanent or transient genetic change induced in a cell following introduction of new nucleic acid (i.e., nucleic acid exogenous to the cell). Genetic change is often accomplished by incorporation of the new nucleic acid into the genome of the cardiac cell, or by transient or stable maintenance of the new nucleic acid as an extrachromosomal element. Where the cell is a eukaryotic cell, a permanent genetic change is often achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

Unless the context indicates otherwise, the features of the invention can be used in any combination. Any feature or combination of features set forth can be excluded or omitted. Certain features of the invention, which are described in separate embodiments may also be provided in combination in a single embodiment. Features of the invention, which are described in a single embodiment may also be provided separately or in any suitable sub-combination. All combinations of the embodiments are disclosed herein as if each and every combination were individually disclosed. All sub-combinations of the embodiments and elements are disclosed herein as if every such sub-combination were individually disclosed.

Unless defined otherwise, all technical and scientific terms used herein have the same meaning as commonly understood by one of ordinary skill in the art to which this invention belongs. The detailed description is divided into sections only for the reader's convenience and disclosure found in any section may be combined with that in another section. Although any methods and materials similar or equivalent to those described herein can also be used in the practice or testing of the present invention, the exemplary methods and materials are now described. All publications mentioned herein are incorporated by reference to disclose and describe the methods and/or materials in connection with which the publications are cited. Reference to a publication is not an admission that the publication is prior art.

The singular forms “a,” “an,” and “the” include plural referents unless the context clearly dictates otherwise. For example, reference to “a recombinant AAV virion” includes a plurality of such virions and reference to “the cardiac cell” includes one or more cardiac cells.

The conjunction “and/or” means both “and” and “or,” and lists joined by “and/or” encompasses all possible combinations of one or more of the listed items.

The term “vector” refers to a macromolecule or complex of molecules comprising a polynucleotide or protein to be delivered to a cell.

“AAV” is an abbreviation for adeno-associated virus. The term covers all subtypes of AAV, except where a subtype is indicated, and to both naturally occurring and recombinant forms. The abbreviation “rAAV” refers to recombinant adeno-associated virus. “AAV” includes AAV or any subtype. “AAV5” refers to AAV subtype 5. “AAV9” refers to AAV subtype 9. The genomic sequences of various serotypes of AAV, as well as the sequences of the native inverted terminal repeats (ITRs), Rep proteins, and capsid subunits may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), NC_001829 (AAV4), U89790 (AAV4), NC_006152 (AAV5), AF513851 (AAV7), AF513852 (AAV8), NC_006261 (AAV8), and AY530579 (AAV9), Publications describing AAV include Srivistava et al. (1983) J. Virol. 45:555; Chiorini et al. (1998) J. Virol. 71:6823; Chiorini et al. (1999) J. Virol. 73:1309; Bantel-Schaal et al. (1999) J. Virol. 73:939; Xiao et al. (1999). J. Virol. 73:3994; Muramatsu et al. (1996) Virol. 221:208; Shade et al. (1986) J. Virol. 58:921; Gao et al. (2002) Proc. Nat. Acad. Sci. USA 99: 11854; Moris et al. (2004) Virology 33:375-383; Int'l Pat. Publ Nos. WO2018/222503A1, WO2012/145601A2, WO2000/028061A2, WO1999/61601A2, and WO1998/11244A2; U.S. patent application Ser. Nos. 15/782,980 and 15/433,322; and U.S. Pat. Nos. 10,036,016, 9,790,472, 9,737,618, 9,434,928, 9,233,131, 8,906,675, 7,790,449, 7,906,111, 7,718,424, 7,259,151, 7,198,951, 7,105,345, 6,962,815, 6,984,517, and 6,156,303.

An “AAV vector” or “rAAV vector” as used in the art to refer either to the DN A packaged into in the rAAV virion or to the rAAV virion itself, depending on context. As used herein, unless otherwise apparent from context, rAAV vector refers to a nucleic acid (typically a plasmid) comprising a polynucleotide sequence capable of being packaged into an rAAV virion, but with the capsid or other proteins of the rAAV virion. Generally, an rAAV vector comprises a heterologous polynucleotide sequence (i.e., a polynucleotide not of AAV origin) and one or two AAV inverted terminal repeat sequences (ITRs) flanking the heterologous polynucleotide sequence. Only one of the two ITRs may be packaged into the rAAV and yet infectivity of the resulting rAAV virion may be maintained. See Wu et al. (2010) Mol Ther. 18:80. An rAAV vector may be designed to generate either single-stranded (ssAAV) or self-complementary (scAAV). See McCarty D. (2008) Mo. Ther. 16:1648-1656; WO2001/11034; WO2001/92551: WO2010/129021.

An “rAAV virion” refers to an extracellular viral particle including at least one viral capsid protein (e.g., VP1) and an encapsidated rAAV vector (or fragment thereof), including the capsid proteins.

For brevity and clarity, the disclosure refers to “capsid protein” or “capsid proteins.” Those skilled in the art understand that such references refer to VP1, VP2, or VP3, or combinations of VP1, VP2, and VP3. As in wild-type AAV and most recombinant expression systems VP1, VP2, and VP3 are expressed from the same open reading frame, engineering of the sequence that encodes VP3 inevitably alters the sequences of the C-terminal domain of VP1 and VP2. One may also express the capsid proteins from different open reading frames, in which case the capsid of the resulting rAAV virion could contain a mixture of wild-type and engineered capsid proteins, and mixtures of different engineered capsid proteins.

Positions with a sequence alignment are generally denotes in terms of a reference sequence. Unless otherwise specified, amino acid positions in the engineered capsid proteins disclosed herein are numbered according to the VP1 sequence of AAV9 provided as SEQ ID NO: 1. Positions may be determined using a best fit alignment of a sequence of interest to a reference sequence. An insertion “at” a position means inserting sequence between that amino acid position and the preceding position in the alignment. The term “about” allows for substitutions or insertions in positions near to the reference position. Those of skill in the art can used techniques such as structural modeling to determine suitable nearby positions (e.g., by identifying the residues in the loop region exposed on the surface of the capsid).

The term “inverted terminal repeats” or “ITRs” as used herein refers to AAV viral cis-elements named so because of their symmetry. These elements are essential for efficient multiplication of an AAV genome. Without being bound by theory, it is believed that the minimal elements indispensable for ITR function are a Rep-binding site and a terminal resolution site plus a variable palindromic sequence allowing for hairpin formation. The disclosure contemplates that alternative means of generating an AAV genome may exist or may be prospectively developed to be compatible with the capsid proteins of the disclosure.

“Helper virus functions” refers to functions encoded in a helper virus genome which allow AAV replication and packaging.

“Packaging” refers to a series of intracellular events that result in the assembly of an rAAV virion including encapsidation of the rAAV vector. AAV “rep” and “cap” genes refer to polynucleotide sequences encoding replication and encapsidation proteins of adeno-associated virus. AAV rep and cap are referred to herein as AAV “packaging genes.” Packaging requires either a helper virus itself or, more commonly in recombinant systems, helper virus function supplied by a helper-free system (i.e., one or more helper plasmids).

A “helper virus” for AAV refers to a virus that allows AAV (e.g., wild-type AAV) to be replicated and packaged by a mammalian cell. The helper viruses may be an adenovirus, herpesvirus, or poxvirus, such as vaccinia.

An “infectious” virion or viral particle is one that comprises a competently assembled viral capsid and is capable of delivering a polynucleotide component into a cell for which the virion is tropic. The term does not necessarily imply any replication capacity of the virus.

“Infectivity” refers to a measurement of the ability of a virion to inflect a cell. Infectivity can be expressed as the ratio of infectious viral particles to total viral particles. Infectivity is general determined with respect to a particular cell type. It can be measured both in vivo or in vitro. Methods of determining the ratio of infectious viral particle to total viral particle are known in the art. See, e.g., Grainger et al. (2005) Mol. Ther. 11:S337 (describing a TCID₅₀ infectious titer assay); and Zolotukhin et al. (1999) Gene Ther. 6:973.

The terms “parental capsid” or “parental sequence” refer to a reference sequence from which a particle capsid or sequence is derived. Unless otherwise specified, parental sequence refers to the sequence of the wild-type capsid protein of the same serotype as the engineered capsid protein.

A “replication-competent” virus (e.g., a replication-competent AAV) refers to a virus that is infectious and is also capable of being replicated in an infected cell (i.e., in the presence of a helper virus or helper virus functions). In some embodiments, the rAAV virion of the disclosure comprises a genome that lacks the rep gene, or both the rep and cap genes, and therefore is replication incompetent.

The practice of the present disclosure will employ, unless otherwise indicated, conventional techniques of tissue culture, immunology, molecular biology, cell biology and recombinant DNA, which are within the skill of the art. See, e.g., Sambrook and Russell eds. (2001) Molecular Cloning: A Laboratory Manual. 3^{rd} edition; Ausubel et al. eds. (2007) Current Protocols in Molecular Biology; Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; IRL Press (1986) Immobilized Cells and Enzymes; Perbal (1984) A Practical Guide to Molecular Cloning; Miller and Cakos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (2002) Cold Spring Harbor Laboratory Press; Sohail (2004) Gene Silencing by RNA Interference: Technology and Application (CRC Press); and Sell (2013) Stem Cells Handbook.

The terms “nucleic acid” and “polynucleotide” are used interchangeably and refer to a polymeric form of nucleotides of any length, either deoxyribonucleotides or ribonucleotides, or analogs thereof. Non-limiting examples of polynucleotides include linear and circular nucleic acids, messenger RNA (mRNA), cDNA, recombinant polynucleotides, vectors, probes, and primers. Unless otherwise specified or required, any embodiment of the invention described herein that is a polynucleotide encompasses both the double-stranded form and each of two complementary single-stranded forms known or predicted to make up the double-stranded form.

The terms “polypeptide” and “protein. “are used interchangeably herein and refer to a polymeric form of amino acids of any length, which can include genetically coded and non-genetically coded amino acids, chemically or biochemically modified or derivatized amino acids, and polypeptides having modified peptide backbones. The terms also encompass an amino acid polymer that has been modified; for example, disulfide bond formation, glycosylation, lipidation, phosphorylation, or conjugation with a labeling component.

The term “peptide” refers to a short polypeptide, e.g., a peptide having between about 4 and 30 amino acid residues.

The term “isolated” means separated from constituents, cellular and otherwise, in which the virion, cell, tissue, polynucleotide, peptide, polypeptide, or protein is normally associated in nature. For example, an isolated cell is a cell that is separated form tissue or cells of dissimilar phenotype or genotype.

As used herein. “sequence identity” or “identity” refers to the percentage of number of amino acids that are identical between a sequence of interest and a reference sequence. Generally, identity is determined by aligning the sequence of interest to the reference sequence, determining the number of amino acids that are identical between the aligned sequences, dividing that number by the total number of amino acids in the reference sequence, and multiplying the result by 100 to yield a percentage. Sequences can be aligned using various computer programs, such BLAST, available at ncbi.nlm.nih.gov. Other techniques for alignment are described in Methods in Enzymology, vol. 266: Computer Methods for Macromolecular Sequence Analysis (1996); and Meth. Mol. Biol. 70: 173-187 (1997); J. Mol. Biol. 48: 44. Skill artisans are capable of choosing an appropriate alignment method depending on various factors including sequence length, divergence, and the presence of absence of insertions or deletions with respect to the reference sequence.

“Recombinant,” as applied to a polynucleotide means that the polynucleotide is the product of various combinations of cloning, restriction or ligation steps, and other procedures that result in a construct that is distinct from a polynucleotide found in nature, or that the polynucleotide is assembled from synthetic oligonucleotides. A “recombinant” protein is a protein produced from a recombinant polypeptide. A recombinant virion is a virion that comprises a recombinant polynucleotide and/or a recombinant protein, e.g., a recombinant capsid protein.

A “gene” refers to a polynucleotide containing at least one open reading frame that is capable of encoding a particular protein after being transcribed and translated. A “gene product” is a molecule resulting from expression of a particular gene. Gene products may include, without limitation, a polypeptide, a protein, an aptamer, an interfering RNA, or an mRNA. Gene-editing systems (e.g., a CRISPR/Cas system) may be described as one gene product or as the several gene products required to make the system (e.g., a Cas protein and a guide RNA).

A “short hairpin RNA,” or shRNA, is a polynucleotide construct used to express an siRNA.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements include transcriptional regulatory sequences such as promoters and/or enhancers.

A “promoter” is a DNA sequence capable under certain conditions of binding RNA polymerase and initiating transcription of a coding region usually located downstream (in the 3′ direction) from the promoter. The term “tissue-specific promoter” as used herein refers to a promoter that is operable in cells of a particular organ or tissue, such as the cardiac tissue.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “polynucleotide cassette” refers to the portion of a vector genome between the inverted terminal repeats (ITRs). A polynucleotide cassette can comprise polynucleotide sequences encoding any genetic element whose delivery to a target cell is desired, including but not limited to a coding sequence for a gene, a promoter, or a repair template for gene editing. Unless otherwise specified, the expression cassette of an AAV vector includes only the polynucleotide between (and not including) the ITRs.

An “expression vector” is a vector comprising a coding sequence which encodes a gene product of interest used to effect the expression of the gene product in target cells. An expression vector comprises control elements operatively linked to the coding sequence to facilitate expression of the gene product.

The term “expression cassette” refers to a polynucleotide cassette comprising a coding sequence which encodes a gene product of interest used to effect the expression of the gene product in target cells. Unless otherwise specified, the expression cassette of an AAV vector includes only the polynucleotides between (and not including) the ITRs.

The term “gene delivery” or “gene transfer” as used herein refers to methods or systems for reliably inserting foreign nucleic acid sequences, e.g., DNA, into host cells. Such methods can result in transient expression of non-integrated transferred DNA, extra-chromosomal replication, and expression of transferred replicons (e.g., episomes), or integration of transferred genetic material into the genomic DNA of host cells.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid is an rAAV that includes a nucleic acid not normally included in a naturally-occurring AAV.

The terms “genetic alteration” and “genetic modification” (and grammatical variants thereof) are used interchangeably herein to refer to a process wherein a genetic element (e.g., a polynucleotide) is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a vector.

A cell is said to be “stably” altered, transduced, genetically modified, or transformed with a polynucleotide sequence if the sequence is available to perform its function during extended culture of the cell in vitro. Generally, such a cell is “heritably” altered (genetically modified) in that a genetic alteration is introduced which is also inheritable by progeny of the altered cell.

The term “transfection” is as used herein refers to the uptake of an exogenous nucleic acid molecule by a cell. A cell has been “transfected” when exogenous nucleic acid has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acid molecules into suitable host cells.

The term “transduction” is as used herein refers to the transfer of an exogenous nucleic acid into a cell by a recombinant virion, in contrast to “infection” by a wild-type virion. When infection is used with respect to a recombinant virion, the terms “transduction” and “infectious” are synonymous, and therefore “infectivity” and “transduction efficiency” are equivalent and can be determined using similar methods.

The phrase “assessed in a primate” refers to testing by methods described in the Examples or variations upon them. Assessment may be done using a population of rAAV virions having a common capsid protein screen or pooled testing by re-screening.

Unless otherwise specified, all medical terminology is given the ordinary meaning of the term used by medical professional as, for example, in Harrison's Principles of Internal Medicine, 15 ed., which is incorporated by reference in its entirety for all purposes, in particular the chapters on cardiac or cardiovascular diseases, disorders, conditions, and dysfunctions.

“Treatment,” “treating,” and “treat” are defined as acting upon a disease, disorder, or condition with an agent to reduce or ameliorate harmful or any other undesired effects of the disease, disorder, or condition and/or its symptoms.

“Administration,” “administering” and the like, when used in connection with a composition of the invention refer both to direct administration (administration to a subject by a medical professional or by self-administration by the subject) and/or to indirect administration (prescribing a composition to a patient). Typically, an effective amount is administered, which amount can be determined by one of skill in the art. Any method of administration may be used. Administration to a subject can be achieved by, for example, intravenous, intraarterial, intramuscular, intravascular, or intramyocardial delivery.

As used herein the term “effective amount” and the like in reference to an amount of a composition refers to an amount that is sufficient to induce a desired physiologic outcome (e.g., reprogramming of a cell or treatment of a disease). An effective amount can be administered in one or more administrations, applications, or dosages. Such delivery is dependent on a number of variables including the time period which the individual dosage unit is to be used, the bioavailability of the composition, the route of administration, etc. It is understood, however, that specific amounts of the compositions (e.g., rAAV virions) for any particular subject depends upon a variety of factors including the activity of the specific agent employed, the age, body weight, general health, sex, and diet of the subject, the time of administration, the rate of excretion, the composition combination, severity of the particular disease being treated and form of administration.

The terms “cardiac pathology” or “cardiac dysfunction” are used interchangeably and refer to any impairment in the heart's pumping function. This includes, for example, impairments in contractility, impairments in ability to relax (sometimes referred to as diastolic dysfunction), abnormal or improper functioning of the heart's valves, diseases of the heart muscle (sometimes referred to as cardiomyopathies), diseases such as angina pectoris, myocardial ischemia and/or infarction characterized by inadequate blood supply to the heart muscle, infiltrative diseases such as amyloidosis and hemochromatosis, global or regional hypertrophy (such as may occur in some kinds of cardiomyopathy or systemic hypertension), and abnormal communications between chambers of the heart.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit risk ratio.

The term “purified” as used herein refers to material that has been isolated under conditions that reduce or eliminate the presence of unrelated materials, i.e., impurities, including native materials from which the material is obtained. For example, purified rAAV vector DNA is preferably substantially free of cell or culture components, including tissue culture components, contaminants, and the like.

The terms “regenerate,” “regeneration” and the like as used herein in the context of injured cardiac tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises generation of cardiomyocytes.

The term “therapeutic gene” as used herein refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that partially or wholly correct a genetic deficiency in a cell or mammal.

As used herein, the term “functional cardiomyocyte” refers to a differentiated cardiomyocyte that is able to send or receive electrical signals. In some embodiments, a cardiomyocyte is said to be a functional cardiomyocyte if it exhibits electrophysiological properties such as action potentials and/or Ca transients.

As used herein, a “differentiated non-cardiac cell” can refer to a cell that is not able to differentiate into all cell types of an adult organism (i.e., is not a pluripotent cell), and which is of a cellular lineage other than a cardiac lineage (e.g., a neuronal lineage or a connective tissue lineage). Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones, and connective tissue in an organism, but not germ cells.

As used herein, the term “totipotent” means the ability of a cell to form all cell lineages of an organism. For example, in mammals, only the zygote and the first cleavage stage blastomeres are totipotent.

As used herein, the term “pluripotent” means the ability of a cell to form all lineages of the body or soma. For example, embryonic stem cells are a type of pluripotent stem cells that are able to form cells from each of the three germs layers, the ectoderm, the mesoderm, and the endoderm. Pluripotent cells can be recognized by their expression of markers such as Nanog and Rex1.

As used herein, the term “multipotent” refers to the ability of an adult stem cell to form multiple cell types of one lineage. For example, hematopoietic stem cells are capable of forming all cells of the blood cell lineage, e.g., lymphoid and myeloid cells.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “reprogramming” or “transdifferentiation” refers to the generation of a cell of a certain lineage (e.g., a cardiac cell) from a different type of cell (e.g., a fibroblast cell) without an intermediate process of dc-differentiating the cell into a cell exhibiting pluripotent stem cell characteristics.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium, or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac a-actinin. The term “cardiomyocyte” as used herein is an umbrella term comprising any cardiomyocyte subpopulation or cardiomyocyte subtype, e.g., atrial, ventricular and pacemaker cardiomyocytes.

The term “cardiomyocyte-like cells is intended to mean cells sharing features with cardiomyocytes, but which may not share all features. For example, a cardiomyocyte-like cell may differ from a cardiomyocyte in expression of certain cardiac genes.

The term “culture” or “cell culture” means the maintenance of cells in an artificial, in vitro environment. A “cell culture system” is used herein to refer to culture conditions in which a population of cells may be grown as monolayers or in suspension. “Culture medium” is used herein to refer to a nutrient solution for the culturing, growth, or proliferation of cells. Culture medium may be characterized by functional properties such as, but not limited to, the ability to maintain cells in a particular state (e.g, a pluripotent state, a quiescent state, etc.), to mature cells—in some instances, specifically, to promote the differentiation of progenitor cells into cells of a particular lineage (e.g., a cardiomyocyte).

As used herein, the term “expression” or “express” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample.

The term “induced cardiomyocyte” or the abbreviation “iCM” refers to a non-cardiomyocyte (and its progeny) that has been transformed into a cardiomyocyte (and/or cardiomyocyte-like cell). The methods of the present disclosure can be used in conjunction with any methods now known or later discovered for generating induced cardiomyocytes, for example, to enhance other techniques.

The term “induced pluripotent stem cell-derived cardiomyocytes” as used herein refers to human induced pluripotent stem cells that have been differentiated into cardiomyocyte-like cells. Exemplary methods for prepared iPS-CM cells are provided by Karakikes et al. Circ. Res. 2015 Jun. 19; 117(1): 80-88.

The terms “human cardiac fibroblast” and “mouse cardiac fibroblast” as used herein refer to primary cell isolated from the ventricles of the adult heart of a human or mouse, respectively, and maintain in culture ex vivo.

The term “non-cardiomyocyte” as used herein refers to any cell or population of cells in a cell preparation not fulfilling the criteria of a “cardiomyocyte” as defined and used herein. Non-limiting examples of non-cardiomyocytes include somatic cells, cardiac fibroblasts, non-cardiac fibroblasts, cardiac progenitor cells, and stem cells.

As used herein “reprogramming” includes transdifferentiation, dedifferentiation and the like.

As used herein, the term “reprogramming efficiency” refers to the number of cells in a sample that are successfully reprogrammed to cardiomyocytes relative to the total number of cells in the sample.

The term “reprogramming factor” as used herein includes a factor that is introduced for expression in a cell to assist in the reprogramming of the cell from one cell type into another. For example, a reprogramming factor may include a transcription factor that, in combination with other transcription factors and/or small molecules, is capable of reprogramming a cardiac fibroblast into an induced cardiomyocyte. Unless otherwise clear from context, a reprogramming factor refers to a polypeptide that can be encoded by an AAV-delivered polynucleotide. Reprogramming factors may also include small molecules.

The term “stem cells” refer to cells that have the capacity to self-renew and to generate differentiated progeny. The term “pluripotent stem cells” refers to stem cells that can give rise to cells of all three germ layers (endoderm, mesoderm, and ectoderm), but do not have the capacity to give rise to a complete organism.

As used herein, the term “equivalents thereof” in reference to a polypeptide or nucleic acid sequence refers to a polypeptide or nucleic acid that differs from a reference polypeptide or nucleic acid sequence, but retains essential properties (e.g., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, deletions, additions, fusions, and truncations in the polypeptide encoded by the reference sequence. Generally, differences are limited so that the sequences of the reference polypeptide and the variant are closely similar overall and, in many regions, identical.

As used herein, the term “progenitor cell” refers to a cell that is committed to differentiate into a specific type of cell or to form a specific type of tissue. A progenitor cell, like a stem cell, can further differentiate into one or more kinds of cells, but is more mature than a stem cell such that it has a more limited/restricted differentiation capacity.

The term “genetic modification” refers to a permanent or transient genetic change induced in a cell following introduction of new nucleic acid (i.e., nucleic acid exogenous to the cell). Genetic change can be accomplished by incorporation of the new nucleic acid into the genome of the cardiac cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

The term “stem cells” refer to cells that have the capacity to self-renew and to generate differentiated progeny. The term “pluripotent stem cells” refers to stem cells that can give rise to cells of all three germ layers (endoderm, mesoderm, and ectoderm), but do not have the capacity to give rise to a complete organism. In some embodiments, the compositions for inducing cardiomyocyte phenotype can be used on a population of cells to induce reprogramming. In other embodiments, the compositions induce a cardiomyocyte phenotype.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency. See, for example, Takahashi et al. (2007) Cell 131(5):861-872, Kim et al. (2011) Proc. Natl. Acad. Sci. 108(19): 7838-7843, Sell (2013) Stem Cells Handbook.

The term “transduction efficiency” refers to the percentage of cells transduced with at least one AAV genome. For example, if 1×10⁶ cells are exposed to a virus and 0.5×10⁶ cells are determined to contain at least one copy of the AAV genome, then the transduction efficiency is 50%. An illustrative method for determining transduction efficiency is flow cytometry. For example, the percentage of GFP+ cells is a measure of transduction efficiency when the AAV genome comprises a polynucleotide encoding green fluorescence protein (GFP).

The term “selectivity” refers to the ratio of transduction efficiency for one cell type over another, or over all other cell types.

The term “infectivity” refers to the ability of an AAV virion to infect a cell, in particularly an in vivo cell. Infectivity therefore is a function of, at least, biodistribution and neutralizing antibody escape.

Unless stated otherwise, the abbreviations used throughout the specification have the following meanings: AAV, adeno-associated virus, rAAV, recombinant adeno-associated virus; AHCF, adult human cardiac fibroblast; APCF, adult pig cardiac fibroblast, a-MHC-GFP; alpha-myosin heavy chain green fluorescence protein; CF, cardiac fibroblast; cm, centimeter; CO, cardiac output; EF, ejection fraction; FACS, fluorescence activated cell sorting; GFP, green fluorescence protein; GMT, Gata4, Mef2c and Tbx5; GMTc, Gata4, Mef2c, Tbx5, TGF-βi, WNTi; GO, gene ontology; hCF, human cardiac fibroblast; iCM, induced cardiomyocyte; kg, kilogram: pg, microgram; pl, microliter, mg, milligram; ml, milliliter; MI, myocardial infarction; msec, millisecond; min, minute; MyAMT, Myocardin, AscII, Mef2c and Tbx5; MyA, Myocardin and AscII; MyMT, Myocardin, Mef2c and Tbx5; MyMTc, Myocardin, Mef2c, Tbx5, TGF-βi, WNTi; MR1, magnetic resonance imaging: PBS, phosphate buffered saline; PBST, phosphate buffered saline, triton: PFA, paraformaldehyde; qPCR, quantitative polymerase chain reaction; qRT-PCR, quantitative reverse transcriptase polymerase chain reaction; RNA, ribonucleic acid; RNA-seq. RNA sequencing: RT-PCR, reverse transcriptase polymerase chain reaction; see, second; SV, stroke volume; TGF-β, transforming growth factor beta; TGF-βi, transforming growth factor beta inhibitor, WNT, wingless-Int; WNTi, wingless-Int inhibitor; YFP, yellow fluorescence protein; 4F, Gata4, Mef2c, TBX5, and Myocardin; 4Fe, Gata4, Mef2c, TBX5, and Myocardin+TGF-βi and WNTi; 7F, Gata4, Mef2c, and Tbx5, Essrg, Myocardin, Zfpm2, and Mesp1; 7Fe, Gata4, Mef2c, and Tbx5, Essrg, Myocardin. Zfpm2, and Mesp1+TGF-β and WNTi.

The amino acid abbreviations used herein are abbreviations commonly used in the art, and as follows (wherein the last letter indicates the abbreviation used):
Alanine—Ala—A
Arginine—Arg—R
Asparagine—Asn—N
Aspartic acid—Asp—D
Cysteine—Cys—C
Glutamic acid—Glu—E
Glutamine—Gln—Q
Glycine—Gly—G
Histidine—His—H
Isoleucine—Ile—I
Leucine—Leu—L
Lysine—Lys—K
Methionine—Met—M
Phenylalanine—Phe—F
Proline—Pro—P
Serine—Ser—S
Threonine—Thr—T
Tryptophan—Trp—W
Tyrosine—Tyr—Y
Valine—Val—V

Reference to amino acid substitutions are in the format commonly used in the art. E.g., reference to “N452K” substitution, indicates that at position number 452 of the reference sequence, the wild type amino acid in front of the number (here “N”) has been substituted with the amino acid following the number (here “K”).

The term “conservative amino-acid substitutions” refers to substitutions of amino acid residues that share similar sidechain physical properties with the residues being substituted. Conservative substitutions include polar for polar residues, non-polar for non-polar residues, hydrophobic for hydrophobic residues, small for small residues, and large for large residues. Conservative substitutions further comprise substitutions within the following groups: {S, T}, {A, G}, {F, Y}, {R, H, K, N, E}, {S, T, N, Q}, {C, U, O, P, A}, and {A, V, I, L, M, F, Y, W}.

SEQUENCES

>ZC377 (SEQ ID NO: 488):
MAADGYLPDWLEDNISEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYEDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTIKGSGQNQQTLKFSVAGPSNMAVQGRNYIPGPSYRQQRVSTTVTQNNNSEFAWP
GASSWALNGRNSLMNPGPAMASHKEGEDRFFPLSGSLIFQKQGTGRDNVDADKVMITNEEEIKT
TNPVATESYGQVATNHQANYGQAQTGWVQNQGILPGMVWQDRDVYIQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC378 (SEQ ID NO: 489):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINASGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHQANYGQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC379 (SEQ ID NO: 490):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDQSQAVGRSSFYCLEYFPSQMLRTGNNFQFSYEFENVPFHSSYAHSQSLDRLMNPLID
QYLYYLSKTINGTGQNQQTIKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQANYGQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC380 (SEQ ID NO: 491):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGLNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQANYGQAQTGWVQNQGILPGMVWQDRDVYLQGPIWAKIPHTDQNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC381 (SEQ ID NO: 492):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTIANDNKLIQTLKFSVAGPSNMAVQGRNYIPGPSYRQQRVSTTVTQNNNSEFAWPG
ASSWALNGRNSLMNPGPAMASHKEGEDRFFPLSGSLIFGKQGTGRDNVDADKVMITNEEEIKTT
NPVATESYGQVATNHQANYGQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC382 (SEQ ID NO: 493):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPCKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NEKLENIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTIVNDNKVIQTLKFSVAGPSNMAVQGRNYIPGPSYRQQRVSTTVTQNNNSEFAWPG
ASSWALNGRNSLMNPGPAMASHKEGEDRFFPLSGSLIFGKQGTGRDNVDADKVMITNEEEIKTT
NPVATESYGQVATNHQANYGQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC383 (SEQ ID NO: 494):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNHQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQANYGQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC384 (SEQ ID NO: 495):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTIANDNKVIQTLKFSVAGPSNMAVQGRNYIPGPSYRQQRVSTTVTQNNNSEFAWPG
ASSWALNGRNSLMNPGPAMASHKEGEDRFFPLSGSLIFGKQGTGRDNVDADKVMITNEEEIKTT
NPVATESYGQVATNHQANYGQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC385 (SEQ ID NO: 496):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHTSFQAQAQTQWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC386 (SEQ ID NO: 497):
MAADGYLPDWLEDNLSEGIREWWALKPGAPQPKANQQHQDNARGLVLPGYKYLGPGNGLDK
GBPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKESVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHCSAQAQAQTGWVQNQGILPGMVWQDRDVYLQGPIWAKIPHTDGNFH
PSPLMGGFGMKHPPPQILIKNTPVPADPPTAFNKDKLNSFITQYSTQQVSVEIEWELQKENSKRW
NPEIQYTSNYYKSNNVEFAVNTEGVYSEPRPIGTRYLTRNL
>ZC387 (SEQ ID NO: 498):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHVDSLRIAQTGWVQNQGILPGMVWQDRDVYLQGPIWAKIPHTDGNFHP
SPLMGGFGMKHPPPQILIKNTPVPADPPTAFNKDKINSFITQYSTGQVSVEIEWELQKENSKRWN
PEIQYTSNYYKSNNVEFAVNTEGVYSEPRPIGTRYLTRNL
>ZC388 (SEQ ID NO: 499):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVQRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNRQTAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC389 (SEQ ID NO: 500):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NFKLFNIQVKEVTDNNGVKTIANNLTSTVQVFIDSDYQLPYVLGSAFEGCLPPPPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHTGTSIIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC390 (SEQ ID NO: 501):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHLSNFNSAQTGWVQNQGILPGMVWQDRDVYLQGPIWAKIPHTDGNFH
PSPIMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC391 (SEQ ID NO: 502):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHCTLNSIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC392 (SEQ ID NO: 503):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVADVQQKPGSQIQTQWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC393 (SEQ ID NO: 504):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNMNRVNAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC394 (SEQ ID NO: 505):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNNVISGAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC395 (SEQ ID NO: 506):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVQRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSNSVQSAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC396 (SEQ ID NO: 507):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHQSPIAQAQAQTGWVQNQGILPGMVWQDRDVYLQGPIWAKIPHTDGN
FHPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC397 (SEQ ID NO: 508):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTQNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHLSKVFDAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC398 (SEQ ID NO: 509):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQSAITQAQAQTQWVQNQGILPGMVWQDRDVYLQGPIWAKIPHTDGN
FHPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC399 (SEQ ID NO: 510):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSSTFQGAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC400 (SEQ ID NO: 511):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNSIQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC401 (SEQ ID NO: 512):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFQGNLGRAVFQAK
KRLLEPLGLVEEAAKTAPCKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHMMTTARAQTGWVQNQGILPGMVWQDRDVYLQGPIWAKIPHTDQNE
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC402 (SEQ ID NO: 513):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQGAYAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC403 (SEQ ID NO: 514):
MAADGYLPDWLEDNLSEGIREWWALKPGAPQPKANQQHQDNARGLVLPGYKYLGPGNQLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVALNKQSAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC404 (SEQ ID NO: 515):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHENTVSIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC405 (SEQ ID NO: 516):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKBVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHVSSFTSÅQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC406 (SEQ ID NO: 517):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHPSIHQGAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC407 (SEQ ID NO: 518):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSTINFRAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC408 (SEQ ID NO: 519):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFCKQGTGRDNVDADKVMITNEEEIKT
TNPVATESYGQVATNHQHYSAQAQAQTGWVQNQGILPGMVWQDRDVYLQGPIWAKIPHTDG
NFHPSPLMGGFGMKHPPPQILIKNTPVPADPPTAFNKDKLNSFITQYSTGQVSVEIEWELQKENS
KRWNPEIQYTSNYYKSNNVEFAVNTEGVYSEPRPIGTRYLTRNL
>ZC409 (SEQ ID NO: 520):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNKQTAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC410 (SEQ ID NO: 521):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
QYLTLNDQSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHSSIFNSAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC411 (SEQ ID NO: 522):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHAGNYNNAQTGWVQNQGILPGMVWQDRDVYLQGPIWAKIPHTDGNF
HPSPLMGGFQMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC412 (SEQ ID NO: 523):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKBVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVAEVQQSSMSQAQTDWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC413 (SEQ ID NO: 524):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLENIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVAANVQSAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC414 (SEQ ID NO: 525):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNYQQAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC415 (SEQ ID NO: 526):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHQSVQGAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC416 (SEQ ID NO: 527):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHGSILTHAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC417 (SEQ ID NO: 528):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQLFSKNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC418 (SEQ ID NO: 529):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKBVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVAANMQSAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC419 (SEQ ID NO: 530):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEBAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNQQIAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC420 (SEQ ID NO: 531):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNTYHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTGQVSVEIEWELQKENS
KRWNPEIQYTSNYYKSNNVEFAVNTEGVYSEPRPIGTRYLTRNL
>ZC421 (SEQ ID NO: 532):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHCDPLHIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC422 (SEQ ID NO: 533):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHTSVISIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC423 (SEQ ID NO: 534):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQLASAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC424 (SEQ ID NO: 535):
MAADGYLPDWLEDNLSEGIREWWALKPGAPQPKANQQHQDNARGLVLPGYKYLGPGNGLDK
GBPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQVTSAQAQAQTGWVQNQGILPGMVWQDRDVYLQGPIWAKIPHTDG
NFHPSPLMGGFGMKHPPPQILIKNTPVPADPPTAFNKDKINSFITQYSTGQVSVEIEWELQKENS
KRWNPEIQYTSNYYKSNNVEFAVNTEGVYSEPRPIGTRYLTRNL
>ZC425 (SEQ ID NO: 536):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHHSRVEIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC426 (SEQ ID NO: 537):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVEQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHTSFTWTAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC427 (SEQ ID NO: 538):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFCKQGTGRDNVDADKVMITNEEEIKT
TNPVATESYGQVATNHQSAPTQAQAQTGWVQNQGILPGMVWQDRDVYLQGPIWAKIPHTDGN
FHPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC428 (SEQ ID NO: 539):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNSTYLGAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC429 (SEQ ID NO: 540):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQIAQAQAQTGWVQNQGILPGMVWQDRDVYLQGPIWAKIPHTDQNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC430 (SEQ ID NO: 541):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVEQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQAISAQAQAQTGWVQNQGILPGMVWQDRDVYLQGPIWAKIPHTDGN
FHPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC431 (SEQ ID NO: 542):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASQGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHLSVVYNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC432 (SEQ ID NO: 543):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHMHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTGQVSVEIEWELQKENS
KRWNPEIQYTSNYYKSNNVEFAVNTEGVYSEPRPIGTRYLTRNL
>ZC433 (SEQ ID NO: 544):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVQRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHETSRLNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC434 (SEQ ID NO: 545):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVAFNWQSAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC435 (SEQ ID NO: 546):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHNTVMLGAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC436 (SEQ ID NO: 547):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHESSMLNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC437 (SEQ ID NO: 548):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHASITSSAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC438 (SEQ ID NO: 549):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVARNEQSAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC439 (SEQ ID NO: 550):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHANLYQMAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC440 (SEQ ID NO: 551):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQFATAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC441 (SEQ ID NO: 552):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNORNSLMNPGPAMASHKEGEDRFFPLSGSLIFGKQGTGRDNVDADKVMITNEEEIKT
TNPVATESYGQVATNFNHQSAQAQAQTGWVQNQGILPGMVWQDRDVYLQGPIWAKIPHTDGN
FHPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC442 (SEQ ID NO: 553):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHMSHQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC443 (SEQ ID NO: 554):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQWMSAQAQAQTGWVQNQGILPGMVWQDRDVYLQGPIWAKIPHTDG
NFHPSPLMGGFGMKHPPPQILIKNTPVPADPPTAFNKDKINSFITQYSTGQVSVEIEWELQKENS
KRWNPEIQYTSNYYKSNNVEFAVNTEGVYSEPRPIGTRYLTRNL
>ZC444 (SEQ ID NO: 555):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQSGQQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC445 (SEQ ID NO: 556):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVEQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSSAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC446 (SEQ ID NO: 557):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHTTKTMFAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC447 (SEQ ID NO: 558):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSSIIYSAQTGWVQNQGILPGMVWQDRDVYLQGPIWAKIPHIDGNFHPS
PLMGGFGMKHPPPQILIKNTPVPADPPTAFNKDKLNSFITQYSTGQVSVEIEWELQKENSKRWNP
EIQYTSNYYKSNNVEFAVNTEGVYSEPRPIGTRYLTRNL
>ZC448 (SEQ ID NO: 559):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHMLLKSNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC449 (SEQ ID NO: 560):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKESVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHESMQAQAQTGWVQNQGILPGMVWQDRDVYLQGPIWAKIPHTDGNF
HPSPLMGGFGMKHPPPQILIKNTPVPADPPTAFNKDKINSFITQYSTGQVSVEIEWELQKENSKR
WNPEIQYTSNYYKSNNVEFAVNTEGVYSEPRPIGTRYLTRNL
>ZC450 (SEQ ID NO: 561):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKESVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQMLSAQAQAQTGWVQNQGILPGMVWQDRDVYLQGPIWAKIPHTDG
NFHPSPLMGGFGMKHPPPQILIKNTPVPADPPTAFNKDKLNSFITQYSTGQVSVEIEWELQKENS
KRWNPEIQYTSNYYKSNNVEFAVNTEGVYSEPRPIGTRYLTRNL
>ZC451 (SEQ ID NO: 562):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKESVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSGRDSYAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC452 (SEQ ID NO: 563):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHINVISGAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC453 (SEQ ID NO: 564):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHVSNQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC454 (SEQ ID NO: 565)
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNTKLAIAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC455 (SEQ ID NO: 566):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVEQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSSSYNNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC456 (SEQ ID NO: 567):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNATHQSAQAQAQTGWVQNQGILPGMVWQDRDVYLQGPIWAKIPHTDG
NFHPSPLMGGFGMKHPPPQILIKNTPVPADPPTAFNKDKLNSFITQYSTGQVSVEIEWELQKENS
KRWNPEIQYTSNYYKSNNVEFAVNTEGVYSEPRPIGTRYLTRNL
>ZC457 (SEQ ID NO: 568):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHLRDNISAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC458 (SEQ ID NO: 569):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHSSFSVGAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC459 (SEQ ID NO: 570):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEBEIKT
TNPVATESYGQVATNHVNRNLSAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC460 (SEQ ID NO: 571):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHHNPSINAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC461 (SEQ ID NO: 572):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQDARAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC462 (SEQ ID NO: 573):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHICDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKBVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNDQRAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC463 (SEQ ID NO: 574):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNVQTAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC464 (SEQ ID NO: 575):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVAPNRQSAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC465 (SEQ ID NO: 576):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNRQIAQAQAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC466 (SEQ ID NO: 577):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHEDNIRRAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC467 (SEQ ID NO: 578):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHNRNGLLAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC468 (SEQ ID NO: 579):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHESTSVRAQTGWVQNQGILPGMVWQDRDVYLQGPIWAKIPHTDGNFH
PSPLMGGFGMKHPPPQILIKNTPVPADPPTAFNKDKLNSFITQYSTQQVSVEIEWELQKENSKRW
NPEIQYTSNYYKSNNVEFAVNTEGVYSEPRPIGTRYLTRNL
>ZC469 (SEQ ID NO: 580):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASQGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHNIRTEMAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC470 (SEQ ID NO: 581):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHQTLFNSAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC471 (SEQ ID NO: 582):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHHSWQAQAQTGWVQNQGILPGMVWQDRDVYLQGPIWAKIPHTDQNF
HPSPLMGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL
>ZC472 (SEQ ID NO: 583):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFQKQGTGRDNVDADKVMITNEEEIKT
TNPVATESYGQVATNHSTKSLIAQTGWVQNQGILPGMVWQDRDVYLQGPIWAKIPHTDQNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC473 (SEQ ID NO: 584):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSEGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNQRNSLMNPGPAMASHKEGEDRFFPLSGSLIFGKQGTGRDNVDADKVMITNEEEIKT
TNPVATESYGQVATNHQKLLVNAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC474 (SEQ ID NO: 585):
MAADGYLPDWLEDNLSEGIREWWALKPGAPQPKANQQHQDNARGLVLPGYKYLGPGNGLDK
GBPVNAADAAALEHDKAYDQQLKAGDNPYLKYNHADAEFQERLKEDTSFGGNLGRAVEQAK
KRLLEPLGLVEEAAKTAPGKKRPVEQSPQEPDSSAGIGKSGAQPAKKRINFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
QYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHLSVSSIAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC475 (SEQ ID NO: 586):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHESPRDWQRLINNNWGFRPKRL
NFKLFNIQVKBVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHVSNLYGAQTGWVQNQGILPGMVWQDRDVYLQGPIWAKIPHTDGNFH
PSPLMGGFGMKHPPPQILIKNTPVPADPPTAFNKDKLNSFITQYSTGQVSVEIEWELQKENSKRW
NPEIQYTSNYYKSNNVEFAVNTEGVYSEPRPIGTRYLTRNL
>ZC476 (SEQ ID NO: 587):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNRQMAQAQAQTGWVQNQGILPGMVWQDRDVYLQGPIWAKIPHTDGNF
HPSPLMGGFGMKHPPPQILIKNTPVPADPPTAFNKDKLNSFITQYSTGQVSVEIBWELQKENSKR
WNPEIQYTSNYYKSNNVEFAVNTEGVYSEPRPIGTRYLTRNL
>ZC477 (SEQ ID NO: 588):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NEKLFNIQVKEVTDNNGVKTIANNLTSTVQVFTDSDYQLPYVLGSAHEGCLPPFPADVEMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEEEIKT
TNPVATESYGQVATNHEDIIRSAQTGWVQNQGILPGMVWQDRDVYLQGPIWAKIPHTDGNFHP
SPLMGGFGMKHPPPQILIKNTPVPADPPTAFNKDKLNSFITQYSTGQVSVEIEWELQKENSKRWN
PEIQYTSNYYKSNNVEFAVNTEGVYSEPRPIGTRYLTRNL
>ZC478 (SEQ ID NO: 589):
MAADGYLPDWLEDNLSEGIREWWALKPGAPQPKANQQHQDNARGLVLPGYKYLGPGNGLDK
GEPVNAADAAALEHDKAYDQQLKAGDNPYLKYNHADAEFQERLKEDTSFGGNLGRAVFQAK
KRLLEPLGLVEEAAKTAPGKKRPVEQSPQEPDSSAGIGKSGAQPAKKRLNFGQTGDTESVPDPQ
PIGEPPAAPSGVGSLTMASGGGAPVADNNEGADGVGSSSGNWHCDSQWLGDRVITTSTRTWAL
PTYNNHLYKQISNSTSGGSSNDNAYFGYSTPWGYFDFNRFHCHFSPRDWQRLINNNWGFRPKRL
NFKLFNIQVKEVTDNNGVKTIANNLTSTVQVFTDSDYQLPYVLGSAHEGCLPPFPADVFMIPQY
GYLTLNDGSQAVGRSSFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVSTTVTQNNNSEFAWP
GASSWALNGRNSLMNPGPAMASHKEGEDRFFPLSGSLIFGKQGTGRDNVDADKVMITNEBEIKT
TNPVATESYGQVATNHCSTSIRAQTGWVQNQGILPGMVWQDRDVYLQGPIWAKIPHTDGNFHP
SPLMGGFGMKHPPPQILIKNTPVPADPPTAFNKDKINSFITQYSTGQVSVEIEWELQKENSKRWN
PEIQYTSNYYKSNNVEFAVNTEGVYSEPRPIGTRYLTRNL

### Additional Sequences

| | | SEQ |
|---|---|---|
| | | ID |
| Description | Sequence | NO |
| Recombinant | MAADGYLPDWLEDNLSEGIREWWALKPGAPQPKANQQHQDNARG | 705 |
| AAV capsid | LVLPGYKYLGPGNGLDKGEPVNAADAAALEHDKAYDQQLKAGDN | |
| protein ZC373 | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHENTVSIAQTGWVQN | |
| | QGILPQMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| Recombinant | MAADGYLPDWLEDNLSEGIREWWALKPGAPQPKANQQHQDNARG | 706 |
| AAV capsid | LVLPQYKYLQPGNGLDKGEPVNAADAAALEHDKAYDQQLKAGDN | |
| protein ZC374 | PYLKYNHADAEFQERLKEDTSFGGNLGRAVFQAKKRLLEPLGLVEE | |
| | AAKTAPGKKRPVEQSPQEPDSSAGIGKSGAQPAKKRINFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQTLFNSAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| Recombinant | MAADGYLPDWLEDNLSEGIREWWALKPGAPQPKANQQHQDNARG | 707 |
| AAV capsid | LVLPGYKYLGPGNGLDKGEPVNAADAAALEHDKAYDQQLKAGDN | |
| protein ZC375 | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHNSTYLGAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| Recombinant | MAADGYLPDWLEDNLSEGIREWWALKPGAPQPKANQQHQDNARG | 708 |
| AAV capsid | LVLPGYKYLGPGNGLDKGEPVNAADAAALEHDKAYDQQLKAGDN | |
| protein ZC376 | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHGSILTHAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| AAV capsid | MAADGYLPDWLEDNLSEGIREWWALKPGAPQPKANQQHQDNARG | 709 |
| protein ACE5 | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIIGSGQNQQTL | |
| | KFSVAGPSNMAVQGRNYIPGPSYRQQRVSTTVTQNNNSEFAWPGAS | |
| | SWALNGRNSLMNPGPAMASHKEGEDRFFPLSGSLIFGKQGTGRDNV | |
| | DADKVMITNEEEIKTTNPVATESYGQVATNHQANYGQAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| AAV capsid | MAADGYLPDWLEDNLSEGIREWWALKPGAPQPKANQQHQDNARG | 710 |
| protein ACE10 | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTINGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHEDNIRSAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| Recombinant | X₁-X₂-X₃-X₄- | 711 |
| AAV capsid | wherein X₁ is S, T or N; X₂ is T, L, or I; X₃ is V, F, Y, or L; and X₄ is A, N, | |
| protein partial | L or T | |
| VR-VIII | | |
| Recombinant | NTVS | 712 |
| AAV capsid | | |
| protein partial | | |
| ZC373 VR-VIII | | |
| Recombinant | TLFN | 713 |
| AAV capsid | | |
| protein partial | | |
| ZC374 VR-VIII | | |
| Recombinant | STYL | 714 |
| AAV capsid | | |
| protein partial | | |
| ZC375 VR-VIII | | |
| Recombinant | SILT | 715 |
| AAV capsid | | |
| protein partial | | |
| ZC376 VR-VIII | | |
| Recombinant | MTTA | 716 |
| AAV capsid | | |
| protein partial | | |
| ZC531 VR-VIII | | |
| Recombinant | STSI | 717 |
| AAV capsid | | |
| protein partial | | |
| ZC533 VR-VIII | | |
| Recombinant | -X₁-X₂-X₃-X₄-X₅-X₆ | 718 |
| AAV capsid | wherein X₁ is Q, E, N, G, M, or C; X₂ is S, N, T, or M; X₃ is A, T, L, I, or S; | |
| protein partial | X₄ is Q, V, F, Y, L, or I; X₅ is A, S, N, L, T, or I; and X₆ is I, S, Q, G, H, or | |
| VR-VIII | R | |
| Recombinant | ENTVSI | 719 |
| AAV capsid | | |
| protein partial | | |
| ZC373 VR-VIII | | |
| Recombinant | QTLFNS | 720 |
| AAV capsid | | |
| protein partial | | |
| ZC374 VR-VIII | | |
| Recombinant | NSTYLG | 721 |
| AAV capsid | | |
| protein partial | | |
| ZC375 VR-VIII | | |
| Recombinant | GSILTH | 722 |
| AAV capsid | | |
| protein partial | | |
| ZC376 VR-VIII | | |
| Recombinant | MMTTAR | 723 |
| AAV capsid | | |
| protein partial | | |
| ZC531 VR-VID | | |
| Recombinant | CSTSIR | 724 |
| AAV capsid | | |
| protein partial | | |
| ZC532 VR-VIII | | |
| Recombinant | EDNIRS | 725 |
| AAV capsid | | |
| protein partial | | |
| ZC536 VR-VIII | | |
| Recombinant | ATNHEDNIRSAQTG | 726 |
| AAV capsid | | |
| protein ZC536 | | |
| VR-VIII | | |
| Recombinant | KGSGQNQQT | 727 |
| AAV capsid | | |
| protein VR-IV | | |
| Recombinant | ATNH-X₁-X₂-X₃-X₄-X₅-X₆-AQTG | 728 |
| AAV capsid | X₁ is Q, E, N, G, M, C, V, or T; X₂ is S, N, T, M, G, or D; X₃ is A, T, L, I, | |
| protein partial | K, S, N or V; X₄ is Q, V, F, Y, L, T, S, I, R, or Q; X₅ is A, S, N, L, T, I, or | |
| VR-VIII | R, and X₆ is Q, I, S, G, H or R | |
| Recombinant | -X₁-X₂-X₃-X₄-X₅-X₆-X₇-X₈-X₉- | 729 |
| AAV capsid | wherein X₁ is K, G, S or V; X₂ is Y, Q or I; X₃ is H, W, V, or I; X₄ is K or | |
| protein VR-IV | N; X₅ is S, G or I; X₆ is G or R; X₇ is A, P or V; X₈ is A or R; and/or X₉ is Q | |
| | or D | |
| Recombinant | -X₁-X₂-X₃-X₄-X₅-X₆-X₇-X₈-X₉- | 730 |
| AAV capsid | wherein X₁ is K and X₂-X₉ are any amino acid | |
| protein VR-IV | | |
| Recombinant | -X₁-X₂-X₃-X₄- | 731 |
| AAV capsid | wherein X₁ is S, N, T, M, G, or D; X₂ is A, T, L, I, K, S, N or V; X₃ is Q, V, | |
| protein partial | F, Y, L, T, S, I, R, or Q; and X₄ is A, S, N, L, T, I, or R | |
| VR-VIII | | |
| Recombinant | -X₁-X₂-X₃-X₄- | 732 |
| AAV capsid | wherein X₁ is S, N, T, M, G, or D; X₂ is T, L, I, K, S, N or V; X₃ is V, F, Y, | |
| protein partial | L, T, S, I, R, or Q; and X₄ is A, S, N, L, T, I, or R. | |
| VR-VIII | | |
| Recombinant | -X₁-X₂-X₃-X₄- | 733 |
| AAV capsid | wherein X₁ is S, N, M, or T; X₂ is A, T, L, or I; X₃ is Q, V, F, Y, T, S, or L; | |
| protein partial | and X₄ is A, S, N, L, I, or T (SEQ ID NO: 733) | |
| VR-VIII | | |
| Recombinant | -X₁-X₂-X₃-X₄- | 734 |
| AAV capsid | wherein X₁ is S, N, M, or T; X₂ is T, L, or I; X₃ is V, F, Y, T, S, or L; and X₄ | |
| protein partial | is A, S, N, L, I, or T | |
| VR-VIII | | |
| Recombinant | -X₁-X₂-X₃-X₄-X₅-X₆ | 735 |
| AAV capsid | wherein X₁ is Q, E, N, G, M, C, V, or T; X₂ is S, N, T, M, G, or D; X₃ is A, | |
| protein partial | T, L, I, K, S, N or V; X₄ is V, F, Y, L, T, S, I, R, or Q; X₅ is A, S, N, L, T, I, | |
| VR-VIII | or R, and X₆ is Q, I, S, G, H or R | |
| Recombinant | -X₁-X₂-X₃-X₄-X₅-X₆ | 736 |
| AAV capsid | wherein X₁ is Q, E, N, G, M, C, V, or T; X₂ is S, N, T, M, G, or D; X₃ is T, | |
| protein partial | L, I, K, S, N or V; X₄ is V, F, Y, L, T, S, I, R, or Q; X₅ is A, S, N, L, T, I, or | |
| VR-VIII | R, and X₆ is I, S, G, H or R | |
| Recombinant | -X₁-X₂-X₃-X₄-X₅-X₆ | 737 |
| AAV capsid | wherein X₁ is Q, B, N, M, C, or G; X₂ is S, N, M, or T; X₃ is A, T, L, or I; X₄ | |
| protein partial | is Q, V, F, Y, T, S, or L; X₅ is A, S, N, L, I, or T; and X₆ is I, S, G, R, or H | |
| VR-VIII | | |
| Recombinant | -X₁-X₂-X₃-X₄-X₅-X₆ | 738 |
| AAV capsid | wherein X₁ is E, N, M, C, or G; X₂ is S, N, M, or T; X₃ is T, L, or I; X₄ is V, | |
| protein partial | F, Y, T, S, or L; X₅ is A, S, N, L, I, or T; and X₆ is I, S, G, R, or H | |
| VR-VIII | | |
| Recombinant | ATNH-X₁-X₂-X₃-X₄-X₅-X₆-AQTG | 739 |
| AAV capsid | wherein X₁ is Q, E, N, G, M, or C; X₂ is S, N, T, or M; X₃ is A, T, L, I, or S; | |
| protein VR-VIII | X₄ is Q, V, F, Y, L, or I; X₅ is A, S, N, L, T, or I; and X₆ is I, S, Q, G, H, or | |
| | R | |
| Recombinant | ATNH-(X)ₙAQTG | 740 |
| AAV capsid | wherein ₙ is 4-8, and X represents any of the 20 standard amino acids | |
| protein VR-VIII | | |
| Partial protein | GAYA | 741 |
| of AAV capsid | | |
| Partial protein | TKLA | 742 |
| of AAV capsid | | |
| Partial protein | SSFT | 743 |
| of AAV capsid | | |
| Partial protein | DNIR | 744 |
| of AAV capsid | | |
| Partial protein | NVIS | 745 |
| of AAV capsid | | |
| Partial protein | GTSI | 746 |
| of AAV capsid | | |
| Partial protein | DARA | 747 |
| of AAV capsid | | |
| Partial protein | SAQA | 748 |
| of AAV capsid | | |
| Partial protein | QGAYAQ | 749 |
| of AAV capsid | | |
| Partial protein | NTKLAI | 750 |
| of AAV capsid | | |
| Partial protein | VSSFTS | 751 |
| of AAV capsid | | |
| Partial protein | NNVISG | 752 |
| of AAV capsid | | |
| Partial protein | TGTSII | 753 |
| of AAV capsid | | |
| Partial protein | QANYGQ | 754 |
| of AAV capsid | | |
| Partial protein | QDARAQ | 755 |
| of AAV capsid | | |
| Partial protein | QSAQAQ | 756 |
| of AAV capsid | | |
| Partial protein | KYHKSGAAQ | 757 |
| of AAV capsid | | |
| Partial protein | KQVNGRPRD | 758 |
| of AAV capsid | | |
| Partial protein | QHYSAQAQ | 759 |
| of AAV capsid | | |
| Recombinant | -X₁-X₂-X₃-X₄- | 760 |
| AAV capsid | wherein X₁ is S, M, D, N, G, A, T, R, or I; X₂ is T, N, V, A, L, I, S, R, or P; | |
| protein partial | X₃ is Y, T, S, I, V, F, L, R, N, D, G, or Q; and X₄ is L, A, I, R, S, G, N, T, V, | |
| VR-VIII | Q, F, E, or Y | |
| Recombinant | -X₁-X₂-X₃-X₄- | 761 |
| AAV capsid | wherein X₁ is S, M, D, N, G, or A; X₂ is T, N, V, or A; X₃ is Y, T, S, I, or V; | |
| protein partial | and X₄ is L, A, I, R, S, or G | |
| VR-VIII | | |
| Recombinant | -X₁-X₂-X₃-X₄-X₅-X₆- | 762 |
| AAV capsid | wherein X₁ is N, M, C, E, G, S, V, A, T, H, L, or Q; X₂ is M, D, N, G, A, T, | |
| protein partial | R, I, or S; X₃ is T, N, V, L, I, S, R, P, or A; X₄ is Y, T, S, I, V, F, L, R, N, D, | |
| VR-VIII | G, or Q; X₅ is L, I, R, S, G, N, T, V, Q, F, E, Y, or A, and X₆ is G, R, S, I, H, | |
| | N, Y, L, M, or Q | |
| Recombinant | -X₁-X₂-X₃-X₄-X₅-X₆- | 763 |
| AAV capsid | wherein X₁ is N, M, C, E, G, S, V, A, T, H, or L; X₂ is M, D, N, G, A, T, R, | |
| protein partial | or I; X₃ is T, N, V, L, I, S, R, or P; X₄ is Y, T, S, I, V, F, L, R, N, D, or G; X₅ | |
| VR-VIII | is L, I, R, S, G, N, T, V, Q, F, E, or Y, and X₆ is G, R, S, I, H, N, Y, L, or M | |
| Recombinant | -X₁-X₂-X₃-X₄- X₅-X₆- | 764 |
| AAV capsid | wherein X₁ is E, N, G, M, C, V, or T; X₂ is N, T, M, G, or D; X₃ is T, L, I, | |
| protein partial | K, S, N or V; X₄ is V, F, Y, L, T, S, I, R; X₅ is S, N, L, T, I, or R, and X₆ is I, | |
| VR-VIII | S, G, H or R | |
| Recombinant | -X₁-X₂-X₃-X₄-X₅-X₆- | 765 |
| AAV capsid | wherein X₁ is E, N, M, C, or Q; X₂ is A, M, G, D, N, or S; X₃ is T, N, V, or | |
| protein partial | A; X₄ is V, Y, T, S, I, or Q; X₅ is S, G, L, I, R, or A; and X₆ is I, S, G, R, or | |
| VR-VIII | Q | |
| Recombinant | -X₁-X₂-X₃-X₄-X₅-X₆- | 766 |
| AAV capsid | wherein X₁ is E, N, M, or C; X₂ is A, M, G, D, or N; X₃ is T, N, or V; X₄ is | |
| protein partial | V, Y, T, S, or I; X₅ is S, G, L, I, or R; and X₆ is I, S, G, or R | |
| VR-VIII | | |
| ZC531 | MAADGYLPDWLEDNLSEGIREWWALKPGAPQPKANQQHQDNARG | 767 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHMMTTARAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| ZC532 | MAADGYLPDWLEDNLSEGIREWWALKPGAPQPKANQQHQDNARG | 768 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHCSTSIRAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC533 | MAADGYLPDWLEDNLSEGIREWWALKPGAPQPKANQQHQDNARG | 769 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQGAYAQAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| ZC534 | MAADGYLPDWLEDNLSEGIREWWALKPGAPQPKANQQHQDNARG | 770 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHNTKLAIAQTGWVQN | |
| | QGILPGMVWQDRDVYLQQPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC535 | MAADGYLPDWLEDNLSEGIREWWALKPGAPQPKANQQHQDNARG | 771 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHVSSFTSAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC536 | MAADGYLPDWLEDNLSEGIREWWALKPGAPQPKANQQHQDNARG | 772 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRIMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHEDNIRSAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC537 | MAADGYLPDWLEDNLSEGIREWWALKPGAPQPKANQQHQDNARG | 773 |
| | LVLPGYKYLGPGNGLDKGEPVNAADAAALEHDKAYDQQLKAGDN | |
| | PYLKYNHADAEFQERLKEDTSFQGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQSAQAQAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| ZC538 | MAADGYLPDWLEDNLSEGIREWWALKPGAPQPKANQQHQDNARG | 774 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYEDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHNNVISGAQTGWVQN | |
| | QGILPQMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC539 | MAADGYLPDWLEDNLSEGIREWWALKPGAPQPKANQQHQDNARG | 775 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEBIKTTNPVATESYGQVATNHTGTSIIAQTGWVQN | |
| | QGILPGMVWQDRDVYLQGPIWAKIPHTDGNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| ZC540 | MAADGYLPDWLEDNLSEGIREWWALKPGAPQPKANQQHQDNARG | 776 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQWMSAQAQAQTG | |
| | WVQNQGILPGMVWQDRDVYLQGPIWAKIPHTDGNFHPSPLMGGFG | |
| | MKHPPPQILIKNTPVPADPPTAFNKDKINSFITQYSTGQVSVEIEWEL | |
| | QKENSKRWNPEIQYTSNYYKSNNVEFAVNTEGVYSEPRPIGTRYLTR | |
| | NL | |
| ZC541 | MAADGYLPDWLEDNLSEGIREWWALKPGAPQPKANQQHQDNARG | 777 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQDARAQAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| ZC542 | MAADGYLPDWLEDNLSEGIREWWALKPGAPQPKANQQHQDNARG | 778 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHQHYSAQAQAQTGW | |
| | VQNQGILPGMVWQDRDVYLQGPIWAKIPHTDGNFHPSPLMGGFGM | |
| | KHPPPQILIKNTPVPADPPTAFNKDKLNSFITQYSTGQVSVEIEWELQ | |
| | KENSKRWNPEIQYTSNYYKSNNVEFAVNTEGVYSEPRPIGTRYLTRN | |
| | L | |
| ZC369 | MAADGYLPDWLEDNLSEGIREWWALKPGAPQPKANQQHQDNARG | 779 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLQLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHFSPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHNIRTEMAQTGWVQ | |
| | NQGILPGMVWQDRDVYLQGPIWAKIPHTDGNFHPSPLMGGFGMKH | |
| | PPPQILIKNTPVPADPPTAFNKDKLNSFITQYSTGQVSVEIEWELQKEN | |
| | SKRWNPEIQYTSNYYKSNNVEFAVNTEGVYSEPRPIGTRYLTRNL | |
| ZC370 | MAADGYLPDWLEDNLSEGIREWWALKPGAPQPKANQQHQDNARG | 780 |
| | LVLPGYKYLGPGNGLDKGEPVNAADAAALEHDKAYDQQLKAGDN | |
| | PYLKYNHADAEFQERLKEDTSFGGNLGRAVFQAKKRLLEPLGLVEE | |
| | AAKTAPGKKRPVEQSPQEPDSSAGIGKSGAQPAKKRLNFGQTGDTES | |
| | VPDPQPIGEPPAAPSGVGSLTMASGGGAPVADNNEGADGVGSSSGN | |
| | WHCDSQWLGDRVITTSTRTWALPTYNNHLYKQISNSTSGGSSNDNA | |
| | YFGYSTPWGYFDFNRFHCHESPRDWQRLINNNWGFRPKRLNFKLFN | |
| | IQVKEVTDNNGVKTIANNLTSTVQVFTDSDYQLPYVLGSAHEGCLPP | |
| | FPADVFMIPQYGYLTLNDGSQAVGRSSFYCLEYFPSQMLRTGNNFQF | |
| | SYEFENVPFHSSYAHSQSLDRLMNPLIDQYLYYLSKTIKGSGQNQQT | |
| | LKFSVAGPSNMAVQGRNYIPGPSYRQQRVSTTVTQNNNSEFAWPGA | |
| | SSWALNGRNSLMNPGPAMASHKEGEDRFFPLSGSLIFGKQGTGRDN | |
| | VDADKVMITNEEEIKTTNPVATESYGQVATNHSTTNFRAQTGWVQN | |
| | QGILPQMVWQDRDVYLQGPIWAKIPHTDQNFHPSPLMGGFGMKHPP | |
| | PQILIKNTPVPADPPTAFNKDKLNSFITQYSTGQVSVEIEWELQKENS | |
| | KRWNPEIQYTSNYYKSNNVEFAVNTEGVYSEPRPIGTRYLTRNL | |
| Recombinant | -X₁-X₂-X₃-X₄-X₅-X₆-X₇- | 781 |
| AAV capsid | wherein X₁ is R, or H; X₂ is N, M, C, E, G, S, V, A, T, H, L, or Q; X₃ is | |
| protein partial | M, D, N, G, A, T, R, I, or S; X₄ is T, N, V, L, I, S, R, P, or A; X₅ is Y, T, S, | |
| VR-VIII | I, V, F, L, R, N, D, G, or Q; X₆ is L, I, R, S, G, N, T, V, Q, F, E, Y, or A, | |
| | and X₇ is G, R, S, I, H, N, Y, L, M, or Q | |

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the claims. Changes therein and other uses which are encompassed within the spirit of the disclosure as defined by the scope of the claims will occur to those skilled in the art.

### Example 1: Cellular Model of PKP2 Depletion

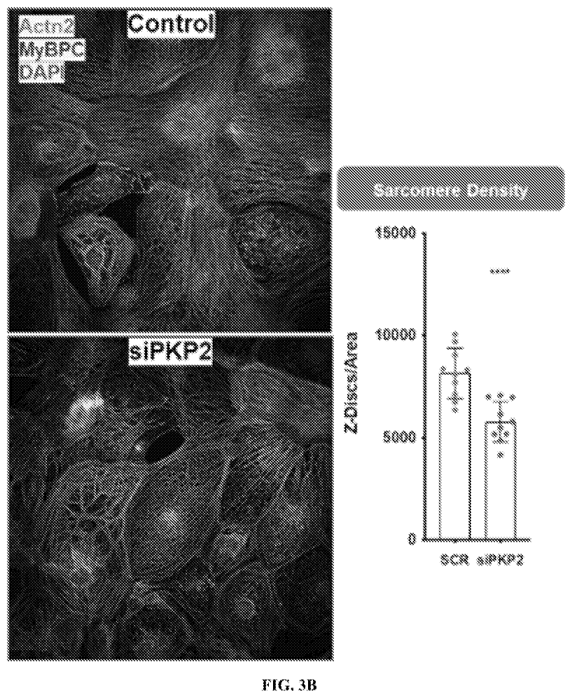

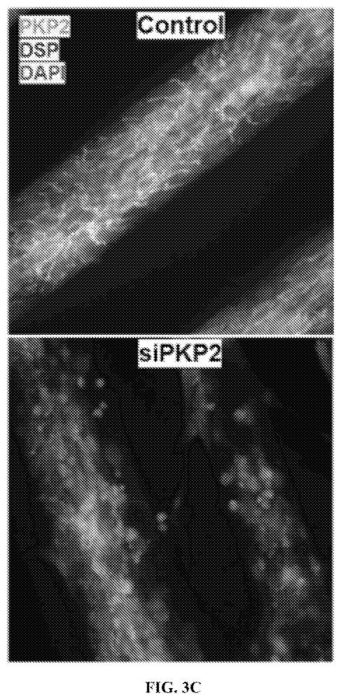

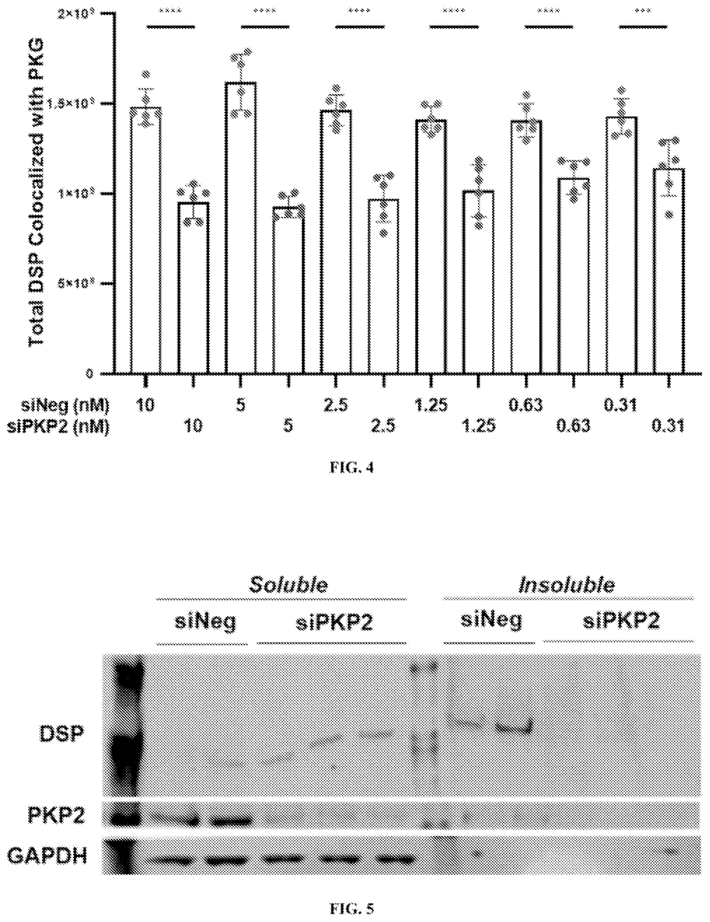

As an initial proof of concept, a cellular model of depletion of PKP2 was created using siRNA. PKP2 was depleted in in induced pluripotent stem cell-derived cardiomyocytes (iPSCM). Acute silencing of PKP2 by siRNAs was performed using siRNAs purchased from Invitrogen including both siPKP2 and negative control siRNA (4390843 Silencer Select Negative Control No. I siRNA; 4392420 Assay Id s531202 Silencer Select Pre-Designed siRNA #1; 4392420 Assay Id s531203 Silencer Select Pre-Designed siRNA #2; 4392420 Assay Id s531204 Silencer Select Pre-Designed siRNA #3; and 4392420 Assay Id s10585 Silencer Select Pre-Designed siRNA #4), This silencing led to disappearance of DSP from the cellular membrane at day 8 as shown by immunofluorescence at FIG. 3A, The DSP membrane localization was quantitatively measured (FIG. 4) which illustrated a significant reduction in DSP-PKG co-localization. A reduction of sarcomere density was also observed by immunofluorescence (FIG. 3B). In addition, a disarray of cell compaction in patterned iPSCM was seen by immunofluorescence (FIG. 3C).

An immunoblot of siPKP2 iPSCM lysate was performed showing that a reduced total amount of DSP protein from the desmosomes is detected mainly in the insoluble fraction of cells were PKP2 is silenced (FIG. 5).

### Example 2: AAV9-PKP2 Rescues PKP2 Depletion Phenotype

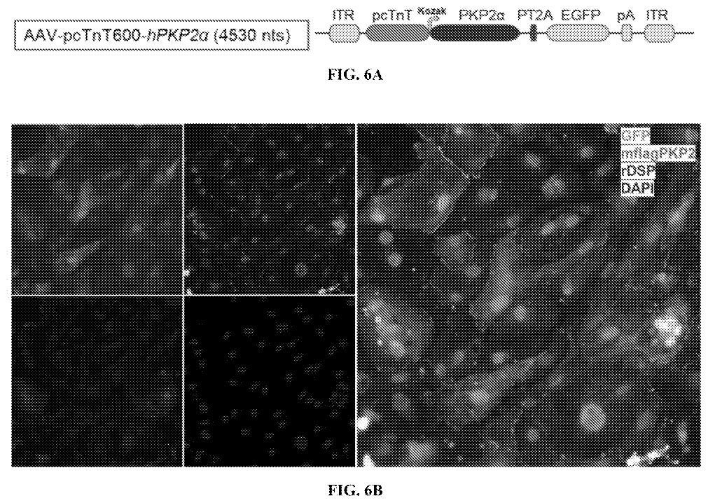

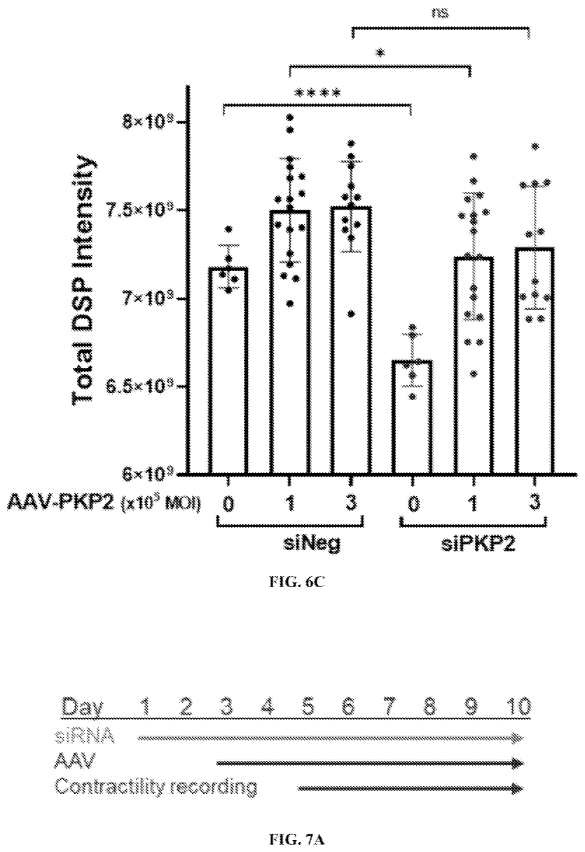

By delivering AAV9 variant CR9-01 flag-tagged PKP2 expression driven by 600 nt cardiac troponin (TnT) promoter with GFP to identify transduced cells (FIG. 6A), re-localization of DSP back to the membrane in PKP2 silenced iPSCM was observed (FIG. 6B), thereby restoring desmosome structure. PKP2 transgene was codon optimized to resist siRNA-mediated silencing. Due to a technical difficulty, it was not possible accurately quantify how much DSP was specifically localized to the membrane where cellular junction occurs and desmosomes exist. Therefore, the total cellular DSP intensity, instead of an amount of DSP localized to membrane, was quantified.

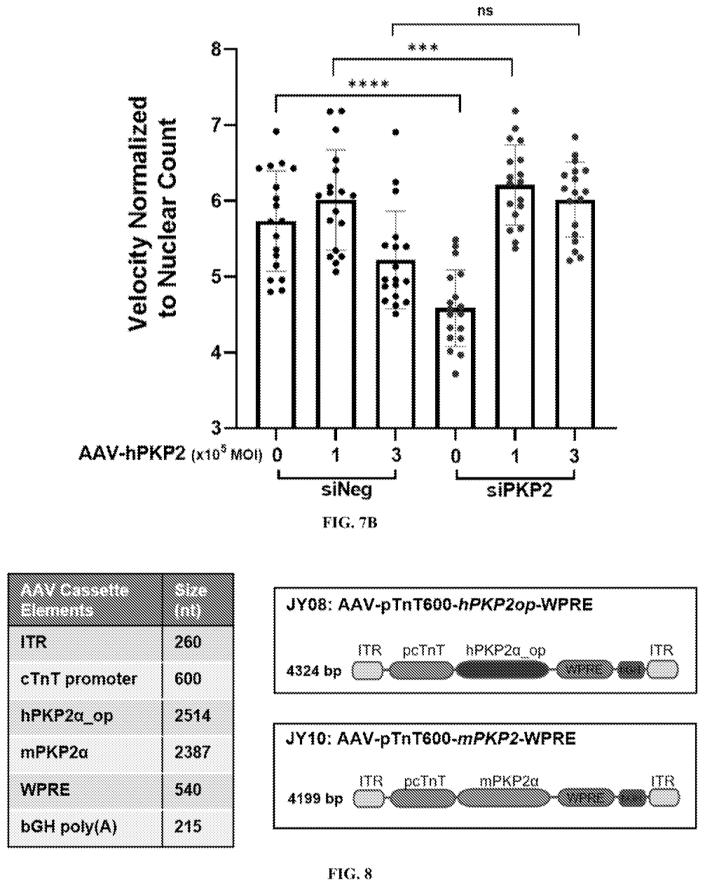

To demonstrate that PKP2 transgene could functionally restore the contractility of cardiomyocytes, bright field-based contraction of iPSCM was recorded by SONY imaging and videos were analyzed by DANA Solutions Pulse analysis software. An experimental timeline is shown in FIG. 7A. In this experiment, siRNA was used to deplete endogenous PKP2 expression in iPSCM cells on day 1. Two siRNA concentrations, 5 and 1.25 nM, were used for cither siRNA negative control or siPKP2. Two siPKP2 #3 and #4 were combined to silence the transcript. On day 3, an AAV PKP2 was used to transduce depleted cells resulting in a rescue of contraction velocity was observed in iPSCM in response to PKP2 transgene expression (FIG. 7B). Contractility was recorded at days 3, 4, 5, 6, 7, and 8 post AAV transduction. Contraction velocity was averaged from three 96-well plates and from cells transduced with either AAV 300K MOI or 100K MOI, respectively, at both 5 and 1.25 nM siRNAs. The velocity value was further normalized to the average nuclear count corresponding to 300K or 100K MOI, respectively.

### Example 3: Treatment with Second Generation PKP2α AAV9

A second generation AAV expression cassette was developed for expressing human or mouse PKP2α. The second generation cassette included a Woodchuck Hepatitis Virus Posttranscriptional Regulatory Element (WPRE) and a bovine growth hormone polyadenylation signal (bGH poly(A)). The second generation vector is illustrated in FIG. 8.

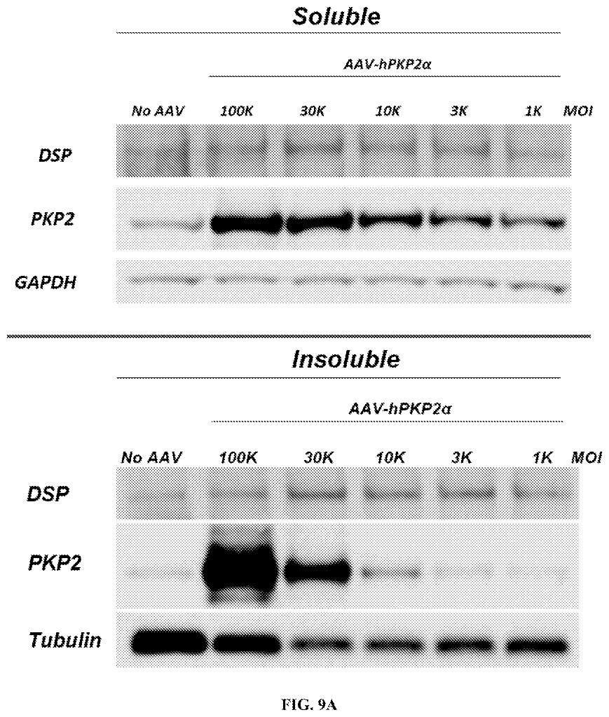

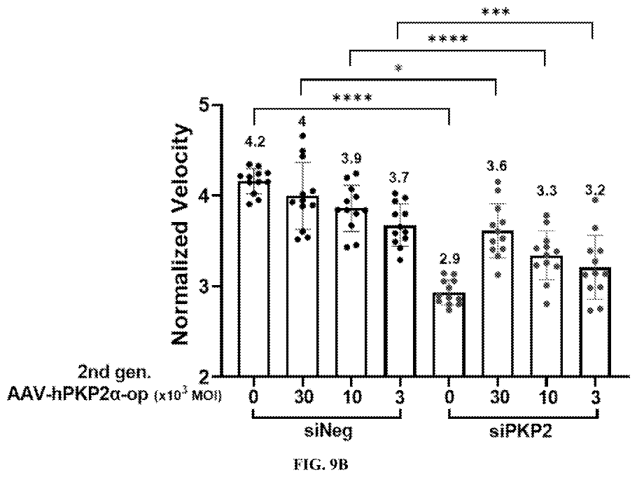

Transgene rescue studies were conducted in PKP2 silenced iPSC cardiomyocytes. Preliminary results suggested that the second generation AAV-hPKP2u partially rescued contraction velocity post PKP2 silencing in iPSC cardiomyocytes. FIG. 9A shows results where human PKP2u transgene was expressed in iPSC cardiomyocytes in a dose-dependent fashion by different MOI (multiplicity of infection, the average number of virus particles infecting each cells). PKP2 and DSP (desmoplakin) expression were evaluated in both soluble and insoluble fractions of cells at 3 days post AAV transduction. FIG. 9B shows that human PKP2α transgene showed a partial rescue of contraction velocity post PKP2 silencing at 30K MOI as indicated by Student t test with a p value of 0.0103 in contrast to a p value of <0.0001 without AAV PKP2 transgene.

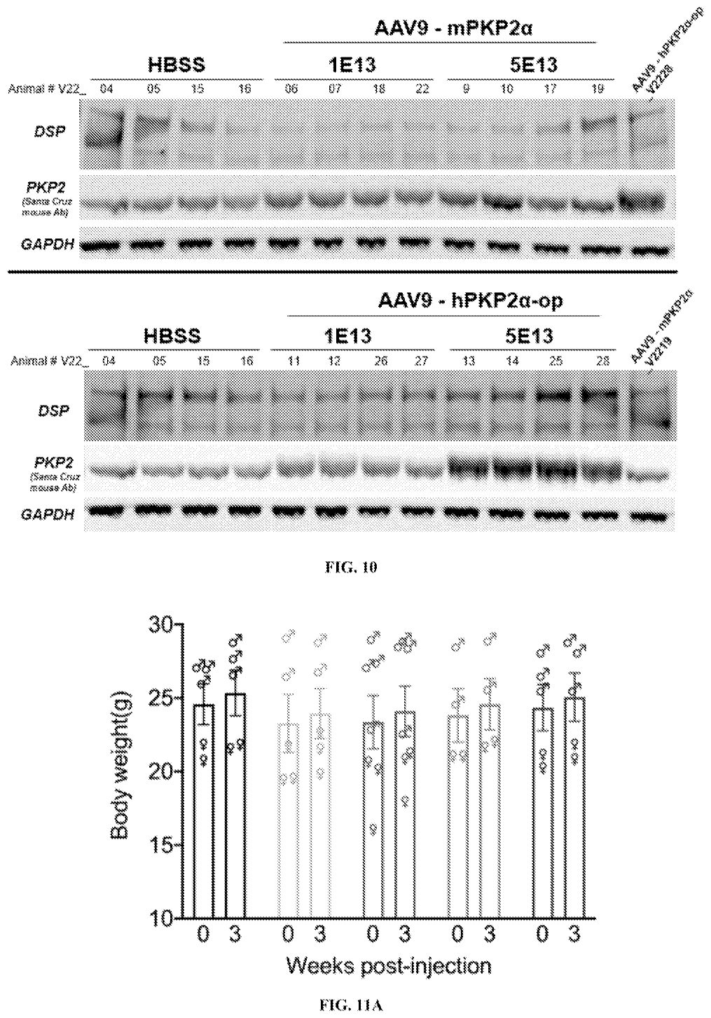

An experiment was conducted to study expression analysis of the second generation AAV9 human and mouse PKP2α in 12 week-old C57BL/6 animals. The results of this experiment are shown in FIG. 10. Animals were retro-orbital intravenously administrated with AAV9-PKP2α at doses of 1E13 and 5E13 vg/kg, respectively. Heart LV tissues were harvested at 3.5 weeks post injection. Soluble fraction of LV tissues were analyzed here with Western blot. The upper panel shows expression of endogenous mouse PKP2α in HBSS control mice and expression of both endogenous and transduced mouse PKP2α at two AAV9 injected doses, 1E13 and 5E13, respectively. The lower panel shows corresponding expression analysis of transduced human PKP2α, a slightly larger homolog. This human homolog is codon optimized. There were no adverse cardiac event observed at 3.5 weeks post AAV injection by echocardiogram.

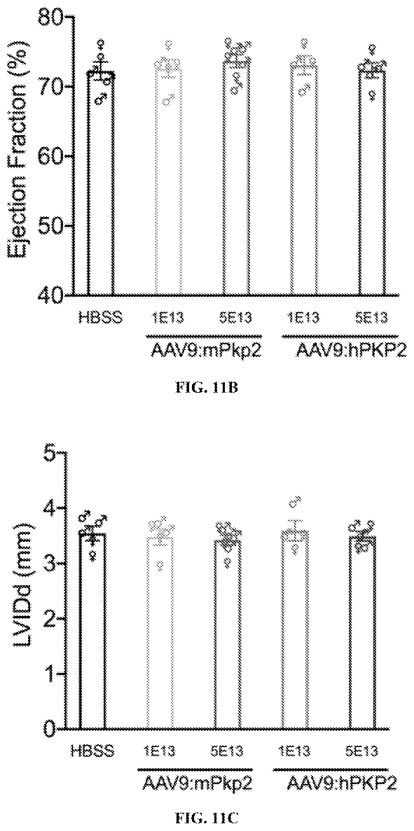

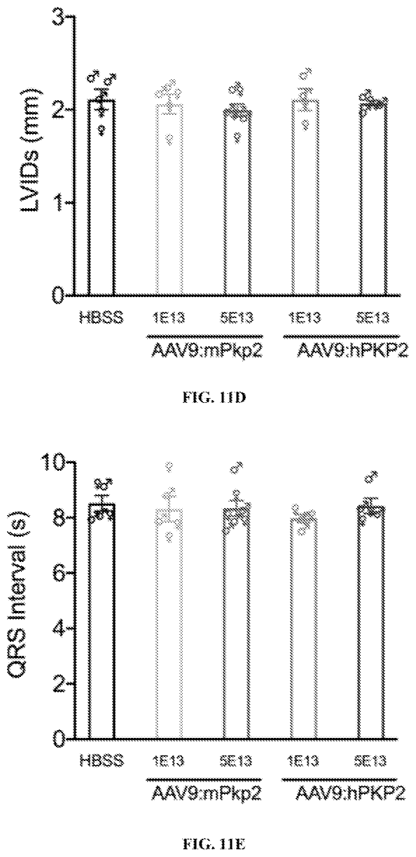

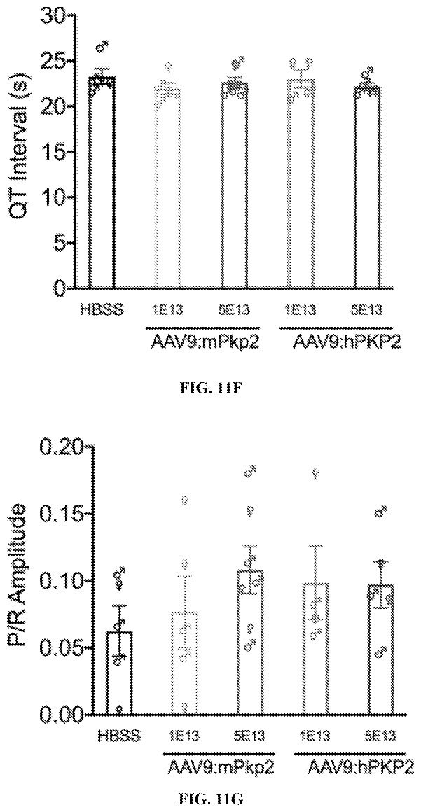

FIGS. 11A-11G show pilot expression safety studies of second generation AAV9 human and mouse PKP2u in 12 week-old C57BL/6 animals did not show any adverse cardiac event at 3 weeks post AAV injection by echocardiogram. Animals were retro-orbital intravenously administrated with AAV9-PKP2α at doses of 1E13 and 5E13 vg/kg, respectively. FIG. 11A shows body weight before AAV9 injection and body weight at 3 weeks post AAV9 injection. FIG. 11B shows heart function measured by percentage of ejection fraction at 3 weeks post AAV9 injection of either mouse or human PKP2α. FIGS. 11C and 11D show LV structure measured by both internal diameters end diastole and systole. FIG. 11E-11F show electrophysiology activity measured by QRS, QT interval, and P/R amplitude.

### Example 4: PKP2-cKO ARVC Mouse Model Characterization

Four wild-type and seven PKP2-cKO ARVC mice, αMYHC-Cre-ER(T2), PKP2^{fl/fl} at approximately 3 months of age were intraperitoneally injected for four consecutive days with tamoxifen (20 mg/ml in corn oil 100 μl/mice (approximately 75 mg/kg)). Baseline readings of body weight, echocardiography, and EKG were collected before tamoxifen induction. All readings post-tamoxifen induction were recorded weekly including echocardiography of B-mode, M-mode (RV, LV), and structure (LV internal diameters) and 30-minute ECG for quantifying arrythmias and evaluating electrophysiological parameters. Terminal tissues, including heart and lung, were collected at the end of the study.

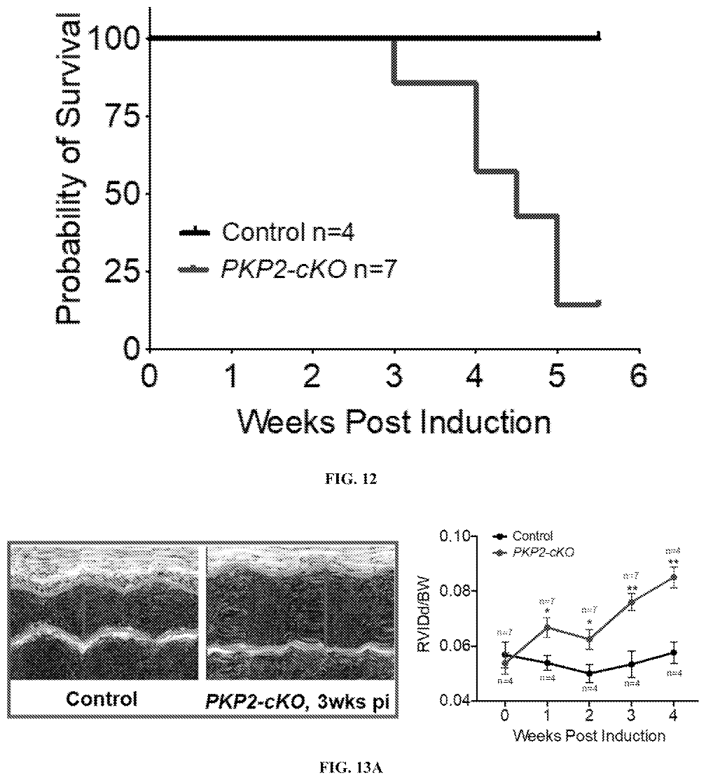

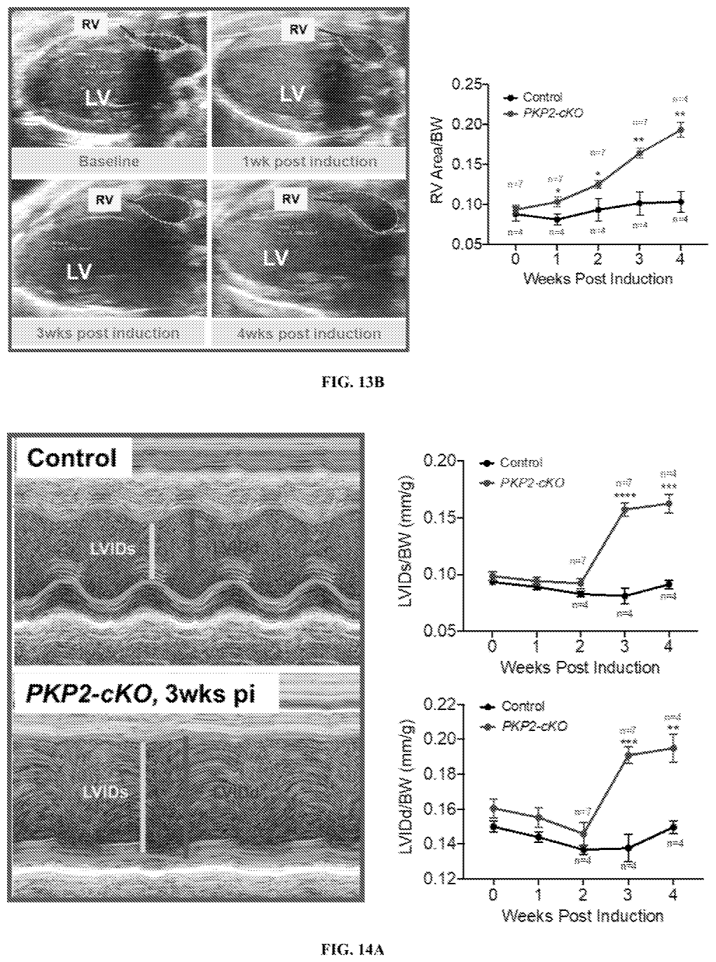

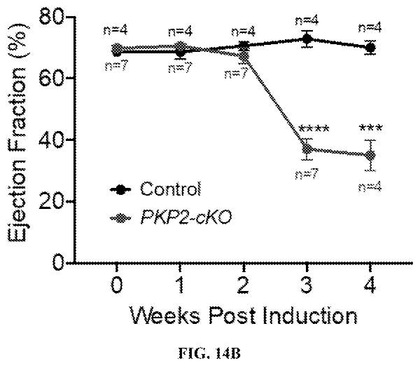

A survival analysis was performed on the mice (FIG. 12). The Kaplan-Meier survival curve showed a sharp decline of survival of PKP2-cKO mice three weeks post-tamoxifen induction, with only one animal reaching six weeks post-tamoxifen induction. Animals showed severe clinical symptoms including sudden death, edema, reduced activity, less tolerance to isoflurane three weeks post-induction.

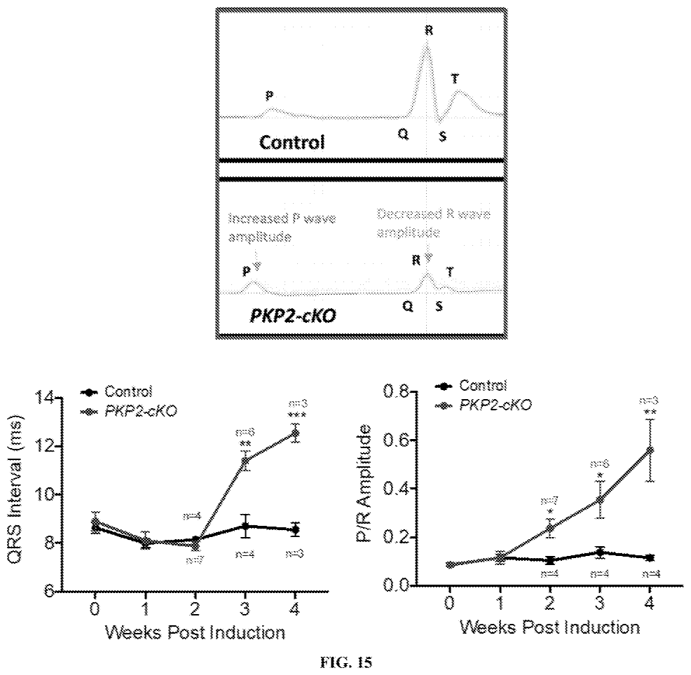

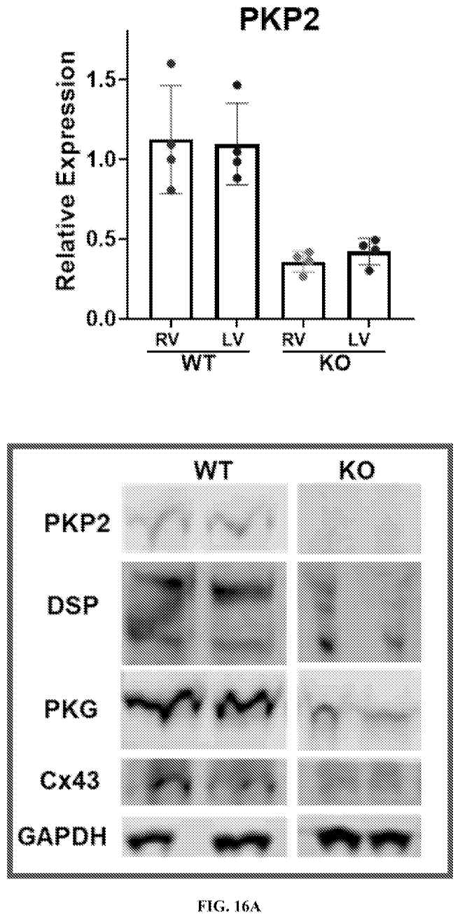

PKP2-cKO mice developed RV dilated cardiomyopathy at as early as one week post-tamoxifen induction. FIG. 13A, in the left panel, shows that at three weeks post-tamoxifen induction, PKP2-cKO mice developed an increased RV internal dimension at end-diastole (RVIDd). The right panel of FIG. 13A summarizes the continuous increases in RVIDd normalized to body weight during four weeks of tamoxifen induction. FIG. 13B, in the left panel, shows images of weekly increases in RV area suggesting RV dilation. The right panel of FIG. 13B summarizes the RV area increases normalized to body weight. P value: Student's t-test. Error bar: s.e.m. *P<0.05, **P<0.01 Vs. Control.

PKP2-cKO mice developed LV dilated cardiomyopathy post-tamoxifen induction. FIG. 14A, left panel, shows that at three weeks post-tamoxifen induction, PKP2-cKO developed an increased LV internal dimension at end-systole (LVIDs) and end diastole (LVIDd). The right panel of FIG. 14A summarizes the continuous increases in LVIDs and LVIDd normalized to body weight during four weeks of tamoxifen induction. FIG. 14B shows LV performance as measured by % ejection fraction sharply declined after two weeks post-tamoxifen induction. P value: Student's t-test. Error bar: s.e.m. *P<0.05, **P<0.01, ***P<0.001 Vs. Control.

PKP2-cKO mice developed prolonged QRS interval and increased P/R amplitude ratio suggesting ventricular conduction disturbance and intraventricular block. FIG. 15 top panel shows that at three weeks post-tamoxifen induction, PKP2-cKO mice developed an increased P wave amplitude and decreased R wave amplitude. The bottom left graph in FIG. 15 shows the continuous increases in QRS interval and the lower right graph shows the increase in P/R amplitude ratio during four weeks of tamoxifen induction. P value: Student's t-test. Error bar: s.e.m. *P<0.05, **P<0.01, ***P<0.001 Vs. Control.

PKP2-cKO mice developed spontaneous premature ventricular contractions (PVCs). Table 5 shows data obtained during 30 minutes of continuous recording, PVCs were nearly absent at one week, whereas occasional extra systoles were detected in all the PKP2-cKO animals at two weeks. The occurrence of PVCs increased further at later times with a majority of animals showing over 100 PVCs. Starting from three weeks, sudden cardiac death was observed in PKP2-cKO animals.

TABLE 5
PKP2-cKO ARVC Mouse Model PVC

| | |
|---|---|
| Animal | Week Post Tamoxifen induction |

| ID | Week-1 | Week-2 | Week-3 | Week-4 |
|---|---|---|---|---|
| 121 | 0 | 5 | >100 | Died |
| 125 | 0 | 12 | 12 | Died |
| 130 | 0 | 2 | Died | Died |
| 137 | 0 | 66 | >100 | N/A |
| 138 | 0 | 20 | >100 | Died |
| 150 | 0 | 1 | 11 | >100 |
| 152 | 4 | 5 | >100 | >100 |

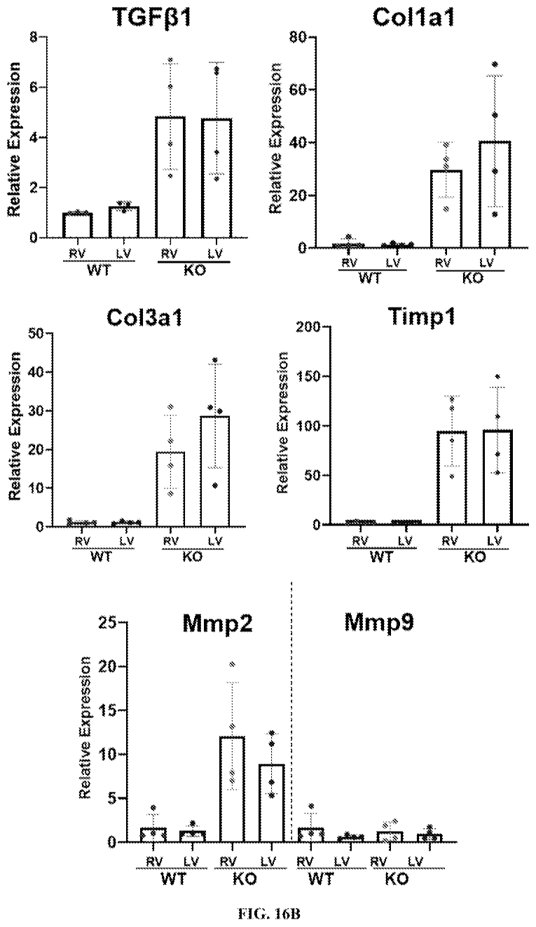

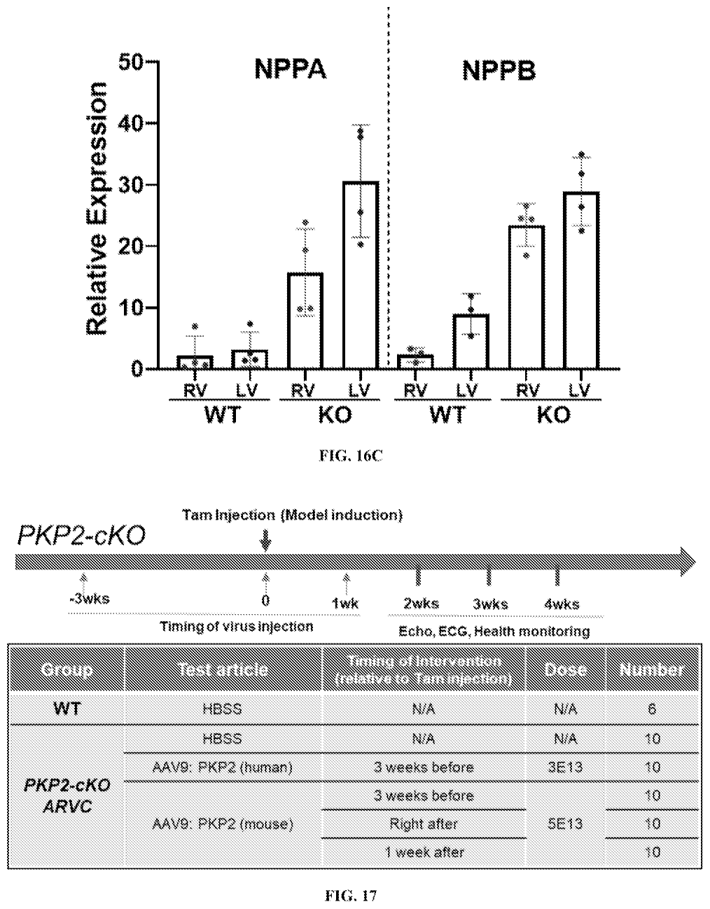

PKP2-cKO mice showed enhanced expression of fibrosis, tissue remodeling genes, and heart failure markers. FIG. 16A, top panel, shows PKP2 mRNA expression in both RV and LV of wild type and PKP2-cKO mice. Red and blue dots represent each individual mouse. The bottom panel of FIG. 16A shows representative immunoblots of reduction in LV protein levels of PKP2, DSP, and PKG in desmosome and Cx43 in gap junction. FIG. 16B shows that PKP2-cKO mice showed enhanced expression of fibrosis genes, TGFβ1, Col1a1, and Col3a1, and tissue remodeling genes, Timp1 and Mmp2. FIG. 16C shows PKP2-cKO mice showed enhanced expression of heart failure markers, NPPA and NPPB.

### Example 5: PKP2 Gene Therapy Efficacy in PKP2-cKO ARVC Mouse Model

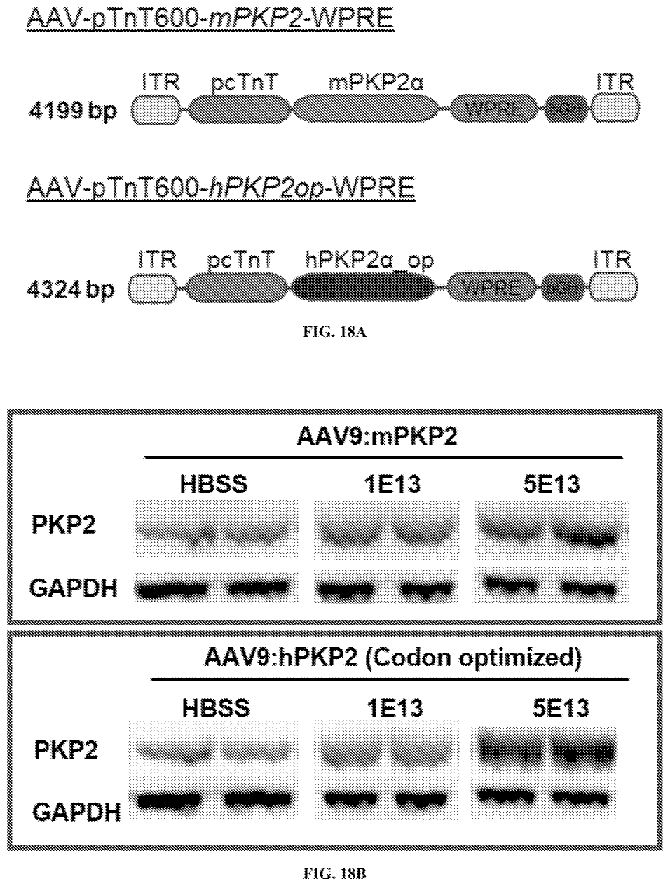

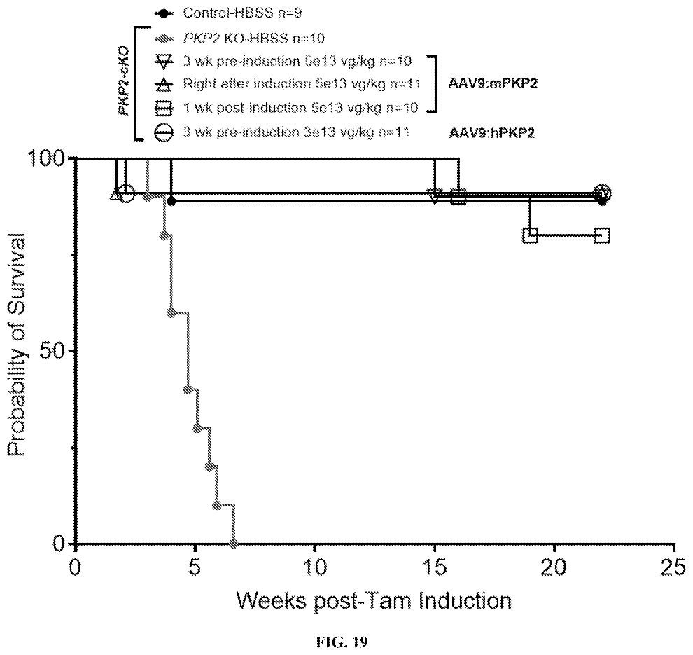

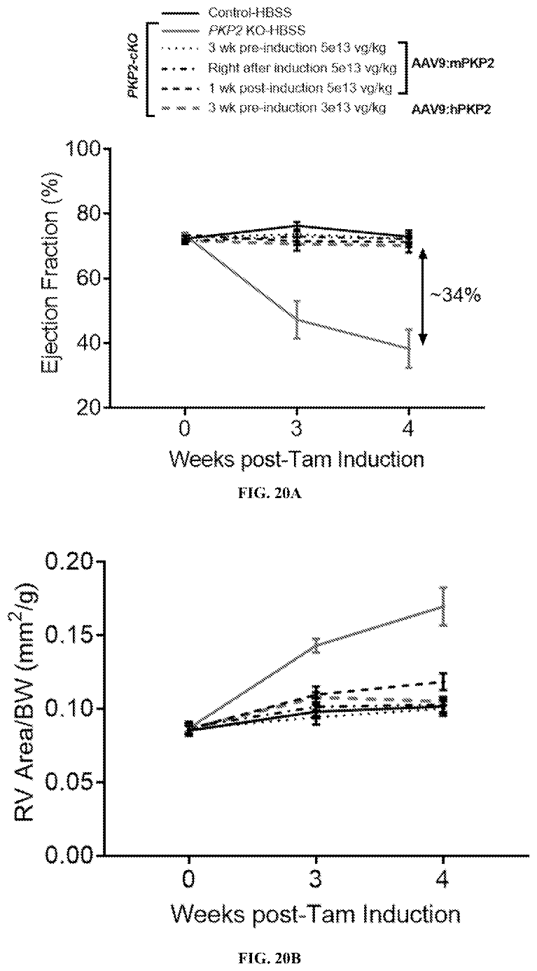

FIG. 17 shows the experimental design used to evaluate PKP2 efficacy as a gene therapy target using the PKP2-cKO ARVC mouse model. A total of six individual treatment groups were included in the studies and all groups were tamoxifen treated for three consecutive days. The treatment groups are as follows: six wildtype mice treated with HBSS buffer; ten PKP2-cKO ARVC mice treated with HBSS buffer; ten PKP2-cKO ARVC mice treated with 3E13 vg/kg of AAV9:hman PKP2 at three weeks before tamoxifen induction; ten PKP2-cKO ARVC mice treated with 5E13 vg/kg of AAV9:mouse PKP2 at three weeks before tamoxifen induction; ten PKP2-cKO ARVC mice treated with 5E13 vg/kg of AAV9:mouse PKP2 right after tamoxifen induction; and ten PKP2-cKO ARVC mice with 5E13 vg/kg of AAV9:mouse PKP2 at one week after tamoxifen induction.

Baseline recordings of body weight, echocardiography, and EKG were collected before tamoxifen induction. All readings post tamoxifen induction were recorded weekly including echocardiography of B-mode, M-mode (RV, LV) and structure (LV internal diameters), and 30-minute ECG for quantifying arrythmias and evaluating electrophysiological parameters. Terminal tissues (heart and lung) will be collected at the end of the study.

AAV9:PKP2 protein expression was detected in wildtype mouse LV heart tissue. FIG. 18A shows a schematic representation of the second generation AAV expression cassette of human and mouse PKP2α. FIG. 18B shows representative immunoblots conducted to show expression of mouse and human PKP2 at three weeks post retro-orbital injection of AAV9:PKP2 (full blots are shown in FIG. 10). A total of five C57BL6 wildtype mice at eight weeks of age were injected for each treatment: HBSS, 1E13 vg/kg, or 5E13 vg/kg.

A Kaplan-Meier survival curve showed that AAV9:PKP2 extended life span of PKP2-cKO mice after 6 weeks post-tamoxifen induction in all AAV9:PKP2 treated groups. Both human and mouse PKP2 demonstrated efficacy in extending life span of treated PKP2-cKO mice. In FIG. 19, the red line is PKP2-cKO mice treated with HBSS buffer showing a sharp decline after three weeks post-tamoxifen induction. In contrast, all AAV9-PKP2 treated mice survived until 20 weeks post-tamoxifen induction.

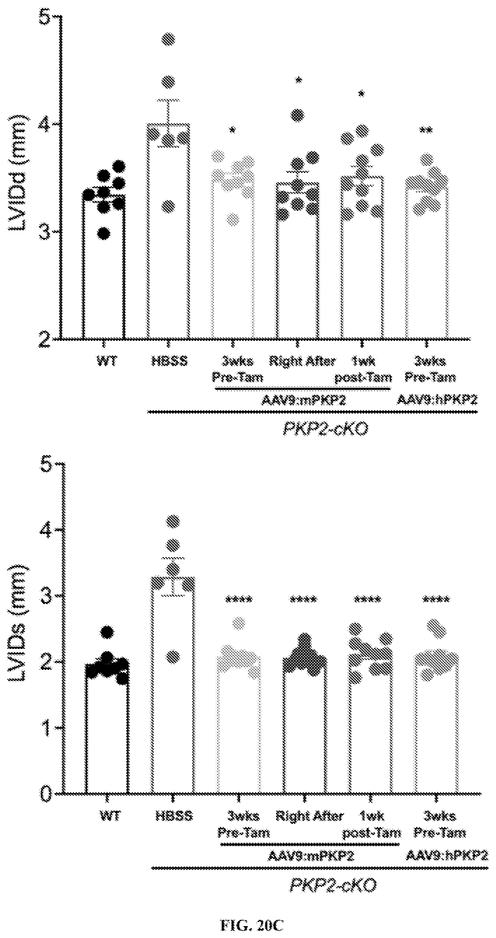

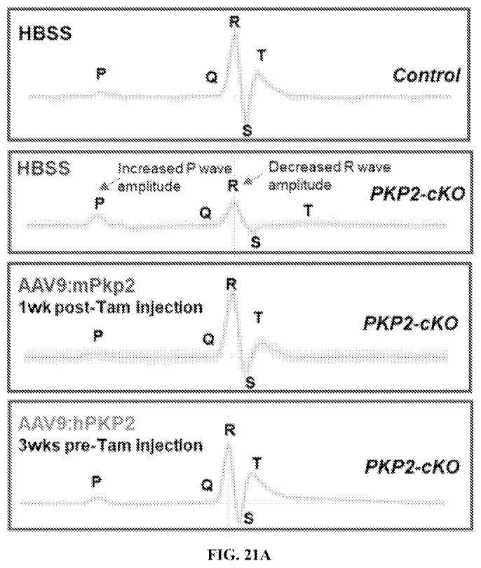

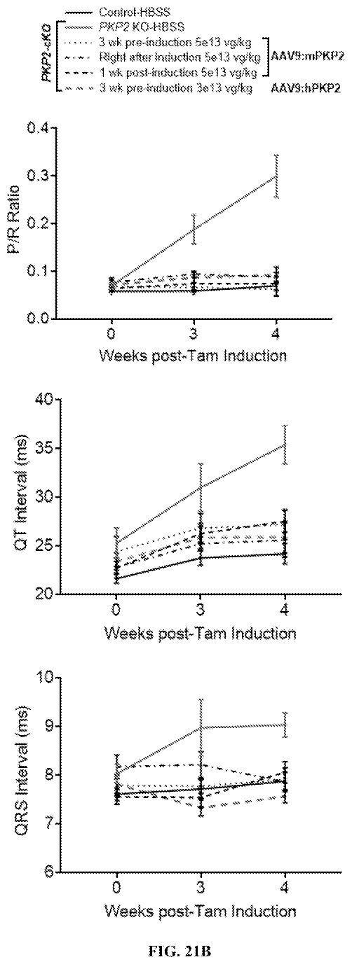

AAV9:PKP2 treatment of PKP2-cKO mice showed efficacy in reducing RV and LV dilation and maintaining cardiac function. FIG. 20A shows AAV9:PKP2 treatment prevented a decline in percent ejection fraction compared to HBSS-treated mice (shown in the red line). FIG. 20B shows AAV9:PKP2 treatment showed a reduction of RV dilation at weekly bases as estimated by RV area normalized to body weight. FIG. 20C shows at four weeks post-tamoxifen induction, AAV9:PKP2 treatment significantly reduced LV dilation of PKP2-cKO mice as measured by both LV internal dimension at end-diastole (LVIDd) (top graph) and LV internal dimension at end-systole (LVIDs) (bottom graph), both normalized by body weight. Error bar: s.e.m. *P<0.05, **P<0.01, ***P<0.00I Vs. Control.

AAV9:PKP2 treatment also significantly improved ECG parameters of PKP2-cKO mice. FIG. 21A shows examples of raw ECG traces which showed a significant improvement of electrophysiological behaviors of AAV9:PKP2 treated PKP2-cKO mouse hearts. FIG. 21B shows AAV9:PKP2 treatment showed significant improvement of P/R ratio (top graph), QT interval (middle graph), and QRS interval (bottom graph) as compared to PKP2-cKO mice treated with HBSS shown in red lines.

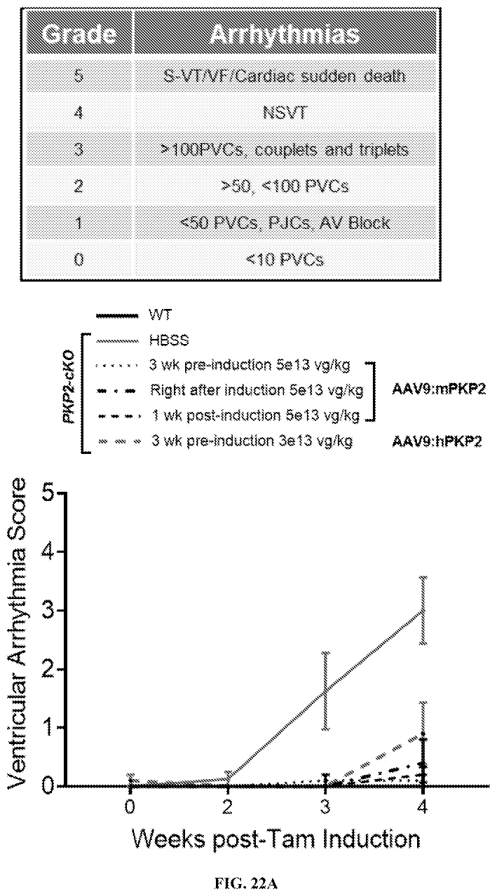

AAV9:PKP2 treatment also significantly reduced arrhythmias in PKP2-cKO mice. FIG. 22A (top) shows a table with a grading chart to categorize severity of spontaneous arrhythmias during 30 minutes of recording in anesthetized PKP2-cKO mice. Premature ventricular contractions (IPVCs), premature junctional complexes (PJCs), AV block (atrioventricular block), non-sustained ventricular tachycardia (NSVT), supraventricular tachycardia (S-VT), and ventricular fibrillation. FIG. 22A (bottom) summarizes averaged scores based on the grading chart showing amelioration of arrhythmias in AAV9:PKP2 treated PKP2-cKO mice. FIG. 22B shows a distribution of individual mice in each treatment group at four weeks post-tamoxifen induction. AAV9:PKP2 treatment showed a reduction of both arrhythmia event frequency and severity as indicated by improved arrhythmia scores when compared to PKP2-cKO mice treated with HBSS buffer shown in the red bar.

### Example 6: PKP2 Gene Therapy Efficacy Studies Using AAV9:hPKP2 in PKP2-cKO ARVC Mouse Model

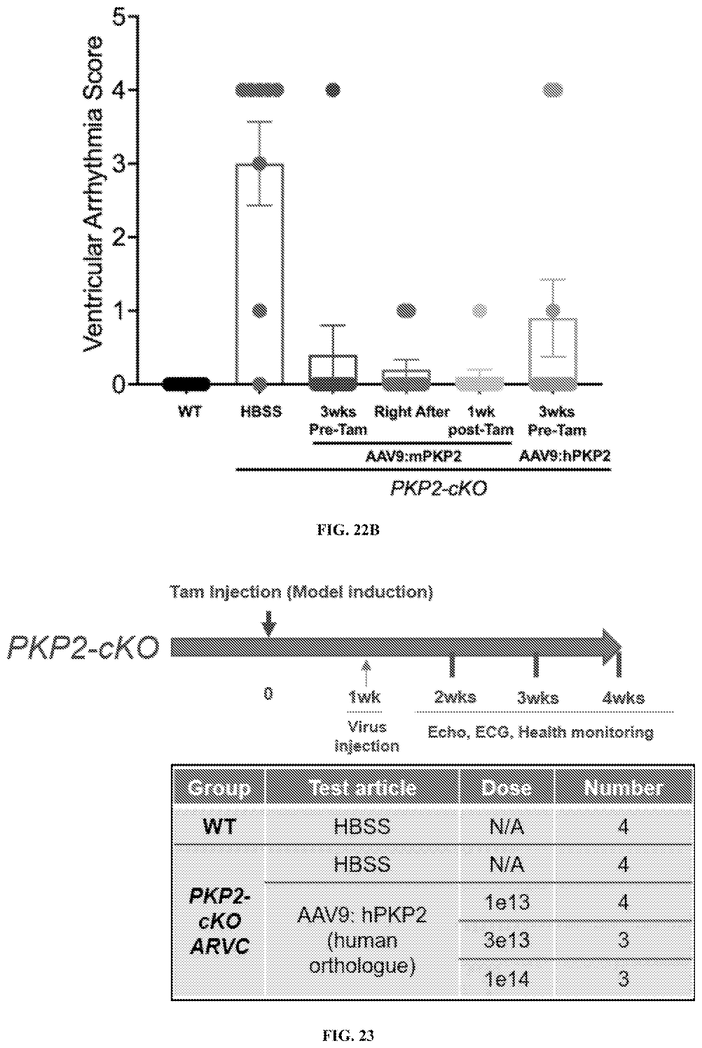

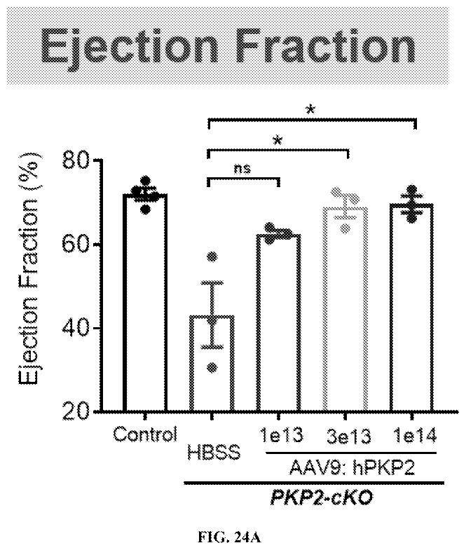

Efficacy of human PKP2 as a gene therapy target was determined using PKP2-cKO ARVC mouse model. A total of five individual treatment groups were included in the study. All groups were treated with tamoxifen for three consecutive days. The groups included: four wildtype mice treated with HBSS buffer; four PKP2-cKO ARVC mice treated with HBSS buffer; four PKP2-cKO ARVC mice treated with 1E13 vg/kg of AAV9:human PKP2 treated at one week after tamoxifen induction; three PKP2-cKO ARVC mice treated with 3E13 vg/kg of AAV9:human PKP2 treated at one week after tamoxifen induction; and three PKP2-cKO ARVC mice treated with 1E14 vg/kg of AAV9:human PKP2 treated one week after tamoxifen induction. Baseline recordings of body weight, echocardiography, and EKG were collected before tamoxifen induction. All readings post tamoxifen induction were recorded weekly including echocardiography of B-mode, M-mode (RV, LV), and structure (LV internal diameters) and 30-min ECG for quantifying arrythmias and evaluating electrophysiological parameters. Terminal heart tissues were collected at four weeks post tamoxifen induction. FIG. 23 illustrates the experimental design.

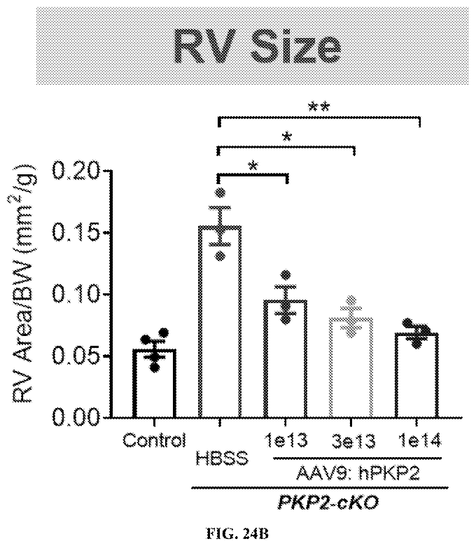

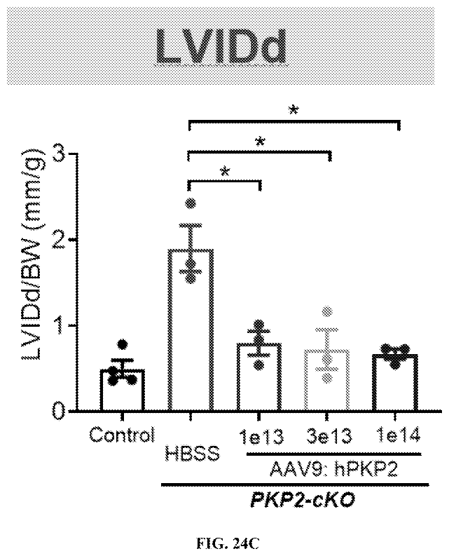

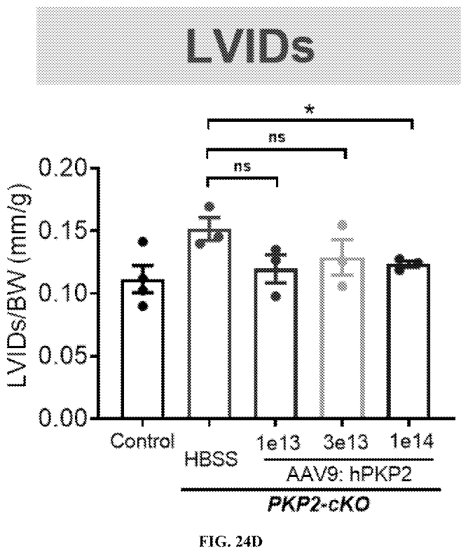

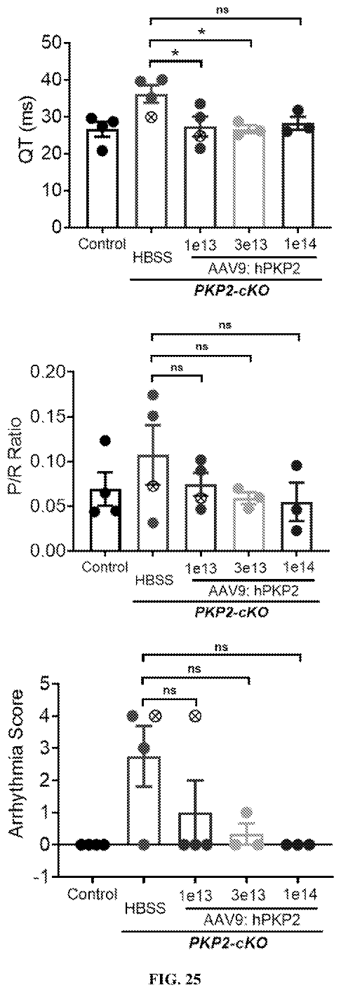

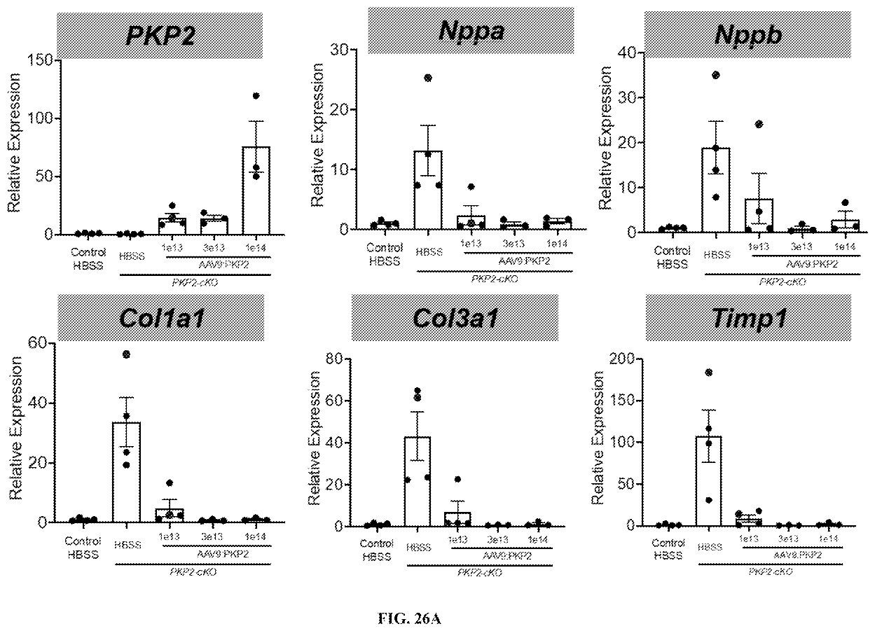

AAV9:hPKP2 treatment of PKP2-cKO mice showed efficacy in reducing right ventricle (RV) and left ventricle (LV) dilation and maintaining cardiac function at four weeks post tamoxifen induction. FIGS. 24A-24D show results for this assay. AAV9:hPKP2 treatment prevented decline of percent ejection fraction compared to HBSS-treated mice (FIG. 24A). AAV9:hPKP2 treatment showed reduction of RV dilation as estimated by RV area normalized to body weight (FIG. 2413). AAV9:hPKP2 treatment significantly reduced LV dilation of PKP2-cKO mice as measured by both LV internal dimension at end-diastole (LVIDd) (FIG. 24C) and LV internal dimension at end-systole (LVIDs) (FIG. 24D) both normalized to body weight. P value was determined by Student's-test, error bar: s.e.m. *P<0.05, **P<0.01 compared with control.

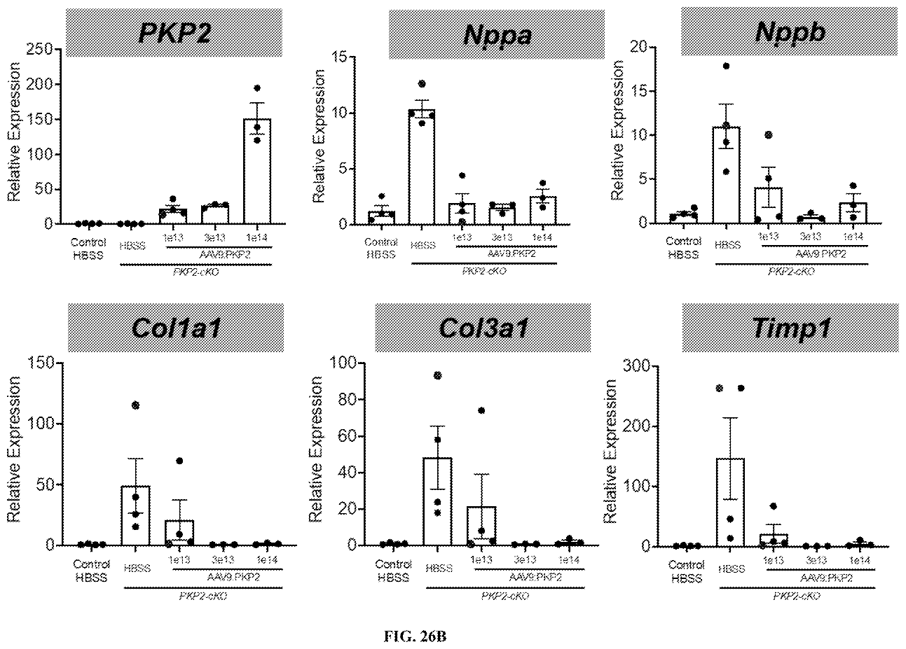

AAV9:hPKP2 treatment showed a significant reduction at four weeks tamoxifen induction in QT interval (FIG. 25 top panel), a trending reduction in P/R ratio (FIG. 25 middle panel), and a trending reduction in arrhythmias (FIG. 25 bottom panel) in PKP2-cKO mice as compared to PKP2-cKO mice treated with HBSS. P value was determined by Student's t-test, error bar is s.e.m. *P<−0.05 compared with control. Due to a small number of animals included in this study, P/R ratio and arrhythmia score did not reach statistical significance. In addition, one animal in the HBSS treated group reached humane endpoint at about 4 weeks post-tamoxifen induction after ECG recording. One animal in the 1e13 dosage group was found dead before echocardiography. Overall, the results of this study suggest that the optimal efficacious dose is 3e 13 vg/kg.

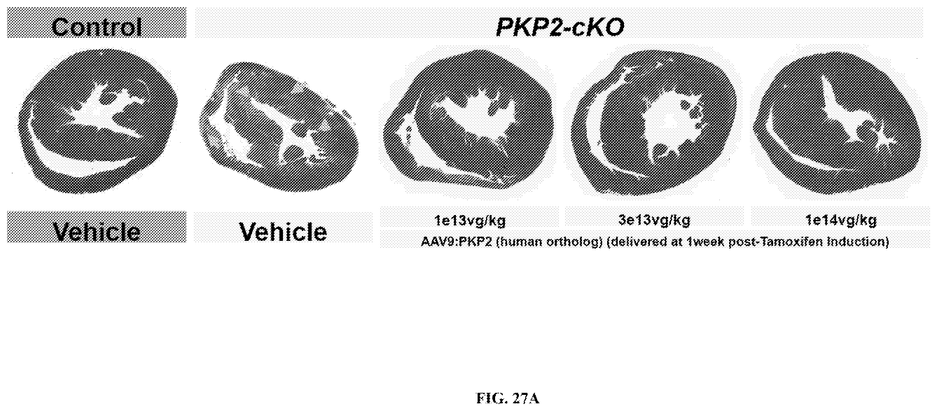

AAV9:hPKP2 treatment of PKP2-cKO mice showed efficacy in reducing expression of heart failure markers, fibrosis and tissue remodeling genes in both right ventricle (FIG. 26A) and left ventricle (FIG. 26B) at four weeks post tamoxifen induction. Endogenous and transgene mRNA levels of PKP2 were estimated in wildtype control, PKP2-cKO, and AAV9:hPKP2 transduced hearts respectively. Heart failure markers are NPAA and NPPB. Fibrosis genes are Col1a1 and Col3a1. The tissue remodeling gene is Timp1.

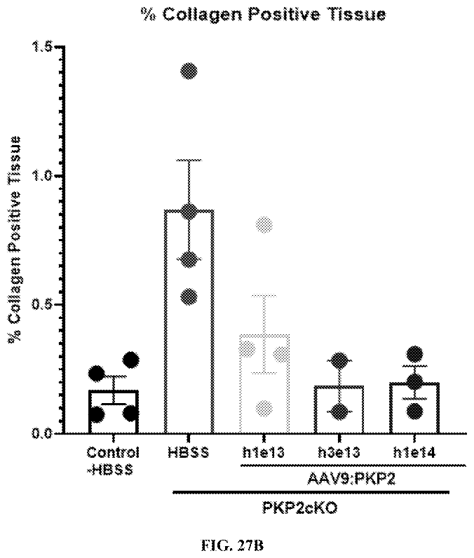

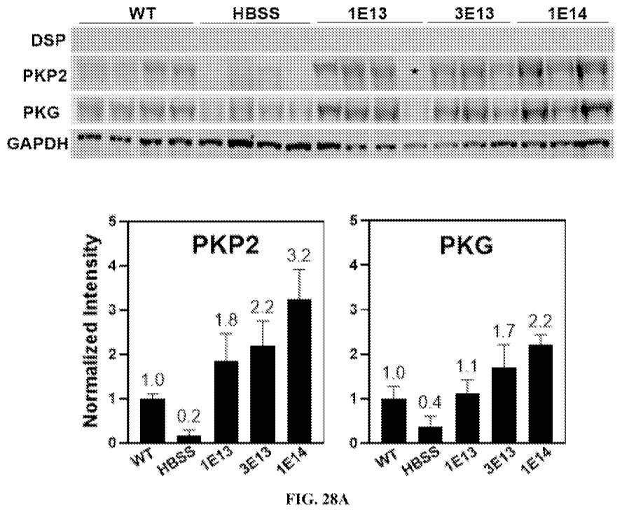

AAV9:hPKP2 treatment of PKP2-cKO mice showed efficacy in reducing fibrosis development in both right ventricle and left ventricle at four weeks post tamoxifen induction (FIG. 27A). Muscle staining is shown in red color and trichrome staining of fibrosis is in blue color. Arrows highlight areas with significant fibrosis in PKP2-cKO mouse heart. Collagen was quantified in FIG. 27B, showing that treatment with the AAV9:hPKP2 reduced collagen nearly to control levels.

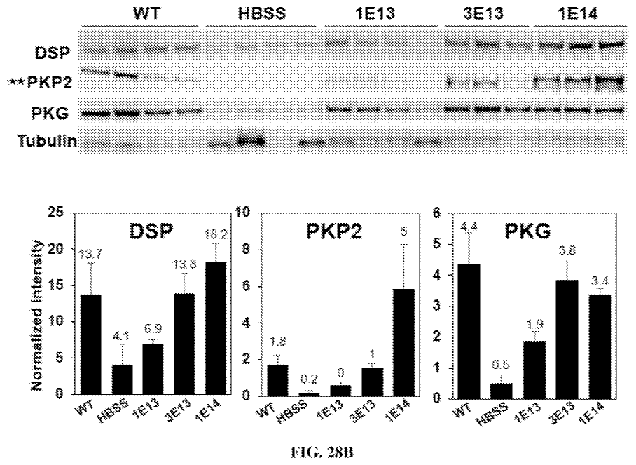

AAV9:hPKP2 treatment of PKP2-cKO mice showed a dose-dependent expression of human PKP2 transgene. The total expression level of PKP2 and other desmosome proteins, DSP and PKG, were estimated it both soluble (FIG. 28A) and insoluble (FIG. 28B) fractions of LV tissue at four weeks post tamoxifen induction. No DSP is detected in the soluble fractions. Note *This animal was found dead before echocardiography. **PKP2 protein intensity is normalized to tubulin intensity on the same Western blot. For simplicity, only one of the two tubulin blots is shown in FIGS. 28A-B. PKP2 is shown as two bands, the endogenous mouse PKP2 and the larger human PKP2.

### Example 7: AAV Capsid Engineering Through Directed Evolution in NHP

This Examples discloses directed evolution of the AAV9 capsid to identify variants have higher heart transduction and/or high heart-to-liver trafficking ratio (“liver detargeting”). In clinical use, higher heart transduction may result in more efficient therapeutic gene delivery, and thus lead to better efficacy at the same dosage or lower dosage requirement to reach the desired efficacy. Higher heart-to-liver ratio may reduce toxicity and adverse effects related to high liver viral load, while having heart gene delivery unaffected or improved.

### Library Generation and AAV Selection

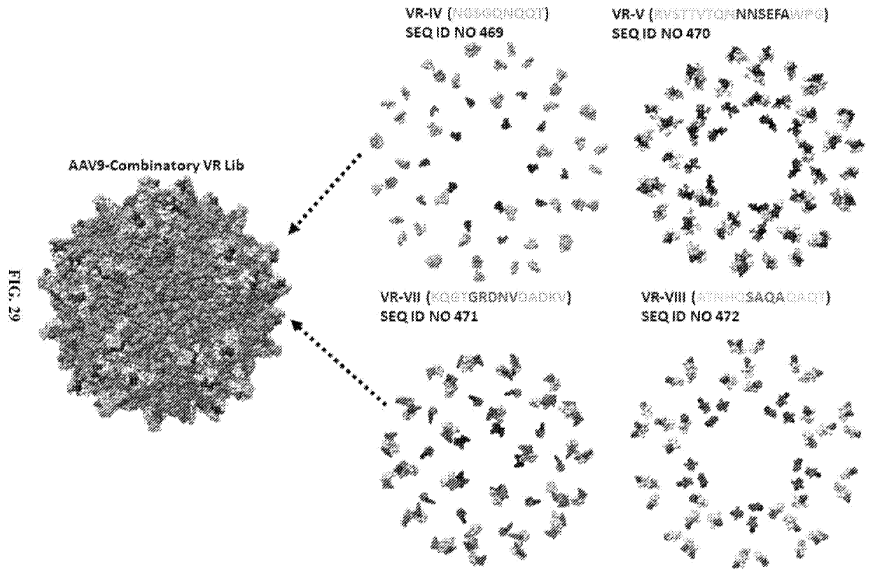

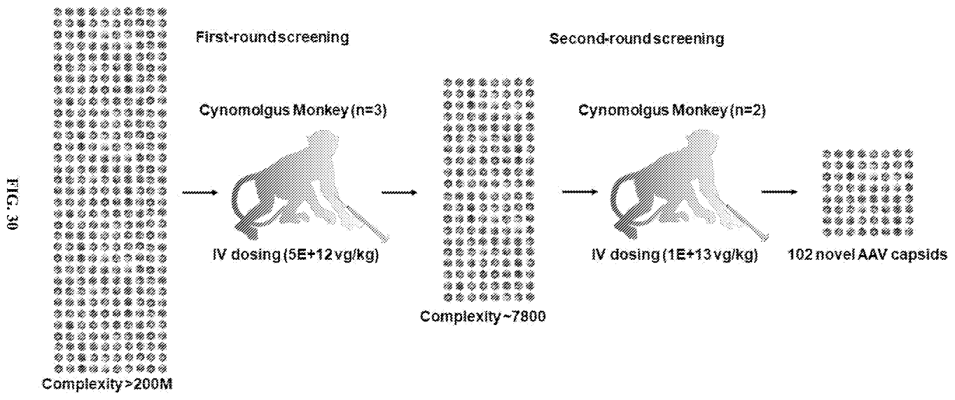

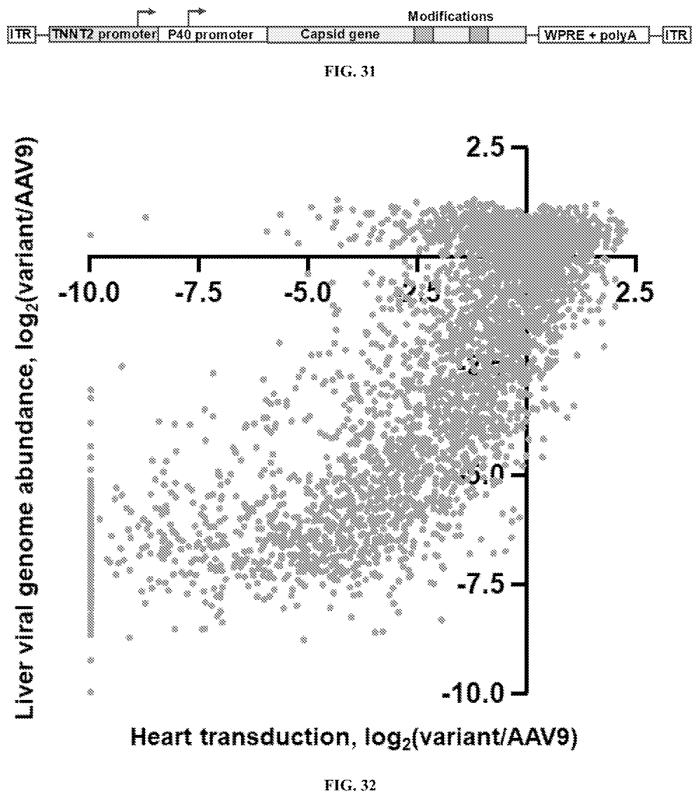

Variable regions (VR-IV and VR-VIII sites) on the AAV9 capsid are shown in FIG. 29. A library screening strategy (FIG. 30) was employed to identify novel AAV capsid variants for better heart gene delivery through systemic administration. A library of more than 200 million capsid variants was generating through synthesis of pools of oligonucleotides encoding random amino-acid residues or designed insertions and substitutions at selected positions in viral proteins (VP) of the AAV9 capsid. The pools were cloned into the capsid (cap) gene in the recombinant vector genome depicted in FIG. 31 that places the capsid (cap) gene under the control of a P40 promoter to express the capsid proteins VP1, VP2, and VP3 in in vitro, thereby generating an infections virions with the modified VP proteins. Upstream of the P40 promoter, a shortened cardiac troponin (TNNT2) promoter, described in US 2022/0031866 A1, was included to drive the expression of the capsid mRNA transcript in cardiomyocytes in vivo, which enables detection of viral mRNA from the heart. This design facilitates selection of only those AAV virions that both traffic to the desired organ (heart) and express the transgene in the cells. When injecting into a subject, the cap gene is expressed in the heart if the encapsulating AAV virion traffics to the heart, enters the cells, and deliver the transgene to the nucleus. Accordingly, the mRNA expressed in the heart should be mRNA corresponding to AAV virions having the desired tropism. Off-target trafficking can be detected by DNA, Trafficking of the AAV virion toa target causes vector genome DNA to be present in that issue.

The library was formed by pooling seven sub-libraries, listed in Table 6. In sub-library 1, positions in the VR-IV site in the capsid sequence of a variant AAV9 capsid were randomized; the variant AAV9 capsid had the artificial sequence ANYG (replacing the native SAQA sequence) in its in VR-VIII site. In sub-libraries 2 through 7, positions in the VR-VIII site of the AAV9 capsid were randomized.

TABLE 6
Variant libraries

| Sub-Library No. | Description |
|---|---|
| 1 | SAQA sequence at positions 586-589 (VR-VIII site) replaced with |
| | ANYG |
| | Positions 452-458 (VR-IV site) replaced with NNNNNNN, where |
| | N represents a random amino acid |
| 2 | Two amino acids inserted at each of positions 582-588 |
| | All 2800 possible variants |
| | (seven positions × 400 possible two amino acid insertions) |
| 3 | Positions 581-594 replaced with sequences predicted by machine |
| | learning algorithm |
| | 200 variants |
| 4 | Positions 582-584, 583-585, 584-586, 585-587, 586-588, or |
| | 587-589 replaced with NXN, where N represents a random amino acid, |
| | and X represents the amino acid in the reference sequence |
| | All 2394 possible variants |
| 5 | Single amino acid substitution at each of positions 581-594 |
| | All 266 possible variants |
| 6 | Position 585-590 replaced with six residue random sequence |
| 7 | Single amino acid substitution at position 587 and single amino |
| | acid insertion at position 589 |

As shown in FIG. 29 and FIG. 30, these initial librarian, which theoretically included more than 200 million cap gene sequences, were packaged using conventional HEK293T AAV production system, where the Adenovirus helper plasmid, the AAV2 REP plasmid, and the CAP library plasmids were transfected into HEK293T cells. Cell lysate was collected after 72 hours and AAV virions were purified by iodixanol gradient ultracentrifugation. The resulting pool of AAV virions were intravenously injected into three Cynomolgus monkeys 5E112 viral genomes per kilogram body weight. At 4-weeks post-injection, animals were sacrificed, and biopsies were taken from the heart and liver. RNA was isolated from heart samples and viral transcripts were amplified and sequenced by Next-Generation Sequencing to detect novel variants that transduced heart. DNA was isolated from liver samples and viral genome sequences were amplified and sequenced by Next-Generation Sequencing (NGS) to detect variants that infected liver. Using computational analysis of the sequencing results, approximately 7800 variants were selected for having high heart-transduction, high heart-to-liver ratio, or both.

A second-round screening was performed by re-synthesizing and cloning DNA sequences encoding the selected 7800 variants, expressing AAV virions, and injecting the resulting AAV pool into two Cynomolgus monkeys intravenously at 1E-+-13 viral genomes per kilogram body weight. At 4-weeks post-injection, animals were sacrificed, and biopsies were taken from the heart and liver. RNA was isolated from heart samples and viral transcripts were amplified and sequenced by Next-Generation Sequencing to detect novel variants that transduced heart. DNA was isolated from liver samples and viral genome sequences were amplified and sequenced by Next-Generation Sequencing (NGS) to detect variants that infected liver. After NGS, computational analysis of second-round screen identified 102 novel AAV capsids as superior to AAV9 in heart transduction, heart-to-liver ratio, or both.

### Screening Results

FIG. 32 plots the data from the second-round screening. All the detectable variants (including two or more synonymous codon replicates for each variant) are plotted. Heart transduction and liver infection measurements were obtained by deep sequencing viral transcripts recovered from heart biopsies or viral genome sequences recovered from liver biopsies, identifying the variant coding sequence on each Next-Generation Sequencing reads, counting the appearance of each unique variant and normalizing the read count of each unique variant to the total number of reads from that biopsy. We also normalized the frequency of each unique variant to its abundance in the input virus library. We report the frequency of each unique variant relative to the frequency of the AAV9 control present in the sample input virus library and calculating the log 2 value of the resulting ratio. Heart mRNA abundance (a measure of infection of cardiomyocytes) is plotted against liver DNA abundance (a measure of trafficking to the liver).

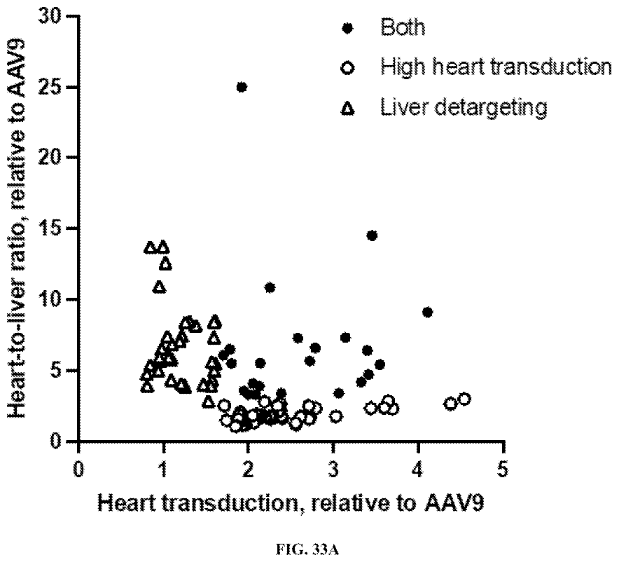

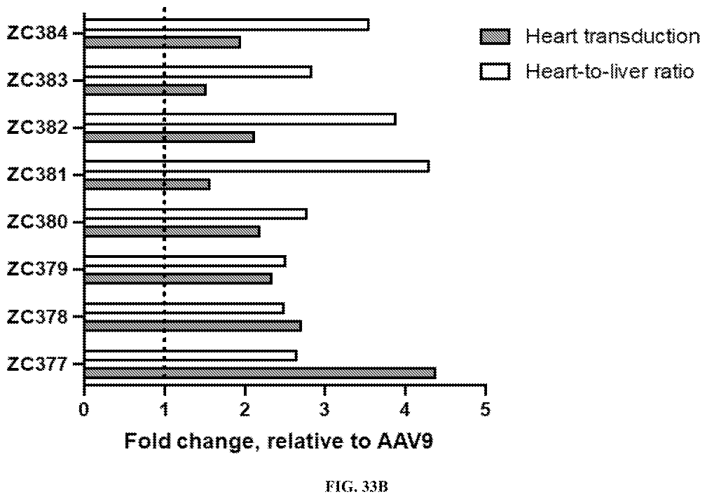

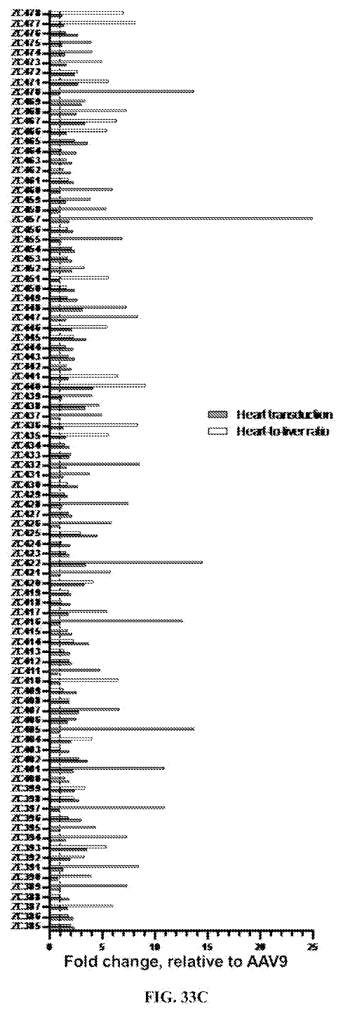

FIGS. 33A-33C plot 102 variants selected as having the desired properties (high heart transduction relative to AAV9, high heart-to-liver ratio relative to AAV9, or both).

FIG. 33A plots heart transduction measurements of the 102 selected variants on x-axis and heart-to-liver ratios on y-axis. Heart-to-liver ratio was calculated by dividing heart transduction measurement by liver infection measurement of each variant. All the values are shown as fold-change relative to AAV9. Variants with improved heart transduction are shown as open circles. Variants with improved heart-to-liver ratio (“liver detargeting”) are shown as open triangles. Variants with both improved heart transduction and improved heart-to-liver ratio are shown as filled circles.

FIG. 33B shows the subset of variants from the sub-library no. 1 in Table 6 with both randomized VR-IV (amino acids 452 to 458 of AAV9 VP1) and substituted VR-VIII (amino acids 586-589 of AAV9 VP1), The measurements are the same as those in FIG. 33A.

FIG. 33C shows novel variants with modified VR-VIII (amino acids 581 to 594 on AAV9 VP1). The measurements are the same as those in FIG. 33A.

### Re-Testing in Mouse Model

Selected AAV variants were tested in mice to confirm performance relative to AAV9.

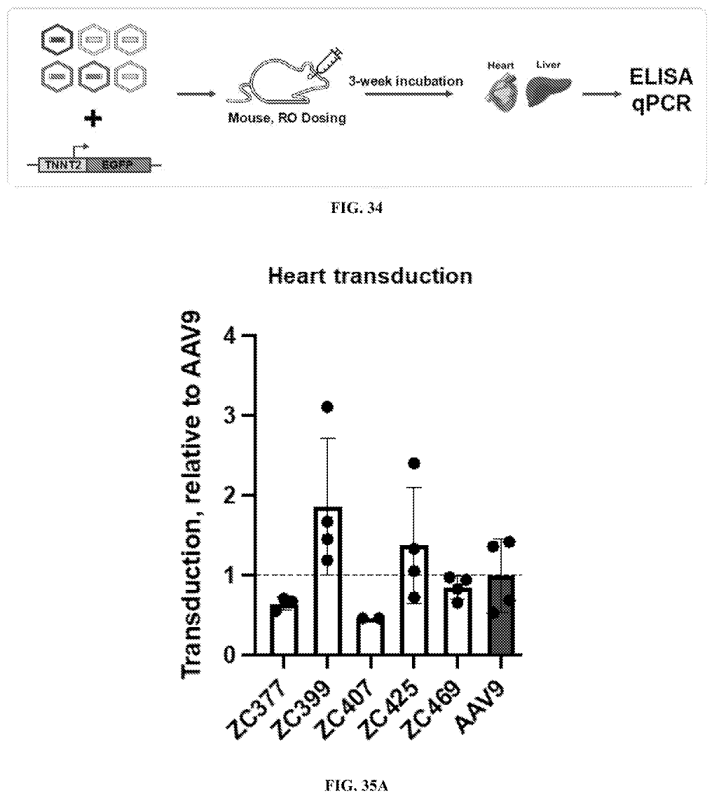

As shown in FIG. 34, five selected capsid sequence, ZC377, ZC399. ZC407, ZC425, and ZC469, and a reference AAV9 capsid sequence were used to produce AAV virions having a vector genome encoding EGFP driven by TNNT2 promoter. The resulting AAV virions were each injected into 6-week-old mice at 6E+12 viral genomes per kilogram body weight through retro-orbital administration, one capsid per animal. At three weeks, animals were sacrificed, and heart and liver were harvested. Heart transduction was measured by ELISA detecting EGFP protein in heart lysates and liver viral load was measure by qPCR targeting EGFP coding sequence in DNA recovered from liver samples.

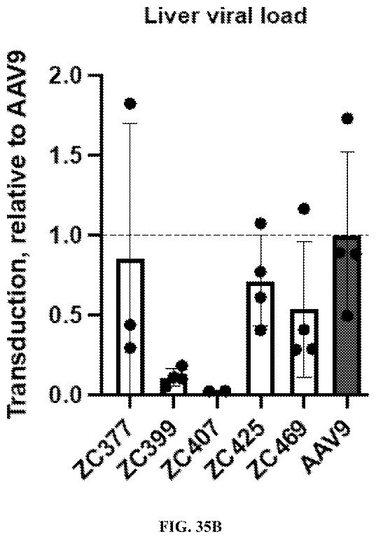

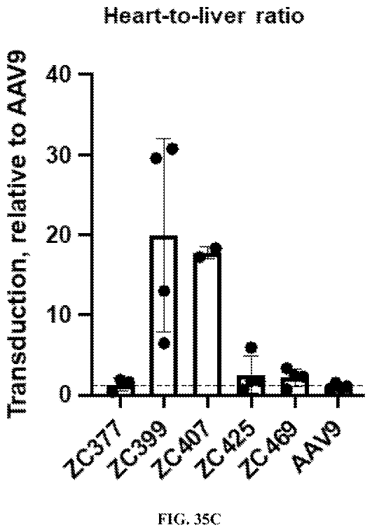

FIGS. 35A-35C show heart transduction (FIG. 35A), liver viral load (FIG. 35B), and heart-to-liver ratio (FIG. 35C) measurements of the selected variants and AAV9 reference. All the numbers are shown as fold-change relative to AAV9 control. Each dot represent data from one animal. ZC399 shows −1.8-fold average heart transduction compared to AAV9. ZC399 and ZC407 show lower liver viral load and −20-fold heart-to-liver ratio compared to AAV9.

### Analysis of Sequences of Selected Capsid Variants

The sequences of the selected variants are provided in Table 7 and Table 8.

TABLE 7
VR-IV variants

| | VP1 | | | | |
|---|---|---|---|---|---|
| Identi- | SEQ | | VR-IV | | VR-VIII |
| fier | ID | VR-IV | SEQ ID | VR-VIII | SEQ ID |
| No. | NO: | (452-458) | NO: | (581-594) | NO: |
| ZC377 | 488 | KGSGQNQ | 590 | ATNHQANYGQAQTG | 598 |
| ZC378 | 489 | NASGQNQ | 591 | ATNHQANYGQAQTG | 598 |
| ZC379 | 490 | NGTGQNQ | 592 | ATNHQANYGQAQTG | 598 |
| ZC380 | 491 | NGSGLNQ | 593 | ATNEQANYGQAQTG | 598 |
| ZC381 | 492 | ANDNKLI | 594 | ATNHQANYGQAQTG | 598 |
| ZC382 | 493 | VNDNKVI | 595 | ATNHQANYGQAQTG | 598 |
| ZC383 | 494 | NGSGQNH | 596 | ATNHQANYGQAQTG | 598 |
| ZC384 | 495 | ANDNKVI | 597 | ATNHQANYGQAQTG | 598 |

TABLE 8
VI-VIII variants

| | VP1 | | |
|---|---|---|---|
| Identifier | SEQ ID | | VR- VII |
| No. | NO: | VR-VIII (581-594) | SEQ ID NO: |
| ZC385 | 496 | ATNHTSFQAQAQTG | 599 |
| ZC386 | 497 | ATNHCSAQAQAQTG | 600 |
| ZC387 | 498 | ATNHVDSLRIAQTG | 601 |
| ZC388 | 499 | ATNRQTAQAQAQTG | 602 |
| ZC389 | 500 | ATNHTGTSITAQTG | 603 |
| ZC390 | 501 | ATNHLSNFNSAQTG | 604 |
| ZC391 | 502 | ATNHCTLNSIAQTG | 605 |
| ZC392 | 503 | ADVQQKPGSQIQTQ | 606 |
| ZC393 | 504 | ATNHNMNRVNAQTG | 607 |
| ZC394 | 505 | ATNHNNVISGAQTG | 608 |
| ZC395 | 506 | ATNHSNSVQSAQTG | 609 |
| ZC396 | 507 | ATNHQSPIAQAQAQTG | 610 |
| ZC397 | 508 | ATNHLSKVFDAQTG | 611 |
| ZC398 | 509 | ATNHQSAITQAQAQTG | 612 |
| ZC399 | 510 | ATNASSTFQGAQTG | 613 |
| ZC400 | 511 | ATNHNSIQAQAQTG | 614 |
| ZC401 | 512 | ATNHMMTTARAQTG | 615 |
| ZC402 | 513 | ATNHQGAYAQAQTG | 616 |
| ZC403 | 514 | ALNKQSAQAQAQTG | 617 |
| ZC404 | 515 | ATNHENTVSIAQTG | 618 |
| ZC405 | 516 | ATNHVSSFTSAQTG | 619 |
| ZC406 | 517 | ATNHPSIHQGAQTG | 620 |
| ZC407 | $18 | ATNHSTTNFRAQTG | 621 |
| ZC408 | 519 | ATNHQHYSAQAQAQTG | 622 |
| ZC409 | 520 | ATNKQTAQAQAQTG | 623 |
| ZC410 | 521 | ATNHSSIFNSAQTG | 624 |
| ZC411 | 522 | ATNHAGNYNNAQTG | 625 |
| ZC412 | 523 | AEVQQSSMSQAQTD | 626 |
| ZC413 | 524 | AANVQSAQAQAQTG | 627 |
| ZC414 | 525 | ATNYQQAQAQAQTG | 628 |
| ZC415 | 526 | ATNHQSVQGAQAQTG | 629 |
| ZC416 | 527 | ATNHGSILTHAQTG | 630 |
| ZC417 | 528 | ATNHQLFSKNAQTG | 631 |
| ZC418 | 529 | AANMQSAQAQAQTG | 632 |
| ZC419 | 530 | ATNQQIAQAQAQTG | 633 |
| ZC420 | 531 | ATNTYHQSAQAQAQTG | 634 |
| ZC421 | 532 | ATNHCDPLHIAQTG | 635 |
| ZC422 | 533 | ATNHISVISIAQTG | 636 |
| ZC423 | 534 | ATNHQLASAQAQTG | 637 |
| ZC424 | 535 | ATNHQVTSAQAQAQTG | 638 |
| ZC425 | 536 | ATNHHSRVEIAQTG | 639 |
| ZC426 | 537 | ATNHTSFTWTAQTG | 640 |
| ZC427 | 538 | ATNHQSAPTQAQAQTG | 641 |
| ZC428 | 539 | ATNHNSTYLGAQTG | 642 |
| ZC429 | 540 | ATNHQIAQAQAQTG | 643 |
| ZC430 | 541 | ATNHQAISAQAQAQTG | 644 |
| ZC431 | 542 | ATNHLSVVYNAQTG | 645 |
| ZC432 | 543 | ATNHMHQSAQAQAQTG | 646 |
| ZC433 | 544 | ATNHETSRLNAQTG | 647 |
| ZC434 | 545 | AFNWQSAQAQAQTG | 648 |
| ZC435 | 546 | ATNHNTVMLGAQTG | 649 |
| ZC436 | 547 | ATNHESSMLNAQTG | 650 |
| ZC437 | 548 | ATNHASITSSAQTG | 651 |
| ZC438 | 549 | ARNEQSAQAQAQTG | 652 |
| ZC439 | 550 | ATNHANLYQMAQTG | 653 |
| ZC440 | 551 | ATNHQFATAQAQTG | 654 |
| ZC441 | 552 | ATNFNHQSAQAQAQTG | 655 |
| ZC442 | 553 | ATNHMSHQAQAQTG | 656 |
| ZC443 | 554 | ATNHQWMSAQAQAQTG | 657 |
| ZC444 | 555 | ATNHQSGQQAQAQTG | 658 |
| ZC445 | 556 | ATNHSSADAQAQTG | 659 |
| ZC446 | 557 | ATNHTTKTMFAQTG | 660 |
| ZC447 | 558 | ATNHSSIIYSAQTG | 661 |
| ZC448 | 559 | ATNHMLLKSNAQTG | 662 |
| ZC449 | 560 | ATNHESMQAQAQTG | 663 |
| ZC450 | 561 | ATNHQMLSAQAQAQTG | 664 |
| ZC451 | 562 | ATNHSGRDSYAQTG | 665 |
| ZC452 | 563 | ATNHINVISGAQTG | 666 |
| ZC453 | 564 | ATNHVSNQAQAQTG | 667 |
| ZC454 | 565 | ATNHNTKLAIAQTG | 668 |
| ZC455 | 566 | ATNHSSSYNNAQTG | 669 |
| ZC456 | 567 | ATNATHQSADAQAQTG | 670 |
| ZC457 | 568 | ATNHLRDNISAQTG | 671 |
| ZC458 | 569 | ATNHSSFSVGAQTG | 672 |
| ZC459 | 570 | ATNHVNRNLSAQTG | 673 |
| ZC460 | 571 | ATNHHNPSINAQTG | 674 |
| ZC461 | 572 | ATNHQDARAQAQTG | 675 |
| ZC462 | 573 | ATNDQRAQAQAQTG | 676 |
| ZC463 | 574 | ATNVQTAQAQAQTG | 677 |
| ZC464 | 575 | APNRQSAQAQAQTG | 678 |
| ZC465 | 576 | ATNRQIAQAQAQTG | 679 |
| ZC466 | 577 | ATNHEDNIRRAQTG | 680 |
| ZC467 | 578 | ATNHNRNGLLAQTG | 681 |
| ZC468 | 579 | ATNHESTSVRAQTG | 682 |
| ZC469 | 580 | ATNHNIRTEMAQTG | 683 |
| ZC470 | 581 | ATNHQTLFNSAQTG | 684 |
| ZC471 | 582 | ATNHHSWQAQAQTG | 685 |
| ZC472 | 583 | ATNHSTKSLIAQTG | 686 |
| ZC473 | 584 | ATNHQKLLVNAQTG | 687 |
| ZC474 | 585 | ATNHLSVSSIAQTG | 688 |
| ZC475 | 586 | ATNHVSNLYGAQTG | 689 |
| ZC476 | 587 | ATNRQMAQAQAQTG | 690 |
| ZC477 | 588 | ATNHEDIIRSAQTG | 691 |
| ZC478 | 589 | ATNHCSTSIRAQTG | 692 |

Variants having insertions in the VR-VIII site were manually aligned to show the insertions. Results are shown in Table 9.

TABLE 9

| Identi- | | SEQ | Insertion | |
|---|---|---|---|---|
| fier | | ID | position | |
| No. | Alignment | NO: | (after) | Insertion |
| ZC396 | N--H--Q--SPIA--Q | 693 | 586 | PI |
| ZC398 | N--H--Q--S--AITQ | 694 | 587 | IT |
| ZC408 | N--H--QHYS--A--Q | 695 | 585 | HY |
| ZC420 | NTYH--Q--S--A--Q | 696 | 583 | TY |
| ZC424 | N--H--QVTS--A--Q | 697 | 585 | VT |
| ZC427 | N--H--Q--S--APTQ | 698 | 587 | PT |
| ZC430 | N--H--QAIS--A--Q | 699 | 585 | AI |
| ZC432 | N--HMHQ--S--A--Q | 700 | 584 | MH |
| 20441 | NFNH--Q--S--A--Q | 701 | 583 | FN |
| 2C443 | N--H--QWMS--A--Q | 702 | 585 | WM |
| 2C450 | N--N--QMLS--A--Q | 703 | 585 | ML |
| 20456 | NATH--Q--S--A--Q | 704 | 583 | AT |

Selected variant sequences will be further tested as described below.

### Prophetic Example 8: Confirmatory Re-Screening in Primates

Selected AAV variants are tested in primates to confirm performance relative to AAV9. To perform the experiment with further animals, a pool-injection-and-screen strategy analogous to the original screening protocol is used.

All the 102 novel capsids are packaged individually using transgene cassette carrying barcoded EGFP reporter driven by TNNT2 promoter. The resulting AAV virions are pooled and injected into Cynomolgus monkeys through intravenous administration. At 4-week post-injection, animals are sacrificed, and biopsies taken from heart and liver, as well as other tissues. RNA is isolated from heart samples. The barcoded region on the viral transcript is amplified and sequenced by Next-Generation Sequencing. The heart transduction ability of each variant is quantified with normalized read counts of corresponding barcodes. DNA is isolated from liver samples. The barcoded region on the viral transgene is processed in the same way as the heart RNA samples. The liver tropism of each variant is quantified with normalized read counts of corresponding barcodes.

A similar study in mouse is performed in parallel.

### Example 9: Neutralizing Antibody Prevalence Study

Selected capsids are assayed against pooled human IgG samples, as well as individual human serum samples. The ability to escape from neutralization by pooled IgG and increased percentage of sero-negative human individuals indicate potential wider patient coverage than AAV9.

### Example 10: Biodistribution Study in Primates

Selected capsid and AAV9 control are tested in a biodistribution study In Cynomolgus monkeys. Each animal receives one test article and each test article is tested on three animals. The transduction and viral genome distribution is examined in various organs and tissues.

### Example 11: Novel AAV Capsids with Improved Transduction Properties

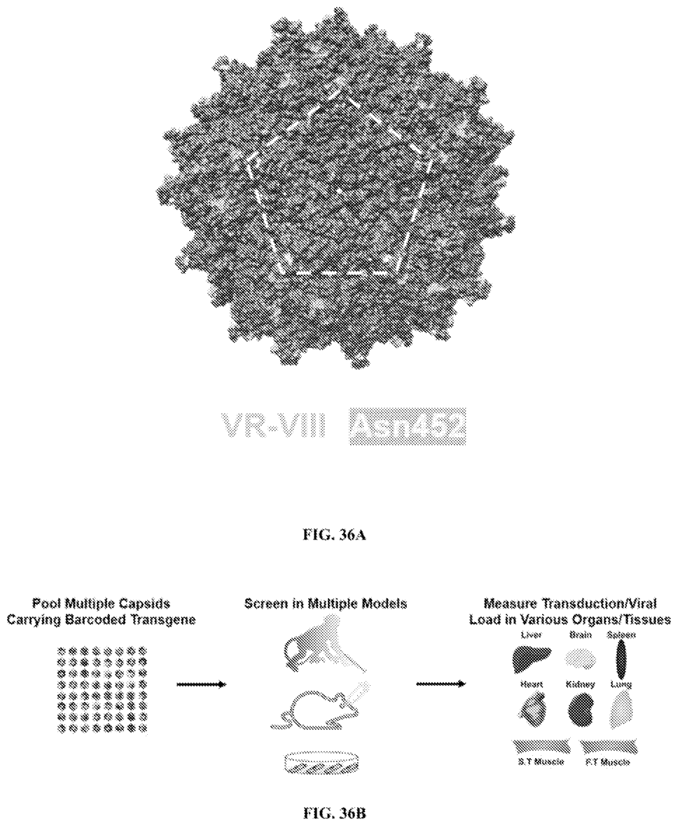

The purpose of this study was to identify novel AAV capsid variants that have superior properties, such as improved transduction. Capsids with modified VR-VIII (amino acids 585 to 590, AAV9 VP1 numbering) were further modified at amino acid 452 (AAV9 VP1 numbering, Asparagine/Asn/N452) (FIG. 36A; Mutations in Tables 8 and 9), The resulting capsid variants were packaged individually using barcoded transgene cassettes, pooled together, and tested in non-human primates (cynomolgus monkey/Macaca fascicularis/Cyno), CD-1 mice, and human iPSC-derived cardiomyocytes (iPSC-CMs). The doses provided herein represent the total amount of virus in the pool. Cynomolgus monkeys and pigs were administered virus at 1E+13 vg/kg via intravenous bolus administration and tissue was collected 4-weeks after injection. Two groups of mice were administered virus, three mice at 1E+13 vg/kg and three mice at 5E+13 vg/kg via retro-orbital administration and tissue was collected 18-days post injection. For the iPSC-CMs, two populations of cells were administered virus at different doses, 1.6E44 vg/cell and 1.6E4+vg/cell and samples were collected 4 days later. The transduction/viral load levels of capsids variants in different organs (such as heart, liver, brain, skeletal muscle) and iPSC-CMs were measured by quantifying the barcodes by next-generation sequencing (NGS) (FIG. 36B). The average of the two dose level groups for the mice and iPSCs is shown.

Experiments with some of the identified mutated capsids from the screen are shown in FIG. 37. In particular, ZC404 (SEQ ID NO: 618), ZC470 (SEQ ID NO: 684), ZC428 (SEQ ID NO:642), are ZC416 (SEQ ID NO: 630) are variants with VR-VIII modifications. Their transduction and viral load levels in Cyno heart, Cyno liver, mouse heart, mouse liver, and human iPSC-CMs are shown as white bars in FIG. 37. Introduction of the N42K mutation into these capsids (Tables 8 and 9) resulted in four combinatory capsids, ZC373 (SEQ ID NO: 705), ZC374 (SEQ ID NO: 706), ZC375 (SEQ ID NO: 707), and ZC376 (SEQ ID NO: 708), and their transduction viral load levels in Cyno heart, Cyno liver, mouse heart, mouse liver, and human iPSC-CMs was measured, with the results shown in FIG. 37 as dark/shaded bars. All these combinatory capsids showed improved transduction/viral load (particularly in the heart) compared to the original VR-VIII variants, showing that N452K, can enhance the transduction of AAV9-based capsids regardless of what modifications have been made in other regions of the capsid.

A capsid (ZC537) with N452K mutation as the only modification was also generated. In addition, capsids ZC531, ZC532, ZC533, ZC534, ZC535, ZC536, ZC538I, ZC539, ZC540, ZC541, ZC542 with N452K mutation in addition to other mutations in VR-VIII were generated.

TABLE 8
Novel VP1 Capsids

| Capsid | | | | |
|---|---|---|---|---|
| Identi- | Posi- | VR-VIII | VR-VIII | |
| fier | tion | Alignment | SEQ ID | VPI Capsid |
| No. | 452 | (581-594) | NO: | SEQ ID NO: |
| ZC373 | K | ATNHENTVSIAQTG | 618 | 705 |
| ZC374 | K | ATNHQTLFNSAQTG | 684 | 706 |
| ZC375 | K | ATNHNSTYLGAQTG | 642 | 707 |
| ZC376 | K | ATNHGSILTHAQTG | 630 | 708 |
| ZC404 | N | ATNHENTVSIAQTG | 618 | 515 |
| ZC470 | N | ATNHQTLFNSAQTG | 684 | 581 |
| ZC428 | N | ATNHNSTYLGAQTG | 642 | 539 |
| ZC416 | N | ATNHGSILTHAQTG | 630 | 527 |
| ZC531 | K | ATNHMMTTARAQTG | 615 | 767 |
| ZC532 | K | ATNHCSTSIRAQTG | 692 | 768 |
| ZC533 | K | ATNHQGAYAQAQTG | 616 | 769 |
| ZC534 | K | ATNHNTKLAIAQTG | 668 | 770 |
| ZCS35 | K | ATNHVSSFTSAQTG | 619 | 771 |
| ZC536 | K | ATNHEDNIRSAQTG | 726 | 772 |
| ZC537 | K | ATNHQSAQAQAQTG | 5 | 773 |
| ZC538 | K | ATNHNNVISGAQTG | 608 | 774 |
| ZC539 | K | ATNHTGTSIIAQTG | 603 | 775 |
| ZC540 | K | ATNHQWMSAQAQAQTG | 657 | 776 |
| ZC541 | K | ATNHQDARAQAQTG | 675 | 777 |
| ZC542 | K | ATNHQHYSAQAQAQTG | 622 | 778 |
| AAV9 | | ATNHQSAQAQAQTG | 5 | 1 |

VP1 capsid sequence comprises SEQ ID NO: 1 modified at positions between 581-594 as indicated in this table

TABLE 9
VR-VIII and N452 Substitutions in Certain Novel VP1 Capsids*

| Capsid | | | | | | | |
|---|---|---|---|---|---|---|---|
| Identifier | Position | Position | Position | Position | Position | Position | Position |
| No. | 585 | 586 | 587 | 588 | 589 | 590 | 452 |
| ZC373 | Q58SE | S586N | A587T | Q588V | A589S | QS90I | N452K |
| ZC374 | | S586T | A587L | Q588F | A589N | Q590S | N452K |
| ZC375 | Q585N | | AS87T | Q588Y | A589L | Q590G | N452K |
| ZC376 | Q585G | | AS87I | Q588L | AS89T | Q590H | N452K |
| ZC404 | Q585E | SS86N | AS87T | Q588V | AS89S | Q590I | |
| ZC470 | | SS86T | AS87L | Q588F | AS89N | Q590S | |
| ZC428 | Q585N | | A587T | Q588Y | A589L | Q590G | |
| ZC416 | Q585G | | A587I | Q588L | A589T | Q590H | |
| ZC531 | Q585M | S586M | A587T | Q588T | | QS90R | N452K |
| ZC532 | Q58SC | | A587T | Q588S | A589I | QS90R | N452K |
| ZC533 | | S586G | | Q588Y | | | N452K |
| ZC534 | Q585N | S586T | A587K | Q588L | | Q590I | N452K |
| ZC535 | Q585V | | A587S | Q588F | AS89T | Q590S | N452K |
| ZC536 | Q585E | S586D | A587N | Q588I | A589R | Q590S | N452K |
| ZC537 | | | | | | | N452K |
| ZC538 | Q585N | S586N | A587V | Q588I | A589S | Q590G | N452K |
| ZC539 | Q58ST | S586G | A587T | Q588S | A589I | Q590I | N452K |
| ZC541 | | S586D | | Q588R | | | N452K |
| ZC369 | Q585N | S586I | AS87R | Q588T | A589E | Q590M | N452K |
| ZC370 | Q585S | S586T | A587T | Q588N | AS89F | Q590R | N452K |
| AAV9 | | | | | | | |

*No entry in the table = no substitution
Note:
all of the capsids in Table 9 have (i) ATNH at positions 581, 582, 583 and 584, respectively, and (ii) AQTG at positions 591, 592, 593 and 594, respectively.

Accordingly, the identified capsids comprise the indicated amino acids at indicated positions of VR-VIII (where the only or last amino acid corresponds to the unmodified AAV9 amino acid):

| 581 | 582 | 583 | 584 | 585 | 586 | 587 | 588 | 589 | 590 | 591 | 592 | 593 | 594 |
|---|---|---|---|---|---|---|---|---|---|---|---|---|---|
| A | T | N | H | E, N, | N, T, | T, L, | V, F, | S, N, | I, S, | A | Q | T | G |
| | | | | G, M, | M, G, | I, K, | Y, L, | L, T, | G, H, | | | | |
| | | | | C, V, | D, S | S, N, | T, S, | I, R, | R, Q | | | | |
| | | | | T, Q | | V, A | I, R, | A | | | | | |
| | | | | | | | Q | | | | | | |

TABLE 11
VR-VIII Insertions in Certain Novel VP1 Capsids

| Capsid | | | | |
|---|---|---|---|---|
| Identi- | Posi- | VR-VIII | VR-VIII | |
| fier | tion | Alignment | SEQ ID | |
| No. | 452 | (581-594) | NO: | Comments |
| ZC540 | K | ATNHQWMSAQAQAQTG | 657 | Insertion of |
| | | | | WM before |
| | | | | position 586 |
| | | | | (between |
| | | | | positions 585 |
| | | | | and 586 of |
| | | | | SEQ ID |
| | | | | NO: 1) |
| ZC542 | K | ATNHQHYSAQAQAQTG | 622 | Insertion of |
| | | | | HY before |
| | | | | position 586 |
| | | | | (between |
| | | | | positions 585 |
| | | | | and 586 of |
| | | | | SEQ ID |
| | | | | NO: 1) |
| AAV9 | N | ATNHQ SAQAQAQTG | 5 | |

### Example 12: Characterizing Novel AAV Capsids in Multiple Mammalian Models

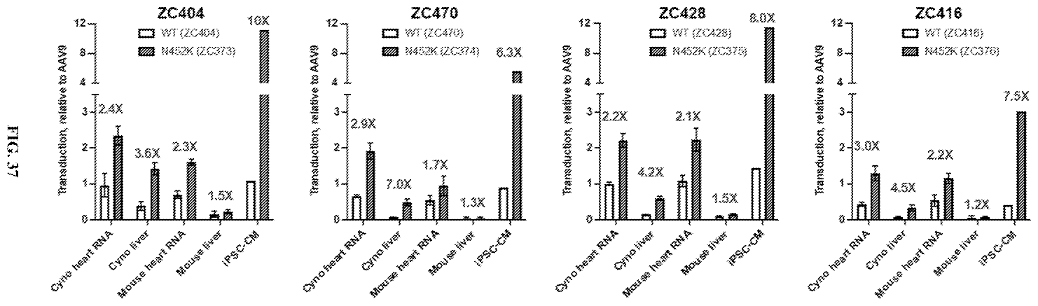

The purpose of this study was to compare the performance of the novel AAV capsids described above in multiple models including non-human primate, mouse, pig, and in vitro human iPSC-CMs. The novel AAV capsids and control capsids were packaged individually, and barcoded transgene cassettes were used to enable pooled next-generation sequencing based Capsid transduction assays (FIG. 38). The viruses were pooled together targeting equal viral genome ratio and the pool was tested in viva in Cynomolgus Monkey, CD-1 mice, and pig, as well as in vitro on human iPSC-derived cardiomyocytes. Animals and cells were administered virus, and tissue was collect, as described in Example 11. To measure transduction efficiency and/or viral load, heart tissue, liver tissue, and iPSC-CM % were collected, followed by RNA and DNA extraction. The barcoded region was amplified from RNA and DNA samples and sequenced by next-generation sequencing. The RNA or DNA raw read count of each barcode was normalized to the total read number in the sequencing run and to the abundance in the initial virus pool. The average measurement of multiple barcodes belonging to the same capsid was calculated to determine the transduction efficiency or viral load of the capsid.

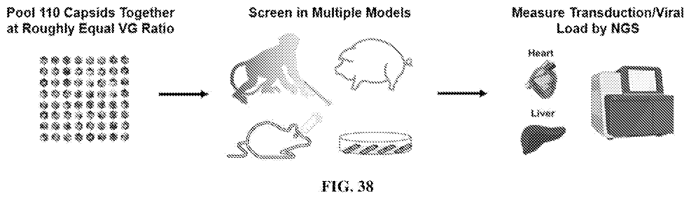

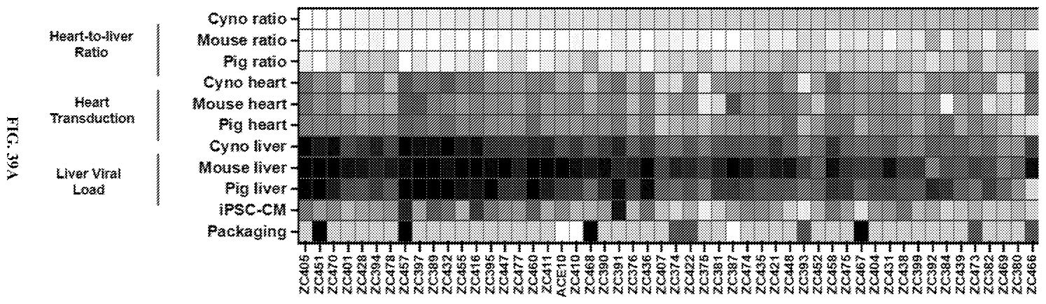

Heart transduction was measured with RNA signal in the heart tissue. Liver viral load was measured with DNA signal in the liver tissue. Heart-to-liver ratio was determined by dividing heart transduction by liver viral load. Transduction efficiency on iPSC-CMs was determined by RNA signal. Packaging scores were determined by virus yield in HEK293T production system. The average measurements of 4 animals, 3 animals, 6 animals, or 2 multiplicities of infection were shown for Cynomolgus monkey, mouse, pig, and iPSC-CMs, respectively (FIGS. 39A and 39B). FIGS. 39A and 39B represent the whole dataset heatmap. Each column on the heatmap represents one capsid and each row represents one sample type. White color means higher value and dark color means lower value, with the median grayscale representative of wildtype AAV9 control. The capsids are ranked from left to right by their heart-to-liver ratios in Cynomolgus monkey. AAV9-1, AAV9-2, and AAV9-3 are all wildtype AAV9 capsids that served as control replicates. CR9-10, TN47-10 and TN44-07 are as disclosed in WO 2021/216456 A2 (by reference to the same capsid name), the disclosures of which are specifically incorporated by reference herein. The sequences of other capsids are disclosed herein.

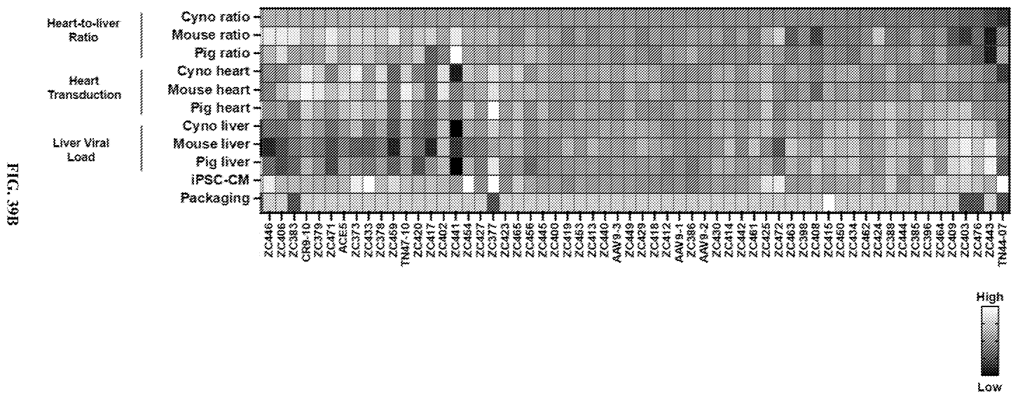

Exemplary novel capsids were selected from the screen in FIGS. 39A and 39B (Table 10) to evaluate transduction efficiency across different species. The heart-to-liver ratio, heart transduction, and liver viral load measurements of four novel capsids and AAV9 control in Cynomolgus monkey (light gray bars), mouse (white bars), and pig (dark bars) were evaluated relative to the performance of wildtype AAV9 control (FIG. 40). Animals were administered virus, and tissue was collected, as described above. These ratios are shown specifically for non-human primates (NHP) in FIG. 41 (Cynomolgus monkey). The novel capsids showed improved heart-to-liver ratio in all three species, demonstrating species consistency. Furthermore, in NHP, the novel capsids showed improved heart-to-liver ratio, at least comparable heart transduction, and less liver viral load, relative to AAV9.

TABLE 10
Capsids Used in the Study

| | VP1 Capsid |
|---|---|
| Identifier No. | SEQ ID NO |
| ZC373 | 705 |
| ZC374 | 706 |
| ZC375 | 707 |
| ZC376 | 708 |
| ACE5 | 709 |
| ACE10 | 710 |
| AAV9-1/2/3 | 1 |
| CR9-10 | 404 |
| TN44-07 | 457 |
| TN47-10 | 458 |
| ZC377 | 488 |
| ZC378 | 489 |
| ZC379 | 490 |
| ZC380 | 491 |
| ZC381 | 492 |
| ZC382 | 493 |
| ZC383 | 494 |
| ZC384 | 495 |
| ZC385 | 496 |
| ZC386 | 497 |
| ZC387 | 498 |
| ZC388 | 499 |
| ZC389 | 500 |
| ZC390 | 501 |
| ZC391 | 502 |
| ZC392 | 503 |
| ZC393 | 504 |
| ZC394 | 505 |
| ZC395 | 506 |
| ZC396 | 507 |
| ZC397 | 508 |
| ZC398 | 509 |
| ZC399 | 510 |
| ZC400 | 511 |
| ZC401 | 512 |
| ZC402 | 513 |
| ZC403 | 514 |
| ZC404 | 515 |
| ZC405 | 516 |
| ZC406 | 517 |
| ZC407 | 518 |
| ZC408 | 519 |
| ZC409 | 520 |
| ZC410 | 521 |
| ZC411 | 522 |
| ZC412 | 523 |
| ZC413 | 524 |
| ZC414 | 525 |
| ZC415 | 526 |
| ZC416 | 527 |
| ZC417 | 528 |
| ZC418 | 529 |
| ZC419 | 530 |
| ZC420 | 531 |
| ZC421 | 532 |
| ZC422 | 533 |
| ZC423 | 534 |
| ZC424 | 535 |
| ZC425 | 536 |
| ZC427 | 538 |
| ZC428 | 535 |
| ZC429 | 540 |
| ZC431 | 542 |
| ZC432 | 543 |
| ZC433 | 544 |
| ZC434 | 545 |
| ZC435 | 546 |
| ZC436 | 547 |
| ZC438 | 549 |
| ZC439 | 550 |
| ZC440 | 551 |
| ZC441 | 552 |
| ZC442 | 553 |
| ZC443 | 554 |
| ZC444 | 555 |
| ZC445 | 556 |
| ZC446 | 557 |
| ZC447 | 558 |
| ZC448 | 559 |
| ZC449 | 560 |
| ZC450 | 561 |
| ZC451 | 562 |
| ZC452 | 563 |
| ZC453 | 564 |
| ZC454 | 565 |
| ZC455 | 566 |
| ZC456 | 567 |
| ZC457 | 568 |
| ZC458 | 569 |
| ZC459 | 570 |
| ZC460 | 571 |
| ZC461 | 572 |
| ZC462 | 573 |
| ZC463 | 574 |
| ZC464 | 575 |
| ZC465 | 576 |
| ZC466 | 577 |
| ZC467 | 578 |
| ZC468 | 579 |
| ZC469 | 580 |
| ZC470 | 581 |
| ZC471 | 582 |
| ZC472 | 583 |
| ZC473 | 584 |
| ZC474 | 585 |
| ZC475 | 586 |
| ZC476 | 587 |
| ZC477 | 588 |
| ZC478 | 589 |

### Example 13: Top Novel Capsids Show Superior Performance when Administered Individually

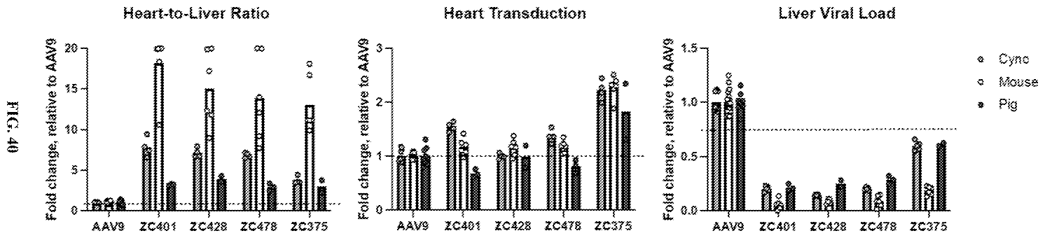

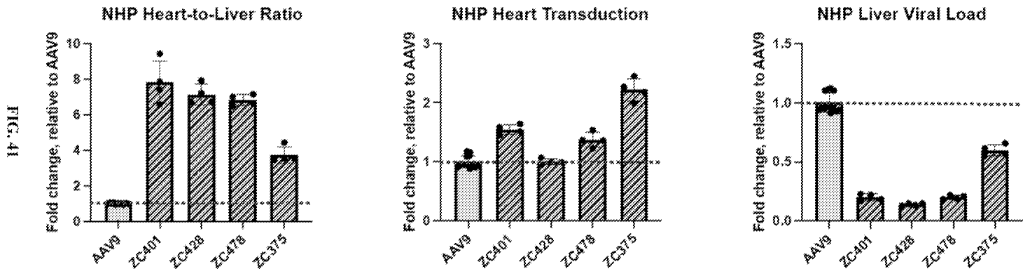

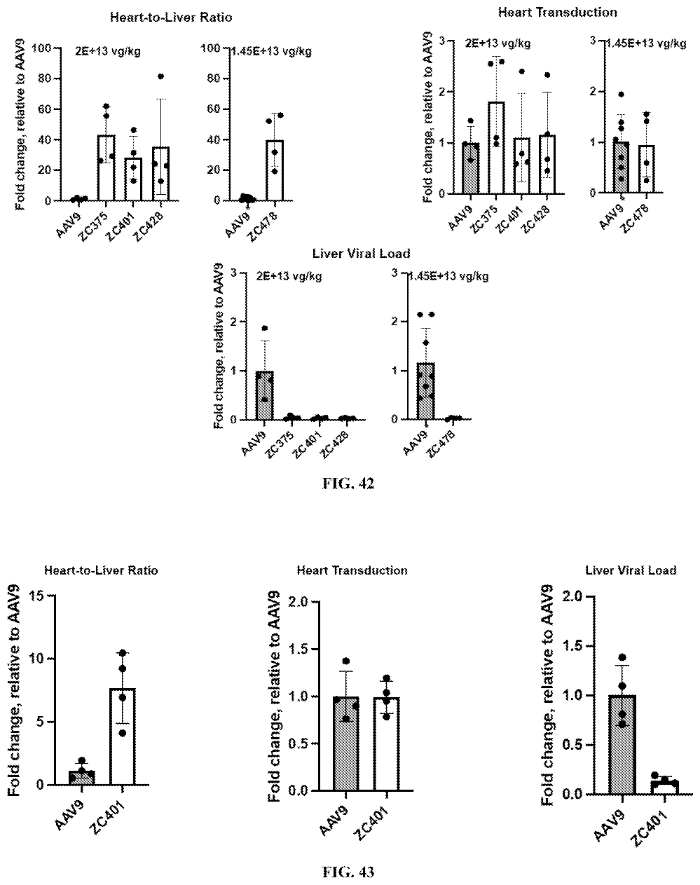

To study whether the pooled capsid comparison results can predict performance in individual animal injections (one test article per animal), the top four novel capsids and AAV9 were tested in CD-1 mice using retro-orbital injection. The viruses were administered at 2E-+13 vg/kg for ZC375. ZC401, and ZC428, and 1.45E+13 vg/kg for ZC478. Dosage matched AAV9 controls were included. Animals were sacrificed at day 18 post injection. Heart transduction was measured by RT-qPCR based quantification of transgene mRNA expression in the heart. Liver viral load was measured by qPCR-based quantification of transgene DNA copies in the liver. Heart-to-liver ratio was determined by dividing heart transduction by liver viral load. All four novel capsids showed improved heart-to-liver ratio in this individual test, consistent with pooled test results (FIG. 42).

To test whether the superior performance of the novel capsids was CD-1 mouse strain specific, ZC401 and AAV9 were evaluated in a second mouse strain, C57BL/6NCrl from Charles River Laboratories. The viruses were administered at 2E+13 vg/kg by retro-orbital injection. Animals were sacrificed at day 18 post injection. Transduction was measured as described above. Novel capsid ZC401 demonstrated improved hear-to-liver ratio, consistent with CD-1 strain results (FIG. 43).

| | VR-VIII | | |
|---|---|---|---|
| Identifier | Alignment | VR-VIII | Capsid SEQ |
| No. | (581-594) | SEQ ID NO: | ID NO: |
| ZC375 | ATNHNSTYLGAQTG | 642 | 707 |
| ZC401 | ATNHMMTTARAQTG | 615 | 512 |
| ZC428 | ATNHNSTYLGAQTG | 642 | 539 |
| ZC478 | ATNHCSTSIRAQTG | 692 | $89 |

### Example 14: Novel Capsid ZC401 Achieved Increased Heart Transduction without Liver Overload

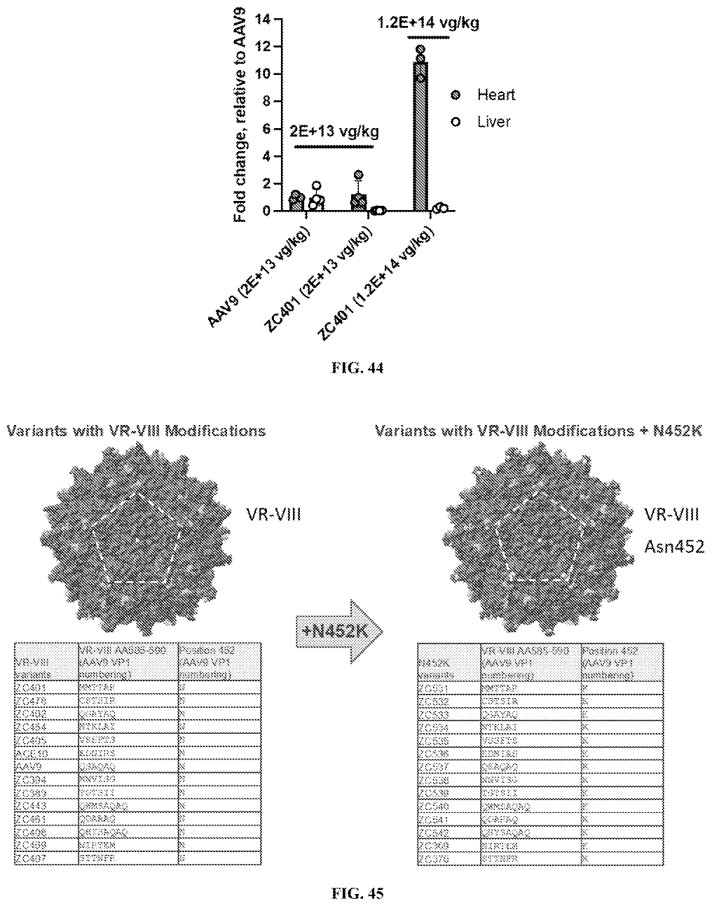

While Novel capsids with improved heart-to-liver ratio can reduce liver burden without compromising heart transduction, they can also enable higher safe dosage at which heart transduction is improved and liver viral load is still lower compared to AAV9 at regular dosage. To test the latter application, a proof-of-concept study was performed comparing ZC401 and AAV9 in CD-1 mice. The viruses were administered at 2E+13 vg/kg (AAV9 and ZC401) or 1.2E+14 vg/kg (ZC401) by retro-orbital injection. Animals were sacrificed at day 18 post injection. Heart transduction was measured by RT-qPCR based quantification of transgene mRNA expression in the heart. Liver viral load was measured by qPCR-based quantification of transgene DNA copies in the liver. Heart-to-liver ratio was determined by dividing heart transduction by liver viral load. Measured values are fold change relative to AAV9. Novel capsid ZC401 at 1.2E+-14 vg/kg dose showed 8× heart transduction level compared to AAV9 at 2E+13 vg/kg, while having just 21% of its liver viral load (FIG. 44).

The data described herein characterized 102 capsids in NHPs, mice, pigs and human iPSC-derived cardiomyocytes (hiPSC-CMs) and identified multiple novel AAV capsids with superior properties including improved heart-to-liver ratio, improved cardiomyocyte transduction, and excellent consistency between different species. Together, these novel AAV capsids allow for more efficacious and safer gene therapies for cardiac disorders.

### Example 15: Characterizing AAV9 Capsids with N452K Substitution in Multiple Mammalian Models

To test the compatibility of N452K substitution with AAV9-based capsid variants and characterize how N452K affects transduction efficiency, 14 additional N452K-containing variants were generated and tested by comparing them to parental wildtype AAV9 or AAV9-based VR-VIII substitution variants. The purpose of this study was to compare the performance of AAV capsids with N452K substitution (some of which were described in Example 11 above, and further described in FIG. 45) with parental AAV9 capsids (including wild-type AAV9 and AAV9 capsids with VR-VIII substitutions) in multiple models including non-human primate, mouse, and in vitro human iPSC-CMs.

The capsids were administered and tested in vivo in Cynomolgus Monkey and C57BL6NCrl mice, as well as in vitro on human iPSC-derived cardiomyocytes as described in Examples 11 and 12 except the following dosing was used: cynomolgus monkeys were dosed at 1.6E+13 vg/kg; mice were dosed at 3E+13 vg % kg, and iPSC-CMs were dosed at 10E+4 vg/cell and 10E+5 vg/cell. Transduction in heart or liver was measured as described in Example 12. The average measurements of 2 animals, 4 animals, or 2 multiplicities of infection are shown for Cynomolgus monkey, mouse, and iPSC-CMs, respectively. Routes of administration, type of tissue collected and tissue collection timing was the same as in Examples 11 and 12.

FIG. 46 represents heatmap data showing efficiency of transduction in various tissue samples. Each column on the heatmap represents one capsid and each row represents one sample type. Lighter color means higher value and darker color means lower value, with the median grayscale representative of wildtype AAV9 control. AAV9 is a wildtype capsid that served as a control. Together, this data demonstrates the viability and transduction efficiency of the new capsids in vitro and in vivo.

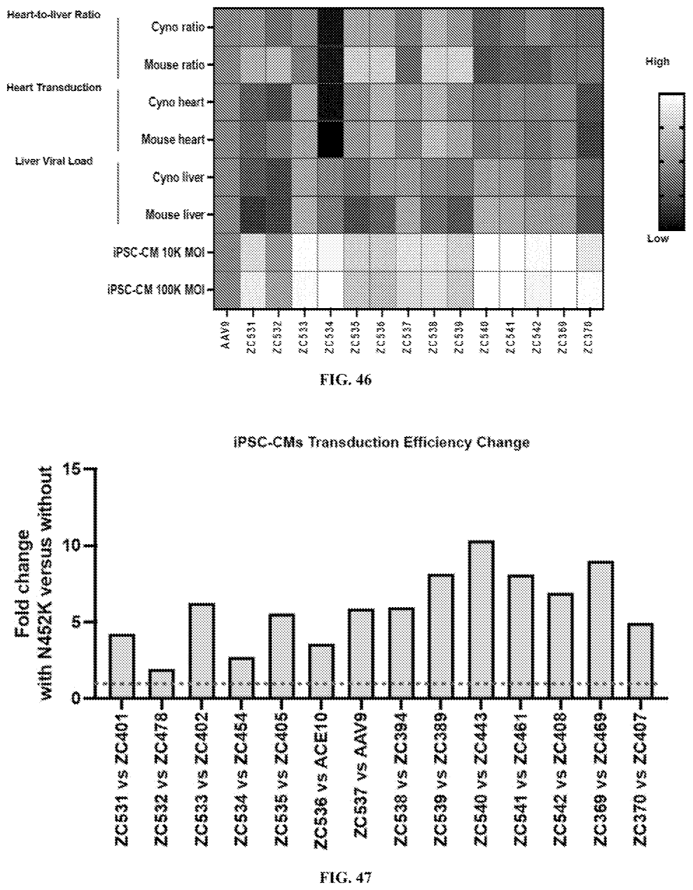

Next, transduction efficiency was evaluated in iPSC-CMs to compare transduction of controls without an N452K mutation and their counterpart capsids with an N452K mutation. FIG. 47 shows increased transduction in every capsid with an N452K mutation compared to the control showing overall improved transduction efficiency in cardiomyocytes.

The heart-to-liver ratio, heart transduction, and liver viral load measurements of four newly generated capsids in Cynomolgus monkey were evaluated relative to the performance of wildtype AAV9 control (FIG. 48). Animals were administered virus, and tissue was collected, as described above. The ZC536 and ZC538 capsids showed improved heart-to-liver ratio and increased heart transduction was observed for each of the capsids relative to AAV9.

### Example 16: Biodistribution and Transduction of Newly Generated AAV9 Capsids in Non-Human Primates

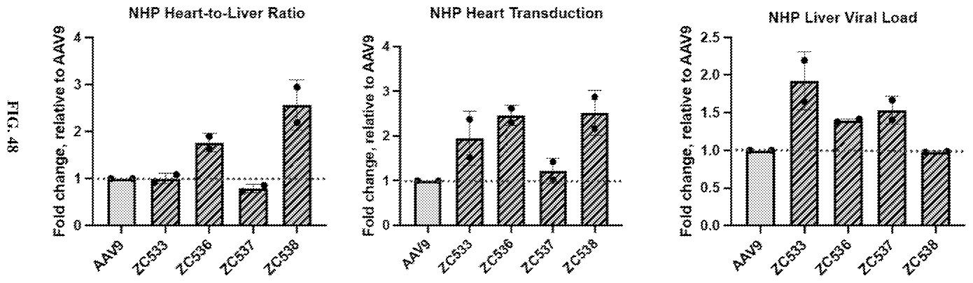

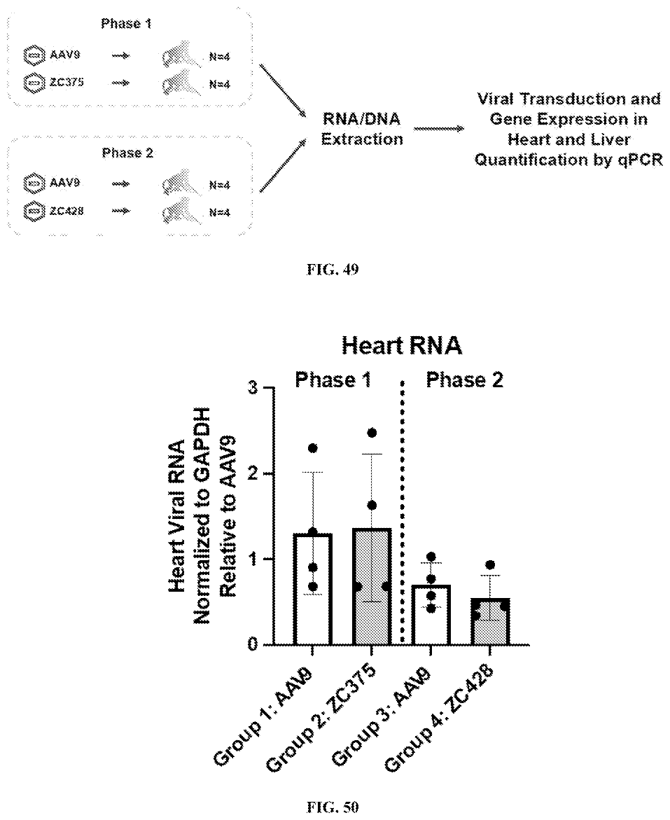

To characterize the performance of capsids described in Example 13 individually in NHPs (one test article per animal), NHPs were administered a single injection of AAV9. ZC375, or ZC428 at 6E+13 vg/kg systemically. The study was divided into two phases (according to the experimental design depicted in FIG. 49) and in each phase, one novel capsid and AAV9 control were tested with 4 Cynomolgus Monkeys per test article. Animals were sacrificed at 28-day post injection. RNA and DNA were extracted from heart and liver tissues, followed by RT-qPCR based quantification of viral transgene mRNA expression and qPCR-based quantification of viral DNA genome load.

Viral transgene expression levels in the heart were measured by RT-qPCR analysis on RNA samples and normalized to the average of all AAV9 data points. Both ZC375 and ZC428 show comparable transgene expression in the heart compared to their matched AAV9 control (FIG. 50). Each measured sample represents one individual animal for which 4 heart biopsy samples were analyzed and averaged.

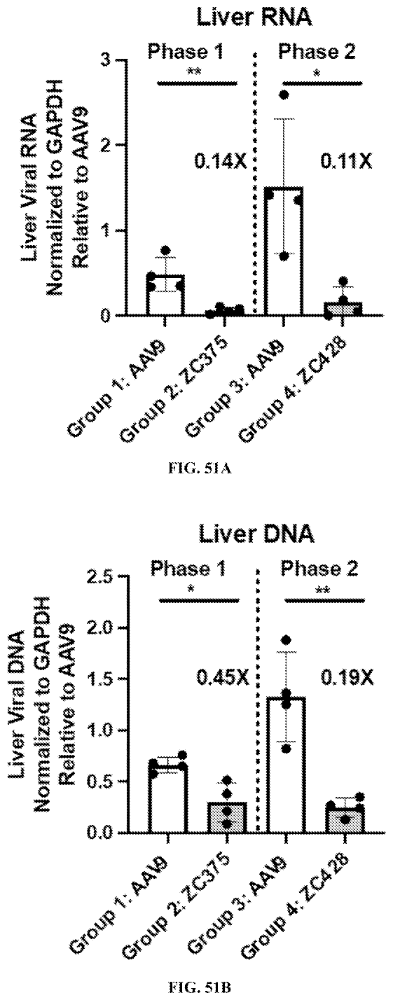

Viral transgene expression in the liver from the NHP were measured by RT-qPCR analysis on RNA samples and normalized to the average of all AAV9 data points. Viral genome load levels were measured by qPCR analysis on DNA samples and normalized to the average of all AAV9 data points. ZC375 and ZC428 show reduced transduction in the liver at both RNA and DNA levels compared to their matched AAV9 control (FIGS. 51A and 51B). Each measured sample represents one individual animal for which 2 liver biopsy samples were analyzed and averaged.

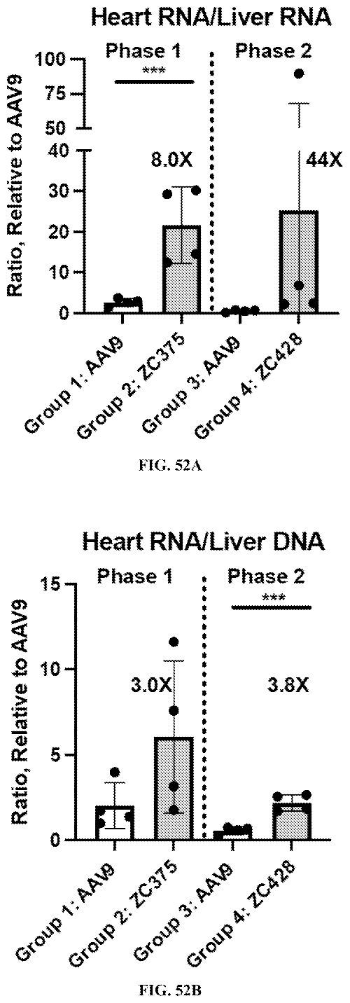

Comparison between heart transduction to liver transduction ratios from the NHP biodistribution and transduction study above was calculated. Using either heart RNA-based and liver RNA-based measurements (FIG. 52A), or heart RNA-based and liver DNA-based measurements (FIG. 52B) it was demonstrated that ZC375 and ZC428 had an improved heart-to-liver ratio compared to their matched AAV9 control. Together, this data demonstrates improved transduction efficiency and heart-to-liver ratio in NHP for both the ZC375 and ZC428 capsids compared to a wild-type AAV9.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein may be employed. It is intended that the following claims define the scope of the disclosure and that methods and structures within the scope of these claims and their equivalents be covered thereby.

What is claimed is:

1. A recombinant adeno-associated virus (rAAV) virion, comprising a capsid protein and a plakophilin-2 (PKP2) expression cassette, wherein the capsid protein shares, or comprises a sequence sharing, at least 80% amino acid sequence identity to an AAV9 VP3 reference sequence according to SEQ ID NO: 487, and wherein the capsid protein comprises, relative to reference sequence SEQ ID NO: 1:
an amino acid insertion at position 584, or between positions 583 and 584, comprising one or more of an asparagine (N), a threonine (T), a tyrosine (Y), phenylalanine (F), and an alanine (A);
an amino acid insertion at position 585, or between positions 584 and 585, comprising one or more of a histidine (H) and a methionine (M):
an amino acid insertion at position 586, or between positions 585 and 586, comprising one or more of a histidine (H), a tyrosine (Y), a valine (V), a threonine (T), an alanine (A), an isoleucine (I), a tryptophan (W), a methionine (M), and a leucine (L);
an amino acid insertion at position 587, or between positions 586 and 587, comprising one or more of an isoleucine (I) and a proline (P);
an amino acid insertion at position 588, or between positions 587 and 588, comprising one or more of an isoleucine (I), a threonine (T), and a proline (P);
an amino acid insertion at position 589, or between positions 588 and 589, comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587O, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

2. The rAAV virion of claim 1, wherein the capsid protein comprises one, two, three, four or more substitutions or insertions in the VR-VIII site.

3. The rAAV virion of claim 2, wherein the capsid protein comprises, relative to reference SEQ ID NO:1, one, two, three, four or more substitutions or insertions at positions from 584 to 590 in the VR-VIII site, or one, two, three, four or more substitutions or insertions at positions from $85 to 590 in the VR-VIII site.

4. The rAAV virion of any one of claims 1-3, wherein the capsid protein comprises, relative to reference sequence SEQ ID NO: 1:
(i) one or more amino acid substitutions selected from the group consisting of T582D, T582E, N583V, H584Q, S586K, A587P, A587S, Q588G, Q588M, A589S, A591I, G594Q, and G594D;
(ii) one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R;
(iii) one or more amino acid substitutions selected from the group consisting of Q585C, Q585S, S586I, A587V and A587O; or
(iv) one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586O, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L.

5. The rAAV virion of any one of claims 1-4, wherein the capsid protein: (i) is cardiotrophic, (ii) exhibits increased transduction efficiency in cardiac cells compared to the parental sequence, (iii) exhibits decreased transduction efficiency in liver cells compared to the parental sequence, and/or (iv) exhibits increased selectivity for the cardiac cells over liver cells compared to the parental sequence.

6. The rAAV virion of any one of claims 1-5, wherein the capsid protein comprises, relative to reference sequence SEQ ID NO: 1, one or more amino acid substitutions selected from the group consisting of N452K, N452A, N452V, N452I, G453A, G453N, S454T, S454D, G455N, Q456L, Q456K, N457L, N457V, Q458I, and Q458H.

7. The rAAV virion of any one of claims 1-5, wherein the capsid protein comprises, relative to reference sequence SEQ ID NO: 1, at position 452 an amino acid selected from the group consisting of: K and N.

8. The rAAV virion of any one of claims 1-5, wherein the capsid protein comprises, relative to reference sequence SEQ ID NO: 1, an amino acid substitution N452K.

9. The rAAV virion of any one of claims 1-8, wherein the capsid protein comprises, relative to reference sequence SEQ ID NO: 1:
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, T, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q.

10. The rAAV virion of any one of claims 1-5, wherein the capsid protein comprises, relative to reference sequence SEQ ID NO: 1:
at position 452 an amino acid selected from the group consisting of: K and N;
at position 584 an amino acid selected from the group consisting of: R and H;
at position 585 an amino acid selected from the group consisting of: N, M, C, E, G, S, V, A, T, H, L and Q;
at position 586 an amino acid selected from the group consisting of: M, D, N, G, A, T, R, I and S;
at position 587 an amino acid selected from the group consisting of: T, N V, V L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

11. The rAAV virion of any one of claims 1-8, wherein the capsid protein comprises, relative to reference sequence SEQ ID NO: 1:
at position 584 amino acid R:
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G:
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M.

12. The rAAV virion of any one of claims 1-5, wherein the capsid protein comprises, relative to reference sequence SEQ ID NO: 1, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M.

13. The rAAV virion of any one of claims 1-8, wherein the capsid protein comprises, relative to reference sequence SEQ ID NO: 1:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q.

14. The rAAV virion of any one of claims 1-5, wherein the capsid protein comprises, relative to reference sequence SEQ ID NO: 1:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

15. The rAAV virion of any one of claims 1-8, wherein the capsid protein comprises, relative to reference sequence SEQ ID NO: 1:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or
at position 590 an amino acid selected from the group consisting of: I, S, G, H and R.

16. The rAAV virion of any one of claims 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, G, M, C, V and T;
(iii) at position 586 an amino acid selected from the group consisting of: N, T, M, G, and D;
(iv) at position 587 an amino acid selected from the group consisting of: T, L, I, K, S, N and V;
(v) at position 588 an amino acid selected from the group consisting of: V, F, Y, L, T, S, I and R;
(vi) at position 589 an amino acid selected from the group consisting of: S, N, L, T, I and R; and
(vii) at position 590 an amino acid selected from the group consisting of: I, S, G, H and R.

17. The rAAV virion of any one of claims 1-8, wherein the capsid protein comprises, relative to reference sequence SEQ ID NO: 1:
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q.

18. The rAAV virion of any one of claims 1-5, wherein the capsid protein comprises, relative to reference sequence SEQ ID NO: 1:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

19. The rAAV virion of any one of claims 1-8, wherein the capsid protein comprises, relative to reference sequence SEQ ID NO: 1:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or
at position 590 an amino acid selected from the group consisting of: I, S, G, and R.

20. The rAAV virion of any one of claims 1-5, wherein the capsid protein comprises, relative to reference sequence SEQ ID NO: 1, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N:
(iv) at position 587 an amino acid selected from the group consisting of: T, N, and V;
(v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

21. The rAAV virion of any one of claims 1-20, wherein the capsid protein comprises, relative to reference sequence SEQ ID NO: 1:
at position 452 an amino acid selected from the group consisting of: K and N; and
at position 587 amino acid substitution A587T; and optionally comprises amino acid N or R at one, two or more positions selected from the group consisting of: 584, 585, 586, 588, 589, and 590.

22. The rAAV virion of any one of claims 1-21, wherein the capsid protein comprises, relative to reference sequence SEQ ID NO: 1:
at position 452 an amino acid selected from the group consisting of: K and N; and
amino acid N or R at one, two or more positions selected from the group consisting of: 584, 585, 586, 588, 589, and 590.

23. The rAAV virion of any one of claims 1-22, wherein the capsid protein comprises, relative to reference sequence SEQ ID NO: 1:
at position 452 an amino acid selected from the group consisting of: K and N; and
amino acid S at two or more positions selected from the group consisting of: 585, 586, 587, 588, 589 and 590.

24. The rAAV virion of any one of claims 1-23, wherein the capsid protein comprises, relative to reference sequence SEQ ID NO: 1:
at position 452 an amino acid selected from the group consisting of: K and N; and
at three, four, five or six positions in the region 585-590 of the VR-VIII site, amino acids selected from the group consisting of: N, S, T, R, and I.

25. The rAAV virion of claim 24, wherein the capsid protein comprises, relative to reference sequence SEQ TD NO: 1:
at three, four, five or six positions in the region 585-590 of the VR-VIII site, amino acids selected from the group consisting of: N, S, T, and R.

26. The rAAV virion of any one of claims 1-5, wherein the capsid protein comprises, relative to reference sequence SEQ ID NO: 1, amino acid substitutions Q585E, S586N, A587T, Q588V, A599S, Q590I, and N452K.

27. The rAAV virion of any one of claims 1-5, wherein the capsid protein comprises, relative to reference sequence SEQ ID NO: 1, amino acid substitutions S586T, A587L, Q588F, A589N, Q590S, and N452K.

28. The rAAV virion of any one of claims 1-5, wherein the capsid protein comprises, relative to reference sequence SEQ ID NO: 1, amino acid substitutions Q585N, A587T, Q588Y, A589L, Q590G, and N452K.

29. The rAAV virion of any one of claims 1-5, wherein the capsid protein comprises, relative to reference sequence SEQ ID NO: 1, amino acid substitutions Q585G, A587I, Q588L, A589T, Q590H, and N452K.

30. The rAAV virion of any one of claims 1-5, wherein the capsid protein comprises, relative to reference sequence SEQ ID NO: 1, amino acid substitutions Q585M, S586M, A587T, Q588T, and Q590R; and amino acid N at position 452.

31. The rAAV virion of any one of claims 1-5, wherein the capsid protein comprises, relative to reference sequence SEQ ID NO: 1, amino acid substitutions Q585N, A587T, Q588Y, A589L, and Q590G; and amino acid N at position 452.

32. The rAAV virion of any one of claims 1-5, wherein the capsid protein comprises, relative to reference sequence SEQ ID NO: 1, amino acid substitutions Q585C, A587T, Q588S, A589I, and Q590R; and amino acid N at position 452.

33. The rAAV virion of any one of claims 1-5, wherein the capsid protein comprises, relative to reference sequence SEQ ID NO: 1, amino acid substitutions Q585E, S586D, A587N, Q588I, A589R, and Q590S; and amino acid N at position 452.

34. The rAAV virion of any one of claims 1-5, wherein the capsid protein comprises, relative to reference sequence SEQ ID NO: 1, amino acid substitutions Q585E, S586D, A587N, Q588I, A589R, Q590S, and N452K.

35. The rAAV virion of any one of claims 1-5, wherein the capsid protein comprises, relative to reference sequence SEQ ID NO: 1, amino acid substitutions Q585N, S586N, A587V, Q588I, A589S, Q590G, and N452K.

36. The rAAV virion of any one of claims 1-5, wherein the capsid protein comprises, relative to reference sequence SEQ ID NO: 1, amino acid substitutions S586G and Q588Y; and amino acid N at position 452.

37. The rAAV virion of any one of claims 1-5, wherein the capsid protein comprises, relative to reference sequence SEQ ID NO: 1, amino acid substitutions S586A, A587N, Q588Y, A589G, and N452K.

38. The rAAV virion of any one of claims 1-37, wherein the capsid protein comprises, relative to reference sequence SEQ ID NO:1, amino acids ATN at positions 581-583, and amino acids AQTG at positions 591-594.

39. The rAAV virion of any one of claims 1-37, wherein the capsid protein comprises, relative to reference sequence SEQ ID NO:1, amino acids ATNH at positions 581-584, and amino acids AQTG at positions 591-594.

40. The rAAV virion of any one of claims 1-5, wherein the capsid protein comprises, relative to reference sequence SEQ ID NO:1:
(i) amino acid sequence ATNHENTVSIAQTG at the VR-VIII positions 581-594, and amino acid K at the VR-IV position 452;
(ii) amino acid sequence ATNHQTLFNSAQTG at the VR-VIII positions 581-594, and amino acid K at the VR-IV position 452;
(iii) amino acid sequence ATNHNSTYLGAQTG at the VR-VIII positions 581-594, and amino acid K at the VR-IV position 452;
(iv) amino acid sequence ATNHGSILTHAQTG at the VR-VIII positions 581-594, and amino acid K at the VR-IV position 452;
(v) amino acid sequence ATNHMMTTARAQTG at the VR-VIII positions 581-594, and amino acid N at the VR-IV position 452;
(vi) amino acid sequence ATNHNSTYLGAQTG at the VR-VIII positions 581-594, and amino acid N at the VR-IV position 452;
(vii) amino acid sequence ATNHCSTSIRAQTG at the VR-VIII positions 581-594, and amino acid N at the VR-IV position 452;
(viii) amino acid sequence ATNHEDNIRSAQTG at the VR-VIII positions 581-594, and amino acid N at the VR-IV position 452;
(ix) amino acid sequence ATNHEDNIRSAQTG at the VR-VIII positions 581-594, and amino acid K at the VR-IV position 452;
(x) amino acid sequence ATNHNNVISGAQTG at the VR-VIII positions 581-594, and amino acid K at the VR-IV position 452;
(xi) amino acid sequence ATNHQGAYAQAQTG at the VR-VIII positions 581-594, and amino acid N at the VR-IV position 452;
(xii) amino acid sequence ATNHQANYGQAQTG at the VR-VIII positions 581-594, and amino acid K at the VR-IV position 452;
(xiii) amino acid sequence ATNHNMNRVNAQTG at the VR-VIII positions 581-594, and amino acid N at the VR-IV position 452;
(xiv) amino acid sequence ATNHNNVISGAQTG at the VR-VIII positions 581-594, and amino acid N at the VR-IV position 452;
(xv) amino acid sequence ATNHSNSVQSAQTG at the VR-VIII positions 581-594, and amino acid N at the VR-IV position 452;
(xvi) amino acid sequence ATNHSSTFQGAQTG at the VR-VIII positions 581-594, and amino acid N at the VR-IV position 452;
(xvii) amino acid sequence ATNHVSSFTSAQTG at the VR-VIII positions 581-594, and amino acid N at the VR-IV position 452;
(xviii) amino acid sequence ATNHSTTNFRAQTG at the VR-VIII positions 581-594, and amino acid N at the VR-IV position 452;
(xix) amino acid sequence ATNHSSIFNSAQTG at the VR-VIII positions 581-594, and amino acid N at the VR-IV position 452;
(xx) amino acid sequence ATNHAGNYNNAQTG at the VR-VIII positions 581-594, and amino acid N at the VR-IV position 452;
(xxi) amino acid sequence ATNHTSVISIAQTG at the VR-VIII positions 581-594, and amino acid N at the VR-IV position 452;
(xxii) amino acid sequence ATNHHSRVEIAQTG at the VR-VIII positions 581-594, and amino acid N at the VR-IV position 452;
(xxiii) amino acid sequence ATNHSSIIYSAQTG at the VR-VIII positions 581-594, and amino acid N at the VR-IV position 452;
(xxiv) amino acid sequence ATNHHSGRDSYAQTG at the VR-VIII positions 581-594, and amino acid N at the VR-IV position 452;
(xxv) amino acid sequence ATNHSSSYNNAQTG at the VR-VIII positions 581-594, and amino acid N at the VR-IV position 452;
(xxvi) amino acid sequence ATNHHNPSINAQTG at the VR-VIII positions 581-594, and amino acid N at the VR-IV position 452;
(xxvii) amino acid sequence ATNHNRNGLLAQTG at the VR-VIII positions 581-594, and amino acid N at the VR-IV position 452;
(xxviii) amino acid sequence ATNHHESTSVRAQTG at the VR-VIII positions 581-594, and amino acid N at the VR-IV position 452;
(xxix) amino acid sequence ATNHNIRTEMAQTG at the VR-VIII positions 581-594, and amino acid N at the VR-IV position 452;
(xxx) amino acid sequence ATNHQTLFNSAQTG at the VR-VIII positions 581-594, and amino acid N at the VR-IV position 452;
(xxxi) amino acid sequence ATNHLSVSSIAQTG at the VR-VIII positions 581-594, and amino acid N at the VR-IV position 452;
(xxxii) amino acid sequence ATNHEDIIRSAQTG at the VR-VIII positions 581-594, and amino acid N at the VR-IV position 452;
(xxxiii) amino acid sequence ATNRQTAQAQAQTG at the VR-VIII positions 581-594, and amino acid N at the VR-IV position 452; or
(xxxiv) amino acid sequence ATNRQIAQAQAQTG at the VR-VIII positions 581-594, and amino acid N at the VR-IV position 452.

41. The rAAV virion of any one of claims 1-8, wherein the capsid protein comprises, relative to reference sequence SEQ ID NO: 1:
(i) an amino acid insertion at position 584 comprising one or more of an asparagine (N), a threonine (T), a tyrosine (Y), phenylalanine (F), and an alanine (A);
(ii) an amino acid insertion at position 585 comprising one or more of a histidine (H) and a methionine (M);
(iii) an amino acid insertion at position 586 comprising one or more of a histidine (H), a tyrosine (Y), a valine (V), a threonine (T), an alanine (A), an isoleucine (I), a tryptophan (W), a methionine (M), and a leucine;
(iv) an amino acid insertion at position 587 comprising one or more of an isoleucine (I) and a proline (P);
(v) an amino acid insertion at position 588 comprising one or more of an isoleucine (I), a threonine (T), and a proline (P); and/or
(vi) an amino acid insertion at position 589 comprising one or more of a glycine (G) and a glutamine (Q).

42. The rAAV virion of claim 41, wherein the capsid protein comprises, relative to reference sequence SEQ ID NO: 1:
(i) an amino acid insertion at position 584 consisting of a TY, FN, or AT;
(ii) an amino acid insertion at position 585 consisting of MH;
(iii) an amino acid insertion at position 586 consisting of HY, VT, Al, WM, or ML;
(iv) an amino acid insertion at position 587 consisting of P1; and/or
(v) an amino acid insertion at position 588 consisting of IT or PT.

43. The rAAV virion of any one of claims 1-42, wherein the capsid protein shares, or comprises a sequence sharing, at least 90%, at least 95%, at least 96%, at least 97%, at least 99%, or 100% amino acid sequence identity to an AAV9 VP3 sequence according to SEQ ID NO: 487, except for the specified modifications.

44. The rAAV virion of any one of claims 1-43, wherein the capsid protein shares, or comprises a sequence sharing, at least 90%, at least 95%, at least 96%, at least 97%, at least 99%, or 100% amino acid sequence identity to an AAV9 VP2 sequence according to SEQ ID NO: 486, except for the specified modifications.

45. The rAAV virion of any one of claims 1-44, wherein the capsid protein shares, or comprises a sequence sharing, at least 90%, at least 95%, at least 96%, at least 97%, at least 99%, or 100% amino acid sequence identity to an AAV9 VP1 sequence according to SEQ ID NO: 1, except for the specified modifications.

46. The rAAV virion of any one of claims 1-45, wherein the capsid protein comprises, consists essentially of, or consists of an amino acid sequence at least 85%, 90%, 95%, 96%, 97%, 98%, 99%, or 100% identical to any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774, or a functional fragment thereof.

47. The rAAV virion of any one of claim 1, wherein the capsid protein comprises, consists essentially of or consists of a polypeptide sequence of any one of the group consisting of: SEQ ID NOs: 488, 499, 504, 505, 506, 510, 512, 513, 516, 518, 521, 522, 533, 536, 539, 558, 562, 566, 571, 576, 578, 579, 580, 581, 585, 588, 589, 705, 706, 707, 708, 710, 772, and 774.

48. The rAAV virion of any one of claims 1-47, wherein the rAAV virion transduces heart cells.

49. The rAAV virion of any one of claims 1-48, wherein the rAAV virion transduces cardiomyocytes.

50. The rAAV virion of any one of claims 1-49, wherein the rAAV virion traffics to at least one organ other than the liver.

51. The rAAV virion of any one of claims 1-50, wherein the rAAV virion traffics to the heart.

52. The rAAV virion of any one of claims 1-51, wherein the rAAV virion exhibits a higher heart transduction efficiency than an rAAV virion having an AAV9 VP1 capsid protein according to SEQ ID NO: 1.

53. The rAAV virion of any one of claims 1-52, wherein the rAAV virion exhibits a higher heart-to-liver transduction ratio than an rAAV virion having an AAV9 VP1 capsid protein according to SEQ ID NO: 1, optionally at least 2, 3, 4, 5, 6, 7, 8, 9 or 10 times higher.

54. The rAAV virion of any one of claims 1-53, wherein administration of the rAAV virion to a subject leads to a lower liver viral load than administration of an rAAV virion having an AAV9 VP1 capsid protein according to SEQ ID NO: 1, optionally at least 2, 3, 4, 5, 6, 7, 8, 9 or 10 times lower.

55. The rAAV virion of any one of claims 1-54, wherein the rAAV virion exhibits a higher transduction efficiency, optionally higher heart transduction efficiency, than an rAAV virion having an AAV9 VP1 capsid protein according to SEQ ID NO: 1, assessed in a primate.

56. The rAAV virion of any one of claims 1-55, wherein the rAAV virion exhibits a higher heart-to-liver transduction ratio than an rAAV virion having an AAV9 VP1 capsid protein according to SEQ ID NO: 1, assessed in a primate, optionally at least 2, 3, 4, 5, 6, 7, 8, 9 or 10 times higher.

57. The rAAV virion of any one of claims 1-56, wherein administration of the rAAV virion to a subject leads to a lower liver viral load than administration of an rAAV virion having an AAV9 VP1 capsid protein according to SEQ ID NO: 1, assessed in a primate, optionally at least 2, 3, 4, 5, 6, 7, 8, 9 or 10 times lower.

58. The rAAV virion of any one of claims 1-57, wherein the PKP2 expression cassette comprises a sequence having at least 95% identity to SEQ ID NO: 782 or SEQ ID NO: 783.

59. The rAAV virion of any one of claims 1-58, wherein the PKP2 expression cassette comprises a nucleic acid sequence having at least 95% identity to SEQ ID NO: 786.

60. The rAAV virion of any one of claims 1-59, wherein the PKP2 expression cassette comprises a cardiac specific promoter.

61. The rAAV virion of claim 60, wherein the cardiac specific promoter directs gene expression in the myocardium, the epicardium, or both.

62. The rAAV virion of claim 60 or claim 61, wherein the cardiac specific promoter is a troponin promoter, or an alpha-myosin heavy chain promoter.

63. The rAAV virion of claim 62, wherein the troponin promoter has a nucleic acid sequence having at least 95% identity to SEQ ID NO: 784.

64. The rAAV virion of any one of claims 1-59, wherein the PKP2 expression cassette comprises a PKP2 promoter.

65. The rAAV virion of claim 64, wherein the PKP2 promoter has a nucleic acid sequence having at least 95% identity to SEQ ID NO: 785.

66. The rAAV virion of any one of claims 1-59, wherein the PKP2 expression cassette comprises a constitutive promoter.

67. The rAAV virion of claim 66, wherein the constitutive promoter is a beta-actin promoter.

68. The rAAV virion of any one of claims 1-67, wherein the PKP2 expression cassette comprises a cardiac specific enhancer.

69. The rAAV virion of any one of claims 1-68, wherein the PKP2 expression cassette comprises a 3′ element.

70. The rAAV virion of claim 69, wherein the 3′ element comprises a Woodchuck Hepatitis Virus Posttranscriptional Regulatory Element (WPRE), a bovine growth hormone polyadenylation (bGH polyA) sequence, or a combination thereof.

71. A pharmaceutical composition comprising an rAAV virion according to any one of claims 1-70 and a pharmaceutically acceptable carrier.

72. A method of transducing a cardiac cell, comprising contacting the cardiac cell with an rAAV virion according to any one of claims 1-70, wherein the rAAV virion transduces the cardiac cell.

73. The method of claim 72, wherein the cardiac cell is a cardiomyocyte.

74. The method of claim 72 or claim 73, wherein the rAAV virion exhibits higher transduction efficiency in the cell than an rAAV virion having an AAV9 VP1 capsid protein according to SEQ ID NO: 1.

75. A method of delivering one or more gene products to a cardiac cell, comprising contacting the cardiac cell with an rAAV virion according to any one of claims 1-70.

76. The method of claim 75, wherein the cardiac cell is a cardiomyocyte.

77. A method of treating a heart disease or disorder in an individual in need thereof, comprising administering a therapeutically effective amount of an rAAV virion according to any one of claims 1-70 to the subject, wherein the rAAV virion transduces cardiac tissue.

78. The method of claim 77, wherein the heart disease or disorder is arrhythmogenic right ventricular cardiomyopathy (ARVC) or arrhythmogenic cardiomyopathy (ACM).

79. The method of claim 77 or claim 78, wherein the AAV virion is administered intravenously, intracardially, pericardially, or intraarterially.

80. The method of any one of claims 77-79, wherein the method reverses, reduces, or prevents at least one of fibrofatty tissue replacement, myocardial atrophy, predominant right ventricular dilation, ventricular arrhythmias, sudden cardiac death, or exercise-triggered cardiac events.

81. The method of claim 80, wherein the method reverses, reduces, or prevents fibrofatty tissue replacement in myocardium, epicardium, or both.

82. The method of any one of claims 77-81, wherein the method restores desmosome structure and/or function.

83. The method of any one of claims 77-82, wherein the method restores PKP2 protein and activity levels.

84. The method of any one of claims 77-83, wherein the method restores PKP2 induced gene expression.

85. The method of any one of claims 77-84, wherein the method restores expression of one or more of Ryanodine Receptor 2 (Ryr2), Ankyrin-B (Ank2), Cacnalc (CaV1.2), triadin (Trdn), or calsequestrin-2 (Casq2).

86. The method of any one of claims 77-85, wherein the individual is identified as having at least one variation in a desmosome protein.

87. The method of claim 86, wherein the desmosome protein is PKP2.

88. The method of claim 86 or claim 87, wherein the variation comprises a deletion, an insertion, a single nucleotide variation, or a copy number variation.

89. An rAAV virion according to any one of claims 1-70 for use in a method of treating a heart disease or disorder in an individual in need thereof, wherein the rAAV virion transduces cardiac tissue.

90. The method of claim 89, wherein the heart disease or disorder is arrhythmogenic right ventricular cardiomyopathy (ARVC) or arrhythmogenic cardiomyopathy (ACM).

91. The method of claim 89 or claim 90, wherein the AAV virion is administered intravenously, intracardially, pericardially, or intraarterially.

92. The method of any one of claims 89-91, wherein the method reverses, reduces, or prevents at least one of fibrofatty tissue replacement, myocardial atrophy, predominant right ventricular dilation, ventricular arrhythmias, sudden cardiac death, or exercise-triggered cardiac events.

93. The method of claim 92, wherein the method reverses, reduces, or prevents fibrofatty tissue replacement in myocardium, epicardium, or both.

94. The method of any one of claims 89-93, wherein the method restores desmosome structure and/or function.

95. The method of any one of claims 89-94, wherein the method restores PKP2 protein and activity levels.

96. The method of any one of claims 89-95, wherein the method restores PKP2 induced gene expression.

97. The method of any one of claims 89-96, wherein the method restores expression of one or more of Ryanodine Receptor 2 (Ryr2), Ankyrin-B (Ank2), Cacnalc (CaV1.2), triadin (Trdn), or calsequestrin-2 (Casq2).

98. The method of any one of claims 89-97, wherein the individual is identified as having at least one variation in a desmosome protein.

99. The method of claim 98, wherein the desmosome protein is PKP2.

100. The method of claim 98 or claim 99, wherein the variation comprises a deletion, an insertion, a single nucleotide variation, or a copy number variation.


SEQUENCE LISTING (799 sequences)

SEQ ID NO: 1 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 7 aa, synthetic construct
NGSGQNQ

SEQ ID NO: 3 - protein, 6 aa, synthetic construct
NNSEFA

SEQ ID NO: 4 - protein, 5 aa, synthetic construct
GRDNV

SEQ ID NO: 5 - protein, 14 aa, synthetic construct
ATNHQSAQAQAQTG

SEQ ID NO: 6 - protein, 9 aa, synthetic construct
GYHKSGAAQ

SEQ ID NO: 7 - protein, 9 aa, synthetic construct
VIIKSGAAQ

SEQ ID NO: 8 - protein, 9 aa, synthetic construct
GYHKIGAAQ

SEQ ID NO: 9 - protein, 9 aa, synthetic construct
GYHKSGVAQ

SEQ ID NO: 10 - protein, 9 aa, synthetic construct
VYHKSGAAQ

SEQ ID NO: 11 - protein, 9 aa, synthetic construct
GYHKISAAQ

SEQ ID NO: 12 - protein, 9 aa, synthetic construct
TTVPSSSRY

SEQ ID NO: 13 - protein, 9 aa, synthetic construct
VIIRVVRLS

SEQ ID NO: 14 - protein, 9 aa, synthetic construct
TVLGQNQQT

SEQ ID NO: 15 - protein, 9 aa, synthetic construct
IYHKSGAAQ

SEQ ID NO: 16 - protein, 9 aa, synthetic construct
TVLDKNQQT

SEQ ID NO: 17 - protein, 9 aa, synthetic construct
YSGTDVRYK

SEQ ID NO: 18 - protein, 9 aa, synthetic construct
VTASGKEHR

SEQ ID NO: 19 - protein, 9 aa, synthetic construct
GYRKSGAAQ

SEQ ID NO: 20 - protein, 9 aa, synthetic construct
NRTVSNGSE

SEQ ID NO: 21 - protein, 9 aa, synthetic construct
TVLDRINKT

SEQ ID NO: 22 - protein, 9 aa, synthetic construct
TGVGHLTSA

SEQ ID NO: 23 - protein, 9 aa, synthetic construct
GYHKGGAAQ

SEQ ID NO: 24 - protein, 9 aa, synthetic construct
VIAKSGAAQ

SEQ ID NO: 25 - protein, 9 aa, synthetic construct
GYHKSGAAH

SEQ ID NO: 26 - protein, 9 aa, synthetic construct
FIIKSGAAQ

SEQ ID NO: 27 - protein, 9 aa, synthetic construct
GYHKVVRLS

SEQ ID NO: 28 - protein, 9 aa, synthetic construct
GATRSAVES

SEQ ID NO: 29 - protein, 9 aa, synthetic construct
TVSGQNQQT

SEQ ID NO: 30 - protein, 9 aa, synthetic construct
LSHKSGAAQ

SEQ ID NO: 31 - protein, 9 aa, synthetic construct
SSSGQNQQT

SEQ ID NO: 32 - protein, 9 aa, synthetic construct
SGSGQNQQT

SEQ ID NO: 33 - protein, 9 aa, synthetic construct
SQVNGRPRD

SEQ ID NO: 34 - protein, 9 aa, synthetic construct
GYHKEWCGS

SEQ ID NO: 35 - protein, 9 aa, synthetic construct
VVSSKSLNS

SEQ ID NO: 36 - protein, 9 aa, synthetic construct
GYHKSGAAP

SEQ ID NO: 37 - protein, 9 aa, synthetic construct
DASSREKVR

SEQ ID NO: 38 - protein, 9 aa, synthetic construct
SYHKSGAAQ

SEQ ID NO: 39 - protein, 9 aa, synthetic construct
TANGSQKYL

SEQ ID NO: 40 - protein, 9 aa, synthetic construct
VIIRVGAAQ

SEQ ID NO: 41 - protein, 9 aa, synthetic construct
SSTNKISTA

SEQ ID NO: 42 - protein, 9 aa, synthetic construct
TVLDRIQQT

SEQ ID NO: 43 - protein, 9 aa, synthetic construct
GYHKSGAVQ

SEQ ID NO: 44 - protein, 9 aa, synthetic construct
TVLDQNQQT

SEQ ID NO: 45 - protein, 9 aa, synthetic construct
VNMSSPIKT

SEQ ID NO: 46 - protein, 9 aa, synthetic construct
AAYNSNSAF

SEQ ID NO: 47 - protein, 9 aa, synthetic construct
GYHKSGAAR

SEQ ID NO: 48 - protein, 9 aa, synthetic construct
VIIRVVRLQ

SEQ ID NO: 49 - protein, 9 aa, synthetic construct
RFWTQNQQT

SEQ ID NO: 50 - protein, 9 aa, synthetic construct
SSPRASSAL

SEQ ID NO: 51 - protein, 9 aa, synthetic construct
IIIRVVRLS

SEQ ID NO: 52 - protein, 9 aa, synthetic construct
KSSNLTAMP

SEQ ID NO: 53 - protein, 9 aa, synthetic construct
NLNSDRHSA

SEQ ID NO: 54 - protein, 9 aa, synthetic construct
LSLKSGAAQ

SEQ ID NO: 55 - protein, 9 aa, synthetic construct
TVLDRNQQT

SEQ ID NO: 56 - protein, 9 aa, synthetic construct
GSERVSNSG

SEQ ID NO: 57 - protein, 9 aa, synthetic construct
VIAKIGAAQ

SEQ ID NO: 58 - protein, 9 aa, synthetic construct
VYHKIGAAQ

SEQ ID NO: 59 - protein, 9 aa, synthetic construct
LSYKSGAAQ

SEQ ID NO: 60 - protein, 9 aa, synthetic construct
STVSQPVRT

SEQ ID NO: 61 - protein, 9 aa, synthetic construct
GHHKSGAAQ

SEQ ID NO: 62 - protein, 9 aa, synthetic construct
YAGIDPRYH

SEQ ID NO: 63 - protein, 9 aa, synthetic construct
DRSRKSMCD

SEQ ID NO: 64 - protein, 9 aa, synthetic construct
VIIRSGAAQ

SEQ ID NO: 65 - protein, 9 aa, synthetic construct
GYHKSGGSA

SEQ ID NO: 66 - protein, 9 aa, synthetic construct
VIIKIGAAQ

SEQ ID NO: 67 - protein, 9 aa, synthetic construct
GYHKVVQLS

SEQ ID NO: 68 - protein, 9 aa, synthetic construct
VIIKLVAAQ

SEQ ID NO: 69 - protein, 9 aa, synthetic construct
KVSSHSVCD

SEQ ID NO: 70 - protein, 9 aa, synthetic construct
GYHKRVRLS

SEQ ID NO: 71 - protein, 9 aa, synthetic construct
GYHKSSAAQ

SEQ ID NO: 72 - protein, 9 aa, synthetic construct
GYRKIGAAQ

SEQ ID NO: 73 - protein, 9 aa, synthetic construct
GYHKSGAAC

SEQ ID NO: 74 - protein, 9 aa, synthetic construct
GYRQSGAAQ

SEQ ID NO: 75 - protein, 9 aa, synthetic construct
VIIKLIAAQ

SEQ ID NO: 76 - protein, 9 aa, synthetic construct
VIIRVVRAQ

SEQ ID NO: 77 - protein, 9 aa, synthetic construct
GYHKSGAAW

SEQ ID NO: 78 - protein, 9 aa, synthetic construct
GYHKSGAVS

SEQ ID NO: 79 - protein, 9 aa, synthetic construct
GYHKEWCSS

SEQ ID NO: 80 - protein, 9 aa, synthetic construct
SSSSNRLAD

SEQ ID NO: 81 - protein, 9 aa, synthetic construct
SNNSSSAKF

SEQ ID NO: 82 - protein, 9 aa, synthetic construct
VKLSSTSSS

SEQ ID NO: 83 - protein, 9 aa, synthetic construct
GYHKEWCAQ

SEQ ID NO: 84 - protein, 9 aa, synthetic construct
AGSGQNQQT

SEQ ID NO: 85 - protein, 9 aa, synthetic construct
NPHGTATYL

SEQ ID NO: 86 - protein, 9 aa, synthetic construct
NGSGQNQHT

SEQ ID NO: 87 - protein, 9 aa, synthetic construct
GYHKVGAAQ

SEQ ID NO: 88 - protein, 9 aa, synthetic construct
VIIRVVRLK

SEQ ID NO: 89 - protein, 9 aa, synthetic construct
NSIPSTSKW

SEQ ID NO: 90 - protein, 9 aa, synthetic construct
VIIRVVQLQ

SEQ ID NO: 91 - protein, 9 aa, synthetic construct
SQVNGRPQD

SEQ ID NO: 92 - protein, 9 aa, synthetic construct
NGSGQDQQT

SEQ ID NO: 93 - protein, 9 aa, synthetic construct
GLNSSDRRL

SEQ ID NO: 94 - protein, 9 aa, synthetic construct
IYHKIGAAQ

SEQ ID NO: 95 - protein, 9 aa, synthetic construct
YHKSGAAQL

SEQ ID NO: 96 - protein, 9 aa, synthetic construct
YSGTDVQYK

SEQ ID NO: 97 - protein, 9 aa, synthetic construct
LGSGQNQQT

SEQ ID NO: 98 - protein, 9 aa, synthetic construct
PVSSGADRR

SEQ ID NO: 99 - protein, 9 aa, synthetic construct
EHSTKLNAC

SEQ ID NO: 100 - protein, 9 aa, synthetic construct
NGSDRINKR

SEQ ID NO: 101 - protein, 9 aa, synthetic construct
VIIKGGAAQ

SEQ ID NO: 102 - protein, 9 aa, synthetic construct
GYHRVVRLS

SEQ ID NO: 103 - protein, 9 aa, synthetic construct
VIIRVVRLL

SEQ ID NO: 104 - protein, 9 aa, synthetic construct
VILKSGAAQ

SEQ ID NO: 105 - protein, 6 aa, synthetic construct
LNSMLI

SEQ ID NO: 106 - protein, 6 aa, synthetic construct
NGMSFT

SEQ ID NO: 107 - protein, 6 aa, synthetic construct
HKTFSI

SEQ ID NO: 108 - protein, 6 aa, synthetic construct
SMSNFV

SEQ ID NO: 109 - protein, 6 aa, synthetic construct
ATIPPI

SEQ ID NO: 110 - protein, 6 aa, synthetic construct
NNQFSY

SEQ ID NO: 111 - protein, 6 aa, synthetic construct
NNQFSY

SEQ ID NO: 112 - protein, 6 aa, synthetic construct
NMGHYS

SEQ ID NO: 113 - protein, 6 aa, synthetic construct
SKQMFQ

SEQ ID NO: 114 - protein, 6 aa, synthetic construct
WPSAGV

SEQ ID NO: 115 - protein, 6 aa, synthetic construct
NGGYQC

SEQ ID NO: 116 - protein, 6 aa, synthetic construct
STSPIV

SEQ ID NO: 117 - protein, 6 aa, synthetic construct
SQSGLW

SEQ ID NO: 118 - protein, 6 aa, synthetic construct
VNSQFS

SEQ ID NO: 119 - protein, 6 aa, synthetic construct
SGIEFR

SEQ ID NO: 120 - protein, 6 aa, synthetic construct
SASKFT

SEQ ID NO: 121 - protein, 6 aa, synthetic construct
QLNWTS

SEQ ID NO: 122 - protein, 6 aa, synthetic construct
SMGFPV

SEQ ID NO: 123 - protein, 6 aa, synthetic construct
SSFMGL

SEQ ID NO: 124 - protein, 6 aa, synthetic construct
GSNFHV

SEQ ID NO: 125 - protein, 6 aa, synthetic construct
DMTLYA

SEQ ID NO: 126 - protein, 6 aa, synthetic construct
MGCLFT

SEQ ID NO: 127 - protein, 6 aa, synthetic construct
ALAFNS

SEQ ID NO: 128 - protein, 6 aa, synthetic construct
SKFLFA

SEQ ID NO: 129 - protein, 6 aa, synthetic construct
QDAGLL

SEQ ID NO: 130 - protein, 6 aa, synthetic construct
QDASLL

SEQ ID NO: 131 - protein, 6 aa, synthetic construct
RDDMFS

SEQ ID NO: 132 - protein, 6 aa, synthetic construct
LSRCFQ

SEQ ID NO: 133 - protein, 6 aa, synthetic construct
LSRDFQ

SEQ ID NO: 134 - protein, 6 aa, synthetic construct
QGLTPV

SEQ ID NO: 135 - protein, 6 aa, synthetic construct
QWDVFT

SEQ ID NO: 136 - protein, 6 aa, synthetic construct
PRVSFA

SEQ ID NO: 137 - protein, 6 aa, synthetic construct
QSYYNP

SEQ ID NO: 138 - protein, 6 aa, synthetic construct
RASHLG

SEQ ID NO: 139 - protein, 6 aa, synthetic construct
IILFVP

SEQ ID NO: 140 - protein, 6 aa, synthetic construct
IISFSY

SEQ ID NO: 141 - protein, 6 aa, synthetic construct
LDSMLI

SEQ ID NO: 142 - protein, 6 aa, synthetic construct
NIGHYS

SEQ ID NO: 143 - protein, 6 aa, synthetic construct
NRMSFT

SEQ ID NO: 144 - protein, 6 aa, synthetic construct
NGMSFA

SEQ ID NO: 145 - protein, 6 aa, synthetic construct
IILLLP

SEQ ID NO: 146 - protein, 6 aa, synthetic construct
RMRSLL

SEQ ID NO: 147 - protein, 6 aa, synthetic construct
RRRCRF

SEQ ID NO: 148 - protein, 6 aa, synthetic construct
PKQMFQ

SEQ ID NO: 149 - protein, 6 aa, synthetic construct
LMSNFV

SEQ ID NO: 150 - protein, 6 aa, synthetic construct
GASHLG

SEQ ID NO: 151 - protein, 6 aa, synthetic construct
CASISW

SEQ ID NO: 152 - protein, 6 aa, synthetic construct
SMTTFR

SEQ ID NO: 153 - protein, 6 aa, synthetic construct
AAIPPI

SEQ ID NO: 154 - protein, 6 aa, synthetic construct
PGCESL

SEQ ID NO: 155 - protein, 6 aa, synthetic construct
SMGFAC

SEQ ID NO: 156 - protein, 6 aa, synthetic construct
FLPSLM

SEQ ID NO: 157 - protein, 6 aa, synthetic construct
NGISFT

SEQ ID NO: 158 - protein, 6 aa, synthetic construct
ESSRWA

SEQ ID NO: 159 - protein, 6 aa, synthetic construct
QLYFVP

SEQ ID NO: 160 - protein, 6 aa, synthetic construct
SSNFHV

SEQ ID NO: 161 - protein, 6 aa, synthetic construct
LEFMLI

SEQ ID NO: 162 - protein, 6 aa, synthetic construct
QFDSFD

SEQ ID NO: 163 - protein, 6 aa, synthetic construct
SPVFAC

SEQ ID NO: 164 - protein, 6 aa, synthetic construct
VRLIFD

SEQ ID NO: 165 - protein, 6 aa, synthetic construct
NGMSFI

SEQ ID NO: 166 - protein, 6 aa, synthetic construct
LLFPPI

SEQ ID NO: 167 - protein, 6 aa, synthetic construct
GAGVTG

SEQ ID NO: 168 - protein, 6 aa, synthetic construct
QWMSFT

SEQ ID NO: 169 - protein, 6 aa, synthetic construct
SIGFPV

SEQ ID NO: 170 - protein, 6 aa, synthetic construct
RMQSLL

SEQ ID NO: 171 - protein, 6 aa, synthetic construct
TSALQV

SEQ ID NO: 172 - protein, 6 aa, synthetic construct
SLTHFD

SEQ ID NO: 173 - protein, 6 aa, synthetic construct
QELPFL

SEQ ID NO: 174 - protein, 6 aa, synthetic construct
LYFLLP

SEQ ID NO: 175 - protein, 6 aa, synthetic construct
LSFFFA

SEQ ID NO: 176 - protein, 6 aa, synthetic construct
LSRIFQ

SEQ ID NO: 177 - protein, 6 aa, synthetic construct
DEVILF

SEQ ID NO: 178 - protein, 6 aa, synthetic construct
RAGVAG

SEQ ID NO: 179 - protein, 6 aa, synthetic construct
NGMSLP

SEQ ID NO: 180 - protein, 6 aa, synthetic construct
PFEDFQ

SEQ ID NO: 181 - protein, 6 aa, synthetic construct
QYGSLF

SEQ ID NO: 182 - protein, 6 aa, synthetic construct
NYTFVL

SEQ ID NO: 183 - protein, 6 aa, synthetic construct
MSGYQC

SEQ ID NO: 184 - protein, 6 aa, synthetic construct
NYAFVP

SEQ ID NO: 185 - protein, 6 aa, synthetic construct
RAGVTG

SEQ ID NO: 186 - protein, 6 aa, synthetic construct
WNSMLI

SEQ ID NO: 187 - protein, 6 aa, synthetic construct
IRRFSI

SEQ ID NO: 188 - protein, 6 aa, synthetic construct
NGMSFY

SEQ ID NO: 189 - protein, 6 aa, synthetic construct
IIQFSY

SEQ ID NO: 190 - protein, 6 aa, synthetic construct
NGCLFT

SEQ ID NO: 191 - protein, 6 aa, synthetic construct
RDASLL

SEQ ID NO: 192 - protein, 6 aa, synthetic construct
ADSMLI

SEQ ID NO: 193 - protein, 6 aa, synthetic construct
VDSQFS

SEQ ID NO: 194 - protein, 6 aa, synthetic construct
SIGNFV

SEQ ID NO: 195 - protein, 6 aa, synthetic construct
NGMSLL

SEQ ID NO: 196 - protein, 6 aa, synthetic construct
NYTFVP

SEQ ID NO: 197 - protein, 6 aa, synthetic construct
IRRLVF

SEQ ID NO: 198 - protein, 6 aa, synthetic construct
PMSNFV

SEQ ID NO: 199 - protein, 6 aa, synthetic construct
LWVFPV

SEQ ID NO: 200 - protein, 6 aa, synthetic construct
VRLHFD

SEQ ID NO: 201 - protein, 6 aa, synthetic construct
SMSNLF

SEQ ID NO: 202 - protein, 6 aa, synthetic construct
STSLIV

SEQ ID NO: 203 - protein, 6 aa, synthetic construct
HKTFGI

SEQ ID NO: 204 - protein, 5 aa, synthetic construct
RGNQV

SEQ ID NO: 205 - protein, 5 aa, synthetic construct
VSLNR

SEQ ID NO: 206 - protein, 5 aa, synthetic construct
CDYSV

SEQ ID NO: 207 - protein, 5 aa, synthetic construct
QHGHI

SEQ ID NO: 208 - protein, 5 aa, synthetic construct
LCSLV

SEQ ID NO: 209 - protein, 5 aa, synthetic construct
PTIYV

SEQ ID NO: 210 - protein, 5 aa, synthetic construct
DVIHI

SEQ ID NO: 211 - protein, 5 aa, synthetic construct
AEFYA

SEQ ID NO: 212 - protein, 5 aa, synthetic construct
NSVVC

SEQ ID NO: 213 - protein, 5 aa, synthetic construct
VRSNC

SEQ ID NO: 214 - protein, 5 aa, synthetic construct
LANNI

SEQ ID NO: 215 - protein, 5 aa, synthetic construct
NLQFM

SEQ ID NO: 216 - protein, 5 aa, synthetic construct
EFRDL

SEQ ID NO: 217 - protein, 5 aa, synthetic construct
DFGSL

SEQ ID NO: 218 - protein, 5 aa, synthetic construct
VTNYC

SEQ ID NO: 219 - protein, 5 aa, synthetic construct
WNTNA

SEQ ID NO: 220 - protein, 5 aa, synthetic construct
TESTC

SEQ ID NO: 221 - protein, 5 aa, synthetic construct
SGAAV

SEQ ID NO: 222 - protein, 5 aa, synthetic construct
GGCDI

SEQ ID NO: 223 - protein, 5 aa, synthetic construct
SGSVV

SEQ ID NO: 224 - protein, 5 aa, synthetic construct
SSNAC

SEQ ID NO: 225 - protein, 5 aa, synthetic construct
YNTTV

SEQ ID NO: 226 - protein, 5 aa, synthetic construct
SKCLA

SEQ ID NO: 227 - protein, 5 aa, synthetic construct
SAYTV

SEQ ID NO: 228 - protein, 5 aa, synthetic construct
VRDTV

SEQ ID NO: 229 - protein, 5 aa, synthetic construct
WRSMV

SEQ ID NO: 230 - protein, 5 aa, synthetic construct
AYHGV

SEQ ID NO: 231 - protein, 5 aa, synthetic construct
GMNTI

SEQ ID NO: 232 - protein, 5 aa, synthetic construct
AETSL

SEQ ID NO: 233 - protein, 5 aa, synthetic construct
TLVYV

SEQ ID NO: 234 - protein, 5 aa, synthetic construct
NHDWI

SEQ ID NO: 235 - protein, 5 aa, synthetic construct
TVGIV

SEQ ID NO: 236 - protein, 5 aa, synthetic construct
SLPTV

SEQ ID NO: 237 - protein, 5 aa, synthetic construct
TGILC

SEQ ID NO: 238 - protein, 5 aa, synthetic construct
TDTYI

SEQ ID NO: 239 - protein, 5 aa, synthetic construct
LPVTY

SEQ ID NO: 240 - protein, 5 aa, synthetic construct
GDVYI

SEQ ID NO: 241 - protein, 5 aa, synthetic construct
LYGTV

SEQ ID NO: 242 - protein, 5 aa, synthetic construct
GCEFI

SEQ ID NO: 243 - protein, 5 aa, synthetic construct
SAGLL

SEQ ID NO: 244 - protein, 5 aa, synthetic construct
IKSNI

SEQ ID NO: 245 - protein, 5 aa, synthetic construct
VTTSL

SEQ ID NO: 246 - protein, 5 aa, synthetic construct
AVTSV

SEQ ID NO: 247 - protein, 5 aa, synthetic construct
RDIHI

SEQ ID NO: 248 - protein, 5 aa, synthetic construct
SAISL

SEQ ID NO: 249 - protein, 5 aa, synthetic construct
VASTC

SEQ ID NO: 250 - protein, 5 aa, synthetic construct
IKGLL

SEQ ID NO: 251 - protein, 5 aa, synthetic construct
GSYHT

SEQ ID NO: 252 - protein, 5 aa, synthetic construct
RIGFV

SEQ ID NO: 253 - protein, 5 aa, synthetic construct
NDIYI

SEQ ID NO: 254 - protein, 5 aa, synthetic construct
AVSCV

SEQ ID NO: 255 - protein, 5 aa, synthetic construct
QHNLL

SEQ ID NO: 256 - protein, 5 aa, synthetic construct
VSSCV

SEQ ID NO: 257 - protein, 5 aa, synthetic construct
LNLDV

SEQ ID NO: 258 - protein, 5 aa, synthetic construct
LGATI

SEQ ID NO: 259 - protein, 5 aa, synthetic construct
PVLCV

SEQ ID NO: 260 - protein, 5 aa, synthetic construct
SARHI

SEQ ID NO: 261 - protein, 5 aa, synthetic construct
RATLI

SEQ ID NO: 262 - protein, 5 aa, synthetic construct
PYNHA

SEQ ID NO: 263 - protein, 5 aa, synthetic construct
IGDSI

SEQ ID NO: 264 - protein, 5 aa, synthetic construct
SPMLC

SEQ ID NO: 265 - protein, 5 aa, synthetic construct
YDSTL

SEQ ID NO: 266 - protein, 5 aa, synthetic construct
ALKHV

SEQ ID NO: 267 - protein, 5 aa, synthetic construct
ADLLT

SEQ ID NO: 268 - protein, 5 aa, synthetic construct
NNGHL

SEQ ID NO: 269 - protein, 5 aa, synthetic construct
INSEV

SEQ ID NO: 270 - protein, 5 aa, synthetic construct
SNKTT

SEQ ID NO: 271 - protein, 5 aa, synthetic construct
GSTGL

SEQ ID NO: 272 - protein, 5 aa, synthetic construct
DSDMI

SEQ ID NO: 273 - protein, 5 aa, synthetic construct
TSNFI

SEQ ID NO: 274 - protein, 5 aa, synthetic construct
RNFTT

SEQ ID NO: 275 - protein, 5 aa, synthetic construct
SHKYS

SEQ ID NO: 276 - protein, 5 aa, synthetic construct
VSDIV

SEQ ID NO: 277 - protein, 5 aa, synthetic construct
RVVQA

SEQ ID NO: 278 - protein, 5 aa, synthetic construct
AACAV

SEQ ID NO: 279 - protein, 5 aa, synthetic construct
RGRQI

SEQ ID NO: 280 - protein, 5 aa, synthetic construct
AVANI

SEQ ID NO: 281 - protein, 5 aa, synthetic construct
AGYDL

SEQ ID NO: 282 - protein, 5 aa, synthetic construct
LSEAA

SEQ ID NO: 283 - protein, 5 aa, synthetic construct
MSNYL

SEQ ID NO: 284 - protein, 5 aa, synthetic construct
NFSDN

SEQ ID NO: 285 - protein, 5 aa, synthetic construct
SCCDV

SEQ ID NO: 286 - protein, 5 aa, synthetic construct
LASSV

SEQ ID NO: 287 - protein, 5 aa, synthetic construct
PDHAV

SEQ ID NO: 288 - protein, 5 aa, synthetic construct
KFDII

SEQ ID NO: 289 - protein, 5 aa, synthetic construct
NSSSA

SEQ ID NO: 290 - protein, 5 aa, synthetic construct
HTMHV

SEQ ID NO: 291 - protein, 5 aa, synthetic construct
TLSYC

SEQ ID NO: 292 - protein, 5 aa, synthetic construct
ADTHR

SEQ ID NO: 293 - protein, 5 aa, synthetic construct
SMYSV

SEQ ID NO: 294 - protein, 5 aa, synthetic construct
SVNLV

SEQ ID NO: 295 - protein, 5 aa, synthetic construct
MSGHL

SEQ ID NO: 296 - protein, 5 aa, synthetic construct
KISDT

SEQ ID NO: 297 - protein, 5 aa, synthetic construct
TGLLA

SEQ ID NO: 298 - protein, 5 aa, synthetic construct
AWTTS

SEQ ID NO: 299 - protein, 5 aa, synthetic construct
GGALI

SEQ ID NO: 300 - protein, 5 aa, synthetic construct
SCIEV

SEQ ID NO: 301 - protein, 5 aa, synthetic construct
PPVIC

SEQ ID NO: 302 - protein, 5 aa, synthetic construct
GTYNL

SEQ ID NO: 303 - protein, 4 aa, synthetic construct
NVSY

SEQ ID NO: 304 - protein, 4 aa, synthetic construct
SMVN

SEQ ID NO: 305 - protein, 4 aa, synthetic construct
ANYG

SEQ ID NO: 306 - protein, 4 aa, synthetic construct
NVGT

SEQ ID NO: 307 - protein, 4 aa, synthetic construct
SAYM

SEQ ID NO: 308 - protein, 4 aa, synthetic construct
EKVT

SEQ ID NO: 309 - protein, 4 aa, synthetic construct
TTPG

SEQ ID NO: 310 - protein, 4 aa, synthetic construct
GVYS

SEQ ID NO: 311 - protein, 4 aa, synthetic construct
SYVG

SEQ ID NO: 312 - protein, 4 aa, synthetic construct
LQYN

SEQ ID NO: 313 - protein, 4 aa, synthetic construct
DPAK

SEQ ID NO: 314 - protein, 4 aa, synthetic construct
THFS

SEQ ID NO: 315 - protein, 4 aa, synthetic construct
IGGV

SEQ ID NO: 316 - protein, 4 aa, synthetic construct
SSWN

SEQ ID NO: 317 - protein, 4 aa, synthetic construct
SVYV

SEQ ID NO: 318 - protein, 4 aa, synthetic construct
TLNG

SEQ ID NO: 319 - protein, 4 aa, synthetic construct
NTSN

SEQ ID NO: 320 - protein, 4 aa, synthetic construct
VQYA

SEQ ID NO: 321 - protein, 4 aa, synthetic construct
DQYR

SEQ ID NO: 322 - protein, 4 aa, synthetic construct
MPVS

SEQ ID NO: 323 - protein, 4 aa, synthetic construct
SAQA

SEQ ID NO: 324 - protein, 4 aa, synthetic construct
MTVA

SEQ ID NO: 325 - protein, 4 aa, synthetic construct
TVMG

SEQ ID NO: 326 - protein, 4 aa, synthetic construct
FSSI

SEQ ID NO: 327 - protein, 4 aa, synthetic construct
SLRL

SEQ ID NO: 328 - protein, 4 aa, synthetic construct
SAMG

SEQ ID NO: 329 - protein, 4 aa, synthetic construct
YIKL

SEQ ID NO: 330 - protein, 4 aa, synthetic construct
LMTM

SEQ ID NO: 331 - protein, 4 aa, synthetic construct
QVHL

SEQ ID NO: 332 - protein, 4 aa, synthetic construct
YNSV

SEQ ID NO: 333 - protein, 4 aa, synthetic construct
CVIS

SEQ ID NO: 334 - protein, 4 aa, synthetic construct
RLDG

SEQ ID NO: 335 - protein, 4 aa, synthetic construct
AIMV

SEQ ID NO: 336 - protein, 4 aa, synthetic construct
GTTG

SEQ ID NO: 337 - protein, 4 aa, synthetic construct
ASYT

SEQ ID NO: 338 - protein, 4 aa, synthetic construct
LHVG

SEQ ID NO: 339 - protein, 4 aa, synthetic construct
LQFA

SEQ ID NO: 340 - protein, 4 aa, synthetic construct
VRGD

SEQ ID NO: 341 - protein, 4 aa, synthetic construct
NVMI

SEQ ID NO: 342 - protein, 4 aa, synthetic construct
SLYG

SEQ ID NO: 343 - protein, 4 aa, synthetic construct
GTVG

SEQ ID NO: 344 - protein, 4 aa, synthetic construct
FNSV

SEQ ID NO: 345 - protein, 4 aa, synthetic construct
TRLG

SEQ ID NO: 346 - protein, 4 aa, synthetic construct
LKVL

SEQ ID NO: 347 - protein, 4 aa, synthetic construct
SIRV

SEQ ID NO: 348 - protein, 4 aa, synthetic construct
KIQG

SEQ ID NO: 349 - protein, 4 aa, synthetic construct
QILG

SEQ ID NO: 350 - protein, 4 aa, synthetic construct
QRDA

SEQ ID NO: 351 - protein, 4 aa, synthetic construct
EAVR

SEQ ID NO: 352 - protein, 4 aa, synthetic construct
AITV

SEQ ID NO: 353 - protein, 4 aa, synthetic construct
KESI

SEQ ID NO: 354 - protein, 4 aa, synthetic construct
LMVN

SEQ ID NO: 355 - protein, 4 aa, synthetic construct
INLS

SEQ ID NO: 356 - protein, 4 aa, synthetic construct
GQVS

SEQ ID NO: 357 - protein, 4 aa, synthetic construct
TSLL

SEQ ID NO: 358 - protein, 4 aa, synthetic construct
SSTL

SEQ ID NO: 359 - protein, 4 aa, synthetic construct
YEKF

SEQ ID NO: 360 - protein, 4 aa, synthetic construct
DGKL

SEQ ID NO: 361 - protein, 4 aa, synthetic construct
QVYS

SEQ ID NO: 362 - protein, 4 aa, synthetic construct
QKEG

SEQ ID NO: 363 - protein, 4 aa, synthetic construct
ARDM

SEQ ID NO: 364 - protein, 4 aa, synthetic construct
DNFR

SEQ ID NO: 365 - protein, 4 aa, synthetic construct
SHGL

SEQ ID NO: 366 - protein, 4 aa, synthetic construct
VSVN

SEQ ID NO: 367 - protein, 4 aa, synthetic construct
GLKD

SEQ ID NO: 368 - protein, 4 aa, synthetic construct
QPVF

SEQ ID NO: 369 - protein, 4 aa, synthetic construct
VYSM

SEQ ID NO: 370 - protein, 4 aa, synthetic construct
VMAQ

SEQ ID NO: 371 - protein, 4 aa, synthetic construct
FVGM

SEQ ID NO: 372 - protein, 4 aa, synthetic construct
WSTP

SEQ ID NO: 373 - protein, 4 aa, synthetic construct
SYPV

SEQ ID NO: 374 - protein, 4 aa, synthetic construct
TTYS

SEQ ID NO: 375 - protein, 4 aa, synthetic construct
TVTT

SEQ ID NO: 376 - protein, 4 aa, synthetic construct
KDKT

SEQ ID NO: 377 - protein, 4 aa, synthetic construct
YREL

SEQ ID NO: 378 - protein, 4 aa, synthetic construct
LSHF

SEQ ID NO: 379 - protein, 4 aa, synthetic construct
SPGT

SEQ ID NO: 380 - protein, 4 aa, synthetic construct
LMGT

SEQ ID NO: 381 - protein, 4 aa, synthetic construct
AASL

SEQ ID NO: 382 - protein, 4 aa, synthetic construct
FSNN

SEQ ID NO: 383 - protein, 4 aa, synthetic construct
QARL

SEQ ID NO: 384 - protein, 4 aa, synthetic construct
YHIA

SEQ ID NO: 385 - protein, 4 aa, synthetic construct
ARQD

SEQ ID NO: 386 - protein, 4 aa, synthetic construct
VAYT

SEQ ID NO: 387 - protein, 4 aa, synthetic construct
TPSY

SEQ ID NO: 388 - protein, 4 aa, synthetic construct
MILH

SEQ ID NO: 389 - protein, 4 aa, synthetic construct
LGNV

SEQ ID NO: 390 - protein, 4 aa, synthetic construct
TSIS

SEQ ID NO: 391 - protein, 4 aa, synthetic construct
TMVY

SEQ ID NO: 392 - protein, 4 aa, synthetic construct
LVVG

SEQ ID NO: 393 - protein, 4 aa, synthetic construct
SPLY

SEQ ID NO: 394 - protein, 4 aa, synthetic construct
YKSE

SEQ ID NO: 395 - protein, 4 aa, synthetic construct
FTRL

SEQ ID NO: 396 - protein, 4 aa, synthetic construct
VSYN

SEQ ID NO: 397 - protein, 4 aa, synthetic construct
ERTP

SEQ ID NO: 398 - protein, 4 aa, synthetic construct
FRSE

SEQ ID NO: 399 - protein, 4 aa, synthetic construct
NYTE

SEQ ID NO: 400 - protein, 4 aa, synthetic construct
QTIN

SEQ ID NO: 401 - protein, 4 aa, synthetic construct
DVHR

SEQ ID NO: 402-410 - skipped (9 entries)

SEQ ID NO: 411 - protein, 50 aa, synthetic construct
MAADGYLPDWLEDNLSEGIREWWALKPGAPQPKANQQHQDNARGLVLPGY

SEQ ID NO: 412 - protein, 49 aa, synthetic construct
MSFVDHPPDWLEEVGEGLREFLGLEAGPPKPKPNQQHQDQARGLVLPGY

SEQ ID NO: 413 - protein, 34 aa, synthetic construct
KYLGPGNGLDKGEPVNAADAAALEHDKAYDQQLK

SEQ ID NO: 414 - protein, 34 aa, synthetic construct
NYLGPGNGLDRGEPVNRADEVAREHDISYNEQLE

SEQ ID NO: 415 - protein, 44 aa, synthetic construct
AGDNPYLKYNHADAEFQERLKEDTSFGGNLGRAVFQAKKRLLEP

SEQ ID NO: 416 - protein, 44 aa, synthetic construct
AGDNPYLKYNHADAEFQEKLADDTSFGGNLGKAVFQAKKRVLEP

SEQ ID NO: 417 - protein, 84 aa, synthetic construct
LGLVEEAAKTAPGKKRPVEQSPQEPDSSAGIGKSGAQPAKKRLNFGQTGDTESVPDPQPIGEPPAAPSGVGSLTMASGGGAPVA

SEQ ID NO: 418 - protein, 75 aa, synthetic construct
FGLVEEGAKTAPTGKRIDDHFPKRKKARTEEDSKPSTSSDAEAGPSGSQQLQIPAQPASSLGADTMSAGGGGPLG

SEQ ID NO: 419 - protein, 524 aa, synthetic construct
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 420 - protein, 524 aa, synthetic construct
DNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 421-468 - skipped (48 entries)

SEQ ID NO: 469 - protein, 9 aa, synthetic construct
NGSGQNQQT

SEQ ID NO: 470 - protein, 18 aa, synthetic construct
RVSTTVTQNNNSEFAWPG

SEQ ID NO: 471 - protein, 14 aa, synthetic construct
KQGTGRDNVDADKV

SEQ ID NO: 472 - protein, 13 aa, synthetic construct
ATNHQSAQAQAQT

SEQ ID NO: 473-485 - skipped (13 entries)

SEQ ID NO: 486 - protein, 599 aa, Adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 487 - protein, 534 aa, Adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 488 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 489 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 490 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGTGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 491 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGLNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 492 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIANDNKLIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 493 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIVNDNKVIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 494 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNHQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 495 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIANDNKVIQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 496 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSFQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 497 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 498 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVDSLRIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 499 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 500 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTGTSIIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 501 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSNFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 502 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCTLNSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 503 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQKPGSQIQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 504 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNMNRVNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 505 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 506 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSNSVQSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 507 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 508 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSKVFDAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 509 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAITQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 510 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSTFQGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 511 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSIQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 512 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 513 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 514 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVALNKQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 515 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHENTVSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 516 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSSFTSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 517 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHPSIHQGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 518 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTTNFRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 519 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQHYSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 520 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNKQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 521 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 522 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHAGNYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 523 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAEVQQSSMSQAQTDWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 524 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAANVQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 525 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNYQQAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 526 - protein, 737 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSVQGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 527 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHGSILTHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 528 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLFSKNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 529 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAANMQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 530 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNQQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 531 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNTYHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 532 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCDPLHIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 533 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSVISIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 534 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQLASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 535 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQVTSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 536 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHSRVEIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 537 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSFTWTAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 538 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAPTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 539 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 540 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 541 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQAISAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 542 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSVVYNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 543 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 544 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHETSRLNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 545 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAFNWQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 546 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTVMLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 547 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESSMLNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 548 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHASITSSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 549 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVARNEQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 550 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHANLYQMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 551 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQFATAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 552 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNFNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 553 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMSHQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 554 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQWMSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 555 - protein, 737 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 556 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 557 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTTKTMFAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 558 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIIYSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 559 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMLLKSNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 560 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESMQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 561 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQMLSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 562 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSGRDSYAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 563 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHINVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 564 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSNQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 565 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTKLAIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 566 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSSYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 567 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNATHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 568 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLRDNISAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 569 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSFSVGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 570 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVNRNLSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 571 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHNPSINAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 572 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDARAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 573 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNDQRAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 574 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNVQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 575 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVAPNRQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 576 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 577 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 578 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNRNGLLAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 579 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESTSVRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 580 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNIRTEMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 581 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 582 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHSWQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 583 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTKSLIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 584 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQKLLVNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 585 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSVSSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 586 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSNLYGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 587 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQMAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 588 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDIIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 589 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 590 - protein, 7 aa, synthetic construct
KGSGQNQ

SEQ ID NO: 591 - protein, 7 aa, synthetic construct
NASGQNQ

SEQ ID NO: 592 - protein, 7 aa, synthetic construct
NGTGQNQ

SEQ ID NO: 593 - protein, 7 aa, synthetic construct
NGSGLNQ

SEQ ID NO: 594 - protein, 7 aa, synthetic construct
ANDNKLI

SEQ ID NO: 595 - protein, 7 aa, synthetic construct
VNDNKVI

SEQ ID NO: 596 - protein, 7 aa, synthetic construct
NGSGQNH

SEQ ID NO: 597 - protein, 7 aa, synthetic construct
ANDNKVI

SEQ ID NO: 598 - protein, 14 aa, synthetic construct
ATNHQANYGQAQTG

SEQ ID NO: 599 - protein, 14 aa, synthetic construct
ATNHTSFQAQAQTG

SEQ ID NO: 600 - protein, 14 aa, synthetic construct
ATNHCSAQAQAQTG

SEQ ID NO: 601 - protein, 14 aa, synthetic construct
ATNHVDSLRIAQTG

SEQ ID NO: 602 - protein, 14 aa, synthetic construct
ATNRQTAQAQAQTG

SEQ ID NO: 603 - protein, 14 aa, synthetic construct
ATNHTGTSIIAQTG

SEQ ID NO: 604 - protein, 14 aa, synthetic construct
ATNHLSNFNSAQTG

SEQ ID NO: 605 - protein, 14 aa, synthetic construct
ATNHCTLNSIAQTG

SEQ ID NO: 606 - protein, 14 aa, synthetic construct
ADVQQKPGSQIQTQ

SEQ ID NO: 607 - protein, 14 aa, synthetic construct
ATNHNMNRVNAQTG

SEQ ID NO: 608 - protein, 14 aa, synthetic construct
ATNHNNVISGAQTG

SEQ ID NO: 609 - protein, 14 aa, synthetic construct
ATNHSNSVQSAQTG

SEQ ID NO: 610 - protein, 16 aa, synthetic construct
ATNHQSPIAQAQAQTG

SEQ ID NO: 611 - protein, 14 aa, synthetic construct
ATNHLSKVFDAQTG

SEQ ID NO: 612 - protein, 16 aa, synthetic construct
ATNHQSAITQAQAQTG

SEQ ID NO: 613 - protein, 14 aa, synthetic construct
ATNHSSTFQGAQTG

SEQ ID NO: 614 - protein, 14 aa, synthetic construct
ATNHNSIQAQAQTG

SEQ ID NO: 615 - protein, 14 aa, synthetic construct
ATNHMMTTARAQTG

SEQ ID NO: 616 - protein, 14 aa, synthetic construct
ATNHQGAYAQAQTG

SEQ ID NO: 617 - protein, 14 aa, synthetic construct
ALNKQSAQAQAQTG

SEQ ID NO: 618 - protein, 14 aa, synthetic construct
ATNHENTVSIAQTG

SEQ ID NO: 619 - protein, 14 aa, synthetic construct
ATNHVSSFTSAQTG

SEQ ID NO: 620 - protein, 14 aa, synthetic construct
ATNHPSIHQGAQTG

SEQ ID NO: 621 - protein, 14 aa, synthetic construct
ATNHSTTNFRAQTG

SEQ ID NO: 622 - protein, 16 aa, synthetic construct
ATNHQHYSAQAQAQTG

SEQ ID NO: 623 - protein, 14 aa, synthetic construct
ATNKQTAQAQAQTG

SEQ ID NO: 624 - protein, 14 aa, synthetic construct
ATNHSSIFNSAQTG

SEQ ID NO: 625 - protein, 14 aa, synthetic construct
ATNHAGNYNNAQTG

SEQ ID NO: 626 - protein, 14 aa, synthetic construct
AEVQQSSMSQAQTD

SEQ ID NO: 627 - protein, 14 aa, synthetic construct
AANVQSAQAQAQTG

SEQ ID NO: 628 - protein, 14 aa, synthetic construct
ATNYQQAQAQAQTG

SEQ ID NO: 629 - protein, 15 aa, synthetic construct
ATNHQSVQGAQAQTG

SEQ ID NO: 630 - protein, 14 aa, synthetic construct
ATNHGSILTHAQTG

SEQ ID NO: 631 - protein, 14 aa, synthetic construct
ATNHQLFSKNAQTG

SEQ ID NO: 632 - protein, 14 aa, synthetic construct
AANMQSAQAQAQTG

SEQ ID NO: 633 - protein, 14 aa, synthetic construct
ATNQQIAQAQAQTG

SEQ ID NO: 634 - protein, 16 aa, synthetic construct
ATNTYHQSAQAQAQTG

SEQ ID NO: 635 - protein, 14 aa, synthetic construct
ATNHCDPLHIAQTG

SEQ ID NO: 636 - protein, 14 aa, synthetic construct
ATNHTSVISIAQTG

SEQ ID NO: 637 - protein, 14 aa, synthetic construct
ATNHQLASAQAQTG

SEQ ID NO: 638 - protein, 16 aa, synthetic construct
ATNHQVTSAQAQAQTG

SEQ ID NO: 639 - protein, 14 aa, synthetic construct
ATNHHSRVEIAQTG

SEQ ID NO: 640 - protein, 14 aa, synthetic construct
ATNHTSFTWTAQTG

SEQ ID NO: 641 - protein, 16 aa, synthetic construct
ATNHQSAPTQAQAQTG

SEQ ID NO: 642 - protein, 14 aa, synthetic construct
ATNHNSTYLGAQTG

SEQ ID NO: 643 - protein, 14 aa, synthetic construct
ATNHQIAQAQAQTG

SEQ ID NO: 644 - protein, 16 aa, synthetic construct
ATNHQAISAQAQAQTG

SEQ ID NO: 645 - protein, 14 aa, synthetic construct
ATNHLSVVYNAQTG

SEQ ID NO: 646 - protein, 16 aa, synthetic construct
ATNHMHQSAQAQAQTG

SEQ ID NO: 647 - protein, 14 aa, synthetic construct
ATNHETSRLNAQTG

SEQ ID NO: 648 - protein, 14 aa, synthetic construct
AFNWQSAQAQAQTG

SEQ ID NO: 649 - protein, 14 aa, synthetic construct
ATNHNTVMLGAQTG

SEQ ID NO: 650 - protein, 14 aa, synthetic construct
ATNHESSMLNAQTG

SEQ ID NO: 651 - protein, 14 aa, synthetic construct
ATNHASITSSAQTG

SEQ ID NO: 652 - protein, 14 aa, synthetic construct
ARNEQSAQAQAQTG

SEQ ID NO: 653 - protein, 14 aa, synthetic construct
ATNHANLYQMAQTG

SEQ ID NO: 654 - protein, 14 aa, synthetic construct
ATNHQFATAQAQTG

SEQ ID NO: 655 - protein, 16 aa, synthetic construct
ATNFNHQSAQAQAQTG

SEQ ID NO: 656 - protein, 14 aa, synthetic construct
ATNHMSHQAQAQTG

SEQ ID NO: 657 - protein, 16 aa, synthetic construct
ATNHQWMSAQAQAQTG

SEQ ID NO: 658 - protein, 15 aa, synthetic construct
ATNHQSGQQAQAQTG

SEQ ID NO: 659 - protein, 14 aa, synthetic construct
ATNHSSAQAQAQTG

SEQ ID NO: 660 - protein, 14 aa, synthetic construct
ATNHTTKTMFAQTG

SEQ ID NO: 661 - protein, 14 aa, synthetic construct
ATNHSSIIYSAQTG

SEQ ID NO: 662 - protein, 14 aa, synthetic construct
ATNHMLLKSNAQTG

SEQ ID NO: 663 - protein, 14 aa, synthetic construct
ATNHESMQAQAQTG

SEQ ID NO: 664 - protein, 16 aa, synthetic construct
ATNHQMLSAQAQAQTG

SEQ ID NO: 665 - protein, 14 aa, synthetic construct
ATNHSGRDSYAQTG

SEQ ID NO: 666 - protein, 14 aa, synthetic construct
ATNHINVISGAQTG

SEQ ID NO: 667 - protein, 14 aa, synthetic construct
ATNHVSNQAQAQTG

SEQ ID NO: 668 - protein, 14 aa, synthetic construct
ATNHNTKLAIAQTG

SEQ ID NO: 669 - protein, 14 aa, synthetic construct
ATNHSSSYNNAQTG

SEQ ID NO: 670 - protein, 16 aa, synthetic construct
ATNATHQSAQAQAQTG

SEQ ID NO: 671 - protein, 14 aa, synthetic construct
ATNHLRDNISAQTG

SEQ ID NO: 672 - protein, 14 aa, synthetic construct
ATNHSSFSVGAQTG

SEQ ID NO: 673 - protein, 14 aa, synthetic construct
ATNHVNRNLSAQTG

SEQ ID NO: 674 - protein, 14 aa, synthetic construct
ATNHHNPSINAQTG

SEQ ID NO: 675 - protein, 14 aa, synthetic construct
ATNHQDARAQAQTG

SEQ ID NO: 676 - protein, 14 aa, synthetic construct
ATNDQRAQAQAQTG

SEQ ID NO: 677 - protein, 14 aa, synthetic construct
ATNVQTAQAQAQTG

SEQ ID NO: 678 - protein, 14 aa, synthetic construct
APNRQSAQAQAQTG

SEQ ID NO: 679 - protein, 14 aa, synthetic construct
ATNRQIAQAQAQTG

SEQ ID NO: 680 - protein, 14 aa, synthetic construct
ATNHEDNIRRAQTG

SEQ ID NO: 681 - protein, 14 aa, synthetic construct
ATNHNRNGLLAQTG

SEQ ID NO: 682 - protein, 14 aa, synthetic construct
ATNHESTSVRAQTG

SEQ ID NO: 683 - protein, 14 aa, synthetic construct
ATNHNIRTEMAQTG

SEQ ID NO: 684 - protein, 14 aa, synthetic construct
ATNHQTLFNSAQTG

SEQ ID NO: 685 - protein, 14 aa, synthetic construct
ATNHHSWQAQAQTG

SEQ ID NO: 686 - protein, 14 aa, synthetic construct
ATNHSTKSLIAQTG

SEQ ID NO: 687 - protein, 14 aa, synthetic construct
ATNHQKLLVNAQTG

SEQ ID NO: 688 - protein, 14 aa, synthetic construct
ATNHLSVSSIAQTG

SEQ ID NO: 689 - protein, 14 aa, synthetic construct
ATNHVSNLYGAQTG

SEQ ID NO: 690 - protein, 14 aa, synthetic construct
ATNRQMAQAQAQTG

SEQ ID NO: 691 - protein, 14 aa, synthetic construct
ATNHEDIIRSAQTG

SEQ ID NO: 692 - protein, 14 aa, synthetic construct
ATNHCSTSIRAQTG

SEQ ID NO: 693 - protein, 8 aa, synthetic construct
NHQSPIAQ

SEQ ID NO: 694 - protein, 8 aa, synthetic construct
NHQSAITQ

SEQ ID NO: 695 - protein, 8 aa, synthetic construct
NHQHYSAQ

SEQ ID NO: 696 - protein, 8 aa, synthetic construct
NTYHQSAQ

SEQ ID NO: 697 - protein, 8 aa, synthetic construct
NHQVTSAQ

SEQ ID NO: 698 - protein, 8 aa, synthetic construct
NHQSAPTQ

SEQ ID NO: 699 - protein, 8 aa, synthetic construct
NHQAISAQ

SEQ ID NO: 700 - protein, 8 aa, synthetic construct
NHMHQSAQ

SEQ ID NO: 701 - protein, 8 aa, synthetic construct
NFNHQSAQ

SEQ ID NO: 702 - protein, 8 aa, synthetic construct
NHQWMSAQ

SEQ ID NO: 703 - protein, 8 aa, synthetic construct
NHQMLSAQ

SEQ ID NO: 704 - protein, 8 aa, synthetic construct
NATHQSAQ

SEQ ID NO: 705 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHENTVSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 706 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 707 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 708 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHGSILTHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 709 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIIGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 710 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 711 - skipped

SEQ ID NO: 712 - protein, 4 aa, synthetic construct
NTVS

SEQ ID NO: 713 - protein, 4 aa, synthetic construct
TLFN

SEQ ID NO: 714 - protein, 4 aa, synthetic construct
STYL

SEQ ID NO: 715 - protein, 4 aa, synthetic construct
SILT

SEQ ID NO: 716 - protein, 4 aa, synthetic construct
MTTA

SEQ ID NO: 717 - protein, 4 aa, synthetic construct
STSI

SEQ ID NO: 718 - skipped

SEQ ID NO: 719 - protein, 6 aa, synthetic construct
ENTVSI

SEQ ID NO: 720 - protein, 6 aa, synthetic construct
QTLFNS

SEQ ID NO: 721 - protein, 6 aa, synthetic construct
NSTYLG

SEQ ID NO: 722 - protein, 6 aa, synthetic construct
GSILTH

SEQ ID NO: 723 - protein, 6 aa, synthetic construct
MMTTAR

SEQ ID NO: 724 - protein, 6 aa, synthetic construct
CSTSIR

SEQ ID NO: 725 - protein, 6 aa, synthetic construct
EDNIRS

SEQ ID NO: 726 - protein, 14 aa, synthetic construct
ATNHEDNIRSAQTG

SEQ ID NO: 727 - protein, 9 aa, synthetic construct
KGSGQNQQT

SEQ ID NO: 728 - protein, 14 aa, synthetic construct
  VARIANT 5: note = Q, E, N, G, M, C, V, or T
  VARIANT 6: note = S, N, T, M, G, or D
  VARIANT 7: note = A, T, L, I, K, S, N or V
  VARIANT 8: note = Q, V, F, Y, L, T, S, I, R, or Q
  VARIANT 9: note = A, S, N, L, T, I, or R
  VARIANT 10: note = Q, I, S, G, H or R
ATNHXXXXXXAQTG

SEQ ID NO: 729-738 - skipped (10 entries)

SEQ ID NO: 739 - protein, 14 aa, synthetic construct
  VARIANT 5: note = Q, E, N, G, M, or C
  VARIANT 6: note = S, N, T, or M
  VARIANT 7: note = A, T, L, I, or S
  VARIANT 8: note = Q, V, F, Y, L, or I
  VARIANT 9: note = A, S, N, L, T, or I
  VARIANT 10: note = I, S, Q, G, H, or R
ATNHXXXXXXAQTG

SEQ ID NO: 740 - protein, 16 aa, synthetic construct
  VARIANT 5..12: note = Any of the 20 standard amino acids
  VARIANT 9..12: note = Residues may be deleted
ATNHXXXXXXXXAQTG

SEQ ID NO: 741 - protein, 4 aa, synthetic construct
GAYA

SEQ ID NO: 742 - protein, 4 aa, synthetic construct
TKLA

SEQ ID NO: 743 - protein, 4 aa, synthetic construct
SSFT

SEQ ID NO: 744 - protein, 4 aa, synthetic construct
DNIR

SEQ ID NO: 745 - protein, 4 aa, synthetic construct
NVIS

SEQ ID NO: 746 - protein, 4 aa, synthetic construct
GTSI

SEQ ID NO: 747 - protein, 4 aa, synthetic construct
DARA

SEQ ID NO: 748 - protein, 4 aa, synthetic construct
SAQA

SEQ ID NO: 749 - protein, 6 aa, synthetic construct
QGAYAQ

SEQ ID NO: 750 - protein, 6 aa, synthetic construct
NTKLAI

SEQ ID NO: 751 - protein, 6 aa, synthetic construct
VSSFTS

SEQ ID NO: 752 - protein, 6 aa, synthetic construct
NNVISG

SEQ ID NO: 753 - protein, 6 aa, synthetic construct
TGTSII

SEQ ID NO: 754 - protein, 6 aa, synthetic construct
QANYGQ

SEQ ID NO: 755 - protein, 6 aa, synthetic construct
QDARAQ

SEQ ID NO: 756 - protein, 6 aa, synthetic construct
QSAQAQ

SEQ ID NO: 757 - protein, 9 aa, synthetic construct
KYHKSGAAQ

SEQ ID NO: 758 - protein, 9 aa, synthetic construct
KQVNGRPRD

SEQ ID NO: 759 - protein, 8 aa, synthetic construct
QHYSAQAQ

SEQ ID NO: 760-766 - skipped (7 entries)

SEQ ID NO: 767 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 768 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 769 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 770 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNTKLAIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 771 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSSFTSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 772 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 773 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 774 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 775 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTGTSIIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 776 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQWMSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 777 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQDARAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 778 - protein, 738 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQHYSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 779 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNIRTEMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 780 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTTNFRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 781 - skipped

SEQ ID NO: 782 - DNA, 2514 bp, synthetic construct
  source 1..2514: mol_type = other DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactga

SEQ ID NO: 783 - DNA, 2511 bp, synthetic construct
  source 1..2511: mol_type = other DNA
atggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagat

SEQ ID NO: 784 - DNA, 600 bp, synthetic construct
  source 1..600: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 785 - DNA, 1624 bp, synthetic construct
  source 1..1624: mol_type = other DNA
catctcagcatcatggttggatgtttccacctggctacataagcaagctttacacaaggtgtaatttgcctaaatagtggtccattctattggggtgggagcaattgcttccaggactcacatccatatggctcccacttagccatgtggcctgctgacaaagggtggcggaactgtcactactctgttgtccacgctttcagtcctttggtttcctcttcactccctggacgctcatgtaaaaagggaggccatatacctgtgcattgtgtgtctaagcattcagtgtgtgtctaaaggcagaagggtgtgggtaggaaaacaaagacgagggaagctgcgttctccaaacacttcagacttgagtaagtggggttttgcagcaattgagtgatttgagggaaagtgaacatacaaacccaagcaatcaaagggaatattatcttaataccagggatacatgtttttctttctgcctcttaagtccaaagaggcaaatcaggacaagtggctttggttgtaaactttaaggtcaaggatcctttctgttgagcttagctctcaagttctcagtagtcaactgcggtgaaacataattaatagcacgataaatacaagttgtggaagattcgattgaaagttggaggccctctccgtggatctctctacaaagagcctgtaataaagaggacttaatcaacgttagcagggctatttaaaaagcatcgtctattaaaattcatttcttctctagagcctcttgttggagtttctctgtgtgggtgtgttcgtaagagaggaatgggttagcaagagtactgggtacaatttgtgtatccaagagaaaacagaagctctcaatgaggaagaacatatgtttctgggactgcatctgtgcaaaaagtacatagtcctgacgttgtactaagaaaaaaaacactctctttagaaagtcttttatttcacacgttatcttcttggcacatttccctcatattgccctttccgcctgaccaaatagccctttctcaccctcaggtccaggaaaaccaggaaacgtttccaacagtgcgacaaagcctgactaaccagacatactactcgctcggggatcccggaggcaagcctcagtccaagaacaggagtgactctcgagggctcacctgcctgcagggcagcccctccctgcatcgagcggaaatccatcctgtccagcgcggggcgtgggcagagcggggcgcggccccggcaggcggtatccgctgggactccgacaacgtgcgcgaccccaggcgaaccgcgcccctctccccacctccccgcgggcgggtacaagtctccaggtgtccgcgcgctcagcgggtccggcccgcccccgcccccgcccccgggcccgactgcgcgtgcccggccggagccgcgccccctcctcagggaaggccgggcgtccggcccacgaggccgagctcccccccggcccgggcctctcaccggcgcggggggcgggccaggggcggggccggactcgagcggggcggggctcgcgccagcgcccccagctccgtggcggcttcgcccgcgagtccagaggcaggcgagcagctcggtcgcccccaccggcccc

SEQ ID NO: 786 - DNA, 4324 bp, synthetic construct
  source 1..4324: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagtcatggagaagacccaccttgcagatgtcctcactggggctggcagagccggcaacctgcccaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccagccaccatggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagattaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 787 - DNA, 4662 bp, synthetic construct
  source 1..4662: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaacatctcagcatcatggttggatgtttccacctggctacataagcaagctttacacaaggtgtaatttgcctaaatagtggtccattctattggggtgggagcaattgcttccaggactcacatccatatggctcccacttagccatgtggcctgctgacaaagggtggcggaactgtcactactctgttgtccacgctttcagtcctttggtttcctcttcactccctggacgctcatgtaaaaagggaggccatatacctgtgcattgtgtgtctaagcattcagtgtgtgtctaaaggcagaagggtgtgggtaggaaaacaaagacgagggaagctgcgttctccaaacacttcagacttgagtaagtggggttttgcagcaattgagtgatttgagggaaagtgaacatacaaacccaagcaatcaaagggaatattatcttaataccagggatacatgtttttctttctgcctcttaagtccaaagaggcaaatcaggacaagtggctttggttgtaaactttaaggtcaaggatcctttctgttgagcttagctctcaagttctcagtagtcaactgcggtgaaacataattaatagcacgataaatacaagttgtggaagattcgattgaaagttggaggccctctccgtggatctctctacaaagagcctgtaataaagaggacttaatcaacgttagcagggctatttaaaaagcatcgtctattaaaattcatttcttctctagagcctcttgttggagtttctctgtgtgggtgtgttcgtaagagaggaatgggttagcaagagtactgggtacaatttgtgtatccaagagaaaacagaagctctcaatgaggaagaacatatgtttctgggactgcatctgtgcaaaaagtacatagtcctgacgttgtactaagaaaaaaaacactctctttagaaagtcttttatttcacacgttatcttcttggcacatttccctcatattgccctttccgcctgaccaaatagccctttctcaccctcaggtccaggaaaaccaggaaacgtttccaacagtgcgacaaagcctgactaaccagacatactactcgctcggggatcccggaggcaagcctcagtccaagaacaggagtgactctcgagggctcacctgcctgcagggcagcccctccctgcatcgagcggaaatccatcctgtccagcgcggggcgtgggcagagcggggcgcggccccggcaggcggtatccgctgggactccgacaacgtgcgcgaccccaggcgaaccgcgcccctctccccacctccccgcgggcgggtacaagtctccaggtgtccgcgcgctcagcgggtccggcccgcccccgcccccgcccccgggcccgactgcgcgtgcccggccggagccgcgccccctcctcagggaaggccgggcgtccggcccacgaggccgagctcccccccggcccgggcctctcaccggcgcggggggcgggccaggggcggggccggactcgagcggggcggggctcgcgccagcgcccccagctccgtggcggcttcgcccgcgagtccagaggcaggcgagcagctcggtcgcccccaccggccccatggctgctcctggtgctcctgccgagtacggctacatcagaacagtgctgggccagcagatcctgggacagctggattctagctctctggccctgccttctgaggccaagctgaaactggccggcagttctggaagaggcggccagacagtgaagtccctgcggatccaagaacaggtgcagcagaccctggccagaaagggcagatcttctgtcggcaacggcaacctgcacagaaccagctctgtgcccgagtacgtgtacaatctgcacctggtggaaaacgacttcgtcggcggcagatcccctgtgcctaagacctacgatatgctgaaggccggcaccaccgccacctatgaaggcagatggggaagaggcacagcccagtacagcagccagaaaagcgtggaagagagaagcctgcggcaccctctgcggagactggaaatcagccctgatagcagcccagagagagcccactacacccacagcgactaccagtactcccagagatctcaggccggccacacactgcaccaccaagagtctagaagggccgctctgctggtgcctcctagatacgccagatctgagatcgtgggcgtgtccagagccggcacaacaagcagacagagacacttcgacacctaccaccggcagtatcagcacggcagcgtgtccgataccgtgttcgatagcatccccgccaatcctgctctgctgacataccctagacctggcacctccagatccatgggcaatctgctggaaaaagagaactacctgaccgccggactgaccgtgggacaagttcgacctctggttcctctgcagcccgtgacacagaacagagccagcagaagcagctggcaccagtccagcttccacagcaccagaacactgagagaagctggccctagcgtggccgtggattcttctggtagaagggctcacctgacagttggccaagcagctgcaggcggaagcggaaatctgctgaccgagagaagcaccttcaccgacagccagctgggcaacgccgacatggaaatgacactggaacgggccgtgtccatgctggaagccgatcacatgctgcccagcagaattagcgccgctgccacctttatccagcacgagtgcttccagaagtctgaggcccggaagagagtgaaccagctgagaggcatcctgaagctgctgcagctcctgaaggtgcagaacgaggatgtgcagagggctgtgtgtggggccctgagaaatctggtgttcgaggacaacgacaacaagctggaagtggccgagctgaacggcgtgccaagactgctgcaggttctgaaacagacccgcgacctggaaacaaagaagcagatcaccggcctgctctggaacctgagcagcaacgacaagctgaagaacctgatgatcacagaggccctgctgaccctgacagagaacatcatcatccctttcagcggctggcccgagggcgattaccctaaagctaatggcctgctggacttcgacatcttctacaacgtgaccggctgcctgagaaacatgtctagcgctggcgccgatggcagaaaggccatgagaagatgtgacggcctgatcgacagcctggtgcactatgtgcggggcacaatcgccgattaccagcctgatgataaggccaccgagaactgcgtgtgcatcctgcacaacctgagctaccagctggaagcagagctgcccgagaagtacagccagaacatctacatccagaaccggaacatccagaccgacaacaacaagagcatcggctgcttcggcagccgcagccggaaagtgaaagaacagtaccaggacgtgcccatgcctgaggaaaagtctaaccccaaaggcgtggaatggctgtggcacagcatcgtgatccggatgtacctgagcctgatcgccaagagcgtgcggaattacacccaagaggcatctctgggcgccctgcagaatctgacagcaggatctggccctatgcctacctctgtggctcagaccgtggtgcagaaagagtctggcctgcagcacacccggaagatgctgcatgtgggagatcccagcgtgaagaaaaccgccatcagcctgctgagaaacctgagccggaatctgtctctgcagaatgagatcgccaaagagacactgcccgacctggtgtctatcatccctgacaccgtgcctagcaccgacctgctgattgagacaacagccagcgcctgctacaccctgaacaacatcattcagaactcctaccagaacgcccgcgatctgctgaacacaggcggcatccagaaaatcatggccatctctgccggcgacgcctacgcctctaacaaggcctctaaagccgccagcgtgctgctgtattctctgtgggcccataccgagctgcaccatgcctataagaaggcccagttcaaaaagaccgacttcgtgaacagccggaccgccaaggcctaccactctctgaaagatgtcgacggatccggtaccgattacaaggacgacgatgacaagtgaagcttaataaaagatctttattttcattagatctgtgtgttggttttttgtgtgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 788 - DNA, 4093 bp, synthetic construct
  source 1..4093: mol_type = other DNA
acggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 789 - protein, 837 aa, synthetic construct
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 790 - DNA, 540 bp, synthetic construct
  source 1..540: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 791 - DNA, 221 bp, synthetic construct
  source 1..221: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactgggga

SEQ ID NO: 792 - DNA, 855 bp, synthetic construct
  source 1..855: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactggggactcgagttaagggcgaattcccgataaggatcttcctagagcatggctacgtagataagtagcatggcgggttaatcattaactaca

SEQ ID NO: 793 - protein, 14 aa, synthetic construct
ATNHHSRVEYAQTG

SEQ ID NO: 794 - protein, 4 aa, synthetic construct
AQTG

SEQ ID NO: 795 - protein, 4 aa, synthetic construct
ATNH

SEQ ID NO: 796 - protein, 736 aa, synthetic construct; identical to SEQ ID NO: 1

SEQ ID NO: 797 - protein, 14 aa, synthetic construct
  VARIANT 5: note = E, N, G, M, C, V, T, Q or S
  VARIANT 6: note = N, T, M, G, D, S or I
  VARIANT 7: note = T, L, I, K, S, N, V, A or R
  VARIANT 8: note = V, F, Y, L, T, S, I, R, Q or N
  VARIANT 9: note = S, N, L, T, I, R, A, E or F
  VARIANT 10: note = I, S, G, H, R, Q or M
ATNHXXXXXXAQTG

SEQ ID NO: 798 - protein, 14 aa, synthetic construct
  VARIANT 5: note = E, N, M, C, V, T, or Q
  VARIANT 6: note = N, M, G, D, or S
  VARIANT 7: note = T, S, N, V, or A
  VARIANT 8: note = F, Y, T, S or I
  VARIANT 9: note = S, L, T, I, R, or A
  VARIANT 10: note = I, S, G, R, or Q
ATNHXXXXXXAQTG

SEQ ID NO: 799 - protein, 14 aa, synthetic construct
  VARIANT 5..10: note = Any of the 20 standard amino acids
ATNHXXXXXXAQTG